(19) United States
(12) Patent Application Publication
(10) Pub. No.: US 2006/0122234 A1
(43) Pub. Date: Jun. 8, 2006

(54) Substituted thienyl-hydroxamic acids as histone deacetylase inhibitors

(75) Inventors: Janet Ann Archer, Essex (GB); Walter Bordogna, Essex (GB); Richard James Bull, Essex (GB); David Edward Clark, Essex (GB); Hazel Joan Dyke, Essex (GB); Matthew Iain Andrew Gill, Essex (GB); Neil Victor Harris, Essex (GB); Marco Van Den Heuvel, Essex (GB); Stephen Price, Essex (GB)

Correspondence Address: FOLEY AND LARDNER LLP;SUITE 500, 3000 K STREET NW, WASHINGTON, DC, 20007, US

(73) Assignee: Argenta Discovery Limited

(21) Appl. No.: 10/522,873

(22) PCT Filed: Jul. 24, 2003
(86) PCT No.: PCT/GB03/03168

§ 371 Date: Oct. 4, 2005

(30) Foreign Application Priority Data
Aug. 2, 2002 (GB) 0218040.4
May 7, 2003 (GB) 0310462.7

(51) Int. Cl. A61K 31/4436; A61K 31/433; A61K 31/4245; A61K 31/4178; A61K 31/416; C07D 409/02

(52) U.S. Cl. 514/342; 514/406; 514/397; 546/280.4; 548/315.1; 548/365.7; 514/362; 514/364

(57) ABSTRACT

A compound of formula (I): which can be used in the treatment of diseases associated with histone deacetylase enzymatic activity.

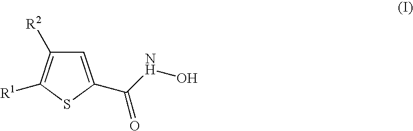

This invention relates to substituted thienyl-hydroxamic acids, their preparation and pharmaceutical compositions containing these compounds for treating diseases associated with histone deacetylase enzymatic activity.

In eukaryotic cells, DNA is tightly associated with histones to form a compact complex called chromatin. The histones, generally highly conserved across eukaryotic species, constitute a family of proteins which are rich in basic amino acids that contact the phosphate groups of DNA.

There are five main classes of histones, H1, H2A, H2B, H3 and H4. Four pairs of each of H2A, H2B, H3 and H4 together form a disk-shaped octomeric protein core, around which DNA is wound (with the basic amino acids of the histones interacting with the negatively charged phosphate groups of the DNA) to form a nucleosome. Approximately 146 base pairs of DNA wrap around a histone core to make up a nucleosome particle, the repeating structural motif of chromatin.

Histone deacetylases (HDACs) are part of transcriptional corepressor complexes and play key roles in regulating chromatin structure. Three different classes of human HDACs have been defined based on their homology to HDACs found in Saccharomnyces cerevisiae. Class I HDACs (HDAC1, 2, 3, and 8) are related to the yeast transcriptional regulator RPD3. Class II HDACs (HDAC4, 5, 6, 7, 9, and 10) are similar to HDA1, another deacetylase in yeast. Class III HDACs are related to the yeast silencing protein SIR2 and are dependent on NAD for enzymatic activity.

Reversible acetylation of histones is a major regulator of gene expression that acts by altering accessibility of transcription factors to DNA. In normal cells, histone deacetylase (HDAC) and histone acetyltransferases (HATs) together control the level of acetylation of histones to maintain a balance. Histone acetylation has a key role in transcriptional activation, whereas deacetylation of histones correlates with the transcriptional repression and silencing of genes [for a review of histone deacetylation see Kouzarides Curr. Opin. Genet. Dev., 9:40-48 (1999); Johnstone R W Nat. Rev. Drug Discov., 1:287-299 (2002)]. Genetic repression may have an important role in neuronal ageing, atrophy and degenerative diseases.

Moreover, histone deacetylases have been shown to regulate the activity of non-histone proteins through the modification of their acetylation level. These include steroid receptors such as estrogen and androgen receptors [Wang et al, J. Biol. Chem., 276:18375-83 (2001), Gaughan et al, J. Biol. Chem., 277: 25904-13 (2002)), transcription factors such as p53, E2F and myoD [Luo et al, Nature, 408:377-381 (2000); Ito et al, EMBO J, 19:1176-1179 (2001); Sartorelli et al, Mol. Cell, 4:725-734 (1999)], and cytoplasmic proteins such as α-tubulin [Hubbert et al, Nature, 417:455-458 (2002)].

There are currently several known inhibitors, both natural and synthetic, of HDAC. Some natural inhibitors include: (i) trapoxin B; (ii) trichostatin A [Yoshida and Beppu, Exper. Cell Res., 177:122-131 (1988)]; and (iii) chlamydocin. Synthetic inhibitors include suberoyl anilide hydroxamic acid [Richon et al., Proc. Natl. Acad. Sci. USA, 95: 3003-3007 (1998)] and phenylbutyrate [Johnstone R W Nat. Rev. Drug Discov., 1:287-299 (2002)].

Trichostatin A has been shown to cause arrest of rat fibroblasts at both G₁ and G₂ phases of the cell cycle, implicating HDAC in cell cycle regulation [Yoshida and Beppu, Exper. Cell Res., 177:122-131 (1988)]. Trichostatin A and suberoyl anilide hydroxarnic acid have been shown to inhibit cell growth, induce terminal differentiation and prevent the formation of tumors in mice [Johnstone R W Nat. Rev. Drug Discov., 1:287-299 (2002)]. Trapoxin, trichostatin, and depudecin have been used to study gene regulation by HDAC-mediated chromatin remodeling [Christian A. Hassig, Stuart L. Schreiber, Curr. Opinion in Chem. Biol., 1997, 1, 300-308; Christian A. Hassig, Jeffrey K. Tong, Stuart L. Schreiber, Chem. & Biol., 1997, 4, 783-789; Christian A. Hassig, Jeffrey K. Tong, Tracey C. Fleischer, Takashi Owa, Phyllis Grable, Donald E. Ayer, Stuart L. Schreiber, Proc. Natl. Acad. Sci., U.S.A., 1998, 95, 3519-3524; Ho Jeong Kwon, Takashi Owa, Christian A. Hassig, Junichi Shimada, Stuart L. Schreiber, Proc. Natl. Acad. Sci., U.S.A. 1998, 95, 3356-3361].

It is an object of the present invention to provide inhibitors of histone deacetylase.

Thus, in one aspect, the present invention provides compounds of formula (I): 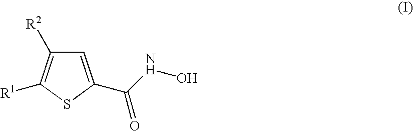
in which
R¹ represents aryl or heteroaryl, each optionally substituted by one or more groups selected from R³, alkylenedioxy, carboxy, cyano, halo, hydroxy, nitro, haloalkyl, haloalkoxy, —C(═O)—R³, —C(═O)—OR³, —C(═Z)—NR⁴R⁵, —NR⁴R⁵, —NR⁶—C(═O)—OR³, —NR⁶—C(═O)—NR⁴R⁵, —NR⁶—C(═Z)—R³, —O—C(═O)—NR⁴R⁵, —NR⁶—SO₂—R³, —OR³, —O—C(═O)R³, —SH, —SR³, —SOR³, —SO₂R³ and —SO₂—NR⁴R⁵;
R² represents hydrogen, chloro, cyano, fluoro, alkoxy, alkyl, or haloalkyl;
R³ represents aryl, heteroaryl, cycloalkyl, cycloalkenyl, heterocycloalkyl or R⁷;
R⁴ and R⁵ independently represent a group selected from hydrogen, alkyl, alkenyl, aryl, heteroaryl, cycloalkyl, cycloalkenyl or heterocycloalkyl, wherein said alkyl or alkenyl are optionally substituted by aryl, heteroaryl, cycloalkyl, cycloalkenyl or heterocycloalkyl; or the group —NR⁴R⁵ may form a cyclic amine;
R⁶ represents hydrogen or lower alkyl;
R⁷ represents alkyl, alkenyl and alkynyl, wherein said alkyl, alkenyl or alkynyl are optionally substituted by one or more groups selected from aryl, heteroaryl, cycloalkyl, cycloalkenyl, heterocycloalkyl, hydroxy, —C(═Z)—NR⁴R⁵, —NR⁴R⁵, —NR⁶—C(═Z)—R⁸, —O—C(═O)—NR⁴R⁵, —NR⁶—C(═O)—OR⁸, —NR⁶—C(═O)—NR⁴R⁵, —NR⁶—SO₂—R⁸, —OR⁸, —SOR⁸, SO₂R⁸ and —SO₂—NR⁴R⁵;
R⁸ represents alkyl, alkenyl or alkynyl, optionally substituted by one or more groups selected from aryl, heteroaryl, cycloalkyl, cycloalkenyl, heterocycloalkyl, hydroxy and halogen; or R⁸ represents aryl, heteroaryl, cycloalkyl, cycloalkenyl or heterocycloalkyl; and
Z is O or S,
and corresponding N-oxides, pharmaceutically acceptable salts, solvates and prodrugs of such compounds.

A second aspect of the invention is a pharmaceutical composition comprising a compound of Formula I or an N-oxide, pharmaceutically acceptable salt, solvate or prodrug thereof, in admixture with a pharmaceutically acceptable carrier or excipient.

A third aspect of the invention is a compound of Formula I or an N-oxide, pharmaceutically acceptable salt, solvate or prodrug thereof for use in therapy.

A fourth aspect of the invention is the use of a compound of Formula I, or an N-oxide, pharmaceutically acceptable salt, solvate or prodrug thereof, in the manufacture of a medicament for the treatment of a disease in which inhibition of histone deacetylase can prevent, inhibit or ameliorate the pathology and/or symptomatology of the disease.

A fifth aspect of the invention is a method for treating a disease in a patient in which inhibition of histone deacetylase can prevent, inhibit or ameliorate the pathology and/or symptomatology of the disease, which method comprises administering to the patient a therapeutically effective amount of compound of Formula I or an N-oxide, pharmaceutically acceptable salt, solvate or prodrug thereof.

A sixth aspect of the invention is a method of inhibiting histone deacetylase in a cell, comprising contacting a cell in which inhibition of histone deacetylase is desired with a compound of Formula I or an N-oxide, pharmaceutically acceptable salt, solvate or prodrug thereof.

A seventh aspect of the invention is a method of preparing a compound of formula I or an N-oxide, pharmaceutically acceptable salt, solvate or prodrug thereof.

An eighth aspect of the invention is a method of making a pharmaceutical composition comprising combining a compound of formula (I), or an N-oxide, pharmaceutically acceptable salt, solvate or prodrug thereof, with a pharmaceutically acceptable carrier or excipient.

For purposes of the present invention, the following definitions as used throughout the description of the invention shall be understood to have the following meanings:

“Compounds of the invention”, and equivalent expressions, are meant to embrace compounds of general formula (I) as hereinbefore described, their N-oxides, their prodrugs, their pharmaceutically acceptable salts and their solvates, where the context so permits.

“Histone deacetylase” and “HDAC” are intended to refer to any one of a family of enzymes that remove acetyl groups from lysine residues of proteins including, but not limited to, histones, transcription factors, steroid receptors and tubulin. Unless otherwise indicated the term histone is meant to refer to any histone protein, including H1, H2A, H2B, H3, H4 and H5 from any species. In one preferred embodiment the histone deacetylase is a human HDAC, including, but not limited to, HDAC-1, HDAC-2, HDAC-3, HDAC-4, HDAC-5, HDAC-6, HDAC-7, HDAC-8, HDAC-9, and HDAC-10. In another preferred embodiment the histone deacetylase is derived from a protozoal or fungal source.

“Patient” includes both human and other mammals.

For purposes of the present invention, the following chemical terms as used above, and throughout the description of the invention, and unless otherwise indicated, shall be understood to have the following meanings:

“Acyl” means an alkyl-CO— group in which the alkyl group is as described herein.

“Alkenyl” as a group or part of a group denotes an aliphatic hydrocarbon group containing a carbon-carbon double bond and which may be straight or branched having from 2 to 12 carbon atoms, preferably 2-6 carbon atoms, in the chain. Exemplary alkenyl groups include ethenyl, and propenyl.

“Alkoxy” means an —O-alkyl group in which alkyl is as defined below. Exemplary alkoxy groups include methoxy and ethoxy.

“Alkoxycarbonyl” means an —C(═O)—O-alkyl group in which alkyl is as defined below. Exemplary alkoxycarbonyl groups include methoxycarbonyl and ethoxycarbonyl.

“Alkyl” as a group or part of a group refers to a straight or branched chain saturated hydrocarbon group having from 1 to 12, preferably 1 to 6, carbon atoms, in the chain. Exemplary alkyl groups include methyl, ethyl, 1-propyl and 2-propyl.

“Alkylamino” means a —NH-alkyl group in which alkyl is as defined above. Exemplary alkylamino groups include methylamino and ethylamino.

“Alkylene” means —(CH₂)ₙ—, where n may be 1 to 3.

“Alkylenedioxy” means a —O-alkylene-O— group in which alkylene is as defined above. Exemplary alkylenedioxy groups include methylenedioxy and ethylenedioxy.

“Alkylsufinyl” means a —SO-alkyl group in which alkyl is as defined above. Exemplary alkylsulfinyl groups include methylsulfinyl and ethylsulfinyl.

“Alkylsufonyl” means a —SO₂-alkyl group in which alkyl is as defined above. Exemplary alkylsulfonyl groups include methylsulfonyl and ethylsulfonyl.

“Alkylthio” means a —S-alkyl group in which alkyl is as defined above. Exemplary alkylthio groups include methylthio and ethylthio.

“Alkynyl” as a group or part of a group means an aliphatic hydrocarbon group containing a carbon-carbon triple bond and which may be straight or branched having from 2 to 6 carbon atoms in the chain. Exemplary alkynyl groups include ethynyl and propynyl.

“Aryl” as a group or part of a group denotes: (i) an optionally substituted monocyclic or multicyclic aromatic carbocyclic moiety of from 6 to 14 carbon atoms, preferably from 6 to 10 carbon atoms, such as phenyl or naphthyl, and in one embodiment preferably phenyl; or (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The aryl group may be substituted by one or more substituent groups.

“Arylalkenyl” means an aryl-alkenyl-group in which the aryl and alkenyl are as previously described. Exemplary arylalkenyl groups include styryl and phenylallyl.

“Arylalkyl” means an aryl-alkyl-group in which the aryl and alkyl moieties are as previously described. Preferred arylalkyl groups contain a C₁₋₄ alkyl moiety. Exemplary arylalkyl groups include benzyl, phenethyl and naphthlenemethyl.

“Arylalkynyl” means an aryl-alkynyl-group in which the aryl and alkynyl are as previously described. Exemplary arylalkynyl groups include phenylethynyl.

“Cyclic amine” means an optionally substituted 3 to 8 membered monocyclic cycloalkyl ring system where one of the ring carbon atoms is replaced by nitrogen and which (i) may optionally contain an additional heteroatom selected from O, S or NR (where R is hydrogen, alkyl, arylalkyl, and aryl) and (ii) may be fused to additional aryl or heteroaryl ring to form a bicyclic ring system. Exemplary cyclic amines include pyrrolidine, piperidine, morpholine, piperazine, indoline. The cyclic amine group may be substituted by one or more substituent groups.

“Cycloalkenyl” means an optionally substituted non-aromatic monocyclic or multicyclic ring system containing at least one carbon-carbon double bond and having from 5 to 10 carbon atoms. Exemplary monocyclic cycloalkenyl rings include cyclopentenyl, cyclohexenyl or cycloheptenyl. The cycloalkenyl group may be substituted by one or more substituent groups.

“Cycloalkenylalkyl” means a cycloalkenyl-alkyl-group in which the cycloalkenyl and alkyl moieties are as previously described. Exemplary cycloalkenylalkyl groups include cyclopentenylmethyl, cyclohexenylmethyl or cycloheptenylmethyl.

“Cycloalkyl” means an optionally substituted saturated monocyclic or bicyclic ring system of from 3 to 12 carbon atoms, preferably from 3 to 8 carbon atoms, and more preferably from 3 to 6 carbon atoms. Exemplary monocyclic cycloalkyl rings include cyclopropyl, cyclopentyl, cyclohexyl and cycloheptyl. The cycloalkyl group may be substituted by one or more substituent groups.

“Cycloalkylalkyl” means a cycloalkyl-alkyl-group in which the cycloalkyl and alkyl moieties are as previously described. Exemplary monocyclic cycloalkylalkyl groups include cyclopropylmethyl, cyclopentylmethyl, cyclohexylmethyl and cycloheptylmethyl.

“Dialkylamino” means a —N(alkyl)₂ group in which alkyl is as defined above. Exemplary dialkylamino groups include dimethylamino and diethylamino.

“Halo” or “halogen” means fluoro, chloro, bromo, or iodo. Preferred are fluoro or chloro.

“Haloalkoxy” means an —O-alkyl group in which the alkyl is substituted by one or more halo atoms. Exemplary haloalkyl groups include trifluoromethoxy and difluoromethoxy.

“Haloalkyl” means an alkyl group which is substituted by one or more halo atoms. Exemplary haloalkyl groups include trifluoromethyl.

“Heteroaryl” as a group or part of a group denotes: (i) an optionally substituted aromatic monocyclic or multicyclic organic moiety of from 5 to 14 ring atoms, preferably from 5 to 10 ring atoms, in which one or more of the ring atoms is/are element(s) other than carbon, for example nitrogen, oxygen or sulfur (examples of such groups include benzimidazolyl, benzoxazolyl, benzthiazolyl, benzofuranyl, benzothienyl, furyl, imidazolyl, indolyl, indolizinyl, isoxazolyl, isoquinolinyl, isothiazolyl, oxazolyl, oxadiazolyl, pyrazinyl, pyridazinyl, pyrazolyl, pyridyl, pyrimidinyl, pyrrolyl, quinazolinyl, quinolinyl, tetrazolyl, 1,3,4-thiadiazolyl, thiazolyl, thienyl and triazolyl groups; (ii) an optionally substituted partially saturated multicyclic heterocarbocyclic moiety in which a heteroaryl and a cycloalkyl or cycloalkenyl group are fused together to form a cyclic structure (examples of such groups include pyrindanyl groups). The heteroaryl group may be substituted by one or more substituent groups.

“Heteroarylalkenyl” means a heteroaryl-alkenyl-group in which the heteroaryl and alkenyl moieties are as previously described. Exemplary heteroarylalkenyl groups include pyridylethenyl and pyridylallyl.

“Heteroarylalkyl” means a heteroaryl-alkyl-group in which the heteroaryl and alkyl moieties are as previously described. Preferred heteroarylalkyl groups contain a lower alkyl moiety. Exemplary heteroarylalkyl groups include pyridylmethyl.

“Heteroarylalkynyl” means a heteroaryl-alkynyl-group in which the heteroaryl and alkynyl moieties are as previously described. Exemplary heteroarylalkenyl groups include pyridylethynyl.

“Heterocycloalkyl” means: (i) an optionally substituted cycloalkyl group of from 4 to 8 ring members which contains one or more heteroatoms selected from O, S or NR; (ii) an optionally substituted partially saturated multicyclic heterocarbocyclic moiety in which an aryl (or heteroaryl ring) and a heterocycloalkyl group are fused together to form a cyclic structure (examples of such groups include dihydrobenzofuranyl, indolinyl and tetrahydroquinolinyl groups); (iii) a cycloalkyl group of from 4 to 8 ring members which contains C(═O)NR and C(═O)NRC(—O) (examples of such groups include succinimidyl and 2-oxopyrrolidinyl). The heterocycloalkyl group may be substituted by one or more substituent groups.

“Heterocycloalkylalkyl” means a heterocycloalkyl-alkyl-group in which the heterocycloalkyl and alkyl moieties are as previously described.

“Lower alkyl” as a group means unless otherwise specified, an aliphatic hydrocarbon group which may be straight or branched having 1 to 4 carbon atoms in the chain, i.e. methyl, ethyl, propyl (n-propyl or isopropyl) or butyl (n-butyl, isobutyl or tertiary-butyl).

“Pharmaceutically acceptable salt” means a physiologically or toxicologically tolerable salt and include, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that may be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that may be formed include hydrochlorides, hydrobromides, phosphates, acetates, citrates, lactates, tartrates, malonates, methanesulphonates and the like.

“Prodrug” means a compound which is convertible in vivo by metabolic means (e.g. by hydrolysis, reduction or oxidation) to a compound of formula (I). For example an ester prodrug of a compound of formula (I) containing a hydroxy group may be convertible by hydrolysis in vivo to the parent molecule. Suitable esters of compounds of formula (I) containing a hydroxy group, are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluenesulphonates, cyclohexylsulphamates and quinates. As another example an ester prodrug of a compound of formula (I) containing a carboxy group may be convertible by hydrolysis in vivo to the parent molecule [Examples of ester prodrugs are those described by F. J. Leinweber, Drug Metab. Res., 18:379 (1987)].

“Saturated” pertains to compounds and/or groups which do not have any carbon-carbon double bonds or carbon-carbon triple bonds.

The cyclic groups referred to above, namely, aryl, heteroaryl, cycloalkyl, cycloalkenyl, heterocycloalkyl and cyclic amine may be substituted by one or more substituent groups. Suitable optional substituents include acyl (e.g. —C(═O)CH₃), alkoxy (e.g., —OCH₃), alkoxycarbonyl (e.g. —C(═O)—OCH₃), alkylamino (e.g. —NHCH₃), alkylenedioxy (e.g. —O—CH₂—O—), alkylsulfinyl (e.g. —SOCH₃), alkylsulfonyl (e.g. —SO₂CH₃), alkylthio (e.g. —SCH₃), amino, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), hydroxy, formyl and nitro. In one embodiment, the optional substituent may further be selected from aryl (optionally substituted with alkoxy, haloalkoxy, halogen, alkyl or haloalkyl), heteroaryl (optionally substituted with alkoxy, haloalkoxy, halogen, alkyl or haloalkyl), heterocycloalkyl, aminoacyl (e.g. CONH₂, CONHCH₃), aminosulphonyl (e.g. SO₂NH₂, SO₂NHCH₃), acylamino (e.g. NHC(═O)CH₃), sulphonylamino (e.g. NHSO₂CH₃), heteroarylalkyl, cyclic amino (e.g. morpholine), aryloxy, heteroaryloxy, arylalkyloxy (e.g. benzyloxy) and heteroarylalkyloxy.

Compounds of the invention may exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application of adaptation of known methods (e.g. asymmetric synthesis).

With reference to formula (I) above, particular and preferred embodiments are described below.

Where R¹ is aryl or heteroaryl substituted by one or more haloalkyl groups, said haloalkyl group is preferably selected from trifluoromethyl. Where R¹ is aryl or heteroaryl substituted by one or more haloalkoxy groups, said haloalkoxy group is preferably selected from trifluoromethoxy or difluoromethoxy.

R¹ may particularly represent optionally substituted phenyl. Preferred groups for R¹ include phenyl or 4-methoxyphenyl.

R¹ may also particularly represent optionally substituted monocyclic heteroaryl, preferably optionally substituted imidazolyl, isoxazolyl, oxadiazolyl, pyrazolyl, pyridinyl, thienyl and pyrimidinyl, more preferably optionally substituted imidazolyl, pyrazolyl, pyridinyl and pyrimidinyl, particularly 2-imidazolyl, 3-pyrazolyl, 2-pyridinyl and 2-pyrimidinyl. In one embodiment, R¹ is optionally substituted 4-imidazolyl. Preferably, where R¹ is heteroaryl, it is preferably attached to the thienyl group of formula (I) above via a ring carbon atom of R¹, and in one embodiment via a ring carbon atom which is adjacent to a heteroatom. Preferred optional substituents include alkyl (preferably lower alkyl) and haloalkyl (preferably trifluoromethyl). Where the optional substituent is alkyl, the alkyl may be substituted, preferably by aryl or heteroaryl which in turn may be optionally substituted as described hereinabove. Particularly preferred substituents are arylalkyl, and heteroarylalkyl. In one embodiment, R¹ represents 1-(2-phenylethyl)-1H-pyrazol-3-yl, 1-benzyl-1H-pyrazol-3-yl, 4-trifluoromethyl-1H-imidazol-2-yl, pyridin-2-yl, 5-trifluoro-methyl-1H-pyrazol-3-yl, 1-methyl-1H-pyrazol-3-yl, 2-methyl-2H-pyrazol-3-yl, 1-methyl-5-trifluoromethyl-1H-pyrazol-3-yl, 2-methyl-5-trifluoromethyl-2H-pyrazol-3-yl, 1H-pyrazol-3-yl, pyridin-4-yl, 5-trifluoromethylisoxazol-3-yl, 3-methyl[1,2,4]oxadiazol-5-yl, or thiophene-2-yl.

R² may particularly represent hydrogen.

Where R² is alkyl, said alkyl group is preferably selected from lower alkyl, preferably methyl. Where R² is alkoxy, said alkoxy group is preferably selected from lower alkoxy, preferably methoxy. Where R² is haloalkyl, said haloalkyl group is preferably selected from trifluoromethyl.

In one embodiment, R³ and R⁸ are independently selected from alkyl, alkenyl, alkynyl, arylalkyl, arylalkenyl, arylalkynyl, heteroarylalkyl, heteroarylalkenyl, heteroarylalkynyl, cycloalkylalkyl, cycloalkenylalkyl, heterocycloalkylalkyl, aryl, heteroaryl, cycloalkyl, cycloalkenyl and heterocycloalkyl.

In one embodiment, R³ and R⁸ are independently selected from alkyl, preferably lower alkyl, preferably methyl or ethyl.

In one embodiment, R⁴ and R⁵ are independently selected from hydrogen, alkyl, alkenyl, aryl, arylalkyl, cycloalkyl, cycloalkenyl, cycloalkylalkyl, heteroaryl, heterocycloalkyl or heteroarylalkyl; or the group —NR⁴R⁵ may form a cyclic amine;

In an alternative embodiment R⁴ and R⁵ are independently selected from hydrogen, alkyl, alkenyl, aryl, arylalkyl, cycloalkyl, cycloalkylalkyl, heteroaryl or heteroarylalkyl; or the group —NR⁴R⁵ may form a cyclic amine;

In a further embodiment, R⁴ and R⁵ are independently selected from hydrogen and alkyl (preferably lower alkyl, preferably methyl).

In one embodiment, R⁷ is alkyl, alkenyl, alkynyl, arylalkyl, arylalkenyl, arylalkynyl, heteroarylalkyl, heteroalkylalkenyl, heteroalkynyl, cycloalkylalkyl, cycloalkenylalkyl or heterocycloalkylalkyl.

In a preferred embodiment, R¹ is substituted by an alkyl, alkenyl or alkynyl group, preferably an alkyl or alkenyl group, preferably an alkyl group (preferably C₁₋₃ alkyl), optionally substituted by one or more groups selected from aryl, heteroaryl, cycloalkyl, cycloalkenyl, heterocycloalkyl, hydroxy, —C(═Z)—NR⁴R⁵, —NR⁴R⁵, —NR⁶—C(═Z)—R⁸, —O—C(═O)—NR⁴R⁵, —NR⁶—C(═O)—OR⁸, —NR⁶—C(═O)—NR⁴R⁵, —NR⁶—SO₂—R⁸, —OR⁸, —SOR⁸, SO₂R⁸ and —SO₂—NR⁴R⁵. In a particularly preferred embodiment, said alkyl, alkenyl or alkyl group is substituted by a group selected from aryl, heteroaryl, cycloalkyl, cycloalkenyl and heterocycloalkyl, and optionally further substituted by a group selected from hydroxy, —C(═Z)—NR⁴R⁵, —NR⁴R⁵, —NR⁶—C(═Z)—R⁸, —O—C(═O)—NR⁴R⁵, —NR⁶—C(═O)—OR⁸, —NR⁶—C(═O)—NR⁴R⁵, —NR⁶—SO₂—R⁸, —OR⁸, —SOR⁸, SO₂R⁸ and —SO₂—NR⁴R⁵. In a further preferred embodiment, said alkyl, alkenyl or alkynyl group is substituted by a group selected from —C(═Z)—NR⁴R⁵, —NR⁶—C(═Z)—R⁸, —O—C(═O)—NR⁴R⁵, —NR⁶—C(═O)—OR⁸ and —NR⁶—C(═O)—NR⁴R⁵, and in one embodiment from —C(═Z)—NR⁴R⁵ and —NR⁶—C(═Z)—R⁸, preferably wherein Z is O, wherein R⁴, R⁵ or R⁸ is a cyclic group as defined herein and/or preferably wherein R⁴ and R⁶ are independently selected from hydrogen. In a further preferred embodiment, an R⁵ or R⁸ group may be selected from optionally substituted aryl, heteroaryl, heterocycloalkyl or alkyl (preferably C₁₋₃ alkyl) substituted by optionally substituted aryl, heteroaryl or heterocycloalkyl, and preferably from a group —(CH₂)ₘ—Ar as defined hereinbelow.

In a further embodiment, R¹ may be substituted by a group X defined hereinbelow.

In one embodiment, R¹ is selected from 3-pyrazolyl substituted by an alkyl group (preferably a C₁₋₃ alkyl group, particularly methyl) which is substituted by a —CO)—NR⁴R⁵ group or a —(SO₂)—NR⁴R⁵ group, preferably by a —(CO)—NR⁴R⁵ group. In this embodiment, R₄ is preferably hydrogen. In this embodiment, R⁵ is preferably optionally substituted aryl, heteroaryl, heterocycloalkyl or alkyl (particularly methyl or ethyl) substituted by optionally substituted aryl, heteroaryl or heterocycloalkyl, and preferably R⁵ is optionally substituted aryl, heteroaryl or heterocycloalkyl. In this embodiment, the alkyl substituent is preferably present at the 1-position of the 3-pyrazolyl. Thus, in one embodiment, R¹ represents 3-pyrazolyl and the compounds of the invention are represented by formula (Ia): 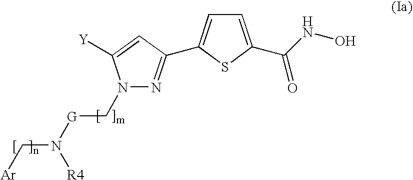
in which Ar is optionally substituted aryl, heteroaryl or heterocycloalkyl;
Y is H, CF₃ or alkyl, preferably H;
G is CO or SO₂, preferably CO;
R⁴ is preferably H;
n is 0, 1 or 2, preferably 0; and
m is 1, 2 or 3, preferably 1.

In a further embodiment, R¹ is selected from 3-pyrazolyl substituted by an alkyl group (preferably a C₂₋₄ alkyl group, preferably ethyl) which is substituted by OR⁸ or NR⁴R⁵. In this embodiment, R⁸ or R⁵ is preferably selected from optionally substituted aryl, heteroaryl and heterocycloalkyl, and from alkyl (preferably a C₁₋₃ alkyl group) substituted by optionally substituted aryl, heteroaryl or heterocycloalkyl. In one embodiment, R⁴ is hydrogen or alkyl, preferably hyrdogen. Thus, in this embodiment, compounds of the invention are represented by formula (Ib) or (1c): 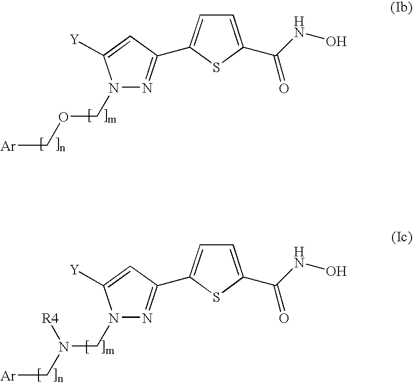
in which Ar and Y are as defined above;
m is 2, 3 or 4, preferably 2;
n is 0, 1, 2 or 3; and
R⁴ is hydrogen or alkyl, preferably hydrogen.

In another embodiment, compounds of the invention are represented by formula (Id): 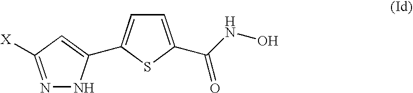
wherein the group X is selected from the group consisting of optionally substituted aryl, optionally substituted heteroaryl, optionally substituted heterocycloalkyl, —C(O)—NR⁴R⁵, —NR⁴R⁵, —NR⁶—C(O)—R⁸, —NR⁶—SO₂—R⁸, —OR⁸, —SO₂—NR⁴R⁵ and alkyl (preferably C₁₋₃ alkyl) substituted by a group selected from optionally substituted aryl, optionally substituted heteroaryl, optionally substituted heterocycloalkyl, —C(O)—NR⁴R⁵, —NR⁴R⁵, —NR⁶—C(O)—R⁸, —NR⁶—SO₂—R⁸, —OR⁸ and —SO₂—NR⁴R⁵ wherein R⁴, R⁵, R⁶ and R⁸ are as previously defined. In the group X, R⁴ and R⁶ are preferably selected from hydrogen and alkyl, preferably hydrogen, and R⁵ and R⁸ are preferably selected from optionally substituted aryl, heteroaryl, heterocycloalkyl or alkyl (preferably C₁₋₃ alkyl) substituted by optionally substituted aryl, heteroaryl or heterocycloalkyl. The group X is preferably selected from:
—(CH₂)ₙCONR⁴(CH₂)ₘAr,
—(CH₂)ₙSO₂NR⁴(CH₂)ₘAr,
—(CH₂)ₙNR⁶CO(CH₂)ₘAr,
—(CH₂)ₙNR⁶SO₂(CH₂)ₘAr,
—(CH₂)ₙNR⁴(CH₂)ₘAr,
—(CH₂)ₙO(CH₂)ₘAr, and
—(CH₂)ₙAr;
Ar, R⁴ and R⁶ are as defined above;
n is 0, 1, 2 or 3; and
m is 0, 1, 2, 3 or 4.

In one embodiment, the value of m in the group X is 0, 1, 2 or 3, and this is referred to herein as group X¹. In the compounds of formula (Id), it is preferred that X is group X¹.

In an alternative embodiment, R¹ is 2-pyridinyl and compounds of the invention are represented by formula (Ie): 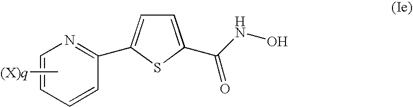
wherein X is as defined above, and may be attached at either the 5 or the 6 position of the pyridine, preferably the 5-position; and
q is either 1 or 2, preferably 1,
wherein preferably X is —(CH₂)ₙCONR⁴(CH₂)ₘAr, preferably wherein n is 1, and preferably wherein R⁴ is H,

In an alternative embodiment, R¹ represents 2-imidazolyl and compounds of the invention are represented by formula (If): 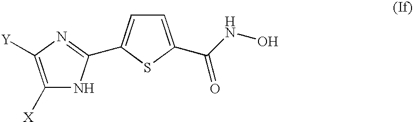
wherein X and Y are as defined above, particularly wherein Y is H or alkyl (preferably methyl).

In an alternative embodiment, the compounds of the present invention are represented by formula (If), wherein Y is CF₃ and the group X is replaced by H.

In an alternative embodiment, R¹ represents 4-imidazolyl and compounds of the invention are represented by formula (Ig): 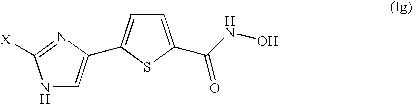
wherein X is as defined above.

In an alternative embodiment, R¹ represents 2-pyrimidinyl and compounds of the invention are represented by formula (Ih): 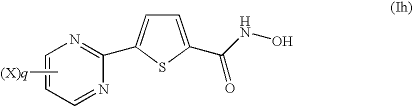
wherein X and q are as defined above, and the substituent X is attached at either the 5 or 6 position of the pyrimidine ring.

In the compounds of the invention, particularly as represented by formula (Ia) to (Ih):

aryl is preferably phenyl;

heteroaryl is preferably quinolinyl (including the N-oxide), isoquinolinyl (including the N-oxide), pyridyl (including the N-oxide), oxadiazolyl, thiadiazolyl, imidazolyl, indolyl, indazolyl, pyrolyl or benzofuranyl; and

heterocycloalkyl is preferably either (i) an optionally substituted saturated multicyclic heterocarbocyclic moiety in which an aryl or heteroaryl ring and a heterocycloalkyl group are fused together to form a cyclic structure, more preferably dihydrobenzo[1,4]dioxinyl, or (ii) piperazinyl substituted on nitrogen by aryl, arylalkyl, heteroarylalkyl or heteroaryl.

The optional substituents which may be present on the aryl, heteroaryl or heterocycloalkyl groups are preferably selected from halogen, CF₃, OCF₃, alkyl, acylamino, arylalkyl, aryloxy, aryl, cyclic amino, heteroaryl, alkylenedioxy and aminosulphonyl.

The heteroaryl or heterocycloalkyl group represented by Ar may be attached through a carbon atom, or in an alternative embodiment is attached through a heteroatom, e.g. 1-imidazolyl or 1-piperazinyl.

In one embodiment compounds of the invention are:

5-(4-trifluoromethyl-1H-imidazol-2-yl)-thiophene-2-carboxylic acid hydroxyamide;

5-(1-benzyl-1H-pyrazol-3-yl)-thiophene-2-carboxylic acid hydroxyamide;

5-(1-phenethyl-1H-pyrazol-3-yl)-thiophene-2-carboxylic acid hydroxyamide;

5-pyridin-2-yl-thiophene-2-carboxylic acid hydroxyamide;

and corresponding N-oxides, pharmaceutically acceptable salts, solvates and prodrugs of such compounds.

In a preferred embodiment compounds of the invention are:
5-[1-(2,3-dihydro-benzo[1,4]dioxin-2-ylmethyl)-1H-pyrazol-3-yl]-thiophene-2-carboxylic acid hydroxyamide;
5-(5-phenethyl-1H-pyrazol-3-yl)-thiophene-2-carboxylic acid hydroxyamide;
5-pyrimidin-2-yl-thiophene-2-carboxylic acid hydroxyamide;
5-(1-benzo[1,3]dioxol-5-ylmethyl-1H-pyrazol-3-yl)-thiophene-2-carboxylic acid hydroxyamide;
5-(1-phenethyl-5-trifluoromethyl-1H-pyrazol-3-yl)-thiophene-2-carboxylic acid hydroxyamide;
5-(4-benzyloxy-pyrimidin-2-yl)-thiophene-2-carboxylic acid hydroxyamide;
5-(2-phenethyl-3H-imidazol-4-yl)-thiophene-2-carboxylic acid hydroxyamide;
5-[1-(5-tert-butyl-[1,2,4]oxadiazol-3-ylmethyl)-1H-pyrazol-3-yl]-thiophene-2-carboxylic acid hydroxyamide;
5-{1-[6-(2,2-dimethyl-propionylamino)-pyridin-2-ylmethyl]-1H-pyrazol-3-yl}-thiophene-2-carboxylic acid hydroxyamide;
5-(5-phenylacetylamino-pyridin-2-yl)-thiophene-2-carboxylic acid hydroxyamide;
5-(1-quinolin-2-ylmethyl-1H-pyrazol-3-yl)-thiophene-2-carboxylic acid hydroxyamide;
5-[5-(2-benzyloxy-ethylamino)-pyridin-2-yl]-thiophene-2-carboxylic acid hydroxyamide;
5-{5-[(2,3-dihydro-benzo[1,4]dioxin-6-ylmethyl)-amino]-pyridin-2-yl}-thiophene-2-carboxylic acid hydroxyamide;
5-{5-[(benzofuran-2-ylmethyl)-amino]-pyridin-2-yl}-thiophene-2-carboxylic acid hydroxyamide;
5-{1-[2-(4-fluoro-benzyloxy)-ethyl]-1H-pyrazol-3-yl}-thiophene-2-carboxylic acid hydroxyamide;
5-(1-phenylcarbamoylmethyl-1H-pyrazol-3-yl)-thiophene-2-carboxylic acid hydroxyamide;
5-[1-(quinolin-8-ylcarbamoylmethyl)-1H-pyrazol-3-yl]-thiophene-2-carboxylic acid hydroxyamide;
5-{1-[(4-fluoro-phenylcarbamoyl)-methyl]-1H-pyrazol-3-yl}-thiophene-2-carboxylic acid hydroxyamide;
5-{1-[(4-oxazol-5-yl-phenylcarbamoyl)-methyl]-1H-pyrazol-3-yl}-thiophene-2-carboxylic acid hydroxyamide;
quinoline-2-carboxylic acid {2-[3-(5-hydroxycarbamoyl-thiophen-2-yl)-pyrazol-1-yl]-ethyl}-amide;
5-{1-[(2-morpholin-4-yl-phenylcarbamoyl)-methyl]-1H-pyrazol-3-yl}-thiophene-2-carboxylic acid hydroxyamide;
5-(1-{[2-(1H-indol-3-yl)-ethylcarbamoyl]-methyl}-1H-pyrazol-3-yl)-thiophene-2-carboxylic acid hydroxyamide;
5-{1-[(2-fluoro-phenylcarbamoyl)-methyl]-1H-pyrazol-3-yl}-thiophene-2-carboxylic acid hydroxyamide;
5-[1-(quinolin-3-ylcarbamoylmethyl)-1H-pyrazol-3-yl]-thiophene-2-carboxylic acid hydroxyamide;
2-(5-hydroxycarbamoyl-thiophen-2-yl)-5-methyl-1H-imidazole-4-carboxylic acid phenethyl-amide;
2-(5-hydroxycarbamoyl-thiophen-2-yl)-5-methyl-1H-imidazole-4-carboxylic acid benzylamide;
5-(6-benzyloxymethyl-pyridin-2-yl)-thiophene-2-carboxylic acid hydroxyamide;
5-{1-[(1H-indol-7-ylcarbamoyl)-methyl]-1H-pyrazol-3-yl}-thiophene-2-carboxylic acid hydroxyamide;
5-{1-[(3-chloro-phenylcarbamoyl)-methyl]-1H-pyrazol-3-yl}-thiophene-2-carboxylic acid hydroxyamide;
5-{1-[(3-methoxy-phenylcarbamoyl)-methyl]-1H-pyrazol-3-yl}-thiophene-2-carboxylic acid hydroxyamide;
5-[1-(1-oxy-quinolin-2-ylmethyl)-1H-pyrazol-3-yl]-thiophene-2-carboxylic acid hydroxyamide;
5-(1-{2-[(benzo[1,3]dioxol-5-ylmethyl)-amino]-ethyl}-1H-pyrazol-3-yl)-thiophene-2-carboxylic acid hydroxyamide;
5-[1-(2-benzylamino-ethyl)-1H-pyrazol-3-yl]-thiophene-2-carboxylic acid hydroxyamide; and corresponding N-oxides, pharmaceutically acceptable salts, solvates and prodrugs of such compounds.

The present invention provides compounds that inhibit HDAC activity according to the tests described in the literature and in the Biological Activity section of this document. The therapeutic application of these compounds is pertinent to any disease that is known to be at least in part mediated by HDAC activity or whose symptoms are known to be alleviated by HDAC inhibitors (such as Trichostatin-A, suberoyl anilide hydroxamic acid, Trapoxin and depudecin). For example, these compounds could be beneficial for the treatment of cancer, psoriasis, fibroproliferative disorders (e.g. liver fibrosis), smooth muscle cell proliferation disorders (e.g. arteriosclerosis, restenosis), inflammatory diseases and conditions treatable by immune modulation (e.g. rheumatoid arthritis, autoimmune diabetes, lupus, allergies), neurodegenerative disorders (e.g. Huntington's disease), diseases involving angiogenesis (e.g. cancer, psoriasis, rheumatoid arthritis, retinal diseases such as diabetic retinopathy, age-related macular degeneration, interstitial keratitis, rubeotic glaucoma), fungal and parasitic infections (e.g. malaria, protozoal infections) and haematopoietic disorders (e.g. anaemia, sickle cell anaemia, thalassemia).

Thus, in one embodiment, the present invention is intended for the treatment of diseases caused by increased cell proliferation. These include, but are not limited to, primary and metastatic cancers of different origin (including those triggered by viral infections such as EBV, HIV, hepatitis B and C and KSHV), fibrosis of the liver, lung, kidney, heart and skin caused by myofibroblasts proliferation and increased production of extracellular matrix proteins [Niki et al, Hepatology, 29:858-67 (1999)], inflammatory diseases and cardiomyocyte hypertrophy [Lu et al., PNAS, 97: 4070-4075 (2000)].

In another embodiment, the invention is also aimed at the treatment of protozoal infections including, but not limited to, malaria, toxoplasmosis and coccidiosis.

In another embodiment, the invention is aimed at the treatment of diseases caused by expanded polyglutamine repeats resulting in histone hypoacetylation including, but not limited to, neurodegenerative disorders such as Huntington's disease.

The compounds of formula I may be used or administered in combination with one or more additional drug(s) and/or procedures (such as radiotherapy in the case of cancer) useful in the treatment of the disorders mentioned above, the components being in the same formulation or in separate formulations for administration simultaneously or sequentially. The additional drug(s) may or may not be HDAC inhbitors.

The thienyl-hydroxamic acids of the present invention may be prepared, for example, by the application or adaptation of methods described herein. They may also be prepared by known organic synthesis methods for example those described by R. C. Larock in Comprehensive Organic Transformations, VCH publishers, 1989.

It may be necessary to protect reactive functional groups (e.g. hydroxy, amino, thio or carboxy) in intermediates used in the preparation of compounds of formula (I) to avoid their unwanted participation in a reaction leading to the formation of compounds of formula (I). Conventional protecting groups, for example those described by T. W. Greene and P. G. M. Wuts in “Protective Groups in Organic Chemistry” John Wiley and Sons. 1999, may be used. In the reaction schemes provided below, all definitions of R¹ to R²¹ are to be understood to include such protected functional groups.

### Preparation of Compounds of Formula (I)

Compounds of formula (I) may be prepared from the corresponding carboxylic acids of formula (II) as shown in Reaction Scheme 1:

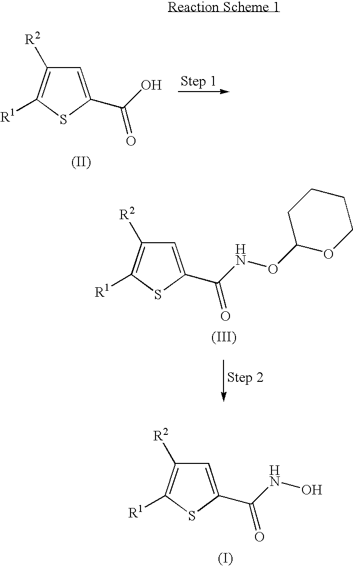

Thus for example a compound of formula (II), wherein R¹ and R² are as hereinbefore defined, is reacted, in step 1, with O-(tetrahydro-2H-pyran-2-yl)hydroxylamine and a suitable coupling agent, such as O-(7-azabenzotriazol-1-yl)-N,N,N′,N′-tetramethyluronium hexafluorophosphate, in the presence of diisopropylethylamine, in an inert solvent, such as dimethylformamide, and at a temperature of about room temperature. The resulting product of formula (III), wherein R¹ and R² are as hereinbefore defined, is reacted, in step 2, with an acid catalyst, such as p-toluene sulfonic acid, in methanol and at a temperature of about room temperature to obtain compounds of formula (I), wherein R¹ and R² are as hereinbefore defined.

Alternatively compounds of formula (I) may be prepared from compounds of formula (II) by reaction with other O-protected hydroxylamines, such as O-(trimethylsilyl)hydroxylamine, O-(t-butyldimethylsilyl)-hydroxylamine, or O-benzylhydroxylamine, followed by a deprotection using a suitable reagent such as tetra-n-butylammonium fluoride or hydrogen in the presence of a palladium (0) catalyst.

Alternatively compounds of formula (I) may be prepared from compounds of formula (II) by reaction with N,O-diprotected hydroxylamines such as O-2,4-dimethoxybenzyl-N-2,4,6-trimethoxybenzyl hydroxylamine, followed by deprotection using a suitable acid such as 10% trifluoroacetic acid in dichloromethane.

Alternatively compounds of formula (I) may be prepared from compounds of formula (II) by reaction with hydroxylamine.

Compounds of formula (I) may also be prepared from the corresponding esters (IV) as shown in Reaction Scheme 2:

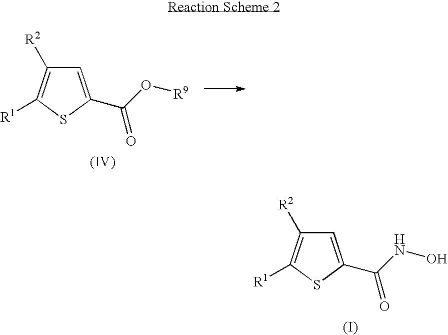

Thus compounds of formula (IV), wherein R¹ and R² are hereinbefore defined and R⁹ is lower alkyl (preferably methyl or ethyl), may be reacted with hydroxylamine hydrochloride in the presence of a base, for example triethylamine, sodium methoxide or potassium hydroxide, in a protic solvent such as methanol or ethanol and using a co-solvent such as N,N-dimethylacetamide if required, at temperatures from room temperature up to the reflux temperature of the solvent to obtain compounds of formula (I), wherein R¹ and R² are as hereinbefore defined.

Compounds of formula (I) may also be prepared by interconversion of other compounds of the invention.

As one example, compounds of formula (I) in which R¹ is heteroaryl containing a nitrogen atom substituted by alkyl, arylalkyl, or heteroarylalkyl (e.g. R¹ is 1-benzyl-1H-pyrazol-3yl) may be prepared by alkylation of the corresponding compounds of formula (I) in which R¹ is heteroaryl containing an unsubstituted imino group (e.g. R¹ is 1H-pyrazol-3-yl) with the appropriate alkyl, arylalkyl- or heteroarylalkyl-halides, preferably bromides, using standard alkylation conditions. The alkylation may for example be carried out in the presence of a base, such as an alkali metal carbonate, e.g. potassium carbonate, or alkali metal hydride, e.g. sodium hydride, in an inert solvent, such as tetrahydrofuran, dimethylformamide or dimethyl sulfoxide, at a temperature from about 0° C. to about

As another example, compounds of formula (I) in which R¹ is heteroaryl containing an N-oxide group (e.g. pyridine-N-oxide) may be prepared by oxidation of compounds of formula (I) in which R¹ is the corresponding non-oxidised heteroaryl. The oxidation may conveniently be carried out by means of reaction with a mixture of hydrogen peroxide and an organic acid, e.g. acetic acid, preferably at or above room temperature, for example at a temperature of about 60-90° C. Alternatively, the oxidation may be carried out by reaction with a peracid, for example peracetic acid or m-chloroperoxybenzoic acid, in an inert solvent such as chloroform or dichloromethane, at a temperature from about room temperature to reflux, preferably at elevated temperature. The oxidation may alternatively be carried out by reaction with hydrogen peroxide in the presence of sodium tungstate at temperatures between room temperature and about 60° C.

Alternatively the oxidative reaction may be carried out using magnesium monoperoxyphthalate hexahydrate in solvents such as dichloromethane and methanol.

The starting materials and intermediates may be prepared by the application or adaptation of methods described herein, or those known in the literature.

### Preparation of Intermediates of Formula (II)

Intermediates of formula (II) may be prepared from compounds of formula (1) as shown in Reaction Scheme 3:

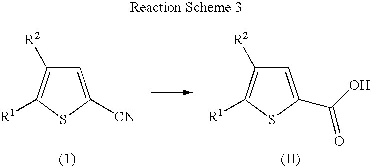

Thus compounds of formula (1), wherein R¹ and R² are as hereinbefore defined, may be reacted with aqueous base, for example sodium hydroxide solution, in a protic solvent, for example methanol or ethanol, at reflux temperature to obtain acids of formula (II), wherein R¹ and R² are as hereinbefore defined.

Intermediates of formula (II) may also be prepared from compounds of formula (IV) as shown in Reaction Scheme 4:

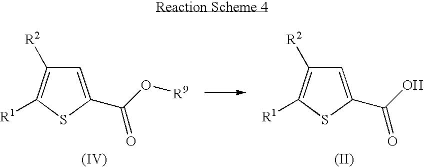

Thus compounds of formula (IV), where R¹, R² and R⁹ are hereinbefore defined, may be reacted with aqueous base, for example sodium hydroxide solution, in a protic solvent, for example methanol or ethanol, at temperatures from room temperature up to reflux temperature to obtain compounds of formula (II), where R¹ and R² are hereinbefore defined.

Intermediates of formula (II) may also be prepared from compounds of formula (2) as shown in Reaction Scheme 5:

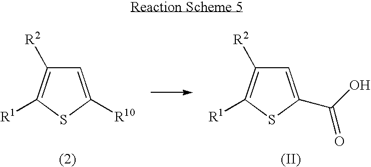

Thus compounds of formula (2), where R¹ and R² are hereinbefore defined and R¹⁰ is hydrogen, bromo, or iodo, may be reacted with an organolithium (for example butyllithium) in an inert solvent (for example diethyl ether or tetrahydrofuran) at temperatures from about room temperature to about −80° C., followed by reaction with carbon dioxide to obtain compounds of formula (II), where R¹ and R² are hereinbefore defined.

### Preparation of Intermediates of Formula (IV)

Intermediates of formula (I) may be prepared from compounds of formula (2) as shown in Reaction Scheme 6:

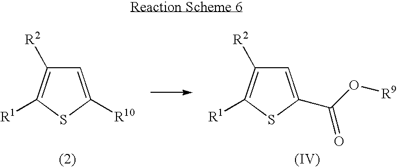

Thus compounds of formula (2), where R¹ and R² are hereinbefore defined and R¹⁰ is hydrogen, bromo, or iodo, may be reacted with an organolithium (for example butyllithium) in an inert solvent (for example diethyl ether or tetrahydrofuran) at temperatures from about room temperature to about −80° C., followed by reaction with an alkyl chloroformate of formula R⁹—O—C(═O)—Cl, wherein R⁹ is as hereinbefore defined, e.g. methyl chloroformate or ethyl chloroformate) to obtain compounds of formula (IV), here R¹, R² and R⁹ are hereinbefore defined.

Alternatively the carbon dioxide can be used in place of the alkyl chloroformate to provide compounds of formula (IV) where R¹ and R² are hereinbefore defined and R⁹ is hydrogen.

### Preparation of Intermediates of Formula (1)

Compounds of formula (1) may be prepared from compounds of formula (3) as shown in Reaction Scheme 7:

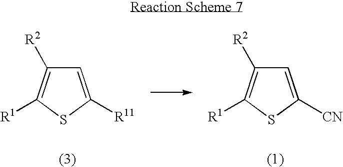

Thus compounds of formula (3), wherein R¹ and R² are as hereinbefore defined and R¹¹ is chloro, bromo or iodo, may be reacted with cuprous cyanide in an inert solvent such as N,N-dimethylformamide, or N-methyl-2-pyrrolidinone, at elevated temperatures from about 100° C. up to the reflux temperature of the solvent to obtain compounds of formula (1), wherein R¹ and R² are as hereinbefore defined.

Alternatively, compounds of formula (1) may be prepared from compounds of formula (3) by reaction with zinc cyanide in the presence of a palladium (0) catalyst, for example tetrakis (triphenylphospine)palladium (0), in an inert solvent, for example N,N-dimethylformamide, at temperatures from about room temperature up to reflux temperature.

### Preparation of Intermediates of Formula (3)

Intermediates of formula (3) may be prepared from compounds of formula (4) as shown in Reaction Scheme 8:

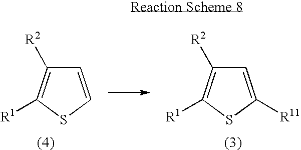

Thus compounds of formula (3), wherein R¹ and R² are as hereinbefore defined and R¹¹ is chloro, bromo or iodo, may be prepared from compounds of formula (4), wherein R¹ and R² are as hereinbefore defined, by reaction with an appropriate halogenating agent, for example bromine, iodine, N-chlorosuccinimide, N-bromosuccinimide, or N-iodosuccinimide.

### General Methods for the Preparation of Compounds of Formulae (II), (IV), (1), and (4)

Common synthetic methods may be applied to compounds of formula (5), where R¹² is hydrogen, carboxy, C(═O)OR⁹ or cyano:

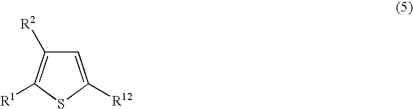

It should be understood that formula (5) is a general formula which comprises compounds of formulae (II), (IV), (1), and (4).

Compounds of formula (5) may be prepared from compounds of formula (6) as shown in Reaction Scheme 9:

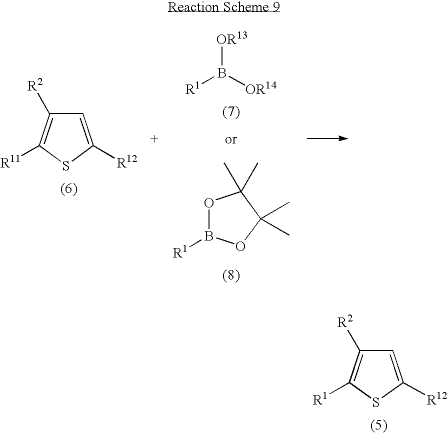

Thus compounds of formula (6), wherein R², R¹¹ and R¹² are as hereinbefore defined, may be coupled with compounds of formula (7), in which R¹ is hereinbefore defined and R¹³ and R¹⁴ are independently hydrogen or lower alkyl, to obtain compounds of formula (5), wherein R¹, R² and R¹² are as hereinbefore defined. The reaction is performed in the presence of a suitable catalyst, such as tetrakis(triphenylphosphine)palladium (0), and a suitable base, such as cesium carbonate in a suitable solvent such as N,N-dimethylformamide at a temperature of from about room temperature up to the reflux temperature of the solvent.

Alternatively the coupling reaction may be carried out using compounds of formula (8), wherein R¹ is as hereinbefore defined.

Compounds of formula (5) may also be prepared from compounds of formula (11) as shown in Reaction Scheme 10:

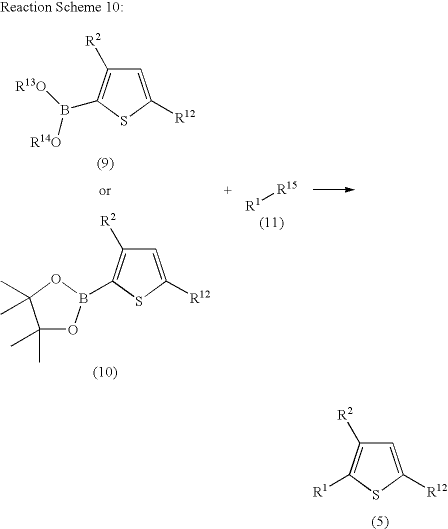

Thus compounds of formula (11), wherein R¹ is as hereinbefore defined and R¹⁵ is chloro, bromo, iodo, or trifluoromethanesulfonyloxy, may be reacted with compounds of formula (9), wherein R² and R¹² are as hereinbefore defined and R¹³ and R¹⁴ are independently hydrogen or lower alkyl, to obtain compounds of formula (5), wherein R¹, R² and R¹² are as hereinbefore defined. The reaction is performed in the presence of a suitable catalyst, such as tetrakis(triphenylphosphine)palladium (0), and a suitable base, such as cesium carbonate, in a suitable solvent, such as N-dimethylformamide, and at a temperature from about room temperature up to the reflux temperature of the solvent.

Alternatively, the coupling reaction may also be carried out using compounds of formula (10) wherein R² and R¹² are as hereinbefore defined.

Compounds of formula (6), wherein R², R¹¹ and R¹² are as hereinbefore defined, may be prepared from compounds of formula (12):— 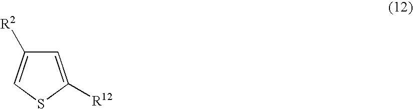
wherein R² and R¹² are as hereinbefore defined, by reaction with a suitable halogenating agent such as bromine, iodine, N-chlorosuccinimide, N-bromosuccinimide, or N-iodosuccinimide.

Compounds of formula (7), wherein R¹, R¹³ and R¹⁴ are as hereinbefore defined, may be obtained from commercial sources. Alternatively, compounds of formula (7), wherein R¹ is as hereinbefore defined and R¹³ and R¹⁴ are both methyl (or ethyl), may be obtained by, for example, the reaction of an organometallic reagent of formula (13):—
R¹-M (13)
where R¹ is as previously defined and M is a metal atom such as lithium or magnesium, with trimethylborate (or triethylborate).

Compounds of formula (8), wherein R¹ is as hereinbefore defined, may be prepared from compounds of formula (11), wherein R¹ and R¹⁵ are as hereinbefore defined, by reaction with bis(pinacolato)diboron in the presence of a suitable catalyst, such as [1,1′-bis(diphenylphosphino)ferrocene]dichloropalladium, and a suitable base, such as potassium acetate in a suitable solvent such as, dioxan at a temperature of from about room temperature up to a temperature of 80° C.

Alternatively, compounds of formula (8), wherein R¹ is as hereinbefore defined, may be prepared from compounds of formula (11), wherein R¹ and R¹⁵ are as hereinbefore defined, by reaction with bis(pinacolato)diboron in the presence of a catalyst, such as palladium acetate, and a suitable base, such as potassium acetate in a suitable solvent such as, dimethyl sulfoxide at a temperature of from about room temperature up to a temperature of 80° C.

Compounds of formula (9) may obtained from commercial sources (e.g. 5-(dihydroxyboryl)-2-thiophenecarboxylic acid), or can be prepared by those skilled in the art. Alternatively compounds of formula (9) may be prepared according to Reaction Scheme 11:

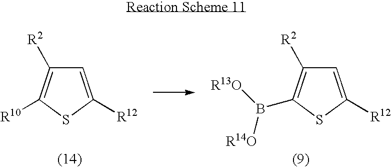

Thus compounds of formula (14), wherein R² and R¹² are as hereinbefore defined and R¹⁰ is hydrogen, bromo, or iodo, may be reacted with an organolithium reagent, for example butyllithium, followed by reaction with trimethylborate (or triethylborate), in an inert solvent such as tetrahydrofuran, at temperatures from about −80° C. to about room temperature to obtain compounds of formula (9), wherein R² and R¹² are as hereinbefore defined and R¹³ and R¹⁴ are both methyl (or ethyl).

Compounds of formula (10), wherein R² and R¹² are as hereinbefore defined, may be prepared from compounds of formula (6), wherein R², R¹¹ and R¹² are as hereinbefore defined, by reaction with bis(pinacolato)diboron in the presence of a suitable catalyst, such as [1,1′-bis(diphenylphosphino)ferrocene]dichloropalladium, and a suitable base, such as potassium acetate in a suitable solvent such as, dioxan at a temperature of from about room temperature up to a temperature of 80° C.

Compounds of formula (11) and (12) may be obtained from commercial sources, or may be prepared using published methods described in the literature.

Compounds of formula (5), wherein R² is as hereinbefore defined, R¹² is hydrogen or cyano and R¹ is 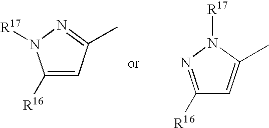
(in which R¹⁶ is hydrogen, trifluoromethyl, alkyl, aryl, heteroaryl, heterocycloalkyl, arylalkyl, heteroarylalkyl, or heterocycloalkylalkyl and R¹⁷ is hydrogen, alkyl, aryl, heteroaryl, heterocycloalkyl, arylalkyl, cycloalkyl, cycloalkylalkyl, heteroarylalkyl or heterocycloalkylalkyl, hereinafter described as compounds of formula (15a) and (15b), may be prepared according to Reaction Scheme 12:

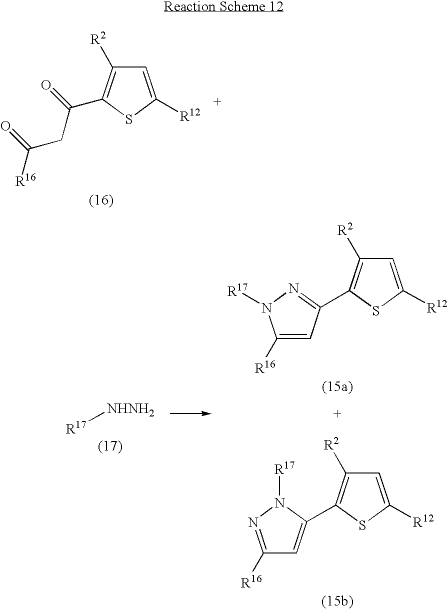

Thus 1,3-diketones of formula (16), wherein R² and R¹⁶ are as hereinbefore defined, and R¹² is hydrogen or cyano, may be reacted with hydrazines of formula (17), wherein R¹⁷ is as hereinbefore defined, to obtain compounds of formula (15a) and (15b). The reaction may be carried out in a protic solvent, for example an alcohol, preferably ethanol, at temperatures from about room temperature up to the reflux temperature of the solvent. It will be recognized that such reactions may give rise to mixtures of the two regioisomers (15a) and (15b), the ratio of which will depend upon the nature of the groups R², R¹⁶, and R¹⁷, and the reaction conditions. Where produced, such regioisomers may be separated by classical techniques such as fractional crystallisation or chromatography.

Compounds of formula (16), wherein R² and R¹⁶ are as hereinbefore defined and R¹² is hydrogen or cyano, may be prepared as shown in Reaction Scheme 13:

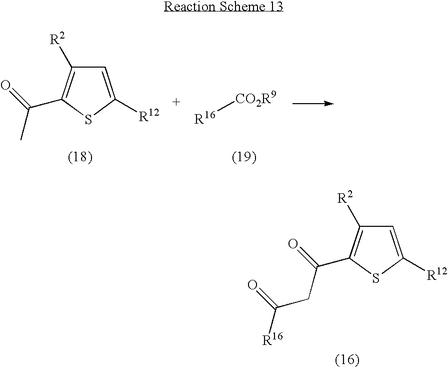

Thus compounds of formula (18), wherein R² is as hereinbefore defined and R¹² is hydrogen or cyano, may be reacted with compounds of formula (19), wherein R¹⁶ is as hereinbefore defined and R⁹ is lower alkyl, to obtain compounds of formula (16). The reaction may conveniently be carried out with a suitable base, for example sodium methoxide, in a protic solvent such as an alcohol, for example methanol, at temperatures of from about room temperature up to the reaction temperature of the solvent.

Compounds of formula (15a) and (15b), where R¹² is hydrogen, carboxy, C(═O)OR⁹ or cyano and R¹⁶ is H, may be prepared as shown in Reaction Scheme 14:

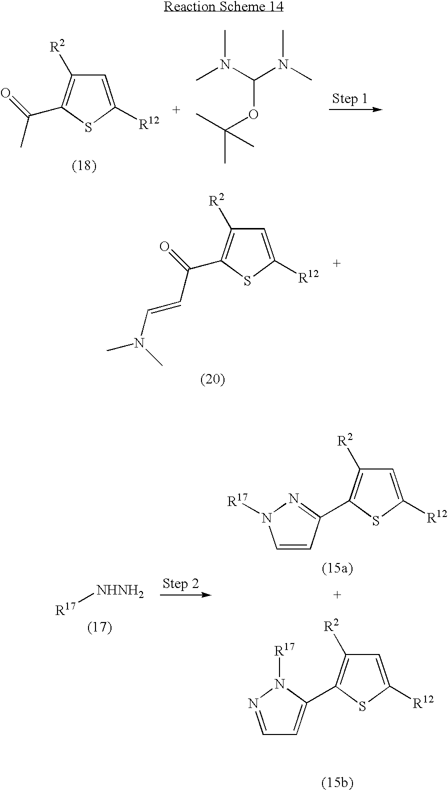

Thus for example compounds of formula (18), wherein R² is as hereinbefore defined and R¹² is hydrogen, carboxy, C(═O)OR⁹ or cyano, may be reacted, in step 1, with tert-butoxybis(dimethylamino)methane in a suitable solvent such as N,N-dimethylformamide at temperatures of from about room temperature up to about the reflux temperature of the solvent. The resulting intermediate of formula (20), wherein R² and R¹² is as hereinbefore defined, may be reacted, in step 2, with hydrazines of formula (17), wherein R¹⁷ is as described hereinbefore, to obtain compounds of formula (15a) and (15b), wherein R², R¹² and R¹⁷ are as hereinbefore described. Step 2 may conveniently be carried out in a protic solvent, for example an alcohol, preferably ethanol, at temperatures from about room temperature up to the reflux temperature of the solvent. It will be recognized that such reactions may give rise to two regioisomers, the ratio of which will depend upon the nature of the groups R² and R¹⁷, and the reaction conditions. Where produced, such regioisomers may be separated by classical techniques such as fractional crystallisation or chromatography.

Compounds of formula (17) and (18) may be obtained from commercial sources, or may be prepared using published methods described in the literature.

Compounds of formula (15a) and (15b), where R¹⁷ is alkyl, arylalkyl, cycloalkylalkyl, heteroarylalkyl, or heterocycloalkylalkyl, alkenyl, alkynyl, arylalkenyl, arylalkynyl, may be prepared as shown in Reaction Scheme 15:

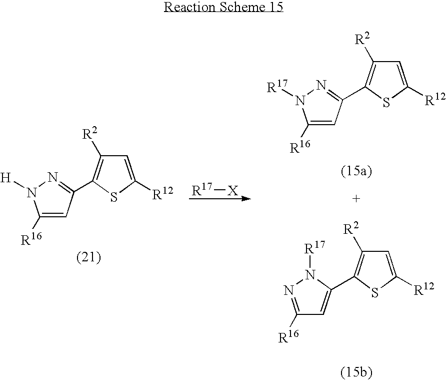

Thus for example compounds of formula (21), wherein R², R¹², and R¹⁶ are as hereinbefore defined, may be reacted with compounds of formula R¹⁷—X, wherein R¹⁷ is alkyl, arylalkyl, cycloalkylalkyl, heteroarylalkyl, or heterocycloalkylalkyl, alkenyl, alkynyl, arylalkenyl, arylalkynyl and X is halo (preferably bromo), —OSO₂C₆H₅ or —OSO₂CH₃, in the presence of a suitable base, for example sodium hydride, in an inert solvent such as N,N-dimethylformamide at temperatures of from about room temperature up to the reflux temperature of the solvent. It will be recognized that such reactions may give rise to two regioisomers, the ratio of which will depend upon the nature of the groups R², R¹⁶, and R¹⁷, and the reaction conditions. Where produced, such regioisomers may be separated by classical techniques such as fractional crystallisation or chromatography.

Compounds of general formula (5), where R¹² is hydrogen, carboxy, C(═O)OR⁹ or cyano, and R¹ is 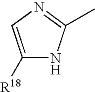
in which R¹⁸ is hydrogen, trifluoromethyl, alkyl, arylalkyl, cycloalkylalkyl, heteroarylalkyl or heterocycloalkylalkyl, hereinafter described as compounds of formula (22), may be prepared as shown in Reaction Scheme 16:

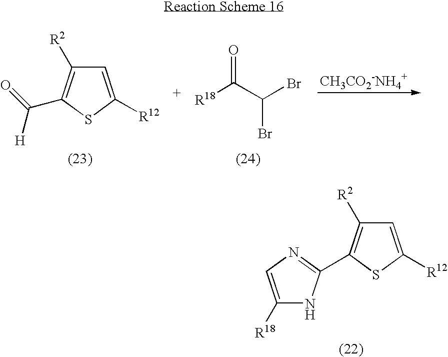

Thus compounds of formula (23), wherein R² and R¹² are as hereinbefore defined, may be reacted with compounds of formula (24), wherein R¹⁸ is as hereinbefore defined, to obtain the said compounds of formula (22). The reaction may conveniently be carried out in an aqueous alcoholic solvent, for example aqueous methanol, in the presence of ammonium acetate, at temperatures of from about room temperature to about the reflux temperature of the solvent.

Compounds of formula (23) and (24) may be obtained from commercial sources, or may be prepared using published methods described in the literature.

Compounds of general formula (5), where R¹² is as hereinbefore defined, and R¹ is 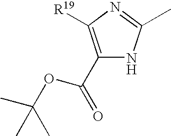
in which R¹⁹ is hydrogen, trifluoromethyl, alkyl, arylalkyl, cycloalkylalkyl, heteroarylalkyl or heterocycloalkylalkyl, hereinafter described as compounds of formula (25), may be prepared as shown in Reaction Scheme 17:

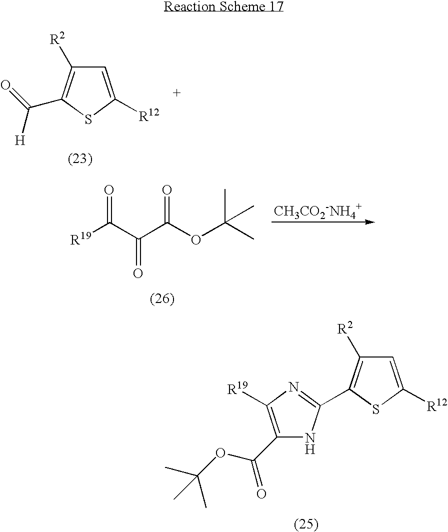

Thus compounds of formula (23), wherein R² and R¹² are hereinbefore described, may be reacted with a compound of formula (26), to obtain the said compounds of formula (25). The reaction may conveniently be carried in a solvent such as acetonitrile, in the presence of ammonium acetate, at temperatures of from about room temperature to about the reflux temperature of the solvent.

Compounds of formula (26) may be prepared using published methods described in the literature, and known to those skilled in the art [Wasserman et al., J. Org. Chem., 2003, 58, 4785-4787].

Compounds of general formula (5), where R² and R¹² are as hereinbefore described, and R¹ is 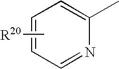
in which R²⁰ is hydrogen, C(═O)OR⁹, halo, CHO, CN, NO₂, NH₂, trifluoroalkyl, alkyl, aryl, heteroaryl, heterocycloalkyl, arylalkyl, heteroarylalkyl, heterocycloalkylalkyl, hereinafter described as compounds of formula (27), may be prepared as shown in Reaction Scheme 18:

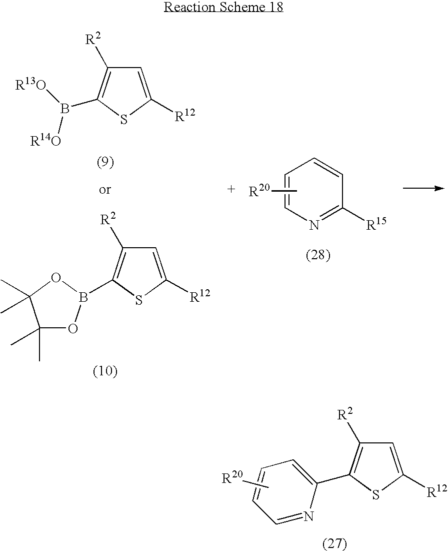

Thus compounds of formula (28), wherein R²⁰ and R¹⁵ are as hereinbefore defined, may be reacted with compounds of formula (9), wherein R², R¹² R¹³ and R¹⁴ are as hereinbefore defined, to obtain compounds of formula (27), wherein R², R¹² and R²⁰ are as hereinbefore defined. The reaction is performed in the presence of a suitable catalyst, such as tetrakis(triphenylphosphine)palladium (0), and a suitable base, such as cesium carbonate, in a suitable solvent, such as N,N-dimethylformamide, and at a temperature from about room temperature up to the reflux temperature of the solvent.

Alternatively, the coupling reaction may also be carried out using compounds of formula (10) wherein R² and R¹² are as hereinbefore defined.

Compounds of formula (28) may be obtained from commercial sources, or may be prepared using published methods described in the literature.

Compounds of formula (5) may be prepared from compounds of formula (29) as shown in Reaction Scheme 19:

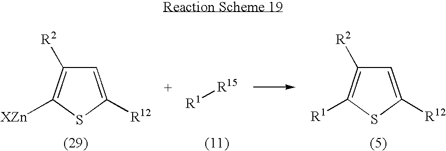

Thus compounds of formula (29), wherein R² and R¹² are as hereinbefore defined, may be coupled with compounds of formula (11), in which R¹⁵ is as hereinbefore defined, to obtain compounds of formula (5), wherein R¹, R² and R¹² are as hereinbefore defined. The reaction is performed in the presence of a suitable catalyst, such as dihydrogen dichlorobis(di-tert-butylphosphinito-κP)palladate (2-), in suitable solvents such as tetrahydrofuran and N-methylpyrrolidinone at a temperature of from about room temperature up to the reflux temperature of the solvent.

Compounds of formula (29) may be prepared using published methods described in the literature, and known to those skilled in the art.

Compounds of formula (5) may be prepared from compounds of formula (6) as shown in Reaction Scheme 20:

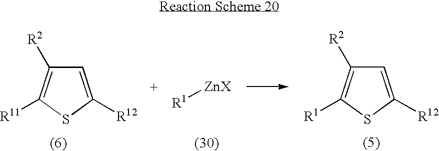

Thus compounds of formula (6), wherein R², R¹¹ and R¹² are as hereinbefore defined, may be coupled with compounds of formula (30), in which R¹ is as hereinbefore defined, to obtain compounds of formula (5), wherein R¹, R² and R¹² are as hereinbefore defined. The reaction is performed in the presence of a suitable catalyst, such as dihydrogen dichlorobis(di-tert-butylphosphinito-κP)palladate (2-), in suitable solvents such as tetrahydrofuran and N-methylpyrrolidinone at a temperature of from about room temperature up to the reflux temperature of the solvent.

Compounds of formula (30) may be prepared using published methods described in the literature, and known to those skilled in the art.

Compounds of formula (5) may be prepared from compounds of formula (31) as shown in Reaction Scheme 21:

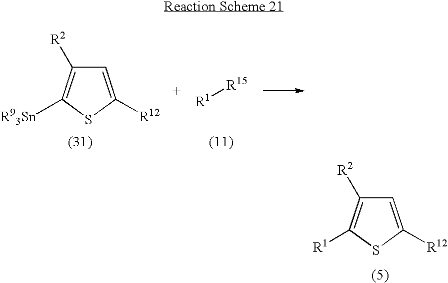

Thus compounds of formula (31), wherein R², R⁹ and R¹² are as hereinbefore defined, may be coupled with compounds of formula (11), in which R¹⁵ is as hereinbefore defined, to obtain compounds of formula (5), wherein R¹, R² and R¹² are as hereinbefore defined. The reaction is performed in the presence of a suitable catalyst, such as tris(dibenzylideneacetone) dipalladium, in a suitable solvent mixture such as N-methylpyrrolidinone at a temperature of from about room temperature up to the reflux temperature of the solvent.

Compounds of formula (31) may be obtained from commercial sources, or may be prepared using published methods described in the literature.

Compounds of formula (5) may be prepared from compounds of formula (6) as shown in Reaction Scheme 22:

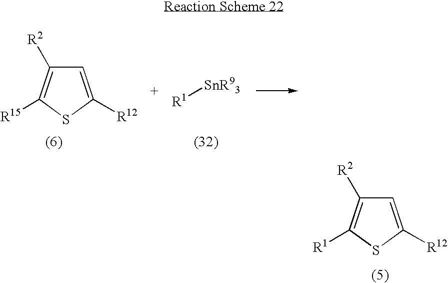

Thus compounds of formula (6), wherein R², R¹² and R¹⁵ are as hereinbefore defined, may be coupled with compounds of formula (32), in which R¹ and R⁹ are as hereinbefore defined, to obtain compounds of formula (5), wherein R¹, R² and R¹² are as hereinbefore defined. The reaction is performed in the presence of a suitable catalyst, such as tris(dibenzylideneacetone) dipalladium, in a suitable solvent such as N-methylpyrrolidinone at a temperature of from about room temperature up to the reflux temperature of the solvent.

Compounds of formula (32) may be obtained from commercial sources, or may be prepared using published methods described in the literature.

Compounds of general formula (5), where R² and R¹² are as hereinbefore described, and R¹ is 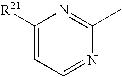
in which R²¹ is hydrogen, trifluoroalkyl, alkyl, aryl, heteroaryl, heterocycloalkyl, arylalkyl, heteroarylalkyl, heterocycloalkylalkyl, hereinafter described as compounds of formula (33), may be prepared as shown in Reaction Scheme 23:

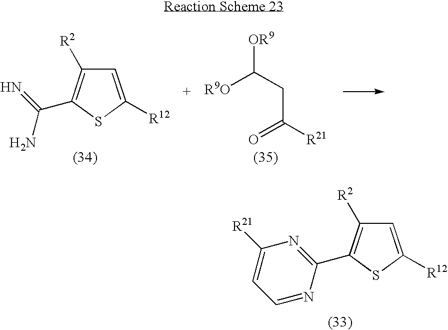

Thus compounds of formula (34), wherein R² and R¹² are as hereinbefore defined, may be reacted with compounds of formula (35), wherein R⁹ and R²¹ is as hereinbefore defined, to obtain compounds of formula (33), wherein R² and R¹² are as hereinbefore defined. The reaction is performed in an alcoholic solvent, such as ethanol, and at a temperature from about room temperature up to the reflux temperature of the solvent.

Compounds of general formula (5), where R¹² is as hereinbefore defined, and R¹ is 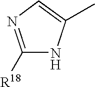
in which R¹⁸ is as hereinbefore defined, hereinafter described as compounds of formula (36), may be prepared as shown in Reaction Scheme 24:

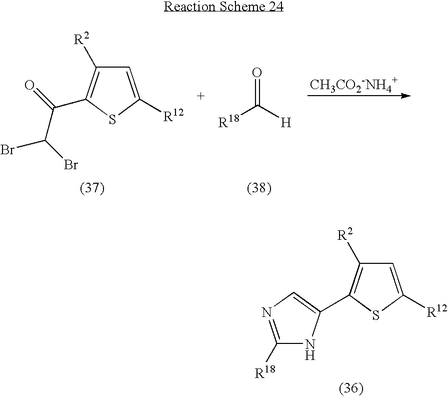

Thus compounds of formula (37), wherein R² and R¹² are as hereinbefore defined, may be reacted with compounds of formula (38), wherein R¹⁸ is as hereinbefore defined, to obtain the said compounds of formula (36). The reaction may conveniently be carried out in an aqueous alcoholic solvent, for example aqueous methanol, in the presence of ammonium acetate, at temperatures of from about room temperature to about the reflux temperature of the solvent.

Compounds of formula (37) may be prepared using published methods described in the literature and compounds of formula (38) may be obtained from commercial sources, or may be prepared using published methods described in the literature [Izawa et al., Bull. Chem. Soc., 1983, 56, 1490-1496].

It will be appreciated that where appropriate functional groups exist, compounds of formula (I) or any preceding intermediates such as intermediates of formula (II), (III), (IV), (1), (2), (3), (4), (5), (15a), (15b), (22), (25) or (27) may be further derivatised by one or more standard synthetic methods employing substitution, oxidation, reduction, or cleavage reactions. Particular substitution approaches include conventional alkylation, arylation, heteroarylation, acylation, sulfonylation, halogenation, nitration, formyalation and coupling procedures.

For example primary amine (—NH₂) groups may be alkylated using a reductive alkylation process employing an aldehyde or a ketone and a borohydride, for example sodium triacetoxyborohydride or sodium cyanoborohydride, in a solvent such as a halogenated hydrocarbon, for example 1,2-dichloroethane, or an alcohol such as ethanol, where necessary in the presence of an acid such as acetic acid at around ambient temperature. Secondary amine (—NH—) groups may be similarly alkylated employing an aldehyde.

In a further example, primary amine or secondary amine groups may be converted into amide groups (—NHCOR′ or —NRCOR′) by acylation. Acylation may be achieved by reaction with an appropriate acid chloride in the presence of a base, such as triethylamine, in a suitable solvent, such as dichloromethane, or by reaction with an appropriate carboxylic acid in the presence of a suitable coupling agent such HATU (O-(7-azabenzotriazol-1-yl)-N,N,N′,N′-tetramethyluronium hexafluorophosphate) in a suitable solvent such as dichloromethane. Similarly, amine groups may be converted into sulphonamide groups (—NHSO₂R′ or —NR″SO₂R′) groups by reaction with an appropriate sulphonyl chloride in the presence of a suitable base, such as triethylamine, in a suitable solvent such as dichloromethane. Primary or secondary amine groups can be converted into urea groups (—NHCONR′R″ or —NRCONR′R″) by reaction with an appropriate isocyanate in the presence of a suitable base such as triethylamine, in a suitable solvent, such as dichloromethane.

An amine (—NH₂) may be obtained by reduction of a nitro (—NO₂) group, for example by catalytic hydrogenation, using for example hydrogen in the presence of a metal catalyst, for example palladium on a support such as carbon in a solvent such as ethyl acetate or an alcohol e.g. methanol. Alternatively, the transformation may be carried out by chemical reduction using for example a metal, e.g. tin or iron, in the presence of an acid such as hydrochloric acid.

In a further example, amine (—CH₂NH₂) groups may be obtained by reduction of nitriles (—CN), for example by catalytic hydrogenation using for example hydrogen in the presence of a metal catalyst, for example palladium on a support such as carbon, or Raney nickel, in a solvent such as an ether e.g. a cyclic ether such as tetrahydrofuran, at a temperature from −78° C. to the reflux temperature of the solvent.

In a further example, amine (—NH₂) groups may be obtained from carboxylic acid groups (—CO₂H) by conversion to the corresponding acyl azide (—CON₃), Curtius rearrangement and hydrolysis of the resultant isocyanate (—N═C═O).

Aldehyde groups (—CHO) may be converted to amine groups (—CH₂NR′R″)) by reductive amination employing an amine and a borohydride, for example sodium triacetoxyborohydride or sodium cyanoborohydride, in a solvent such as a halogenated hydrocarbon, for example dichloromethane, or an alcohol such as ethanol, where necessary in the presence of an acid such as acetic acid at around ambient temperature.

In a further example, aldehyde groups may be converted into alkenyl groups (—CH═CHR″) by the use of a Wittig or Wadsworth-Emmons reaction using an appropriate phosphorane or phosphonate under standard conditions known to those skilled in the art.

Aldehyde groups may be obtained by reduction of ester groups (such as —CO₂Et) or nitriles (—CN) using diisobutylaluminium hydride in a suitable solvent such as toluene. Alternatively, aldehyde groups may be obtained by the oxidation of alcohol groups using any suitable oxidising agent known to those skilled in the art.

Ester groups (—CO₂R′) may be converted into the corresponding acid group (—CO₂H) by acid- or base-catalused hydrolysis, depending on the nature of R. If R is t-butyl, acid-catalysed hydrolysis can be achieved for example by treatment with an organic acid such as trifluoroacetic acid in an aqueous solvent, or by treatment with an inorganic acid such as hydrochloric acid in an aqueous solvent.

Carboxylic acid groups (—CO₂H) may be converted into amides (CONHR′ or —CONR′R″) by reaction with an appropriate amine in the presence of a suitable coupling agent, such as HATU, in a suitable solvent such as dichloromethane.

In a further example, carboxylic acids may be homologated by one carbon (i.e —CO₂H to —CH₂CO₂H) by conversion to the corresponding acid chloride (—COCl) followed by Arndt-Eistert synthesis.

In a further example, —OH groups may be generated from the corresponding ester (e.g. —CO₂R′), or aldehyde (—CHO) by reduction, using for example a complex metal hydride such as lithium aluminium hydride in diethyl ether or tetrahydrofuran, or sodium borohydride in a solvent such as methanol. Alternatively, an alcohol may be prepared by reduction of the corresponding acid (—CO₂H), using for example lithium aluminium hydride in a solvent such as tetrahydrofuran, or by using borane in a solvent such as tetrahydrofuran.

Alcohol groups may be converted into leaving groups, such as halogen atoms or sulfonyloxy groups such as an alkylsulfonyloxy, e.g. trifluoromethylsulfonyloxy or arylsulfonyloxy, e.g. p-toluenesulfonyloxy group using conditions known to those skilled in the art. For example, an alcohol may be reacted with thioyl chloride in a halogenated hydrocarbon (e.g. dichloromethane) to yield the corresponding chloride. A base (e.g. triethylamine) may also be used in the reaction.

In another example, alcohol or phenol groups may be converted to ether groups by coupling a phenol with an alcohol in a solvent such as tetrahydrofuran in the presence of a phosphine, e.g. triphenylphosphine and an activator such as diethyl-, diisopropyl, or dimethylazodicarboxylate. Alternatively ether groups may be prepared by deprotonation of an alcohol, using a suitable base e.g. sodium hydride followed by subsequent addition of an alkylating agent, such as an alkyl halide.

Aromatic halogen substituents in the compounds may be subjected to halogen-metal exchange by treatment with a base, for example a lithium base such as n-butyl or t-butyl lithium, optionally at a low temperature, e.g. around −78° C., in a solvent such as tetrahydrofuran, and then quenched with an electrophile to introduce a desired substituent. Thus, for example, a formyl group may be introduced by using N,N-dimethylformamide as the electrophile. Aromatic halogen substituents may alternatively be subjected to metal (e.g. palladium or copper) catalysed reactions, to introduce, for example, acid, ester, cyano, amide, aryl, heteraryl, alkenyl, alkynyl, thio- or amino substituents. Suitable procedures which may be employed include those described by Heck, Suzuki, Stille, Buchwald or Hartwig.

Aromatic halogen substituents may also undergo nucleophilic displacement following reaction with an appropriate nucleophile such as an amine or an alcohol. Advantageously, such a reaction may be carried out at elevated temperature in the presence of microwave irradiation.

The compositions of the present invention may be formulated in a conventional manner using one or more pharmaceutically acceptable carriers or excipients. Thus, the active compounds of the invention may be formulated for oral, buccal, intranasal, parenteral (e.g., intravenous, intramuscular or subcutaneous) transdermal or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropylmethylcellulose); fillers (e.g. lactose, microcrystalline cellulose or calcium phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, methyl cellulose or hydrogenated edible fats); emulsifying agents (e.g. lecithin or acacia); non-aqueous vehicles (e.g. almond oil, oily esters or ethyl alcohol); and preservatives (e.g. methyl or propyl p-hydroxybenzoates or sorbic acid).

For buccal administration the composition may take the form of tablets or lozenges formulated in conventional manner.

The active compounds of the invention may be formulated for parenteral administration by injection, including using conventional catheterization techniques or infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulating agents such as suspending, stabilising and/or dispersing agents.

Alternatively, the active ingredient may be in powder form for reconstitution with a suitable vehicle, e.g. sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g. dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above is 0.1 to 500 mg of the active ingredient per unit dose which could be administered, for example, 1 to 4 times per day.

The invention will now be described in detail with reference to the following examples. It will be appreciated that the invention is described by way of example only and modification of detail may be made without departing from the scope of the invention.

### EXPERIMENTAL

400 MHz ¹H nuclear magnetic resonance spectra (NMR) were recorded at ambient temperature using a Varian Unity Inova (400 MHz) spectrometer with a triple resonance 5 mm probe. In the NMR chemical shifts (δ) are expressed ppm relative to tetramethylsilane. The following abbreviations have been used: br=broad signal, s=singlet, d=doublet, dd=double doublet, ddd=double double doublet, dt=double triplet, t=triplet, td=triple doublet, q=quartet.

High Pressure Liquid Chromatography—Mass Spectrometry (LCMS) experiments to determine retention times (R_{T}) and associated mass ions were performed using one of the following methods.

Method A: Experiments performed on a Micromass Platform LCT spectrometer with positive ion electrospray and single wavelength UV 254 nm detection using a Higgins Clipeus C18 5 μm 100×3.0 mm column and a 2 ml/minute flow rate. The initial solvent system was 95% water containing 0.1% formic acid (solvent A) and 5% acetonitrile containing 0.1% formic acid (solvent B) for the first minute followed by a gradient up to 5% solvent A and 95% solvent B over the next 14 minutes. The final solvent system was held constant for a further 2 minutes.

Method B: Experiments performed on a Micromass Platform LC spectrometer with positive and negative ion electrospray and ELS/Diode array detection using a Waters XTerra MS C18 3.5 μm 30×4.6 mm column and a 2 ml/minute flow rate. The solvent system was 95% solvent A and 5% solvent B for the first 0.25 minutes followed by a gradient up to 5% solvent A and 95% solvent B over the next 2 minutes. The final solvent system was held constant for a further 0.25 minutes.

Method C: Experiments performed on a Micromass Platform LC spectrometer with positive and negative ion electrospray and ELS/Diode array detection using a Phenomenex Luna C18(2) 30×4.6 mm column and a 2 ml/minute flow rate. The solvent system was 95% solvent A and 5% solvent B for the first 0.50 minutes followed by a gradient up to 5% solvent A and 95% solvent B over the next 4 minutes. The final solvent system was held constant for a further 0.50 minutes.

Reverse Phase High Pressure Liquid Chromatography purification was performed using a Genesis HPLC Column (Ref. 16R10985, 100 mm×22.5 mm) containing C18-7 μm 120A silica.

Reverse Phase purification was performed using a Jones Flashmaster II and IST cartridges (Isolute C18, Octadecyl non-endcapped, sorbent ref: 220).

TLC analysis was performed on Fluka aluminium-backed silica gel/TLC cards (20×20 cm) with layer thickness 0.2 mm, cut to size.

Microwave experiments were carried out using a Personal Chemistry Smith Synthesizer™, which uses a single-mode resonator and dynamic field tuning, both of which give reproducibility and control. Temperature from 40-250° C. can be achieved, and pressures of up to 20 bar can be reached. Two types of vial are available for this processor, 0.5-2.0 mL and 2.0-5.0 mL.

Compounds have been named using Beilstein Autonom software.

### Example 1

### (a) 5-(2-Methyl-5-trifluoromethyl-2H-pyrazol-3-yl)-thiophene-2-carboxylic acid hydroxyamide

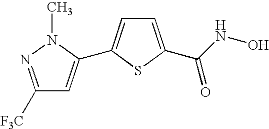

A solution of 5-(2-methyl-5-trifluoromethyl-2H-pyrazol-3-yl)-thiophene-2-carboxylic acid (tetrahydro-pyran-2-yloxy)-amide [29 mg, 0.08 mmol, Reference Example 1(a)] in methanol (0.8 ml) was treated with p-toluene sulfonic acid (0.7 mg, 0.003 mmol). The solution was stirred at room temperature for 1 hour when t.l.c. [ethyl acetate/petroleum ether (b.p. 40-60° C.), 3:2, v/v] indicated complete disappearance of the starting material. The reaction mixture was evaporated under reduced pressure and the residue was partitioned between ethyl acetate and saturated sodium bicarbonate solution. The two phases were separated and the organic phase was washed with water, then dried over sodium sulfate and then evaporated under reduced pressure to give 5-(2-methyl-5-trifluoromethyl-2H-pyrazol-3-yl)-thiophene-2-carboxylic acid hydroxyamide (22 mg, 96%) as a white solid. ¹H NMR (CDCl₃): δ 7.53 (br, 1H), 7.23 (br, 1H), 6.79 (br, 1H), 4.00 (s, 3H). LCMS (Method A): R_{T}=6.45 minutes; 292 (M+H)⁺.

### (b) 5-(2-Methyl-2H-pyrazol-3-yl)-thiophene-2-carboxylic acid hydroxyamide

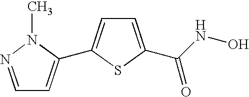

By proceeding in a similar manner to Example 1(a) but using a mixture of 5-(2-methyl-2H-pyrazol-3-yl)-thiophene-2-carboxylic acid (tetrahydro-pyran-2-yloxy)-amide and 5-(1-methyl-1H-pyrazol-3-yl)-thiophene-2-carboxylic acid (tetrahydro-pyran-2-yloxy)-amide [Reference Example 1(b)] there was prepared a mixture of 5-(2-methyl-2H-pyrazol-3-yl)-thiophene-2-carboxylic acid hydroxyamide and 5-(1-methyl-1H-pyrazol-3-yl)-thiophene-2-carboxylic acid hydroxyamide (45 mg, 91%). This was subjected to reverse-phase preparative HPLC (gradient elution, 5% acetonitrile/water to 95% acetonitrile/water over 90 minutes) to provide 5-(2-methyl-2H-pyrazol-3-yl)-thiophene-2-carboxylic acid hydroxyamide (16 mg, 32%) as the more mobile fraction as an off-white solid. ¹H NMR (CD₃OD): δ 7.61 (br, 1H), 7.49 (d, J=2 Hz, 1H), 7.32 (d, j=4 Hz, 1H), 6.53 (d, J=2 Hz, 1H), 3.99 (s, 3H). LCMS (Method A): R_{T}=3.96 minutes; 224 (M+H)⁺.

### (c) 5-(5-Trifluoromethyl-2H-pyrazol-3-yl)-thiophene-2-carboxylic acid hydroxyamide

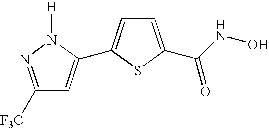

By proceeding in a similar manner to Example 1(a) but using 5-(5-trifluoromethyl-2H-pyrazol-3-yl)-thiophene-2-carboxylic acid (tetrahydro-pyran-2-yloxy)-amide [Reference Example 1(c)] there was prepared 5-(5-trifluoromethyl-2H-pyrazol-3-yl)-thiophene-2-carboxylic acid hydroxyamide (3 mg, 11%) as a fawn coloured solid. ¹H NMR [(CD₃)₂SO]: δ 11.36 (br, 1H), 9.24 (s, 1H), 7.62 (br, 1H), 7.54 (d, J=4.0 Hz, 1H), 7.15 (s, 1H). LCMS (Method A): R_{T}=5.81 minutes; 278 (M+H)⁺.

### (d) 5-(1-Methyl-5-trifluoromethyl-1H-pyrazol-3-yl)-thiophene-2-carboxylic acid hydroxyamide

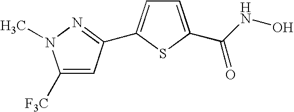

By proceeding in a similar manner to Example 1(a) but using 5-(1-methyl-5-trifluoromethyl-1H-pyrazol-3-yl)-thiophene-2-carboxylic acid (tetrahydro-pyran-2-yloxy)-amide [Reference Example 1(d)] there was prepared 5-(1-methyl-5-trifluoromethyl-1H-pyrazol-3-yl)-thiophene-2-carboxylic acid hydroxyamide as a white solid (8 mg, 95%). ¹H NMR [(CD₃)₂SO]: δ 11.41 (s br, 1H), 9.27 (br, 1H), 7.68 (d br, J=3.9 Hz, 1H), 7.53 (d, J=3.9 Hz, 1H), 7.11 (s, 1H), 4.05 (s, 3H). LCMS (Method A): R_{T}=6.41 minutes; 292 (M+H)⁺.

### (e) 5-(1-Methyl-1H-pyrazol-3-yl)-thiophene-2-carboxylic acid hydroxyamide

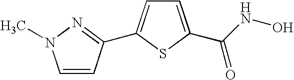

By proceeding in a similar manner to Example 1(a) but using a mixture of 5-(1-methyl-1H-pyrazol-3-yl)-thiophene-2-carboxylic acid (tetrahydro-pyran-2-yloxy)-amide and 5-(2-methyl-2H-pyrazol-3-yl)-thiophene-2-carboxylic acid (tetrahydro-pyran-2-yloxy)-amide [Reference Example 1(b)] there was prepared 5-(1-methyl-1H-pyrazol-3-yl)-thiophene-2-carboxylic acid hydroxyamide (15 mg, 72%) as pale brown oil. ¹H NMR (CD₃OD): δ 7.61 (d, J=2.3 Hz, 1H), 7.52 (br, 1H), 7.32 (d, J=3.9 Hz, 1H), 6.58 (d, J=2.3 Hz, 1H), 3.91 (s, 3H).

### (f) 5-(5-Trifluoromethyl-isoxazol-3-yl]-thiophene-2-carboxylic acid hydroxyamide

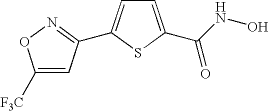

By proceeding in a similar manner to Example 1(a) but using 5-(5-trifluoromethyl-isoxazol-3-yl)-thiophene-2-carboxylic acid (tetrahydro-pyran-2-yloxy)-amide [Reference Example 1(e)] there was prepared 5-(5-trifluoromethyl-isoxazol-3-yl)-thiophene-2-carboxylic acid hydroxyamide (16 mg, 95%) as an off-white solid. ¹H NMR [(CD₃)₂CO]: δ 10.85 (s br, 1H), 8.49 (br, 1H), 7.80 (d, J=3.7 Hz, 1H), 7.76 (br, 1H), 7.75 (s, 1H). LCMS (Method A): R_{T}=6.84 minutes; 279 (M+H)⁺.

### (g) 5-Phenyl-thiophene-2-carboxylic acid hydroxyamide

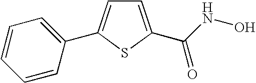

By proceeding in a similar manner to Example 1(a) but using 5-phenyl-thiophene-2-carboxylic acid (tetrahydro-pyran-2-yloxy)-amide [80 mg, Reference Example 1(f)] and washing the white solid obtained after evaporation of the reaction mixture, with water, then twice with dichloromethane, then with saturated sodium bicarbonate solution, then twice with ether and then drying under vacuum there was prepared 5-phenyl-thiophene-2-carboxylic acid hydroxyamide (31 mg, 54%) as a white solid. ¹H NMR [(CD₃)₂SO]: δ 11.20 (s br, 1H), 9.10 (s br, 1H), 7.65 (d, J=8 Hz, 2H), 7.55 (br, 1H), 7.47 (d, J=4.0 Hz, 1H), 7.40 (t, J=8 Hz, 2H), 7.31 (t, J=8 Hz, 1H). LCMS (Method A): R_{T}=6.29 minutes; 220 (M+H4)⁺.

### (h) 5-Pyridin-2-yl-thiophene-2-carboxylic acid hydroxyamide

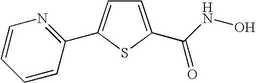

By proceeding in a similar manner to Example 1(a) but using 5-pyridine-2-yl-thiophene-2-carboxylic acid (tetrahydro-pyran-2-yloxy)-amide [228 mg, 0.75 mmol, Reference Example 1(g)] there was prepared 5-pyridin-2-yl-thiophene-2-carboxylic acid hydroxyamide (14 mg, 8%) as a yellow solid. ¹H NMR [(CD₃)₂SO]: δ 11.27 (s, 1H), 9.16 (s, 1H), 8.57 (ddd, J=4.9, 1.7, 0.9 Hz, 1H), 7.96 (dt, J=7.9, 0.9, 0.9 Hz, 1H), 7.87 (td, J=7.9, 7.5, 1.7 Hz, 1H), 7.79 (d, J=4.0 Hz, 1H), 7.62 (br, 1H), 7.34 (ddd, J=7.5, 4.9, 0.9 Hz, 1H). LCMS (Method A): R_{T}=4.11 minutes; 221 (M+H)⁺.

### i) [2,2′]Bithiophenyl-5-carboxylic acid hydroxyamide

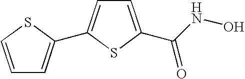

By proceeding in a similar manner to Example 1(a) but using [2,2′]bithiophenyl-5-carboxylic acid (tetrahydro-pyran-2-yloxy)-amide [Reference Example 1(h)] and subjecting the reaction mixture to column chromatography there was prepared [2,2′]bithiophenyl-5-carboxylic acid hydroxyamide (54 mg, 38%) as a brown solid. ¹H NMR [(CD₃)₂SO]: δ 11.27 (s br, 1H), 9.17 (s br, 1H), 7.59 (d, J=5.1 Hz, 1H), 7.55 (br, 1H), 7.41 (d, J=3.4 Hz, 1H), 7.30 (d, J=3.7 Hz, 1H), 7.12 (dd, J=5.1, 3.7 Hz, 1H). LCMS (Method A): R_{T}=5.99 minutes; 226 (M+H)⁺.

### (j) 5-(4-Methoxy-phenyl)-thiophene-2-carboxylic acid hydroxyamide

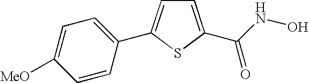

By proceeding in a similar manner to Example 1(a) but using 5-(4-methoxy-phenyl)-thiophene-2-carboxylic acid (tetrahydro-pyran-2-yloxy)-amide [Reference Example 1(i)] there was prepared 5-(4-methoxy-phenyl)-thiophene-2-carboxylic acid hydroxyamide (78 mg, 96%) as a pale yellow solid. ¹H NMR (CD₃OD): δ 7.60 (d, J=8.8 Hz, 2H), 7.53 (br, 1H), 7.26 (d, J=4.0 Hz, 1H), 6.97 (d, J=8.8 Hz, 2H), 3.82 (s, 3H). LCMS (Method A): R_{T}=6.39 minutes; 250 (M+H)⁺.

### (k) 5-(2H-Pyrazol-3-yl)-thiophene-2-carboxylic acid hydroxyamide

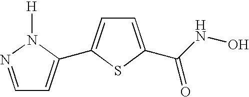

By proceeding in a similar manner to Example 1(a) but using 5-(2H-pyrazol-3-yl)-thiophene-2-carboxylic acid (tetrahydro-pyran-2-yloxy)-amide [120 mg, 0.40 mmol, Reference Example 1(j)] and subjecting the reaction mixture to reverse-phase HPLC (gradient elution, 5% acetonitrile/water to 95% acetonitrile/water over 90 minutes) there was prepared 5-(2H-pyrazol-3-yl)-thiophene-2-carboxylic acid hydroxyamide (79 mg, 92%) as a white solid. ¹H NMR (CD₃OD): δ 7.69 (d, J=2.3 Hz, 1H), 7.54 (br, 1H), 7.36 (d, J=4.0 Hz, 1H), 6.64 (d, J=2.3 Hz, 1H). LCMS (Method A): R_{T}=3.49 minutes; 210 (M+H)⁺.

### (l) 5-(1-Benzyl-1H-pyrazol-3-yl)-thiophene-2-carboxylic acid hydroxyamide

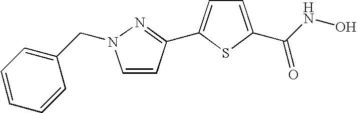

By proceeding in a similar manner to Example 1(a) but using 5-(1-benzyl-1H-pyrazol-3-yl)-thiophene-2-carboxylic acid (tetrahydro-pyran-2-yloxy)-amide [Reference Example 1(k)] and purification of the reaction mixture by preparative reverse-phase HPLC (gradient elution, 5% acetonitrile/water to 95% acetonitrile/water over 90 minutes) there was prepared 5-(1-benzyl-1H-pyrazol-3-yl-thiophene-2-carboxylic acid hydroxyamide (54 mg, 96%) as a pale brown solid. ¹H NMR (CD₃OD): δ 7.66 (d, J=2.3 Hz, 1H), 7.52 (br, 1H), 7.24-7.36 (m, 6H), 6.62 (d, J=2.3 Hz, 1H), 5.35 (s, 2H). LCMS (Method A): R_{T}=6.54 minutes; 300 (M+1)⁺.

### (m) 5-(1-Phenethyl-1H-pyrazol-3-yl)-thiophene-2-carboxylic acid hydroxyamide

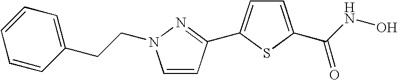

By proceeding in a similar manner to Example 1(a) but using 5-(1-phenethyl-1H-pyrazol-3-yl)-thiophene-2-carboxylic acid (tetrahydro-pyran-2-yloxy)-amide [Reference Example 1(l)] there was prepared 5-(1-phenethyl-1H-pyrazol-3-yl)-thiophene-2-carboxylic acid hydroxyamide (121 mg, 97%) as a pale brown solid. ¹H NMR (CD₃OD): δ 7.53 (br, 1H), 7.39 (d, J=2.3 Hz, 1H), 7.32 (d, J=4.0 Hz, 1H), 7.25 (m, 2H), 7.18 (m, 1H), 7.12 (m, 2H), 6.49 (d, J=2.3 Hz, 1H1), 4.37 (t, J=7.2 Hz, 2H), 3.16 (t, J=7.2 Hz, 2H). LCMS (Method A): R_{T}=7.02 minutes; 314 (M+H)⁺.

### (n) 5-(4-Trifluoromethyl-1H-imidazol-2-yl)-thiophene-2-carboxylic acid hydroxyamide

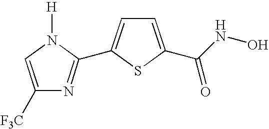

By proceeding in a similar manner to Example 1(a) but using 5-(4-trifluoromethyl-1H-imidazol-2-yl)-thiophene-2-carboxylic acid (tetrahydro-pyran-2-yloxy)-amide [42 mg, 0.15 mmol, Reference Example 1(m)] and triturating the reaction mixture with water there was prepared 5-(4-trifluoromethyl-1H-imidazol-2-yl)-thiophene-2-carboxylic acid hydroxyamide (8 mg, 25%) as a white powder. ¹H NMR [(CD₃)₂SO]: δ 13.42 (s, 1H), 11.32 (s, 1H), 9.21 (s, 1H), 7.96 (s, 1H), 7.60 (s, 2H). LCMS (Method A): R_{T}=4.85 minutes; 278 (N+H)⁺.

### (o) 5-(3-Methyl-[1,2,4]oxadiazol-5-yl)-thiophene-2-carboxylic acid hydroxyamide

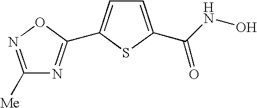

By proceeding in a similar manner to Example 1(a) but using 5-(3-methyl-[1,2,4]oxadiazol-5-yl)-thiophene-2-carboxylic acid (tetrahydro-pyran-2-yloxy)-amide [155 mg, 0.48 mmol, Reference Example 1(n)], filtering the resulting precipitate (which was then washed with methanol) there was prepared 5-(3-methyl-[1,2,4]oxadiazol-5-yl-thiophene-2-carboxylic acid hydroxyamide (65 mg, 60%) as a white solid. ¹H NMR [(CD₃)₂SO]: δ 11.60 (s, 1H), 9.41 (s, 1H), 7.98 (d, J=4.0 Hz, 1H), 7.73 (d br, J=4.0 Hz, 1H), 2.41 (s, 3H). LCMS (Method A): R_{T}=4.26 minutes; 226 (M+H)⁺.

### (p) 5-[1-(2-Benzyloxy-ethyl-1H-pyrazol-3-yl]-thiophene-2-carboxylic acid hydroxyamide

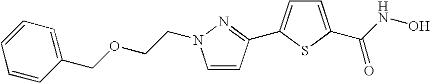

A solution of 5-[1-(2-benzyloxy-ethyl)-1H-pyrazol-3-yl]-thiophene-2-carboxylic acid methyl ester [50 mg, 0.15 mmol, Reference Example 10(a)] in methanol (400 μl), was treated with a suspension of hydroxylamine hydrochloride (70 mg, 1 mmol) and potassium hydroxide (84 mg, 1.5 mmol) in methanol (350 μl). The reaction mixture was stirred overnight in a sealed tube. A further suspension of hydroxylamine hydrochloride (50 mg) and potassium hydroxide (60 mg, 1.5 mmol) in methanol (500 μl) was added to the reaction mixture. After stirring over the weekend the reaction suspension was concentrated to give a yellow waxy solid, to which a citric acid/water (1:1) solution was added, and then this was extracted with ethyl acetate (2×). The organic phases were combined, dried (MgSO₄) and evaporated to give an amber gum which was subjected to reverse-phase preparative HPLC using acetonitrile and water (gradient 20:80 to 95:5, v/v, over 75 minutes) as eluent, to provide 5-[1-(2-benzyloxy-ethyl)-1H-pyrazol-3-yl]-thiophene-2-carboxylic acid hydroxyamide (8 mg) as a faun glass. ¹H NMR [(CD₃)₂SO]: δ 11.18 (s br, 1H), 9.05 (s br, 1H), 7.81 (d, 1H), 7.55 (apparent s br, 1H), 7.37 (d, 1H), 7.22-7.34 (m, 5H), 6.66 (d, 1H), 4.48 (s, 2H), 4.34 (t, 2H), 3.81 (t, 2H). LCMS (Method A): R_{T}=6.54 minutes; 344 (+H)⁺.

### (q) 5-[1-(3-Phenyl-propyl)-H-pyrazol-3-yl]-thiophene-2-carboxylic acid hydroxyamide

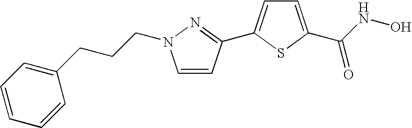

A solution of 5-[1-(3-phenyl-propyl)-1H-pyrazol-3-yl]-thiophene-2-carboxylic acid (tetrahydro-pyran-2-yloxy)-amide [282 mg, 0.68 mmol, Reference Example 1(o)] in methanol (10 ml) was treated with Amberlyst 15 ion exchange resin (664 mg). The mixture was stirred slowly at room temperature overnight then filtered, and the resin was washed several times with methanol. The organic filtrate was concentrated to give a residue which was triturated with diethyl ether followed by ethyl acetate. The ethyl acetate layer from the second trituration was concentrated to give a residue which was subjected to reverse phase purification using acetonitrile and water as eluent, to provide 5-[1-(3-phenyl-propyl)-1H-pyrazol-3-yl]-thiophene-2-carboxylic acid hydroxyamide (31 mg) as a pink gum. ¹H NMR (CD₃OD): δ 7.62 (d, 1H), 7.53 (apparent s br, 1H), 7.33 (d, 1H), 7.26 (apparent t, 2H), 7.18 (apparent d, 2H), 7.16 (apparent t, 1H), 6.58 (d, 1H), 4.15 (t, 2H), 2.61 (t, 2H), 2.18 (m, 2H). LCMS (Method A): R_{T}=7.33 minutes; 328 (M+H)⁺.

### (r) 5-[1-(2,3-Dihydro-benzo[1,4]dioxin-2-ylmethyl)-1H-pyrazol-3-yl]-thiophene-2-carboxylic acid hydroxyamide

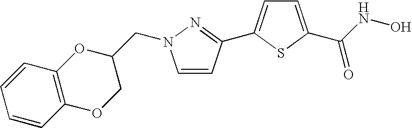

A solution of 5-[1-(2,3-dihydro-benzo[1,4]dioxin-2-ylmethyl)-1H-pyrazol-3-yl]-thiophene-2-carboxylic acid (tetrahydro-pyran-2-yloxy)-amide [65 mg, 0.15 mmol, Reference Example 1(p)] in methanol (4 ml) was treated with p-toluene sulfonic acid (1.4 mg, 0.007 mmol). The solution was stirred at room temperature overnight, and then evaporated to dryness under reduced pressure to provide 5-[1-(2,3-dihydro-benzo[1,4]dioxin-2-ylmethyl)-1H-pyrazol-3-yl]-thiophene-2-carboxylic acid hydroxyamide (46 mg) as a brown solid. ¹H NMR (CD₃OD): δ 7.69 (d, 1H), 7.53 (apparent s br, 1H), 7.35 (d, 1H), 6.80-6.87 (m, 4H), 6.63 (d, 1H), 4.58 (m, 1H), 4.47 (d, 2H), 4.33 (dd, 1H), 3.94 (dd, 1H). LCMS (Method C): R_{T}=2.90 minutes; 358 (M+H)⁺.

### (s) 5-{1-[2-(4-Trifluoromethyl-phenyl-ethyl]-1H-pyrazol-3-yl}-thiophene-2-carboxylic acid hydroxyamide

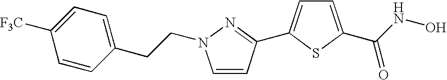

To solution of 5-{1-[2-(4-trifluoromethyl-phenyl)-ethyl]-1H-pyrazol-3-yl}-thiophene-2-carboxylic acid (tetrahydro-pyran-2-yloxy)-amide [226 mg, 0.55 mmol, Reference Example 1(q)] in dichloromethane (2 ml) was added trifluoroacetic acid (1 ml). The mixture was stirred at room temperature for 4 hours, and then concentrated in vacuo. The residue was subjected to reverse phase purification using acetonitrile and water (gradient 5:95 to 95:5, v/v, over 30 minutes) to provide 5-{1-[2-(4-trifluoromethyl-phenyl)-ethyl]-1H-pyrazol-3-yl}-thiophene-2-carboxylic acid hydroxyamide (30 mg) as a white solid. ¹H NMR [(CD₃)₂SO]: δ 11.19 (s br, 1H), 9.10 (s br, 1H), 7.68 (d, 1H), 7.63 (d, 2H), 7.55 (apparent s, 1H), 7.41 (d, 2H), 7.35 (d, 1H), 6.60 (d, 1H), 4.42 (t, 2H), 3.24 (t, 2H). LCMS (Method A): R_{T}=7.75 minutes; 382 (M+H)⁺.

### (t) 5-(1-Benzo[1,3]dioxol-5-ylmethyl-1H-pyrazol-3-yl)-thiophene-2-carboxylic acid hydroxyamide

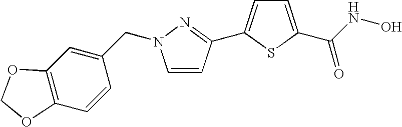

A solution of 5-(1-benzo[1,3]dioxol-5-ylmethyl-1H-pyrazol-3-yl)-thiophene-2-carboxylic acid (tetrahydro-pyran-2-yloxy)-amide [152 mg, 0.35 mmol, Reference Example 1(r)] in methanol (10 ml) was treated with p-toluene sulfonic acid (37 mg, 0.19 mmol), and the solution was stirred at room temperature overnight. The reaction mixture was concentrated under reduced pressure, and subjected to reverse-phase preparative HPLC using acetonitrile and water (gradient 5:95 to 95:5, over 90 minutes) as eluent, to provide 5-(1-benzo[1,3]dioxol-5-ylmethyl-1H-pyrazol-3-yl)-thiophene-2-carboxylic acid hydroxyamide (78 mg). ¹H NMR (CD₃OD): δ 7.63 (d, 1H), 7.52 (apparent s br, 1H), 7.32 (d, 1H), 6.74-6.80 (m, 3H), 6.59 (d, 1H), 5.90 (s, 2H), 5.21 (s, 2H). LCMS (Method A): R_{T}=5.60 minutes; 344 (M+H)⁺.

### (u) 5-{1-[2-(4-Trifluoromethoxy-phenyl-ethyl]-1H-pyrazol-3-yl}-thiophene-2-carboxylic acid hydroxyamide

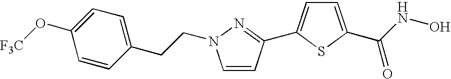

A solution of 5-{1-[2-(4-trifluoromethoxy-phenyl)-ethyl]-1H-pyrazol-3-yl}-thiophene-2-carboxylic acid (tetrahydro-pyran-2-yloxy)-amide [231 mg, 0.47 mmol, Reference Example 1(s)] in methanol (5 ml) was treated with p-toluene sulfonic acid (6.4 mg, 0.03 mmol), and the solution was stirred at room temperature overnight. The reaction mixture was evaporated to dryness under reduced pressure, to provide 5-{1-[2-(4-trifluoromethoxy-phenyl)-ethyl]-1H-pyrazol-3-yl}-thiophene-2-carboxylic acid hydroxyamide (175 mg) as a light brown solid. ¹H NMR (CD₃OD): δ 7.53 (apparent s br, 1H), 7.43 (d, 1H), 7.32 (d, 1H), 7.21 (apparent d, 2H), 7.15 (apparent d, 2H), 6.51 (d, 1H), 4.39 (t, 2H), 3.20 (t, 2H). LCMS (Method A): R_{T}=8.04 minutes; 398 (M+H)⁺.

### (v) 5-{1-[2-(4-Fluoro-phenyl-ethyl]-1H-pyrazol-3-yl}-thiophene-2-carboxylic acid hydroxyamide

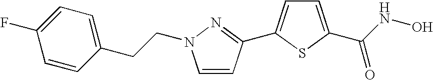

A solution of 5-{1-[2-(4-fluoro-phenyl)-ethyl]-1H-pyrazol-3-yl}-thiophene-2-carboxylic acid (tetrahydro-pyran-2-yloxy)-amide [120 mg, 0.29 mmol, Reference Example 1(t)] in methanol (10 ml) was treated with Amberlyst 15 ion exchange resin (100 mg). The mixture was stirred slowly at room temperature for 1 hour then filtered, and the resin was washed several times with methanol. The organic filtrate was concentrated to give a residue, which was subjected to reverse phase purification using acetonitrile and water (gradient 0:100 to 100:0, v/v, in 10% intervals) as eluent, to provide 5-{1-[2-(4-fluoro-phenyl)-ethyl]-1H-pyrazol-3-yl}-thiophene-2-carboxylic acid hydroxyamide (30.7 mg) as a white solid. ¹H NMR (CD₃OD): δ 11.17 (s br, 1H), 9.10 (s br, 1H), 7.66 (d, 1H), 7.54 (apparent s br, 1H), 7.35 (d, 1H), 7.21 (apparent dd, 2H), 7.09 (apparent t, 2H), 6.59 (d, 1H), 4.35 (t, 2H), 3.11 (t, 2H). LCMS (Method A): R_{T}=5.86 minutes; 332 (M+H)⁺.

### (w) 5-[1-(1-Phenyl-ethyl)-1H-pyrazol-3-yl]-thiophene-2-carboxylic acid hydroxyamide

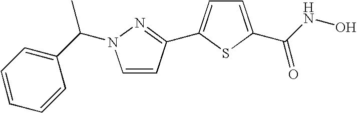

A solution of 5-[1-(1-phenyl-ethyl)-1H-pyrazol-3-yl]-thiophene-2-carboxylic acid (tetrahydro-pyran-2-yloxy)-amide [214 mg, 0.54 mmol, Reference Example 1(u)] in methanol (10 ml) was treated with Amberlyst 15 ion exchange resin (100 mg). The mixture was stirred slowly at room temperature for 1 hour then filtered, and the resin was washed several times with methanol. The organic filtrate was concentrated to give a residue, which was subjected to reverse phase purification twice, using acetonitrile and water (gradient 0:100 to 100:0, v/v, in 10% intervals) as eluent each time, to provide 5-[1-(1-phenyl-ethyl)-1H-pyrazol-3-yl]-thiophene-2-carboxylic acid hydroxyamide (30.5 mg) as a grey gum. ¹H NMR (CD₃OD): δ 7.70 (d, 1H), 7.51 (apparent s br, 1H), 7.30-7.36 (m, 3H), 7.24-7.29 (m, 3H), 6.61 (d, 1H), 5.59 (q, 1H), 1.89 (d, 3H). LCMS (Method A): R_{T}=5.95 minutes; 314 (M+H)⁺.

### (x) 5-[1-(2-Morpholin-4-yl-ethyl)-1H-pyrazol-3-yl]-thiophene-2-carboxylic acid hydroxyamide

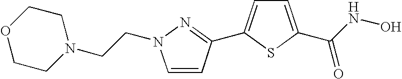

A solution of 5-[1-(2-morpholin-4-yl-ethyl)-1H-pyrazol-3-yl]-thiophene-2-carboxylic acid (tetrahydro-pyran-2-yloxy)-amide [96 mg, 0.24 mmol, Reference Example 1(v)] in methanol (2.4 ml) was treated with Amberlyst 15 ion exchange resin (180 mg). The mixture was stirred slowly at room temperature for 1 hour, then concentrated hydrochloric acid (1.5 ml) was added, and the mixture was stirred for a further 1 hour. The resin was filtered off, washed twice with dioxane, and the filtrate was concentrated to give a colourless glass. The colourless glass was triturated with diethyl ether, dichloromethane and methanol, to provide 5-[1-(2-morpholin-4-yl-ethyl)-1H-pyrazol-3-yl]-thiophene-2-carboxylic acid hydroxyamide (4.8 mg) as a white solid. ¹H NMR (CD₃OD): δ 11.22 (s br, 1H), 10.68 (s br, 1H), 9.12 (s br, 1H), 7.91 (d, 1H), 7.57 (apparent s br, 11), 7.41 (d, 1H), 6.75 (d, 1H), 4.63 (s br, 2H), 3.98 (d br, 2H), 3.74 (t br, 2H), 3.62 (s br, 2H), 3.44 (d br, 2H), 3.14 (s br, 2H). LCMS (Method A): R_{T}=0.38 minutes; 323 (+H)⁺.

### (y) 5-[1-(Tetrahydro-pyran-2-ylmethyl)-1H-pyrazol-3-yl]-thiophene-2-carboxylic acid hydroxyamide

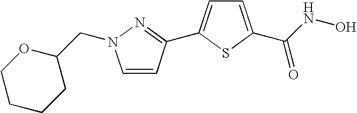

A solution of 5-[1-(tetrahydro-pyran-2-ylmethyl)-1H-pyrazol-3-yl]-thiophene-2-carboxylic acid (tetrahydro-pyran-2-yloxy)-amide [248 mg, 0.63 mmol, Reference Example 1(w)] in methanol (5 ml) was treated with p-toluene sulfonic acid (6 mg, 0.03 mmol), and the solution was stirred at room temperature overnight. An additional amount of p-toluene sulfonic acid (6 mg, 0.03 mmol) was added and the reaction mixture was once again stirred overnight. The reaction mixture was evaporated under reduced pressure and the residue was partitioned between ethyl acetate and saturated sodium bicarbonate solution. The two phases were separated; the organic phase was washed with brine, dried (Na₂SO₄), and evaporated under reduced pressure, to provide 5-[1-(tetrahydro-pyran-2-ylmethyl)-1H-pyrazol-3-yl]-thiophene-2-carboxylic acid hydroxyamide (184 mg) as a white solid. ¹H NMR (CD₃OD): δ 7.62 (d, 1H), 7.52 (apparent s br, 1H), 7.32 (d, 1H), 6.57 (d, 1H), 4.16 (dd, 1H), 4.12 (dd, 1H), 3.93 (m, 1), 3.71 (m, 1H), 3.40 (m, 1H), 1.86 (m, 1H), 1.48-1.66 (m, 4H), 1.27 (m, 1H). LCMS (Method A): R_{T}=4.71 minutes; 308 (M+H)⁺.

### (z) 5-(4-Benzyloxy-pyrimidin-2-yl)-thiophene-2-carboxylic acid hydroxyamide

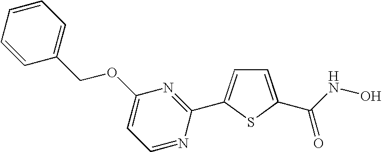

To solution of 5-(4-benzyloxy-pyrimidin-2-yl)-thiophene-2-carboxylic acid (tetrahydro-pyran-2-yloxy)-amide [123 mg, 0.3 mmol, Reference Example 1(x)] in dichloromethane (10 ml) was added trifluoroacetic acid (1 ml). The mixture was stirred at room temperature for 4 hours, and then concentrated in vacuo. The residue was subjected to reverse phase purification using acetonitrile and water (gradient 5:95 to 95:5, v/v, over 30 minutes) as eluent, to provide 5-(4-benzyloxy-pyrimidin-2-yl)-thiophene-2-carboxylic acid hydroxyamide (13 mg) as a gum. ¹H NMR [(CD₃)₂SO]: δ 11.39 (s br, 1H), 9.24 (s br, 1H), 8.56 (d, 1H), 7.93 (d, 1H), 7.65 (d br, 1H), 7.53 (m, 2H), 7.41 (m, 2H), 7.35 (m, 1H), 6.90 (d, 1H), 5.52 (s, 2H). LCMS (Method A): R_{T}=7.22 minutes; 328 (M+H)⁺.

### (aa) 5-(5-Phenethyl-1H-pyrazol-3-yl)-thiophene-2-carboxylic acid hydroxyamide

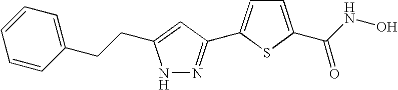 A solution of 5-(5-phenethyl-1H-pyrazol-3-yl)-thiophene-2-carboxylic acid (tetrahydro-pyran-2-yloxy)-amide [200 mg, 0.5 mmol, Reference Example 1(y)] in methanol (20 ml) was treated with p-toluene sulfonic acid (19 mg, 0.1 mmol), and after a short time period (˜30 min) no reaction appeared to have occurred by TLC. An additional amount of p-toluene sulfonic acid (19 mg, 0.1 mmol) was added and the reaction mixture was stirred overnight. The reaction mixture was evaporated under reduced pressure and the residue was partitioned between ethyl acetate and saturated sodium bicarbonate solution. The two phases were separated; the organic phase was washed with brine, dried (Na₂SO₄), and evaporated under reduced pressure, to provide 5-(5-phenethyl-1H-pyrazol-3-yl)-thiophene-2-carboxylic acid hydroxyamide (92 mg) as an off-white solid. ¹H NMR [(CD₃)₂SO]: δ 12.75 (s, 1H), 11.17 (s br, 1H), 9.09 (s br, 1H), 7.53 (apparent s br, 1H), 7.22-7.32 (m, 5H), 7.19 (m, 1H), 6.43 (s, 1H), 2.93 (s br, 4H). LCMS (Method C): R_{T}=2.75 minutes; 314 (M+H)⁺.

### (ab) 5-(2-Phenethyl-3H-imidazol-4-yl)-thiophene-2-carboxylic acid hydroxyamide

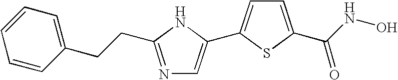

A solution of 5-(2-phenethyl-3H-imidazol-4-yl)-thiophene-2-carboxylic acid (tetrahydro-pyran-2-yloxy)-amide [16 mg, 0.04 mmol, Reference Example 1(z)] in methanol (5 ml) was treated with p-toluene sulfonic acid (30 mg, 0.16 mmol), and the reaction mixture was stirred overnight. The reaction mixture was evaporated under reduced pressure, and the residue was partitioned between ethyl acetate and saturated sodium bicarbonate solution. The two phases were separated; the organic phase was washed with brine, dried (Na₂SO₄), and evaporated under reduced pressure to give a yellow solid. The yellow solid was triturated with diethyl ether and dried under vacuum, to provide 5-(2-phenethyl-3H-imidazol-4-yl)-thiophene-2-carboxylic acid hydroxyamide (2 mg) as yellow solid. ¹H NMR (CD₃OD): δ 7.50 (apparent s br, 1H), 7.21-7.30 (m, 4H), 7.14-7.21 (m, 3H), 4.36 (m, 4H). LCMS (Method A): R_{T}=3.64 minutes; 314 (M+H)⁺.

### (ac) 5-Pyrimidin-2-yl-thiophene-2-carboxylic acid hydroxyamide

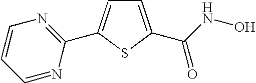

To solution of 5-pyrimidin-2-yl-thiophene-2-carboxylic acid (tetrahydro-pyran-2-yloxy)-amide [392 mg, 1.29 mmol, Reference Example 1(aa)] in dichloromethane (6 ml) was added trifluoroacetic acid (0.12 ml) and water (2 drops). The mixture was stirred at room temperature for 2 hours, after which a precipitate was observed. The precipitate was filtered and washed with dichloromethane and dried under vacuum, to provide 5-pyrimidin-2-yl-thiophene-2-carboxylic acid hydroxyamide (110 mg) as an off-white solid. ¹H NMR [(CD₃)₂SO]: δ 11.39 (s br, 1H), 8.85 (d, 2H), 7.92 (d, 1H), 7.65 (d br, 1H), 7.43 (t, 1H). LCMS (Method A): R_{T}=3.39 minutes; 222 (M+H)⁺.

### (ad) 5-(1-Phenethyl-5-trifluoromethyl-1H-pyrazol-3-yl)-thiophene-2-carboxylic acid hydroxyamide

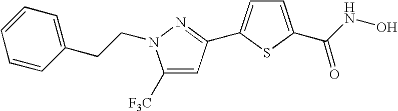

A solution of 5-(1-phenethyl-5-trifluoromethyl-1H-pyrazol-3-yl)-thiophene-2-carboxylic acid (tetrahydro-pyran-2-yloxy)-amide [107 mg, 0.23 mmol, Reference Example 1(ab)] in methanol (10 ml) was treated with p-toluene sulfonic acid (42 mg, 0.22 mmol), and the reaction mixture was stirred overnight. The reaction mixture was evaporated under reduced pressure and the residue was partitioned between ethyl acetate and saturated sodium bicarbonate solution. The two phases were separated; the organic phase was washed with brine, dried (Na₂SO₄), and concentrated under reduced pressure to give 79 mg of a residue. 33 mg of the residues was subjected to reverse-phase preparative HPLC using acetonitrile and water (gradient 5:95 to 45:55, v/v, over 40 minutes; then 45:55 to 90:10, v/v, over the following 30 minutes) as eluent, to provide 5-(1-phenethyl-5-trifluoromethyl-1H-pyrazol-3-yl)-thiophene-2-carboxylic acid hydroxyamide (21.6 mg). ¹H NMR (CD₃OD): δ 7.54 (apparent s br, 1H), 7.41 (d, 1H), 7.26 (m, 2H), 7.19 (m, 1H), 7.14 (m, 2H), 7.05 (s, 1H), 4.44 (t, 2H), 3.22 (t, 2H). LCMS (Method A): R_{T}=7.66 minutes; 382 (M+H)⁺.

### (ae) 5-Pyridin-3-yl-thiophene-2-carboxylic acid hydroxyamide

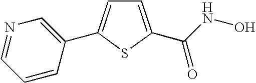

A solution of 5-pyridin-3-yl-thiophene-2-carboxylic acid (tetrahydro-pyran-2-yloxy)-amide [120 mg, 0.39 mmol, Reference Example 1(ac)] in methanol (5 ml) was treated with Amberlyst 15 ion exchange resin (250 mg). The mixture was stirred slowly at room temperature for 3 hours, then 1M hydrochloric acid (1 ml) was added and the mixture was stirred for a further 20 minutes. The resin was filtered off, washed twice with water, and the filtrate was concentrated to give a residue. The residue was dissolved in water (3 ml), then lyophilised to give an off-white solid, which was suspended in a minimal amount of ethanol and diluted with a minimal amount of diethyl ether. The remaining solid was filtered and dried under vacuum, to provide 5-pyridin-3-yl-thiophene-2-carboxylic acid hydroxyamide (13.5 mg) as a grey powder. ¹H NMR [(CD₃)₂SO]: δ 11.40 (s br, 1H), 9.09 (d, 1H), 8.67 (dd, 1H), 8.40 (d, 1H), 7.75 (d, 1H), 7.73 (dd, 1H), 7.69 (d br, 1H). LCMS (Method C): R_{T}=0.33 minutes; 221 (M+H)⁺.

### (af) 5-Pyridin-4-yl-thiophene-2-carboxylic acid hydroxyamide

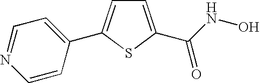

A solution of 5-pyridin-4-yl-thiophene-2-carboxylic acid (tetrahydro-pyran-2-yloxy)-amide [147 mg, 0.48 mmol, Reference Example 1(ad)] in methanol (5 ml) was treated with Amberlyst 15 ion exchange resin (250 mg). The mixture was stirred slowly at room temperature for 2 hours, then 1M hydrochloric acid (1 ml) was added and the mixture was stirred for a further 20 minutes. The resin was filtered off, washed twice with water, and the filtrate was concentrated to give a residue. The residue was dissolved in water (3 ml), then lyophilised to give a pale yellow solid, which was triturated with ethanol and diethyl ether. The remaining solid was filtered and dried under vacuum, to provide 5-pyridin-4-yl-thiophene-2-carboxylic acid hydroxyamide (10.1 mg) as a pale yellow powder. ¹H NMR [(CD₃)₂SO]: δ 11.52 (s br, 1H), 9.37 (s br, 1H), 8.79 (d, 2H), 8.09 (d, 2H), 8.05 (d, 1H), 7.74 (d, 1H). LCMS (Method C): R_{T}=0.35 minutes; 221 (M+H)⁺.

### (ag) 5-(5-Trifluoromethyl-1H-[1,2,4]triazol-3-yl)-thiophene-2-carboxylic acid hydroxyamide

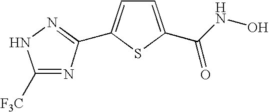

A solution of 5-(5-trifluoromethyl-1H-[1,2,4]triazol-3-yl)-thiophene-2-carboxylic acid (tetrahydro-pyran-2-yloxy)-amide [60 mg, 0.16 mmol, Reference Example 1(ae)] in methanol (5 ml) was treated with p-toluene sulfonic acid (18 mg, 0.09 mmol), and the solution was stirred at room temperature overnight. The reaction mixture was concentrated under reduced pressure and subjected to reverse-phase preparative HPLC using acetonitrile and water (gradient 5:95 to 95:5, v/v, over 100 minutes) as eluent, to provide 5-(5-trifluoromethyl-1H-[1,2,4]triazol-3-yl)-thiophene-2-carboxylic acid hydroxyamide (14 mg). ¹H NMR [(CD₃)₂SO]: δ 11.45 (s br, 1H), 9.29 (s br, 1H), 7.75 (d, 1H), 7.67 (d br, 1H). LCMS (Method A): R_{T}=4.27 minutes; 279 (M+H)⁺.

### (ah) 5-[5-(3-Phenyl-propionylamino)-pyridin-2-yl]-thiophene-2-carboxylic acid hydroxyamide

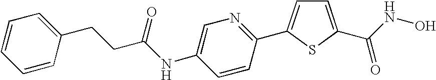

A solution of 5-[5-(3-phenyl-propionylamino)-pyridin-2-yl]-thiophene-2-carboxylic acid methyl ester [69 mg, 0.2 mmol, Reference Example 11(a)] in dioxane (3 ml), was treated with a solution of hydroxylamine hydrochloride (348 mg, 1 mmol) and potassium hydroxide (412 mg, 1.6 mmol) in methanol (2 ml). The reaction mixture was stirred overnight, then concentrated to remove volatile solvent. Citric acid/water (1:1) solution was added to the remaining mixture, which was then extracted with ethyl acetate (4×). The combined organic extracts were washed with saturated sodium bicarbonate solution, and the organic phase was separated, dried (Na₂SO₄), then evaporated under reduced pressure to give a brown solid. The brown solid was subjected to reverse-phase preparative HPLC using acetonitrile and water (gradient 35:65 to 65:35, v/v, over 30 minutes) as eluent, to provide 5-[5-(3-phenyl-propionylamino-pyridin-2-yl]-thiophene-2-carboxylic acid hydroxyamide (8 mg). ¹H NMR [(CD₃)₂SO]: δ 11.22 (s br, 1H), 10.28 (s, 1H), 9.13 (s br, 1H), 8.68 (d, 1H), 8.12 (dd, 1H), 7.90 (d, 1H), 7.65 (d, 1H), 7.57 (apparent s br, 1H), 7.30 (m, 2H), 7.26 (m, 2H), 7.19 (m, 1H), 2.93 (t, 2H), 2.68 (t, 2H). LCMS (Method A): R_{T}=5.77 minutes; 368 (M₂+H)⁺.

### (ai) 4-Methyl-5-(5-trifluoromethyl-1H-pyrazol-3-yl-thiophene-2-carboxylic acid hydroxyamide

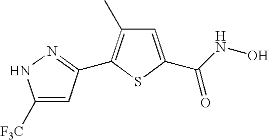

A solution of 4-methyl-5-(5-trifluoromethyl-1H-pyrazol-3-yl)-thiophene-2-carboxylic acid hydroxyamide [224 mg, 0.6 mmol, Reference Example 1(af)] in methanol (20 ml) was treated with p-toluene sulfonic acid (23 mg, 0.12 mmol), and the reaction mixture was stirred for 4 hours. The reaction mixture was evaporated under reduced pressure, and the residue was partitioned between ethyl acetate and saturated sodium bicarbonate solution. The two phases were separated and the organic phase was washed with brine, dried (Na₂SO₄), then evaporated under reduced pressure, to provide 4-methyl-5-(5-trifluoromethyl-1H-pyrazol-3-yl)-thiophene-2-carboxylic acid hydroxyamide (178 mg). ¹H NMR [(CD₃)₂SO]: δ 14.05 (s br, 1H), 11.31 (s br, 1H), 9.21 (s, 1H), 7.50 (s, 1H), 7.01 (s, 1H), 2.30 (s, 3H). LCMS (Method C): R_{T}=2.61 minutes; 583 (M₂+H)⁺.

### (aj) 5-(3-Benzyloxy-phenyl-thiophene-2-carboxylic acid hydroxyamide

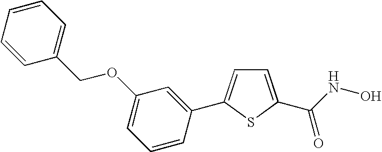

A solution of 5-(3-benzyloxy-phenyl)-thiophene-2-carboxylic acid (tetrahydro-pyran-2-yloxy)-amide [44 mg, 0.11 mmol, Reference Example 1(ag)] in methanol (1 ml) was treated with Amberlyst 15 ion exchange resin (87 mg). The mixture was stirred slowly at room temperature overnight, then the resin was filtered off, washed twice with methanol, and the filtrate was concentrated to give a white solid. The white solid was triturated with diethyl ether and filtered, to provide 5-(3-benzyloxy-phenyl)-thiophene-2-carboxylic acid hydroxyamide (18 mg) as an off-white solid. ¹H NMR (CD₃OD): δ 7.55 (apparent s br, 1H), 7.46 (m, 2H), 7.35-7.40 (m, 3H), 7.32 (t, 1H), 7.31 (m, 1H), 7.24-7.29 (m, 2H), 6.99 (ddd, 1H), 5.14 (s, 2H). LCMS (Method A): R_{T}=7.12 minutes; 326 (M+H)⁺.

### (ak) 5-(5-Phenethylamino-pyridin-2-yl)-thiophene-2-carboxylic acid hydroxyamide

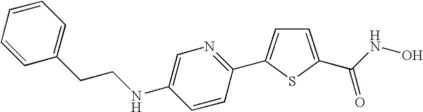

To a solution of 5-(6-phenethylamino-pyridin-2-yl)-thiophene-2-carboxylic acid methyl ester [31 mg, 0.09 mmol, Reference Example 28(a)] in methanol (1.5 ml), was added hydroxylamine hydrochloride (64 mg, 0.91 mmol) followed by potassium hydroxide powder (82 mg, 1.5 mmol). After siring overnight the reaction mixture was diluted with 10% citric acid solution and extracted twice with ethyl acetate. The organic layers were combined and extracted with ethyl acetate (2×). The organic phases were combined and washed with saturated sodium hydrogen carbonate solution, followed by brine, dried (Na₂SO₄), then evaporated to give a residue. The residue was subjected to reverse-phase preparative HPLC using acetonitrile and water (gradient 20:80 to 80:20, v/v, over 60 minutes) as eluent, to provide 5-(6-phenethylamino-pyridin-2-yl)-thiophene-2-carboxylic acid hydroxyamide (22 mg) as a yellow powder. ¹H NMR (CD₃OD): δ 7.85 (s, 1H), 7.81 (s, 1H), 7.60 (apparent s br, 1H), 7.56 (d, 1H), 7.44 (d, 1H), 7.26-7.32 (m, 4H), 7.21 (m, 1H), 3.49 (t, 2H), 2.95 (t, 2H). LCMS (Method A): R_{T}=5.90 minutes; 340 (M+H)⁺.

### (al) 5-(1-Pent-4-ynyl-1H-pyrazol-3-yl)-thiophene-2-carboxylic acid hydroxyamide

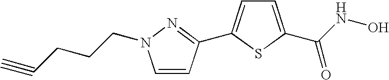

To 5-(1-pent-4-ynyl-1H-pyrazol-3-yl)-thiophene-2-carboxylic acid methyl ester [85 mg, 0.31 mmol, Reference Example 10(b)] was added 0.09M hydroxylamine hydrochloride in methanol solution (800 μl, 2.42 mmol), followed by 0.057M potassium hydroxide in methanol solution (700 μl, 1.55 mmol). After stirring over the weekend the reaction mixture was concentrated, dissolved in ethyl acetate then washed with saturated citric acid and water solution (1:1, v/v), followed by saturated sodium hydrogen carbonate solution. The organic phase was isolated, and evaporated to dryness, to provide 5-(1-pent-4-ynyl-1H-pyrazol-3-yl)-thiophene-2-carboxylic acid hydroxyamide (3.2 mg). LCMS (Method C): R_{T}=2.49 minutes; 276 (M+H)⁺.

### (am) 5-[1-(3-Phenyl-allyl-1H-pyrazol-3-yl]-thiophene-2-carboxylic acid hydroxyamide

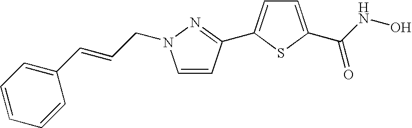

By proceeding in a similar manner to Reference 1(al) but using 5-[1-(3-phenyl-allyl)-1H-pyrazol-3-yl]-thiophene-2-carboxylic acid methyl ester [100 mg, 0.31 mmol, Reference Example 10(c)], there was prepared 5-[1-(3-phenyl-allyl)-1H-pyrazol-3-yl]-thiophene-2carboxylic acid hydroxyamide (8.4 mg). LCMS (Method C): R_{T}=3.01 minutes; 326 (M+H)⁺.

### (an) 5-[1-(3-Phenoxy-propyl)-1H-pyrazol-3-yl]-thiophene-2-carboxylic acid hydroxyamide

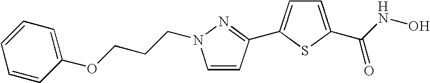

By proceeding in a similar manner to Example 1(al) but using 5-[1-(3-phenoxy-propyl)-1H-pyrazol-3-yl]-thiophene-2-carboxylic acid methyl ester [106 mg, 0.31 mmol, Reference Example 10(d)], there was prepared 5-[1-(3-phenoxy-propyl)-1H-pyrazol-3-yl]-thiophene-2-carboxylic acid hydroxyamide (44 mg). LCMS (Method C): R_{T}=2.99 minutes; 344 (M+H)⁺.

### (ao) 5-[1-(2-Benzoylamino-ethyl)-1H-pyrazol-3-yl]-thiophene-2-carboxylic acid hydroxyamide

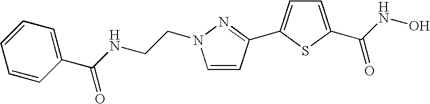

By proceeding in a similar manner to Example 1(al) but using 5-[1-(2-benzoylamino-ethyl)-1H-pyrazol-3-yl]-thiophene-2-carboxylic acid methyl ester [110 mg, 0.31 mmol, Reference Example 10(e)], there was prepared 5-[1-(2-benzoylamino-ethyl)-1H-pyrazol-3-yl]-thiophene-2-carboxylic acid hydroxyamide (3.1 mg). LCMS (Method C): R_{T}=2.34 minutes; 357 (M+H)⁺.

### (ap) 5-(1-Pyridin-4-ylmethyl-1H-pyrazol-3-yl)-thiophene-2-carboxylic acid hydroxyamide

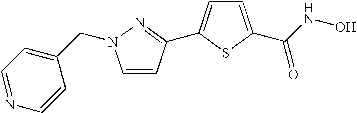

By proceeding in a similar manner to Example 1(al) but using 5-(1-pyridin-4-ylmethyl-1H-pyrazol-3-yl)-thiophene-2-carboxylic acid methyl ester [93 mg, 0.31 mmol, Reference Example 10(f)], there was prepared 5-(1-pyridin-4-ylmethyl-1H-pyrazol-3-yl)-thiophene-2-carboxylic acid hydroxyamide (21 mg). LCMS (Method C): R_{T}=0.36 minutes; 301 (M+H)⁺.

### (aq) 5-[1-(5-tert-Butyl-[1,2,4]oxadiazol-3-ylmethyl)-1H-pyrazol-3-yl]-thiophene-2-carboxylic acid hydroxyamide

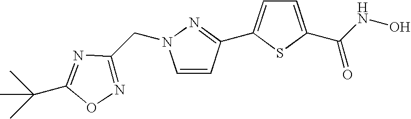

By proceeding in a similar manner to Example 1(al) but using 5-[1-(5-tert-butyl-[1,2,4]oxadiazol-3-ylmethyl)-1H-pyrazol-3-yl]-thiophene-2-carboxylic acid methyl ester [108 mg, 0.31 mmol, Reference Example 10(g)], there was prepared 5-[1-(5-tert-butyl-[1,2,4]oxadiazol-3-ylmethyl)-H-pyrazol-3-yl]-thiophene-2-carboxylic acid hydroxyamide (57 mg). LCMS (Method C): R_{T}=2.73 minutes; 348 (M+H)⁺.

### (ar) 5-[1-(3-Pyrrol-1-yl-propyl)-1H-pyrazol-3-yl]-thiophene-2-carboxylic acid hydroxyamide

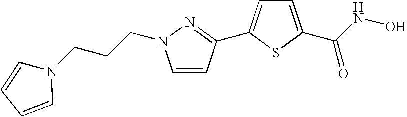

By proceeding in a similar manner to Example 1(al) but using 5-[1-(3-pyrrol-1-yl-propyl)-1H-pyrazol-3-yl]-thiophene-2-carboxylic acid methyl ester [98 mg, 0.31 mmol, Reference Example 10(h)], there was prepared 5-[1-(3-pyrrol-1-yl-propyl)-1H-pyrazol-3-yl]-thiophene-2-carboxylic acid hydroxyamide (62 mg). LCMS (Method C): R_{T}=2.71 minutes; 317 (M+H)⁺.

### (as) 5-(1-But-2-enyl-1H-pyrazol-3-yl-thiophene-2-carboxylic acid hydroxyamide

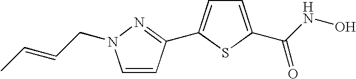

By proceeding in a similar manner to Example 1(al) but using 5-(1-but-2-enyl-1H-pyrazol-3-yl)-thiophene-2-carboxylic acid methyl ester [81 mg, 0.31 mmol, Reference Example 10(i)], there was prepared 5-(1-but-2-enyl-1H-pyrazol-3-yl)-thiophene-2-carboxylic acid hydroxyamide (81 mg). LCMS (Method C): R_{T}=2.53 minutes; 264 (+H)⁺.

### (at) 5-[5-(2-Phenoxy-acetylamino)-pyridin-2-yl]-thiophene-2-carboxylic acid hydroxyamide

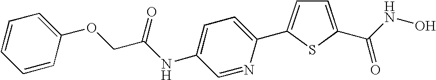

By proceeding in a similar manner to Example 1(ak) but using 5-[5-(2-phenoxy-acetylamino)-pyridin-2-yl]-thiophene-2-carboxylic acid methyl ester [87 mg, 0.23 mmol, Reference Example 11 (b)] and N,N dimethyl acetamide as co-solvent, there was prepared 5-[5-(2-phenoxy-acetylamino)-pyridin-2-yl]-thiophene-2-carboxylic acid hydroxyamide (38 mg) as a yellow solid. ¹H NMR [(CD₃)₂SO]: δ 11.27 (s, 1H), 10.48 (s, 1H), 9.18 (s br, 1H), 8.79 (d, 1H), 8.18 (dd, 1H), 7.95 (d, 1H), 7.69 (d, 1H), 7.60 (d br, 1H), 7.33 (m, 2H), 7.03 (m, 2H), 6.99 (m, 1H), 4.76 (s, 1H). LCMS (Method A): R_{T}=5.61 minutes; 370 (M+H)⁺.

### (au) 5-(5-Phenylacetylamino-pyridin-2-yl-thiophene-2-carboxylic acid hydroxyamide

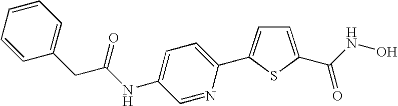

By proceeding in a similar manner to Example 1(ak) but using 5-(5-phenylacetylamino-pyridin-2-yl)-thiophene-2-carboxylic acid methyl ester [94 mg, 0.26 mmol, Reference Example 11(c)], tetrahydrofuran as co-solvent, there was prepared 5-(5-phenylacetylamino-pyridin-2-yl)-thiophene-2-carboxylic acid hydroxyamide (43 mg) as an off-white solid. ¹H NMR [(CD₃)₂SO]: δ 11.19 (s, 1H), 10.50 (s, 1H), 9.11 (s, 1H), 8.66 (d, 1H), 8.09 (dd, 1H), 7.86 (d, 1H), 7.61 (d, 1H), 7.54 (apparent s br, 1H), 7.26-7.32 (m, 4H), 7.21 (m, 11H), 3.65 (s, 2H). LCMS (Method A): R_{T}=5.35 minutes; 354 (M+H)⁺.

### (av) 5-(1-Quinolin-2-ylmethyl-1H-pyrazol-3-yl)-thiophene-2-carboxylic acid hydroxyamide

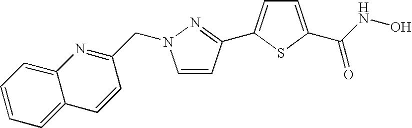

To a mixture of 5-(1-quinolin-2-ylmethyl-1H-pyrazol-3-yl)-thiophene-2-carboxylic acid methyl ester [345 mg, 0.99 mmol, Reference Example 10(j)] in N,N dimethyl acetamide (10 ml), was added hydroxylamine hydrochloride (344 mg, 4.95 mmol) followed by 25% sodium methoxide in methanol solution (1.66 ml, 7.7 mmol). After stirring overnight the reaction mixture was diluted with saturated citric acid solution and extracted twice with ethyl acetate. The organic layers were combined, dried (Na₂SO₄), and concentrated to give a yellow gum, which was subjected to reverse-phase purification using acetonitrile and water (gradient 10:90 to 90:10, v/v). The isolated product was triturated with acetonitrile, to provide 5-(1-quinolin-2-ylmethyl-1H-pyrazol-3-yl)-thiophene-2-carboxylic acid hydroxyamide (43 mg) as a grey powder. ¹H NMR [(CD₃)₂SO]: δ 11.18 (s, 1H), 8.37 (d, 1H), 8.04 (d, 1H), 8.00 (apparent d, 1H), 7.97 (apparent d, 1H), 7.78 (ddd, 1H), 7.61 (ddd, 1H), 7.55 (apparent s br, 1H), 7.40 (d, 1H), 7.24 (d, 1H), 6.78 (d, 1H), 5.66 (s, 2H). LCMS (Method A): R_{T}=4.99 minutes; 351 (M+E)⁺.

### (aw) 5-(5-Benzoylamino-pyridin-2-yl)-thiophene-2-carboxylic acid hydroxyamide

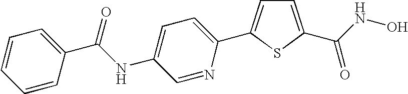

By proceeding in a similar manner to Example 1(ak) but using 5-(5-benzoylamino-pyridin-2-yl)-thiophene-2-carboxylic acid methyl ester [79 mg, 0.23 mmol, Reference Example 11(d)], N,N dimethylacetamide as co-solvent, methanol and water (gradient 10:90 to 30:70, v/v, over 80 minutes) as eluent, there was prepared 5-(5-benzoylamino-pyridin-2-yl-thiophene-2-carboxylic acid hydroxyamide (9 mg) as a brown solid. ¹H NMR [(CD₃)₂SO]: δ 11.24 (s br, 1H), 10.59 (s, 1H), 9.16 (s, 1H), 8.93 (d, 1H), 8.30 (dd, 1H), 7.95-8.05 (m, 3H), 7.71 (d, 1H), 7.52-7.68 (m, 4H). LCMS (Method A): R_{T}=5.20 minutes; 340 (M+H)⁺.

### (ax) N-[6-(5-Hydroxycarbamoyl-thiophen-2-yl)-pyridin-3-yl]-isonicotinamide

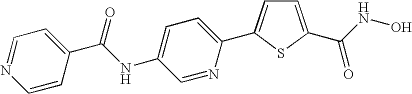

By proceeding in a similar manner to Example 1(ak) but using 5-{5-[(pyridine-4-carbonyl)-amino]-pyridin-2-yl}-thiophene-2-carboxylic acid methyl ester [93 mg, 0.27 mmol, Reference Example 11 (e)], N,N dimethylacetamide as co-solvent, methanol and water (gradient 10:90 to 30:70, v/v, over 80 minutes) as eluent, there was prepared N-[6-(5-hydroxycarbamoyl-thiophen-2-yl)-pyridin-3-yl]-isonicotinamide (11 mg) as a brown solid. ¹H NMR [(CD₃)₂SO]: δ 10.97 (s br, 1H), 10.64 (s, 1H), 8.97 (s br, 1H), 8.91 (d, 1H), 8.80 (d, 2H), 8.25 (dd, 1H), 7.93 (d, 1H), 7.87 (d, 2H), 7.66 (d, 1H), 7.62 (d, 1H). LCMS (Method A): R_{T}=3.71 minutes; 341 (M+H)⁺.

### (ay) 5-{5-[(Quinolin-2-ylmethyl)-amino]-pyridin-2-yl}-thiophene-2-carboxylic acid hydroxyamide

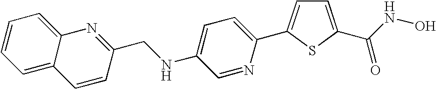

By proceeding in a similar manner to Example 1(ak) but using 5-{5-[(quinolin-2-ylmethyl)-amino]-pyridin-2-yl}-thiophene-2-carboxylic acid methyl ester [98 mg, 0.26 mmol, Reference Example 28(b)] and N,N dimethyl acetamide as co-solvent, methanol and water (gradient 10:90 to 90:10, v/v, over 80 minutes) as eluent, there was prepared 5-{5-[(quinolin-2-ylmethyl)-amino]-pyridin-2-yl}-thiophene-2-carboxylic acid hydroxyamide (8 mg) as a yellow solid. ¹H NMR (CD₃OD): δ 8.78 (d, 1H), 8.21 (d, 1H), 8.15 (d, 1H), 8.05 (s, 1H), 8.00 (t, 1H), 7.88 (d, 1H), 7.80 (t, 1H), 7.73 (d, 1H), 7.54 (apparent s, 1H), 7.47 (apparent s, 1H), 7.32 (apparent s, 1H), 4.94 (s, 2H). LCMS (Method A): R_{T}=4.32 minutes; 377 (M+H)⁺.

### (az) 5-{5-[(2,3-Dihydro-benzo[1,4]dioxin-6-ylmethyl)-amino]-pyridin-2-yl}-thiophene-2-carboxylic acid hydroxyamide

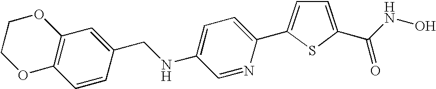

By proceeding in a similar manner to Example 1(ak) but using 5-{5-[(2,3-dihydro-benzo[1,4]dioxin-6-ylmethyl)-amino]-pyridin-2-yl}-thiophene-2-carboxylic acid methyl ester [130 mg, 0.26 mmol, Reference Example 28(c)] and N,N dimethyl acetamide as co-solvent, methanol and water (gradient 10:90 to 90:10, v/v, over 80 minutes) as eluent, there was prepared 5-{5-[(2,3-dihydro-benzo[1,4]dioxin-6-ylmethyl)-amino]-pyridin-2-yl}-thiophene-2-carboxylic acid hydroxyamide (28 mg) as a yellow solid. ¹H NMR (CD₃OD): δ 7.86 (s br, 1H), 7.76 (d, 1H), 7.56 (apparent s br, 1H), 7.50 (d br, 1H), 7.34 (d br, 1H), 6.86 (d, 1H), 6.84 (dd, 1H), 6.79 (d, 1H), 4.32 (s, 2H), 4.20 (s, 4H). LCMS (Method A): R_{T}=4.74 minutes; 384 (M+H)⁺.

### (ba) 5-{5-[(Benzofuran-2-ylmethyl)-amino]-pyridin-2-yl}-thiophene-2-carboxylic acid hydroxyamide

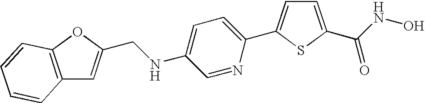

By proceeding in a similar manner to Example 1(ak) but using 5-{5-[(benzofuran-2-ylmethyl)-amino]-pyridin-2-yl}-thiophene-2-carboxylic acid methyl ester [84 mg, 0.23 mmol, Reference Example 28(d)] and N,N dimethyl acetamide as co-solvent, acetonitrile and water (gradient 5:95 to 95:5, v/v, over 90 minutes), there was prepared 5-{5-[(benzofuran-2-ylmethyl)-amino]-pyridin-2-yl}-thiophene-2-carboxylic acid hydroxyamide (18 mg) as a yellow solid. ¹H NMR (CD₃OD): δ 8.03 (s, 1H), 7.81 (d, 1H), 7.55 (s br, 2H), 7.52 (dd, 1H), 7.49 (d, 1H), 7.43 (dd, 1H), 7.24 (dt, 1H), 7.18 (dt, 1H), 6.74 (s, 1H), 4.62 (s, 2H). LCMS (Method A): R_{T}=5.35 minutes; 366 (M+H)⁺.

### (bb) 5-{1-[2-(4-Fluoro-benzyloxy)-ethyl]-1H-pyrazol-3-yl}-thiophene-2-carboxylic acid hydroxyamide

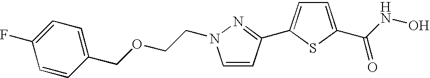

To a mixture of 5-{1-[2-(4-fluoro-benzyloxy)-ethyl]-1H-pyrazol-3-yl}-thiophene-2-carboxylic acid methyl ester [72 mg, 0.2 mmol, Reference Example 29(a)] in N,N dimethyl acetamide (3 ml), was added hydroxylamine hydrochloride (72 mg, 1.0 mmol) followed by 25% sodium methoxide in methanol solution (0.34 ml, 1.56 mmol). After stirring overnight the reaction mixture was concentrated, and the residue was dissolved in ethyl acetate. The resultant solution washed with water and saturated citric solution (1:1, v/v), followed by saturated sodium hydrogen carbonate solution. The organic layer was isolated, dried (MgSO₄), and concentrated to give an orange oil, which was subjected to reverse-phase purification using methanol and water (gradient 5:95 to 95:5, v/v) as eluent, to provide 5-{1-[2-(4-fluoro-benzyloxy)-ethyl]-1H-pyrazol-3-yl}-thiophene-2-carboxylic acid hydroxyamide (8 mg). ¹H NMR (CD₃OD): δ 7.66 (d, 1H), 7.53 (apparent s br, 1H), 7.33 (d, 1H), 7.23 (m, 2H), 6.98 (m, 2H), 6.59 (m, 2H), 4.46 (s, 2H), 4.34 (t, 2H), 3.83 (t, 2H). LCMS (Method A): R_{T}=5.22 minutes; 362 (M+H)⁺.

### (bc) 5-(1-Phenylcarbamoylmethyl-1H-pyrazol-3-yl)-thiophene-2-carboxylic acid hydroxyamide

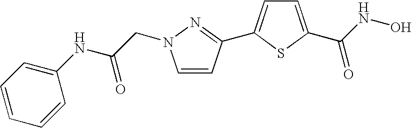

To a solution of 5-(1-phenylcarbamoylmethyl-1H-pyrazol-3-yl)-thiophene-2-carboxylic acid methyl ester [325 mg, 0.95 mmol, Reference Example 10(k)] in N,N dimethyl acetamide (5 ml), was added hydroxylamine hydrochloride (331 mg, 4.76 mmol) followed by 25% sodium methoxide in methanol solution (1.6 ml, 7.43 mmol). After stirring overnight the reaction mixture was diluted with 10% citric acid solution and extracted with ethyl acetate (2×). The organic layers were combined and washed with saturated sodium hydrogen carbonate solution, dried (MgSO₄), then evaporated to give a gum. The gum was subjected to reverse-phase preparative HPLC using acetonitrile and water (gradient 10:90 to 90:10, v/v) as eluent, to provide 5-(1-phenylcarbamoylmethyl-1H-pyrazol-3-yl)-thiophene-2-carboxylic acid hydroxyamide (44 mg) as a white solid. ¹H NMR [(CD₃)₂SO]: δ 11.17 (s br, 1H), 10.35 (s, 1H), 9.10 (s, 1H), 7.85 (d, 1H), 7.59 (m, 2H), 7.55 (apparent s br, 1H), 7.39 (d, 1H), 7.33 (m, 2H), 7.08 (m, 1H), 6.71 (d, 1H), 5.06 (s, 2H). LCMS (Method A): R_{T}=4.58 minutes; 343 (M+H)⁺.

### (bd) 5-(1-{[(Pyridin-2-ylmethyl)-carbamoyl]-methyl}-1H-pyrazol-3-yl)-thiophene-2-carboxylic acid hydroxyamide

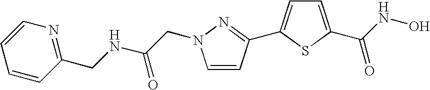

To a solution of 5-(1{[(pyridin-2-ylmethyl)-carbamoyl]-methyl}-1H-pyrazol-3-yl)-thiophene-2-carboxylic acid methyl ester [53 mg, 0.14 mmol, Reference Example 30(a)] in N,N dimethyl acetamide (1 ml), was added hydroxylamine hydrochloride (52 mg, 0.74 mmol) followed by 25% sodium methoxide in methanol solution (0.24 μl, 1.09 mmol). After stirring overnight the reaction mixture was diluted with 10% citric acid solution and extracted with ethyl acetate (2×). The organic phases were combined and washed with saturated sodium hydrogen carbonate solution, followed by brine, dried (Na₂SO₄), then evaporated to give a residue. The residue was subjected to reverse-phase preparative HPLC using acetonitrile and water (gradient 5:95 to 95:5, v/v, over 90 minutes) as eluent, to provide 5-(1-{[(pyridin-2-ylmethyl)-carbamoyl]-methyl}-1H-pyrazol-3-yl)-thiophene-2-carboxylic acid hydroxyamide (18 mg) as a brown solid. ¹H NMR (CD₃OD): δ 8.72 (d, 1H), 8.46 (dt, 1H), 7.97 (d, 1H), 7.86 (apparent t, 1H), 7.74 (d, 1H), 7.52 (apparent s br, 1H), 7.36 (d, 1H), 6.68 (d, 1H), 5.02 (s, 2H), 4.75 (s, 2H). LCMS (Method A): R_{T}=2.56 minutes; 358 (M+H)⁺.

### (be) 5-[1-(Quinolin-8-ylcarbamoylmethyl)-1H-pyrazol-3-yl]-thiophene-2-carboxylic acid hydroxyamide

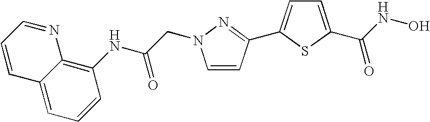

By proceeding in a similar manner to Example 1(bd) but using 5-[1-(quinolin-8-ylcarbamoylmethyl)-1H-pyrazol-3-yl]-thiophene-2-carboxylic acid methyl ester [257 mg, 0.65 mmol, Reference Example 30(b)], there was prepared 5-[1-(quinolin-8-ylcarbamoylmethyl)-1H-pyrazol-3-yl]-thiophene-2-carboxylic acid hydroxyamide (24 mg) as a brown solid. ¹H NMR [(CD₃)₂SO]: δ 11.22 (s, 1H), 10.66 (s, 1H), 9.13 (s br, 1H), 8.82 (dd, 1H), 8.62 (dd, 1H), 8.41 (dd, 1H), 7.97 (d, 1H), 7.70 (dd, 1H), 7.63 (dd, 1H), 7.59 (apparent s br, 1H), 7.59 (apparent t, 1H), 7.47 (d, 1H), 6.79 (d, 1H), 5.35 (s, 2H). LCMS (Method A): R_{T}=3.86 minutes; 394 (M+H)⁺.

### (bf) 5-{1-[(5-Trifluoromethyl-[1,3,4]thiadiazol-2-ylcarbamoyl)-methyl]-1H-pyrazol-3-yl}-thiophene-2-carboxylic acid hydroxyamide

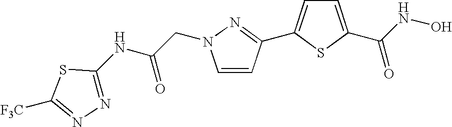

To a solution of 5-{1-[(5-trifluoromethyl-[1,3,4]thiadiazol-2-ylcarbamoyl)-methyl]-1H-pyrazol-3-yl}-thiophene-2-carboxylic acid methyl ester [200 mg, 0.48 mmol, Reference Example 10(1)] in N,N dimethyl acetamide (2 ml) was added hydroxylamine hydrochloride (167 mg, 2.4 mmol), followed by 1.8M potassium hydroxide in methanol solution (211 mg, 3.7 mmol). After stirring overnight the reaction mixture was concentrated, and the residue was dissolved in ethyl acetate. The resultant solution washed with water and saturated citric solution (1:1, v/v), followed by saturated sodium hydrogen carbonate solution. The organic phase was isolated, and evaporated to dryness, to provide 5-{1-[(5-trifluoromethyl-[1,3,4]thiadiazol-2-ylcarbamoyl)-methyl]-1H-pyrazol-3-yl}-thiophene-2-carboxylic acid hydroxyamide. LCMS (Method C): R_{T}=2.61 minutes; 417 (M⁻).

### (bg) 5-{1-[(2-Methoxy-phenylcarbamoyl)-methyl]-1H-pyrazol-3-yl}-thiophene-2-carboxylic acid hydroxyamide

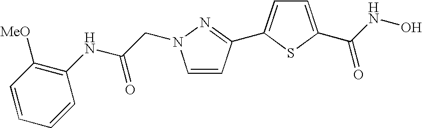

By proceeding in a similar manner to Example 1(bf) but using 5-{1-[(2-methoxy-phenylcarbamoyl)-methyl]-1H-pyrazol-3-yl}-thiophene-2-carboxylic acid methyl ester [178 mg, 0.48 mmol, Reference Example 10(m)] and triturating the final product in methanol, there was prepared 5-{1-[(2-methoxy-phenylcarbamoyl)-methyl]-1H-pyrazol-3-yl}-thiophene-2-carboxylic acid hydroxyamide. LCMS (Method C): R_{T}=2.55 minutes; 371 (M⁻).

### (bh) 5-{1-[(4-Fluoro-phenylcarbamoyl)-methyl]-1H-pyrazol-3-yl}-thiophene-2-carboxylic acid hydroxyamide

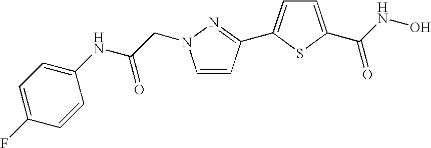

By proceeding in a similar manner to Example 1(bf) but using 5-{1-[(4-fluoro-phenylcarbamoyl)-methyl]-1H-pyrazol-3-yl}-thiophene-2-carboxylic acid methyl ester [172 mg, 0.48 mmol, Reference Example 10(n)], there was prepared 5-{1-[(4-fluoro-phenylcarbamoyl)-methyl]-1H-pyrazol-3-yl}-thiophene-2-carboxylic acid hydroxyamide. LCMS (Method C): R_{T}=2.51 minutes; 359 (M⁻).

### (bi) 5-{1-[(3-Fluoro-phenylcarbamoyl)-methyl]-1H-pyrazol-3-yl}-thiophene-2-carboxylic acid hydroxyamide

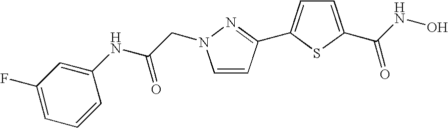

By proceeding in a similar manner to Example 1(bf) but using 5-{1-[(3-fluoro-phenylcarbamoyl)-methyl]-1H-pyrazol-3-yl}-thiophene-2-carboxylic acid methyl ester [172 mg, 0.48 mmol, Reference Example 10(o)], there was prepared 5-{1-[(3-fluoro-phenylcarbamoyl)-methyl]-1H-pyrazol-3-yl}-thiophene-2-carboxylic acid hydroxyamide. LCMS (Method C): R_{T}=2.58 minutes; 359 (M⁻).

### (bj) Quinoline-2-carboxylic acid {2-[3-(5-hydroxycarbamoyl-thiophen-2-yl)-pyrazol-1-yl]-ethyl}-amide

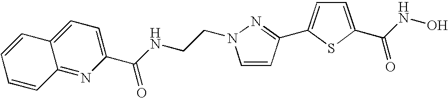

By proceeding in a similar manner to Example 1(bf) but using 5-(1-{2-[(quinoline-2-carbonyl)-amino]-ethyl}-1H-pyrazol-3-yl)-thiophene-2-carboxylic acid methyl ester [195 mg, 0.48 mmol, Reference Example 10(p)] and triturating the final product in methanol, there was prepared quinoline-2-carboxylic acid {2-[3-(5-hydroxycarbamoyl-thiophen-2-yl)-pyrazol-1-yl]-ethyl}-amide. LCMS (Method A): R_{T}=6.41 minutes; 408 (M+H)⁺.

### (bk) 5-[1-(Benzylcarbamoyl-methyl)-1H-pyrazol-3-yl]-thiophene-2-carboxylic acid hydroxyamide

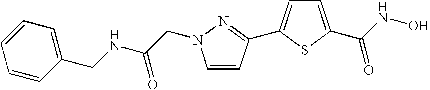

By proceeding in a similar manner to Example 1(bf) but using 5-[1-(benzylcarbamoyl-methyl)-1H-pyrazol-3-yl]-thiophene-2-carboxylic acid methyl ester [170 mg, 0.48 mmol, Reference Example 10(q)] and subjecting the crude material to reverse-phase preparative HPLC using acetonitrile and water (gradient 5:95 to 95:5, v/v, over 90 minutes) as eluent, there was prepared 5-[1-(benzylcarbamoyl-methyl-1H-pyrazol-3-yl]-thiophene-2-carboxylic acid hydroxyamide. LCMS (Method A): R_{T}=5.47 minutes; 357 (M+H)⁺.

### (bl) 5-{1-[(N-Ethyl-N-phenyl-carbamoyl)-methyl]-1H-pyrazol-3-1}-thiophene-2-carboxylic acid hydroxyamide

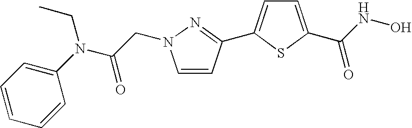

By proceeding in a similar manner to Example 1(bf) but using 5-{1-[(N-ethyl-N-phenyl-carbamoyl)-methyl]-1H-pyrazol-3-yl}-thiophene-2-carboxylic acid methyl ester [177 mg, 0.48 mmol, Reference Example 10(r)] and subjecting the crude material to reverse-phase preparative HPLC using acetonitrile and water (gradient 5:95 to 95:5, v/v, over 90 minutes) as eluent, there was prepared 5-{1-[(N-ethyl-N-phenyl-carbamoyl)-methyl]-1H-pyrazol-3-yl}-thiophene-2-carboxylic acid hydroxyamide. LCMS (Method A): R_{T}=6.41 minutes; 371 (M+H)⁺.

### (bm) 5-{1-[2-(1H-Indol-3-yl)-ethyl]-1H-pyrazol-3-yl}-thiophene-2-carboxylic acid hydroxyamide

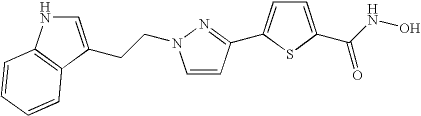

By proceeding in a similar manner to Example 1(bf) but using 5-{1-[2-(1H-indol-3-yl)-ethyl]-1H-pyrazol-3-yl}-thiophene-2-carboxylic acid methyl ester [168 mg, 0.48 mmol, Reference Example 10(s)] and subjecting the crude material to reverse-phase preparative HPLC using acetonitrile and water (gradient 5:95 to 95:5, v/v, over 90 minutes) as eluent, there was prepared 5-{1-[2-(1H-indol-3-yl)-ethyl]-1H-pyrazol-3-yl}-thiophene-2-carboxylic acid hydroxyamide. LCMS (Method A): R_{T}=6.95 minutes; 353 (N+H)⁺.

### (bn) 5-{1-[(2-Trifluoromethoxy-phenylcarbamoyl)-methyl]-1H-pyrazol-3-yl}-thiophene-2-carboxylic acid hydroxyamide

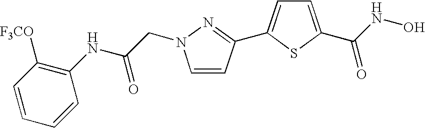

By proceeding in a similar manner to Example 1(bf) but using 5-{1-[(2-trifluoromethoxy-phenylcarbamoyl)-methyl]-1H-pyrazol-3-yl}-thiophene-2-carboxylic acid methyl ester [204 mg, 0.48 mmol, Reference Example 10(t)] and subjecting the crude material to reverse-phase preparative HPLC using acetonitrile and water (gradient 5:95 to 95:5, v/v, over 90 minutes) as eluent, there was prepared 5-{1-[(2-trifluoromethoxy-phenylcarbamoyl)-methyl]-1H-pyrazol-3-yl}-thiophene-2-carboxylic acid hydroxyamide. LCMS (Method A): R_{T}=6.90 minutes; 427 (M+H)⁺.

### (bo) 5-{[1-3-(4-Chloro-phenyl)-propyl]-1H-pyrazol-3-yl}-thiophene-2-carboxylic acid hydroxyamide

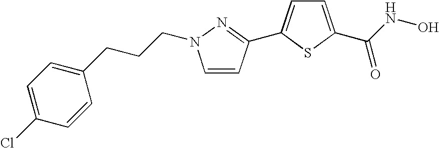

By proceeding in a similar manner to Example 1(bf) but using 5-{1-[3-(4-chloro-phenyl)-propyl]-1H-pyrazol-3-yl}-thiophene-2-carboxylic acid methyl ester [172 mg, 0.48 mmol, Reference Example 10(u)] and subjecting the crude material to reverse-phase preparative HPLC using acetonitrile and water (gradient 5:95 to 95:5, v/v, over 90 minutes) as eluent, there was prepared 5-{1-[3-(4-chloro-phenyl)-propyl]-1H-pyrazol-3-yl}-thiophene-2-carboxylic acid hydroxyamide. ¹H NMR (CD₃OD): δ 7.63 (d, 1H), 7.53 (apparent br, 1H), 7.32 (d, 1H), 7.25 (m, 2H), 7.17 (m, 2H), 6.58 (d, 1H), 4.15 (t, 2H), 2.60 (t, 2H), 2.17 (m, 2H). LCMS (Method A): R_{T}=8.39 minutes; 362 & 364 (M+H)⁺.

### (bp) 5-(1-{[2-(1H-Indol-3-yl)-ethylcarbamoyl]-methyl}-1H-pyrazol-3-yl)-thiophene-2-carboxylic acid hydroxyamide

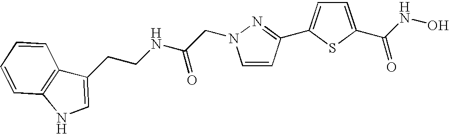

By proceeding in a similar manner to Example 1(bf) but using 5-(1-{[2-(1H-indol-3-yl)-ethylcarbamoyl]-methyl}-1H-pyrazol-3-yl)-thiophene-2-carboxylic acid methyl ester [196 mg, 0.48 mmol, Reference Example 10(v)] and subjecting the crude material to reverse-phase preparative HPLC using acetonitrile and water (gradient 5:95 to 95:5, v/v, over 90 minutes) as eluent, there was prepared 5-(1-{[2-(1H-indol-3-yl)-ethylcarbamoyl]-methyl}-1H-pyrazol-3-yl)-thiophene-2-carboxylic acid hydroxyamide (43 mg). ¹H NMR [(CD₃)₂SO]: δ 11.17 (s br, 1H), 10.82 (s, 1H), 9.10 (s br, 1H), 8.27 (t, 1H), 7.77 (d, 1H), 7.55 (apparent s br, 1H), 7.54 (d, 1H), 7.38 (d, 1H), 7.34 (d, 1H), 7.17 (d, 1H), 7.06 (m, 1H), 6.98 (m, 1H), 6.68 (d, 1H), 4.81 (s, 2H), 3.39 (dt, 2H), 2.85 (t, 2H). LCMS (Method A): R_{T}=6.01 minutes; 410 (M+H)⁺.

### (bq) 5-[1-(Phenethylcarbamoyl-methyl)-1H-pyrazol-3-yl]-thiophene-2-carboxylic acid hydroxyamide

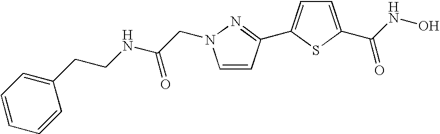

By proceeding in a similar manner to Example 1(bf) but using 5-[1-(phenethylcarbamoyl-methyl)-1H-pyrazol-3-yl]-thiophene-2-carboxylic acid methyl ester [177 mg, 0.48 mmol, Reference Example 10(w)] and subjecting the crude material to reverse-phase preparative HPLC using acetonitrile and water (gradient 5:95 to 95:5, v/v, over 90 minutes) as eluent, there was prepared 5-[1-(phenethylcarbamoyl-methyl)-1H-pyrazol-3-yl]-thiophene-2-carboxylic acid hydroxyamide. ¹H NMR (CD₃OD): δ 7.66 (d, 1H), 7.53 (apparent s br, 1H), 7.35 (d, 1H), 7.24 (m, 2H), 7.18 (m, 2H), 7.15 (m, 1H), 6.64 (d, 1H), 4.82 (s, 2H), 3.45 (t, 2H), 2.79 (t, 2H). LCMS (Method A): R_{T}=5.83 minutes; 371 (M+H)⁺.

### (br) 5-(1-Isoquinolin-1-ylmethyl-1H-pyrazol-3-yl)-thiophene-2-carboxylic acid hydroxyamide

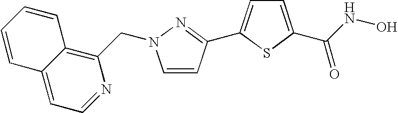

By proceeding in a similar manner to Example 1(bf) but using 5-(1-isoquinolin-1-ylmethyl-1H-pyrazol-3-yl)-thiophene-2-carboxylic acid methyl ester [168 mg, 0.48 mmol, Reference Example 10(x)] and subjecting the crude material to reverse-phase preparative HPLC using acetonitrile and water (gradient 5:95 to 95:5, v/v, over 90 minutes) as eluent, there was prepared 5-(1-isoquinolin-1-ylmethyl-1H-pyrazol-3-yl)-thiophene-2-carboxylic acid hydroxyamide (17 mg). ¹H NMR [(CD₃)₂SO]: δ 11.17 (s br, 1H), 9.08 (s br, 1H), 8.44 (d, 1H), 8.43 (d, 1H), 8.02 (d, 1H), 7.90 (d, 1H), 7.83 (d, 1H), 7.81 (m, 1H), 7.74 (m, 1H), 7.52 (apparent s br, 1H), 7.34 (d, 1H), 6.69 (d, 1H), 6.03 (s, 2H). LCMS (Method A): R_{T}=5.44 minutes; 351 (+H)⁺.

### (bs) 5-{1-[(2-Fluoro-phenylcarbamoyl)-methyl]-1H-pyrazol-3-yl}-thiophene-2-carboxylic acid hydroxyamide

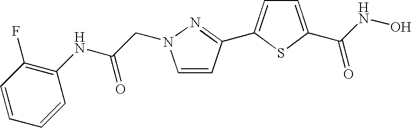

By proceeding in a similar manner to Example 1(bf) but using 5-{1-[(2-fluoro-phenylcarbamoyl)-methyl]-1H-pyrazol-3-yl}-thiophene-2-carboxylic acid methyl ester [172 mg, 0.48 mmol, Reference Example 10(y)] and subjecting the crude material to reverse-phase preparative HPLC using acetonitrile and water (gradient 5:95 to 95:5, v/v, over 90 minutes) as eluent, there was prepared 5-{1-[(2-fluoro-phenylcarbamoyl-methyl]-1H-pyrazol-3-yl}-thiophene-2-carboxylic acid hydroxyamide. ¹H NMR (CD₃OD): δ 7.98 (m, 1H), 7.77 (d, 1H), 7.53 (apparent s br, 1H), 7.38 (d, 1H), 7.10-7.20 (m, 3H), 6.68 (d, 1H), 5.13 (s, 2H). LCMS (Method A): R_{T}=5.77 minutes; 361 (M+H)⁺.

### (bt) 5-[1-(Quinolin-3-ylcarbamoylmethyl)-1H-pyrazol-3-yl]-thiophene-2-carboxylic acid hydroxyamide

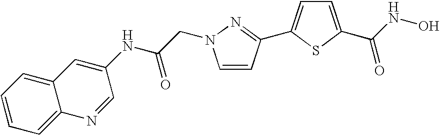

By proceeding in a similar manner to Example 1(ak) but using 5-[1-(quinolin-3-ylcarbamoylmethyl)-1H-pyrazol-3-yl]-thiophene-2-carboxylic acid methyl ester [88 mg, 0.22 mmol, Reference Example 30(c)] and N,N dimethyl acetamide as co-solvent, (gradient 5:95 to 95:5, v/v, over 90 minutes), there was prepared 5-[1-(quinolin-3-ylcarbamoylmethyl)-1H-pyrazol-3-yl]-thiophene-2-carboxylic acid hydroxyamide (13 mg) as a white solid. ¹H NMR [(CD₃)₂SO]: δ 11.20 (s, 1H), 10.89 (s, 1H), 9.08 (s br, 1H), 8.96 (d, 1H), 8.70 (d, 1H), 7.98 (d, 1H), 7.93 (dd, 1H), 7.90 (d, 1H), 7.67 (m, 1H), 7.59 (m, 1H), 7.56 (apparent s br, 1H), 7.41 (d, 1H), 6.74 (d, 1H), 5.18 (s, 2H). LCMS (Method A): R_{T}=4.16 minutes; 394 (M+H)⁺.

### (bu) 5-[1-(Pyridin-3-ylcarbamoylmethyl)-1H-pyrazol-3-yl]-thiophene-2-carboxylic acid hydroxyamide

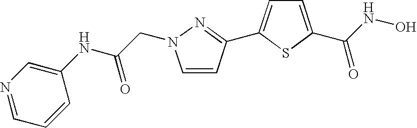

By proceeding in a similar manner to Example 1(bd) but using 5-[1-(pyridin-3-ylcarbamoylmethyl)-1H-pyrazol-3-yl]-thiophene-2-carboxylic acid methyl ester [88 mg, 0.25 mmol, Reference Example 30(d)], methanol and water (gradient 5:95 to 95:5, v/v, over 90 minutes) as eluent, there was prepared 5-[1-(pyridin-3-ylcarbamoylmethyl-1H-pyrazol-3-yl]-thiophene-2-carboxylic acid hydroxyamide (10 mg) as a brown solid. ¹H NMR (CD₃OD): δ 9.34 (apparent s br, 1H), 8.57 (apparent s br, 1H), 8.48 (d, 1H), 7.99 (apparent s, 1H), 7.78 (apparent s, 1H), 7.52 (apparent s, 1H), 7.37 (d br, 1H), 6.69 (apparent s, 1H), 5.18 (apparent s, 2H). LCMS (Method A): R_{T}=2.90 minutes; 344 (M+H)⁺.

### (by) 5-[1-(2-Quinolin-2-yl-ethyl)-1H-pyrazol-3-yl]-thiophene-2-carboxylic acid hydroxyamide

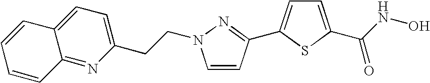

To a solution of 5-[1-(2-quinolin-2-yl-ethyl)-1H-pyrazol-3-yl]-thiophene-2-carboxylic acid methyl ester [200 mg, 0.55 mmol, Reference Example 10(z)] in N,N dimethyl acetamide (3 ml), was added hydroxylamine hydrochloride (191 mg, 2.75 mmol) followed by 25% sodium methoxide in methanol solution (0.93 ml, 4.3 mmol). After stirring for 8 hours the reaction mixture was left to stand over the weekend, then concentrated, and the residue was partitioned between saturated sodium hydrogen carbonate solution and ethyl acetate. The organic layer was isolated, dried (MgSO₄) and evaporated to give a residue, which was subjected to reverse-phase preparative HPLC using acetonitrile and water (gradient 10:90 to 90:10, v/v, over 40 minutes) as eluent, to provide 5-[1-(2-quinolin-2-yl-ethyl-1H-pyrazol-3-yl]-thiophene-2-carboxylic acid hydroxyamide (55 mg) as a glassy gum. ¹H NMR [(CD₃)₂SO]: δ 11.16 (s, 1H), 8.56 (d, 1H), 8.07 (d, 1H), 8.06 (d, 1H), 7.87 (m, 1H), 7.78 (d, 1H), 7.69 (m, 1H), 7.61 (d, 1H), 7.52 (apparent s br, 1H), 7.31 (d, 1H), 6.60 (d, 1H), 4.69 (t, 2H), 3.59 (t, 2H). LCMS (Method A): R_{T}=3.58 minutes; 365 (M+H)⁺.

### (bw) 5-(1-{[(Pyridin-3-ylmethyl)-carbamoyl]-methyl}-1H-pyrazol-3-yl)-thiophene-2-carboxylic acid hydroxyamide

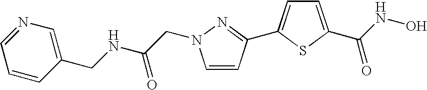

By proceeding in a similar manner to Example 1(ak) but using 5-(1-{[(pyridin-3-ylmethyl)-carbamoyl]-methyl}-1H-pyrazol-3-yl)-thiophene-2-carboxylic acid methyl ester [60 mg, 0.16 mmol, Reference Example 30(e)] and N,N dimethyl acetamide as co-solvent, (gradient 5:95 to 95:5, v/v, over 90 minutes), there was prepared 5-(1-{[(pyridin-3-ylmethyl)-carbamoyl]-methyl}-1H-pyrazol-3-yl)-thiophene-2-carboxylic acid hydroxyamide (3.2 mg) as a brown solid. LCMS (Method A): R_{T}=2.26 minutes; 358 (M+H)⁺.

### (bx) 5-(1-Biphenyl-4-ylmethyl-1H-pyrazol-3-yl)-thiophene-2-carboxylic acid hydroxyamide

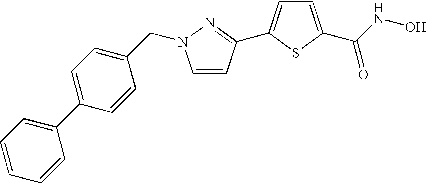

By proceeding in a similar manner to Reference 1(al) but using 5-(1-biphenyl-4-ylmethyl-1H-pyrazol-3-yl)-thiophene-2-carboxylic acid methyl ester [116 mg, 0.31 mmol, Reference Example 10(ac)], there was prepared 5-(1-biphenyl-4-ylmethyl-1H-pyrazol-3-yl)-thiophene-2-carboxylic acid hydroxyamide (33 mg). LCMS (Method C): R_{T}=3.31 minutes; 376 (M+H)⁺.

### (by) 5-{1-[6-(2,2-Dimethyl-propionylamino)-pyridin-2-ylmethyl]-1H-pyrazol-3-yl}-thiophene-2-carboxylic acid hydroxyamide

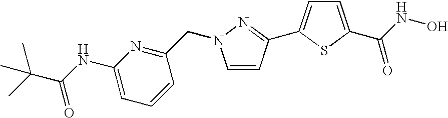

By proceeding in a similar manner to Reference 1(al) but using 5-{1-[6-(2,2-dimethyl-propionylamino)-pyridin-2-ylmethyl]-1H-pyrazol-3-yl}-thiophene-2-carboxylic acid methyl ester [124 mg, 0.31 mmol, Reference Example 10(ad)], there was prepared 5-{1-[6-(2,2-dimethyl-propionylamino)-pyridin-2-ylmethyl]-1H-pyrazol-3-yl}-thiophene-2-carboxylic acid hydroxyamide (42 mg). LCMS (Method C): R_{T}=2.72 minutes; 400 (M+H)⁺.

### (bz) 5-{1-[2-(Biphenyl-4-yloxy)-ethyl]-1H-pyrazol-3-yl}-thiophene-2-carboxylic acid hydroxyamide

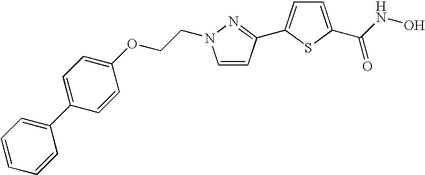

By proceeding in a similar manner to Reference 1(al) but using 5-{1-[2-(biphenyl-4-yloxy)-ethyl]-1H-pyrazol-3-yl}-thiophene-2-carboxylic acid methyl ester [124 mg, 0.31 mmol, Reference Example 10(ae)], there was prepared 5-{1-[2-(biphenyl-4-yloxy)-ethyl]-1H-pyrazol-3-yl}-thiophene-2-carboxylic acid hydroxyamide (6 mg). LCMS (Method C): R_{T}=3.38 minutes; 406 (M+H)⁺.

### (ca) 5-[1-(3-Phenoxy-benzyl-1H-pyrazol-3-yl]-thiophene-2-carboxylic acid hydroxyamide

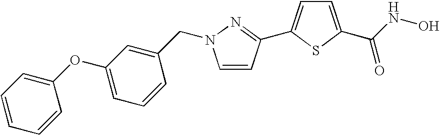

By proceeding in a similar manner to Reference 1(al) but using 5-[1-(3-phenoxy-benzyl)-1H-pyrazol-3-yl]-thiophene-2-carboxylic acid methyl ester [121 mg, 0.31 mmol, Reference Example 10(ag)], there was prepared 5-[1-(3-phenoxy-benzyl)-1H-pyrazol-3-yl]-thiophene-2-carboxylic acid hydroxyamide (12 mg). LCMS (Method C): R_{T}=3.28 minutes; 392 (M+H)⁺.

### (cb) 5-(1-{3-[4-(3-Chloro-phenyl)-piperazin-1-yl]-propyl}-1H-pyrazol-3-yl)-thiophene-2-carboxylic acid hydroxyamide

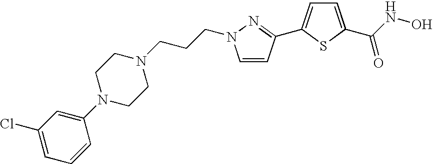

By proceeding in a similar manner to Example 1(bf) but using 5-(1-{3-[4-(3-chloro-phenyl)-piperazin-1-yl]-propyl}-1H-pyrazol-3-yl)-thiophene-2-carboxylic acid methyl ester [213 mg, 0.48 mmol, Reference Example 10(ah)] and subjecting the crude material to reverse-phase preparative HPLC using acetonitrile and water (gradient 5:95 to 95:5, v/v, over 90 minutes) as eluent, there was prepared 5-(1-{3-[4-(3-chloro-phenyl)-piperazin-1-yl]-propyl}-1H-pyrazol-3-yl)-thiophene-2-carboxylic acid hydroxyamide. LCMS (Method A): R_{T}=5.09 minutes; 446 (M+H)⁺.

### (cc) 5-{1-[(4-Morpholin-4-yl-phenylcarbamoyl)-methyl]-1H-pyrazol-3-yl}-thiophene-2-carboxylic acid hydroxyamide

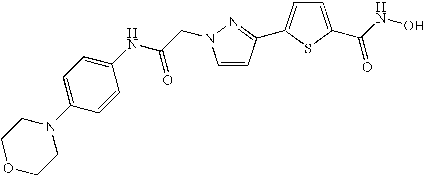

By proceeding in a similar manner to Example 1(bf) but using 5-{1-[(4-morpholin-4-yl-phenylcarbamoyl)-methyl]-1H-pyrazol-3-yl}-thiophene-2-carboxylic acid methyl ester [205 mg, 0.48 mmol, Reference Example 10(ai)] and triturating the final product in methanol, there was prepared 5-{1-[(4-morpholin-4-yl-phenylcarbamoyl)-methyl]-1H-pyrazol-3-yl}-thiophene-2-carboxylic acid hydroxyamide. LCMS (Method C): R_{T}=2.12 minutes; 428 (M+H)⁺.

### (cd) 5-{1-[(2-Morpholin-4-yl-phenylcarbamoyl)-methyl]-1H-pyrazol-3-yl}-thiophene-2-carboxylic acid hydroxyamide

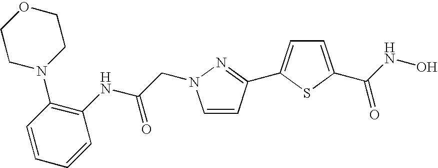

By proceeding in a similar manner to Example 1(bf) but using 5-{1-[(2-morpholin-4-yl-phenylcarbamoyl)-methyl]-1H-pyrazol-3-yl}-thiophene-2-carboxylic acid methyl ester [205 mg, 0.48 mmol, Reference Example 10(aj)] and triturating the final product in methanol, there was prepared 5-{1-[(2-morpholin-4-yl-phenylcarbamoyl)-methyl-1H-pyrazol-3-yl}-thiophene-2-carboxylic acid hydroxyamide. LCMS (Method C): R_{T}=2.54 minutes; 428 (M+H)⁺.

### (ce) 5-{1-[(4-Oxazol-5-yl-phenylcarbamoyl)-methyl]-1H-pyrazol-3-yl}-thiophene-2-carboxylic acid hydroxyamide

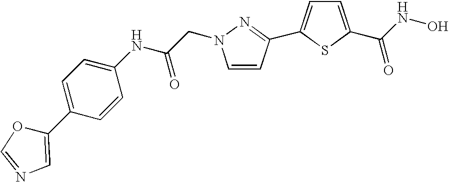

By proceeding in a similar manner to Example 1(bf) but using 5-{1-[(4-oxazol-5-yl-phenylcarbamoyl)-methyl]-1H-pyrazol-3-yl}-thiophene-2-carboxylic acid methyl ester [196 mg, 0.48 mmol, Reference Example 10(ak)] and subjecting the crude material to reverse-phase preparative HPLC using acetonitrile and water (gradient 5:95 to 95:5, v/v, over 90 minutes) as eluent, there was prepared 5-{1-[(4-oxazol-5-yl-phenylcarbamoyl-methyl]-1H-pyrazol-3-yl}-thiophene-2-carboxylic acid hydroxyamide. LCMS (Method A): R_{T}=5.71 minutes; 410 (+H)⁺.

### (cf) 5-{1-[(4-Acetylamino-phenylcarbamoyl)-methyl]-1H-pyrazol-3-yl}-thiophene-2-carboxylic acid hydroxyamide

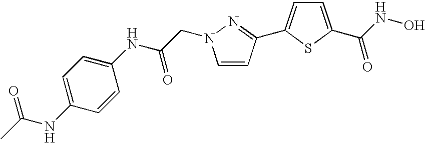

By proceeding in a similar manner to Example 1(bf) but using 5-{1-[(4-acetylamino-phenylcarbamoyl)-methyl]-1H-pyrazol-3-yl}-thiophene-2-carboxylic acid methyl ester [191 mg, 0.48 mmol, Reference Example 10(al)] and triturating the final product in methanol, there was prepared 5-{1-[(4-acetylamino-phenylcarbamoyl)-methyl]-1H-pyrazol-3-yl}-thiophene-2-carboxylic acid hydroxyamide. LCMS (Method A): R_{T}=4.54 minutes; 400 (M+H)⁺.

### (cg) 5-1-(1-Oxy-quinolin-2-ylmethyl)-1H-pyrazol-3-yl]-thiophene-2-carboxylic acid hydroxyamide

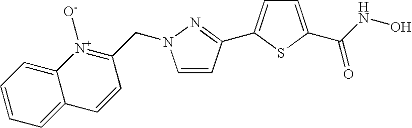

Acetyl chloride (1.93 ml, 21.2 mmol) was added slowly to anhydrous methanol (10 ml) and stirred for 30 minutes, then a solution of 5-[1-(1-oxy-quinolin-2-ylmethyl)-1H-pyrazol-3-yl]-thiophene-2-carboxylic acid (tetrahydro-pyran-2-yloxy)-amide [596 mg, 0.1.32 mmol, Reference Example 1(au)] in methanol (5 ml) was added. The resulting mixture was stirred for 3 hours, concentrated and the residue was subjected to reverse-phase preparative HPLC using acetonitrile and water (gradient 5:95 to 95:5, v/v, over 90 minutes) as eluent, to provide 5-[1-(1-oxy-quinolin-2-ylmethyl)-1H-pyrazol-3-yl]-thiophene-2-carboxylic acid hydroxyamide. ¹H NMR [(CD₃)₂SO]: δ 11.20 (s, 1H), 9.11 (s, 1H), 8.59 (d, 1H), 8.06-8.11 (m, 2H), 7.93 (d, 1H), 7.87 (m, 1H), 7.75 (m, 1H), 7.56 (apparent s br, 1H), 7.43 (d, 1H), 6.91 (d, 1H), 6.81 (d, 1H), 5.75 (s, 2H). LCMS (Method A): R_{T}=5.17 minutes; 367 (M+H)⁺.

### (ch) 5-(1-{2-Oxo-2-[4-(4-trifluoromethyl-pyrimidin-2-yl)-piperazin-1-yl]-ethyl}-1H-pyrazol-3-yl)-thiophene-2-carboxylic acid hydroxyamide

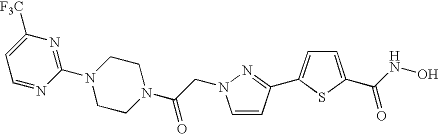

By proceeding in a similar manner to Example 1(bf) but using 5-(1-{2-oxo-2-[4-(4-trifluoromethyl-pyrimidin-2-yl)-piperazin-1-yl]-ethyl}-1H-pyrazol-3-yl)-thiophene-2-carboxylic acid methyl ester [231 mg, 0.48 mmol, Reference Example 10(ap)] and triturating the final product in methanol, there was prepared 5-(1-{2-oxo-2-[4-(4-trifluoromethyl-pyrimidin-2-yl)-piperazin-1-yl]-ethyl}-1H-pyrazol-3-yl)-thiophene-2-carboxylic acid hydroxyamide. ¹H NMR [(CD₃)₂SO]: δ 11.18 (s br, 1H), 9.08 (s br, 1H), 8.73 (d, 1H), 7.74 (d, 1H), 7.54 (apparent s, 1H), 7.37 (d, 1H), 7.08 (d, 1H), 6.69 (d, 1H), 5.26 (s, 2H), 3.89 (t br, 2H), 3.81 (t br, 2H), 3.65 (t br, 2H), 3.60 (t br, 2H). LCMS (Method A): R_{T}=7.21 minutes; 482 (M+H)⁺.

### (ci) 5-(6-{[(Pyridin-3-ylmethyl)-amino]-methyl}-pyridin-2-yl)-thiophene-2-carboxylic acid hydroxyamide

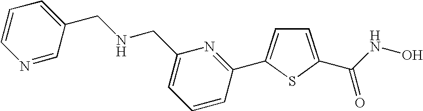

A solution of 5-(6-{[(pyridin-3-ylmethyl)-amino]-methyl}-pyridin-2-yl)-thiophene-2-carboxylic acid (tetrahydro-pyran-2-yloxy)-amide [170 mg, 0.5 mmol, Reference Example 1(ai)] in dichloromethane (4.5 ml) was treated with trifluoroacetic acid (0.5 ml) and a drop of water. The solution was stirred at room temperature overnight, and then concentrated under reduced pressure to give a residue. The residue was subjected to reverse-phase preparative HPLC using acetonitrile and water (gradient 5:95 to 100:0, v/v, over 15 minutes) as eluent, to provide 5-(6-{[(pyridin-3-ylmethyl)-amino]-methyl}-pyridin-2-yl)-thiophene-2-carboxylic acid hydroxyamide. LCMS (Method C): R_{T}=0.35 minutes; 341 (M+H)⁺.

### (cj) 5-{6-[(2-Pyridin-3-yl-ethylamino)-methyl]-pyridin-2-yl}-thiophene-2-carboxylic acid hydroxyamide

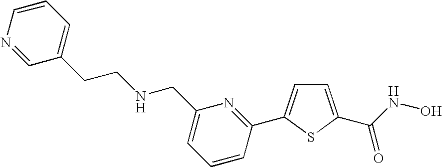

By proceeding in a similar manner to Example 1(ci) but using 5-{6-[(2-pyridin-3-yl-ethylamino)-methyl]-pyridin-2-yl}-thiophene-2-carboxylic acid (tetrahydro-pyran-2-yloxy)-amide [177 mg, 0.5 mmol, Reference Example 1(aj)] and using acetonitrile and water (gradient 5:95 to 100:0, v/v, over 15 minutes) as eluent, there was prepared 5-{6-[(2-pyridin-3-yl-ethylamino-methyl]-pyridin-2-yl}-thiophene-2-carboxylic acid hydroxyamide. LCMS (Method C): R_{T}=0.34 minutes; 355 (M+H)⁺.

### (ck) 5-{6-[(4-Fluoro-benzylamino)-methyl]-pyridin-2-yl}-thiophene-2-carboxylic acid hydroxyamide

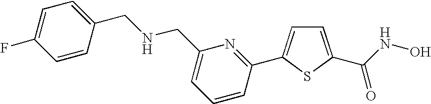

By proceeding in a similar manner to Example 1(ci) but using 5-{6-[(4-fluoro-benzylamino)-methyl]-pyridin-2-yl}-thiophene-2-carboxylic acid (tetrahydro-pyran-2-yloxy)-amide [178 mg, 0.5 mmol, Reference Example 1(ak)] and using acetonitrile and water (gradient 5:95 to 100:0, v/v, over 15 minutes) as eluent, there was prepared 5-{6-[(4-fluoro-benzylamino)-methyl]-pyridin-2-yl}-thiophene-2-carboxylic acid hydroxyamide. LCMS (Method C): R_{T}=1.86 minutes; 358 (M+H)⁺.

### (cl) 5-(6-{[(Benzo[1,3]dioxol-5-ylmethyl)-amino]-methyl}-pyridin-2-yl)-thiophene-2-carboxylic acid hydroxyamide

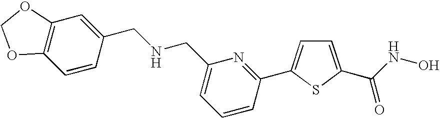

By proceeding in a similar manner to Example 1(ci) but using 5-(6-{[(benzo[1,3]dioxol-5-ylmethyl)-amino]-methyl}-pyridin-2-yl)-thiophene-2-carboxylic acid (tetrahydro-pyran-2-yloxy)-amide [191 mg, 0.5 mmol, Reference Example 1(al)] and using acetonitrile and water (gradient 5:95 to 100:0, v/v, over 15 minutes) as eluent, there was prepared 5-(6-{[(benzo[1,3]dioxol-5-ylmethyl)-amino]-methyl}-pyridin-2-yl)-thiophene-2-carboxylic acid hydroxyamide. LCMS (Method C): R_{T}=1.85 minutes; 384 (M+H)⁺.

### (cm) 5-(6-{[(1H-Benzoimidazol-2-ylmethyl)-amino]-methyl}-pyridin-2-yl)-thiophene-2-carboxylic acid hydroxyamide

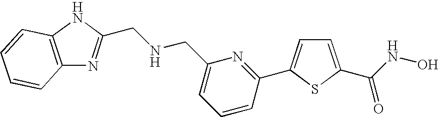

By proceeding in a similar manner to Example 1(ci) but using 5-(6-{[(1H-benzoimidazol-2-ylmethyl)-amino]-methyl}-pyridin-2-yl)-thiophene-2-carboxylic acid (tetrahydro-pyran-2-yloxy)-amide [144 mg, 0.38 mmol, Reference Example 1(am) and using acetonitrile and water (gradient 5:95 to 100:0, v/v, over 15 minutes) as eluent, there was prepared 5-(6-{[(1H-benzoimidazol-2-ylmethyl)-amino]-methyl}-pyridin-2-yl)-thiophene-2-carboxylic acid hydroxyamide. LCMS (Method C): R_{T}=1.70 minutes; 380 (M+H)⁺.

### (cn) 5-{6-[(3-Imidazol-1-yl-propylamino)-methyl]-pyridin-2-yl}-thiophene-2-carboxylic acid hydroxyamide

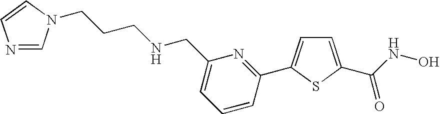

By proceeding in a similar manner to Example 1(ci) but using 5-{6-[(3-imidazol-1-yl-propylamino)-methyl]-pyridin-2-yl}-thiophene-2-carboxylic acid (tetrahydro-pyran-2-yloxy)-amide [178 mg, 0.5 mmol, Reference Example 1(an)] and using acetonitrile and water (gradient 5:95 to 100:0, v/v, over 15 minutes) as eluent, there was prepared 5-{6-[(3-imidazol-1-yl-propylamino)-methyl]-pyridin-2-yl}-thiophene-2-carboxylic acid hydroxyamide. LCMS (Method C): R_{T}=0.33 minutes; 358 (M+H)⁺.

### (co) 5-{6-[(4-Methoxy-phenylamino)-methyl]-pyridin-2-yl}-thiophene-2-carboxylic acid hydroxyamide

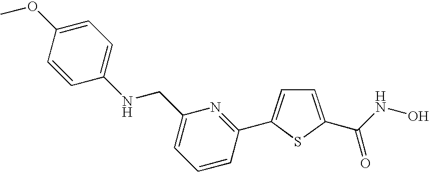

To a slowly stirred suspension of 5-{6-[(4-methoxy-phenylamino)-methyl]-pyridin-2-yl}-thiophene-2-carboxylic acid [129 mg, 0.38 mmol, Reference Example 32(b)], hydroxylamine Wang® resin (380 mg, 1.0 mmol/g loading) and pyridine (81 μl, 1.0 mmol) in dimethylformamide (5 ml) was added O-(7-azabenzotriazol-1-yl)-N,N,N′,N′-tetramethyluronium hexafluorophosphate (159 mg, 0.43 mmol). The mixture was then shaken at room temperature for 4 hours and filtered. The resin was washed alternatively with methanol and dichloromethane (3×) and dried. The resin was treated with 20% trifluoroacetic acid in dichloromethane solution (5 ml), shaken for 30 minutes, filtered, and washed with dichloromethane. The filtrate was concentrated to give a purple oil, which was subjected to reverse-phase preparative HPLC using acetonitrile and water (gradient 5:95 to 5:95, v/v, over 90 minutes) as eluent, to provide 5-{6-[(4-methoxy-phenylamino)-methyl]-pyridin-2-yl}-thiophene-2-carboxylic acid hydroxyamide (8.4 mg) as a brown solid. LCMS (Method C): R_{T}=2.18 minutes; 356 (M+H)⁺.

### (cp) 5-[6-(Methyl-phenethyl-amino)-pyridin-2-yl]-thiophene-2-carboxylic acid hydroxyamide

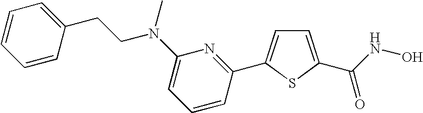

By proceeding in a similar manner to Example 1(co) but using 5-[6-(methyl-phenethyl-amino)-pyridin-2-yl]-thiophene-2-carboxylic acid [100 mg, 0.3 mmol, Reference Example 33(a)] there was prepared 5-[6-(methyl-phenethyl-amino)-pyridin-2-yl]-thiophene-2carboxylic acid hydroxyamide (9.4 mg) as a brown oil. LCMS (Method C): R_{T}=3.50 minutes; 354 (M+H)⁺.

### (cq) 5-{6-[(Methyl-pyridin-3-ylmethyl-amino)-methyl]-pyridin-2-yl}-thiophene-2-carboxylic acid hydroxyamide

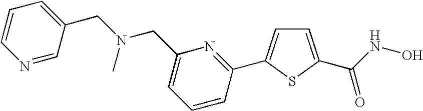

By proceeding in a similar manner to Example 1(co) but using 5-{6-[(methyl-pyridin-3-ylmethyl-amino)-methyl]-pyridin-2-yl}-thiophene-2-carboxylic acid [129 mg, 0.38 mmol, Reference Example 32(c)] there was prepared 5-{6-[(methyl-pyridin-3-ylmethyl-amino)-methyl]-pyridin-2-yl}-thiophene-2-carboxylic acid hydroxyamide (36 mg) as a light brown gum. ¹H NMR (CD₃OD): δ 8.97 (apparent s, 1H), 8.82 (apparent s, 1H), 8.48 (d, 1H), 7.94 (m, 2H), 7.84 (dd, 1H), 7.78 (d, 1H), 7.59 (apparent s, 1H), 7.45 (m, 1H), 4.74 (s, 2H), 4.63 (s, 2H), 3.05 (s, 3H). LCMS (Method A): R_{T}=2.97 minutes; 355 (M+H)⁺.

### (cr) 5-[6-(3,4-Tetrahydro-1H-isoquinolin-2-ylmethyl)-pyridin-2-yl]-thiophene-2-carboxylic acid hydroxyamide

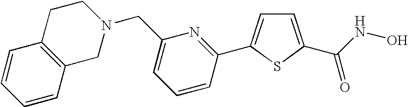

By proceeding in a similar manner to Example 1(co) but using 5-[6-(3,4-tetrahydro-1H-isoquinolin-2-ylmethyl)-pyridin-2-yl]-thiophene-2-carboxylic acid [133 mg, 0.28 mmol, Reference Example 32(d)] there was prepared 5-[6-(3,4-tetrahydro-1H-isoquinolin-2-ylmethyl)-pyridin-2-yl]-thiophene-2-carboxylic acid hydroxyamide (1.7 mg). LCMS (Method C): R_{T}=1.84 minutes; 366 (M+H)⁺.

### (cs) 5-{6-[(Methyl-naphthalen-1-ylmethyl-amino)-methyl]-pyridin-2-yl}-thiophene-2-carboxylic acid hydroxyamide

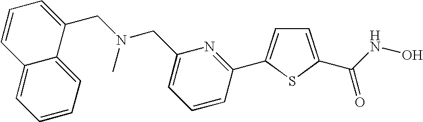

By proceeding in a similar manner to Example 1(co) but using 5-{6-[(methyl-naphthalen-1-ylmethyl-amino)-methyl]-pyridin-2-yl}-thiophene-2-carboxylic acid [92 mg, 0.28 mmol, Reference Example 32(e)] there was prepared 5-{6-[(methyl-naphthalen-1-ylmethyl-amino)-methyl]-pyridin-2-yl}-thiophene-2-carboxylic acid hydroxyamide (7.9 mg). LCMS (Method C): R_{T}=2.05 minutes; 404 (M+H)⁺.

### (ct) 5-[6-(4-Phenethyl-piperazin-1-ylmethyl)-pyridin-2-yl]-thiophene-2-carboxylic acid hydroxyamide

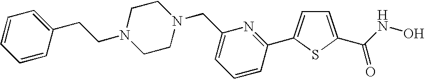

By proceeding in a similar manner to Example 1(co) but using 5-[6-(4-phenethyl-piperazin-1-ylmethyl)-pyridin-2-yl]-thiophene-2-carboxylic acid [114 mg, 0.28 mmol, Reference Example 32(g)] there was prepared 5-[6-(4-phenethyl-piperazin-1-ylmethyl) -pyridin-2-yl]-thiophene-2-carboxylic acid hydroxyamide (1.3 mg). LCMS (Method C): R_{T}=1.86 minutes; 423 (M+H)⁺.

### (cu) 5-[6-(4-Pyridin-2-yl-piperazin-1-ylmethyl-pyridin-2-yl]-thiophene-2-carboxylic acid hydroxyamide

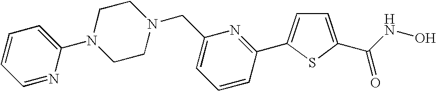

By proceeding in a similar manner to Example 1(co) but using 5-[6-(4-pyridin-2-yl-piperazin-1-ylmethyl)-pyridin-2-yl]-thiophene-2-carboxylic acid [106 mg, 0.28 mmol, Reference Example 32(h)] there was prepared 5-[6-(4-pyridin-2-yl-piperazin-1-ylmethyl)-pyridin-2-yl]-thiophene-2-carboxylic acid hydroxyamide (8.7 mg). LCMS (Method C): R_{T}=1.45 minutes; 396 (M+H)⁺.

### (cv) 2-(5-Hydroxycarbamoyl-thiophen-2-yl)-5-methyl-1H-imidazole-4-carboxylic acid phenethyl-amide

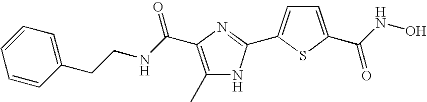

By proceeding in a similar manner to Example 1(bd) but using 5-(5-methyl-4-phenethylcarbamoyl-1H-imidazol-2-yl)-thiophene-2-carboxylic acid methyl ester [39 mg, 0.11 mmol, Reference Example 30(1)], there was prepared 2-(5-hydroxycarbamoyl-thiophen-2-yl)-5-methyl-1H-imidazole-4-carboxylic acid phenethyl-amide (18 mg) as an off-white solid. ¹H NMR (CD₃OD): δ 12.89 (s br, 1H), 11.29 (s, 1H), 9.17 (s br, 1H), 7.76 (t, 1H), 7.57 (apparent s, 1H), 7.48 (d, 1H), 7.31 (m, 2H), 7.25 (m, 2H), 7.21 (m, 1H), 3.46 (m, 2H), 2.83 (t, 2H), 2.50 (s, 3H). LCMS (Method A): R_{T}=5.87 minutes; 371 (M+H)⁺.

### (cw) 2-(5-Hydroxycarbamoyl-thiophen-2-yl)-5-methyl-1H-imidazole-4-carboxylic acid benzylamide

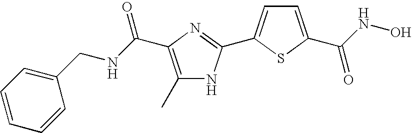

By proceeding in a similar manner to Example 1(bd) but using 5-(4-benzylcarbamoyl-5-methyl-1H-imidazol-2-yl)-thiophene-2-carboxylic acid methyl ester [50 mg, 0.14 mmol, Reference Example 30(m)], there was prepared 2-(5-hydroxycarbamoyl-thiophen-2-yl)-5-methyl-1H-imidazole-4-carboxylic acid benzylamide (18 mg) as a gum. ¹H NMR (CD₃OD): δ 12.94 (s br, 1H), 11.30 (s, 1H), 9.13 (s br, 1H), 8.25 (t, 1H), 7.57 (apparent s, 1H), 7.49 (d, 1H), 7.32 (apparent d, 4H), 7.23 (m, 1H), 4.43 (d, 2H), 2.51 (s, 3H). LCMS (Method A): R_{T}=5.47 minutes; 357 (M+H)⁺.

### (cx) 5-(6-Benzyloxymethyl-pyridin-2-yl)-thiophene-2-carboxylic acid hydroxyamide

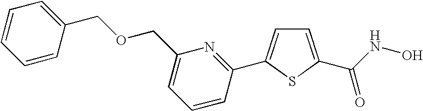

By proceeding in a similar manner to Example 1(bd) but using 5-(6-benzyloxymethyl-pyridin-2-yl)-thiophene-2-carboxylic acid methyl ester [118 mg, 0.35 mmol, Reference Example 29(b)], there was prepared 5-(6-benzyloxymethyl-pyridin-2-yl-thiophene-2-carboxylic acid hydroxyamide (11 mg) as an off-white solid. ¹H NMR (CD₃OD): δ 7.83 (t, 1H), 7.75 (d, 1H), 7.66 (d, 1H), 7.57 (apparent s, 1H), 7.44 (d, 1H), 7.42 (apparent d, 2H), 7.36 (m, 2H), 7.29 (m, 1H), 4.68 (s, 2H), 4.67 (s, 2H). LCMS (Method A): R_{T}=7.68 minutes; 341 (M+H)⁺.

### (cy) 5-[6-(3-Phenyl-propionylamino)-pyridin-2-yl]-thiophene-2-carboxylic acid hydroxyamide

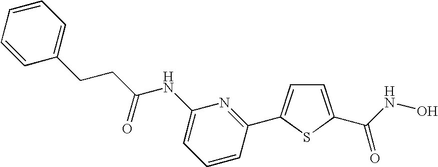

By proceeding in a similar manner to Example 1(a) but using 5-[6-(3-phenyl-propionylamino)-pyridin-2-yl]-thiophene-2-carboxylic acid (tetrahydro-pyran-2-yloxy)-amide [64 mg, 0.14 mmol, Reference Example 1(ap)], and subjecting the crude product to reverse-phase HPLC using acetonitrile and water (gradient, 30:70 to 70:30, v/v, over 40 minutes) as eluent, there was prepared 5-[6-(3-phenyl-propionylamino)-pyridin-2-yl]-thiophene-2-carboxylic acid hydroxyamide (29 mg). ¹H NMR (CD₃OD): δ 8.05 (d, 1H), 7.78 (t, 1H), 7.64 (d, 1H), 7.56 (d, 1H), 7.54 (apparent s, 1H), 7.27 (d, 4H), 7.17 (m, 1H), 3.01 (t, 2H), 2.77 (t, 2H). LCMS (Method A): R_{T}=7.56 minutes; 368 (M+H)⁺.

### (cz) 5-{1-[(3-Methoxy-phenylcarbamoyl)-methyl]-1H-pyrazol-3-yl}-thiophene-2-carboxylic acid hydroxyamide

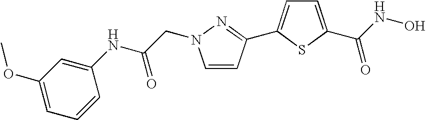

Acetyl chloride (0.8 ml, 11.5 mmol) was added slowly to anhydrous methanol (20 ml) at 0° C. and the resulting solution was stirred for 1 hour, before 5-{1-[(3-methoxy-phenylcarbamoyl)-methyl]-1H-pyrazol-3-yl}-thiophene-2-carboxylic acid (tetrahydro-pyran-2-yloxy)-amide [300 mg, 0.66 mmol, Reference Example 1(aq)] was added. The mixture was allowed to warm to room temperature and stirred for a further 30 minutes then concentrated, to provide 5-{1-[(3-methoxy-phenylcarbamoyl)-methyl]-1H-pyrazol-3-yl}-thiophene-2-carboxylic acid hydroxyamide (130 mg) as a white solid. ¹H NMR (CD₃OD): δ 11.19 (s br, 1H), 10.45 (s, 1H), 7.85 (d, 1H), 7.57 (apparent s br, 1H), 7.39 (d, 1H), 7.32 (t, 1H), 7.23 (t, 1H), 7.12 (m, 1H), 6.71 (d, 1H), 6.66 (m, 1H), 5.06 (s, 2H), 3.72 (s, 3H). LCMS (Method A): R_{T}=5.73 minutes; 373 (M+H)⁺.

### (da) 5-{1-[(3-Chloro-phenylcarbamoyl)-methyl]-1H-pyrazol-3-yl}-thiophene-2-carboxylic acid hydroxyamide

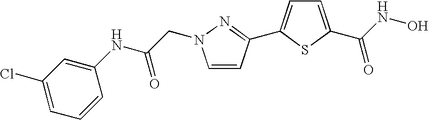

By proceeding in a similar manner to Example 1(bd) but using 5-{1-[(3-chloro-phenylcarbamoyl)-methyl]-1H-pyrazol-3-yl}-thiophene-2-carboxylic acid methyl ester [56 mg, 0.15 mmol, Reference Example 30(g)], there was prepared 5-{1-[(3-chloro-phenylcarbamoyl)-methyl]-1H-pyrazol-3-yl}-thiophene-2-carboxylic acid hydroxyamide (16 mg). ¹H NMR (CD₃OD): δ 11.19 (s, 1H), 10.56 (s, 1H), 9.10 (s br, 1H), 7.85 (d, 1H), 7.80 (t, 1H), 7.55 (apparent s br, 1H), 7.46 (m, 1H), 7.39 (d, 1H), 7.37 (t, 1H), 7.15 (m, 1H), 6.72 (d, 1H), 5.08 (s, 2H). LCMS (Method A): R_{T}=6.63 minutes; 377 (M+H)⁺.

### (db) 5-{1-[(3,5-Difluoro-phenylcarbamoyl)-methyl]-1H-pyrazol-3-yl}-thiophene-2-carboxylic acid hydroxyamide

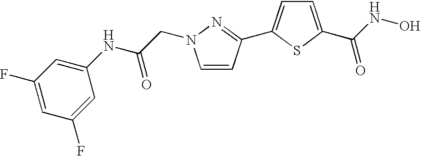

By proceeding in a similar manner to Example 1(bd) but using 5-{1-[(3,5-difluoro-phenylcarbamoyl)-methyl]-1H-pyrazol-3-yl}-thiophene-2-carboxylic acid methyl ester [22 mg, 0.06 mmol, Reference Example 30(h)], there was prepared 5-{1-[(3,5-difluoro-phenylcarbamoyl)-methyl]-1H-pyrazol-3-yl}-thiophene-2-carboxylic acid hydroxyamide (9 mg). ¹H NMR (CD₃OD): δ 11.19 (s, 1H), 10.76 (s, 1H), 9.10 (s, 1H), 7.85 (d, 1H), 7.56 (apparent s br, 1H), 7.40 (d, 1H), 7.31 (m, 2H), 6.96 (tt, 1H), 6.72 (d, 1H), 5.09 (s, 2H). LCMS (Method A): R_{T}=6.46 minutes; 379 (M+H)⁺.

### (dc) 5-{1-[(3-Sulfamoyl-phenylcarbamoyl)-methyl]-1H-pyrazol-3-yl}-thiophene-2-carboxylic acid hydroxyamide

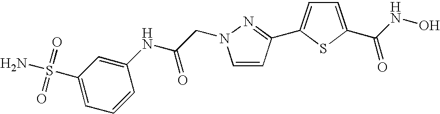

By proceeding in a similar manner to Example 1(bd) but using 5-{1-[(3-sulfamoyl-phenylcarbamoyl)-methyl]-1H-pyrazol-3-yl}-thiophene-2-carboxylic acid methyl ester [63 mg, 0.15 mmol, Reference Example 30(i)], there was prepared 5-{1-[(3-sulfamoyl-phenylcarbamoyl)-methyl]-1H-pyrazol-3-yl}-thiophene-2-carboxylic acid hydroxyamide (6.7 mg). ¹HNMR(CD₃OD): δ 11.18 (s, 1H), 10.67 (s, 1H), 9.09 (s br, 1H), 8.16 (m, 1H), 7.86 (d, 1H), 7.74 (m, 1H), 7.54 (m, 3H), 7.39 (d, 1H), 7.37 (s, 2H), 6.72 (d, 1H), 5.09 (s, 2H). LCMS (Method A): R_{T}=4.41 minutes; 422 (M+H)⁺.

### (dd) 5-{1-[(1H-Indazol-7-ylcarbamoyl)-methyl]-1H-pyrazol-3-yl}-thiophene-2-carboxylic acid hydroxyamide

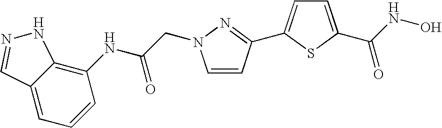

By proceeding in a similar manner to Example 1(bd) but using 5-{1-[(1H-indazol-7-ylcarbamoyl)-methyl]-1H-pyrazol-3-yl}-thiophene-2-carboxylic acid methyl ester [71 mg, 0.19 mmol, Reference Example 30(j)], there was prepared 5-{1-[(1H-indazol-7-ylcarbamoyl)-methyl]-1H-pyrazol-3-yl}-thiophene-2-carboxylic acid hydroxyamide (15 mg). ¹H NMR (CD₃OD): δ 12.69 (s, 1H), 11.20 (s, 1H), 10.29 (s, 1H), 9.11 (s br, 1H), 8.11 (s, 1H), 7.90 (d, 1H), 7.58 (d, 1H), 7.56 (apparent s br, 1H), 7.52 (d br, 1H), 7.40 (d, 1H), 7.09 (t, 1H), 6.74 (d, 1H), 5.18 (s, 2H). LCMS (Method A): R_{T}=5.00 minutes; 383 (M+H)⁺.

### (de) 5-{1-[(1H-Indol-7-ylcarbamoyl)-methyl]-1H-pyrazol-3-yl}-thiophene-2-carboxylic acid hydroxyamide

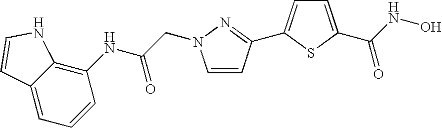

By proceeding in a similar manner to Example 1(bd) but using 5-{1-[(1H-indol-7-ylcarbamoyl)-methyl]-1H-pyrazol-3-yl}-thiophene-2-carboxylic acid methyl ester [57 mg, 0.15 mmol, Reference Example 30(k)], there was prepared 5-{1-[(1H-indol-7-ylcarbamoyl)-methyl]-1H-pyrazol-3-yl}-thiophene-2-carboxylic acid hydroxyamide (10 mg). ¹H NMR (CD₃OD): δ 11.17 (s br, 2H), 9.94 (s, 11H), 9.10 (s br, 11H), 7.88 (d, 11H), 7.60 (d, 1H), 7.56 (apparent s br, 1H), 7.40 (d, 1H), 7.33 (t, 1H), 7.17 (d, 1H), 7.02 (t, 1H), 6.73 (d, 2H), 5.20 (s, 2H). LCMS (Method A): R_{T}=5.17 minutes; 382 (M+H)⁺.

### (df) 5-[6-(3-Phenyl-propylamino pyridin-2-yl]-thiophene-2-carboxylic acid hydroxyamide

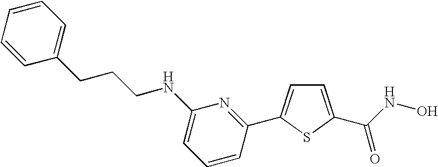

By proceeding in a similar manner to Example 1(a) but using 5-[6-(3-phenyl-propylamino)-pyridin-2-yl]-thiophene-2-carboxylic acid methyl ester [243 mg, 0.56 mmol, Reference Example 28(k)], and subjecting the crude product to reverse-phase HPLC using acetonitrile and water (gradient, 25:75 to 70:30, v/v) as eluent, there was prepared 5-[6-(3-phenyl-propylamino)-pyridin-2-yl]-thiophene-2-carboxylic acid hydroxyamide (105 mg) as a solid. ¹H NMR (CD₃OD): δ 7.64 (apparent t br, 1H), 7.58 (s, 2H), 7.20-7.30 (m, 4H), 7.15 (m, 1H), 7.07 (d, 1H), 6.67 (d, 1H), 4.34 (t, 2H), 2.75 (t, 2H), 1.99 (m, 2H). LCMS (Method A): R_{T}=7.31 minutes; 354 (M+H)⁺.

### (dg) 5-[1-(2-Benzylamino-ethyl)-1H-pyrazol-3-yl]-thiophene-2-carboxylic acid hydroxyamide

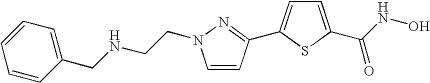

By proceeding in a similar manner to Example 1(co) but using 5-[1-(2-benzylamino-ethyl)-1H-pyrazol-3-yl]-thiophene-2-carboxylic acid [80 mg, 0.24 mmol, Reference Example 6(c)] there was prepared 5-[1-(2-benzylamino-ethyl)-1H-pyrazol-3-yl]-thiophene-2-carboxylic acid hydroxyamide (16 mg) as an orange/brown gum. LCMS (Method C): R_{T}=1.67 minutes; 343 (M+H)⁺.

### (dh) 5-(1-{3-[(Quinolin-2-ylmethyl)-amino]-propyl}-1H-pyrazol-3-yl)-thiophene-2-carboxylic acid hydroxyamide

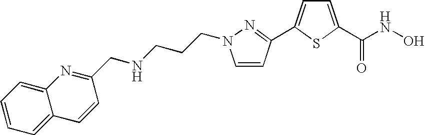

To a slowly stirred suspension of 5-(1-{3-[(quinolin-2-ylmethyl)-amino]-propyl}-1H-pyrazol-3-yl)-thiophene-2-carboxylic acid [118 mg, 0.3 mmol, Reference Example 6(d)], hydroxylamine Wang® resin (300 mg, 1.0 mmol/g loading) and pyridine (150 μl, 0.9 mmol) in dimethylformamide (3 ml) was added O-(7-azabenzotriazol-1-yl)-N,N,N′,N′-tetramethyluronium hexafluorophosphate (129 mg, 0.33 mmol). The mixture was then stirred at room temperature overnight and filtered. The resin was washed alternatively with methanol and dichloromethane (3×) and dried. The resin was treated with 50% trifluoroacetic acid in dichloromethane solution (3 ml), shaken for 30 minutes, filtered, and washed with dichloromethane. The filtrate was concentrated to give a residue, which was triturated with diethyl ether followed by acetonitrile, to provide 5-(1-{3-[(quinolin-2-ylmethyl)-amino]-propyl}-1H-pyrazol-3-yl)-thiophene-2-carboxylic acid hydroxyamide (9.7 mg). LCMS (Method C): R_{T}=1.79 minutes; 408 (M+H)⁺.

### (di) 5-(1-{3-[(Benzo[1,3]dioxol-5-ylmethyl)-amino]-propyl}-1H-pyrazol-3-yl)-thiophene-2-carboxylic acid hydroxyamide

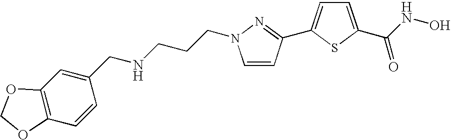

By proceeding in a similar manner to Example 1(dh) but using 5-(1-{3-[(benzo[1,3]dioxol-5-ylmethyl)-amino]-propyl}-1H-pyrazol-3-yl)-thiophene-2-carboxylic acid [116 mg, 0.3 mmol, Reference Example 6(e)] there was prepared 5-(1-{3-[(benzo[1,3]dioxol-5-ylmethyl)-amino]-propyl}-1H-pyrazol-3-yl)-thiophene-2-carboxylic acid hydroxyamide (19.9 mg). LCMS (Method C): R_{T}=1.75 minutes; 401 (M+H)⁺.

### (dj) 5-(1-{2-[(Benzo[1,3]dioxol-5-ylmethyl-amino]-ethyl}-1H-pyrazol-3-yl)-thiophene-2-carboxylic acid hydroxyamide

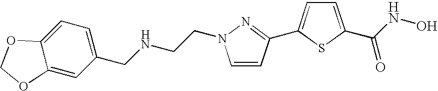

By proceeding in a similar manner to Example 1(dh) but using 5-(1-{2-[(benzo[1,3]dioxol-5-ylmethyl)-amino]-ethyl}-1H-pyrazol-3-yl)-thiophene-2-carboxylic acid [111 mg, 0.3 mmol, Reference Example 6(f)] there was prepared 5-(1-{2-[(benzo[1,3]dioxol-5-ylmethyl)-amino]-ethyl}-1H-pyrazol-3-yl)-thiophene-2-carboxylic acid hydroxyamide (8.9 mg). LCMS (Method C): R_{T}=1.84 minutes; 387 (M+H)⁺.

### (dk) 5-(1-{2-[(Pyridin-4-ylmethyl)-amino]-ethyl}-1H-pyrazol-3-yl)-thiophene-2-carboxylic acid hydroxyamide

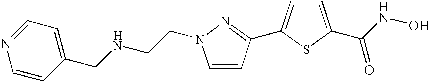

By proceeding in a similar manner to Example 1(dh) but using 5-(1-{2-[(pyridin-4-ylmethyl)-amino]-ethyl}-1H-pyrazol-3-yl)-thiophene-2-carboxylic acid [98 mg, 0.3 mmol, Reference Example 6(g)] there was prepared 5-(1-{2-[(pyridin-4-ylmethyl)-amino]-ethyl}-1H-pyrazol-3-yl)-thiophene-2-carboxylic acid hydroxyamide (4.4 mg). LCMS (Method C): R_{T}=1.47 minutes; 344 (M+H)⁺.

### (dl) 5-{6-[(Benzo[1,3]dioxol-5-ylmethyl-methyl-amino)-methyl]-pyridin-2-yl}-thiophene-2-carboxylic acid hydroxyamide

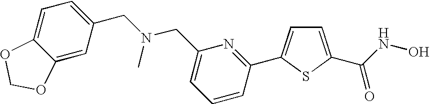

By proceeding in a similar manner to Example 1(a) but using 5-{6-[(benzo[1,3]dioxol-5-ylmethyl-methyl-amino)-methyl]-pyridin-2-yl}-thiophene-2-carboxylic acid (tetrahydro-pyran-2-yloxy)-amide [197 mg, 0.41 mmol, Reference Example 1(at)], and subjecting the crude product to reverse-phase HPLC using acetonitrile and water (gradient, 15:85 to 95:5, v/v) as eluent, there was prepared 5-{6-[(benzo[1,3]dioxol-5-ylmethyl-methyl-amino)-methyl)-pyridin-2-yl}-thiophene-2-carboxylic acid hydroxyamide (31 mg) as a solid. ¹H NMR (CD₃OD): δ 7.91-7.98 (m, 2H), 7.79 (d, 1H), 7.60 (apparent s br, 1H), 7.38 (m, 1H), 7.04-7.08 (m, 2H), 6.91 (d, 1H), 6.01 (s, 2H), 4.52 (apparent d br, 3H), 4.31 (apparent s br, 1H), 2.96 (s, 3H). LCMS (Method A): R_{T}=4.19 minutes; 398 (M+H)⁺.

### Reference Example 1

### a) 5-(2-Methyl-5-trifluoromethyl-2H-pyrazol-3-yl)-thiophene-2-carboxylic acid (tetra-hydro-p ran-2-yloxy)-amide

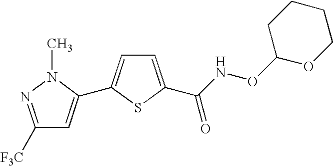

A solution of 5-[2-methyl-5-(trifluoromethyl)-2H-pyrazol-3-yl]thiophene-2-carboxylic acid (80 mg, 0.29 mmol) in dimethylformamide (1.2 ml) was treated with diisopropylethylamine (151 μl, 0.87 mmol), O-(tetrahydro-2H-pyran-2-yl)hydroxylamine (39 mg, 0.33 mmol) and O-(7-azabenzotriazol-1-yl)-N,N,N′,N′-tetramethyluronium hexafluorophosphate (110 mg, 0.29 mmol). The mixture was stirred at room temperature for 4 hours when t.l.c. analysis [ethyl acetate/methanol, 3:1, v/v] indicated complete consumption of the starting carboxylic acid. The reaction mixture was evaporated under reduced pressure and the residue was partitioned between ethyl acetate and saturated sodium bicarbonate solution. The two phases were separated and the organic phase was washed with water, then dried over sodium sulfate and then evaporated under reduced pressure. The crude product was subjected to flash column chromatography on silica eluting with a mixture of ethyl acetate and petroleum ether fraction (b.p. 30-50° C.), (3:2, v/v), to give 5-(2-methyl-5-trifluoromethyl-2H-pyrazol-3-yl]-thiophene-2-carboxylic acid (tetrahydro-pyran-2-yloxy)-amide (85 mg, 78%) as a white solid. LCMS (Method A): R_{T}=8.45 minutes; 376 (M+H)⁺.

### (b) 5-(2-Methyl-2H-pyrazol-3-yl)-thiophene-2-carboxylic acid (tetrahydro-pyran-2-yloxy)-amide and 5-(1-methyl-1H-pyrazol-3-yl)-thiophene-2-carboxylic acid (tetrahydro-pyran-2-yloxy)-amide

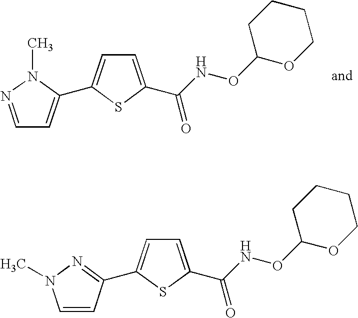

By proceeding in a similar manner to Reference Example 1(a) but using a mixture of 5-(2-methyl-2H-pyrazol-3-yl)-thiophene-2-carboxylic acid and 5-(1-methyl-1H-pyrazol-3-yl)-thiophene-2-carboxylic acid [Reference Example 2(a)] there was prepared a mixture of 5-(2-methyl-2H-pyrazol-3-yl)-thiophene-2-carboxylic acid (tetrahydro-pyran-2-yloxy)-amide and 5-(1-methyl-1H-pyrazol-3-yl)-thiophene-2-carboxylic acid (tetrahydro-pyran-2-yloxy-amide (75 mg, 73%) as a colourless foam. LCMS (Method A): R_{T}=5.95 minutes (minor component) and 6.08 minutes (major component); 308 (M+H)⁺.

### (c) 5-(5-Trifluoromethyl-2H-pyrazol-3-yl)-thiophene-2-carboxylic acid (tetrahydro-pyran-2-yloxy)-amide

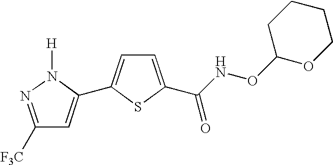

By proceeding in a similar manner to Reference Example 1(a) but using 5-(5-trifluoromethyl-2H-pyrazol-3-yl)-thiophene-2-carboxylic acid there was prepared 5-(5-trifluoromethyl-2H-pyrazol-3-yl)-thiophene-2-carboxylic acid (tetrahydro-pyran-2-yloxy)-amide (51 mg, 52%) as a white solid. LCMS (Method C): R_{T}=3.10 minutes; 362 (M+M⁺.

### (d) 5-(1-Methyl-5-trifluoromethyl-1H-pyrazol-3-yl)-thiophene-2-carboxylic acid (tetra-hydro-pyran-2-yloxy)-amide

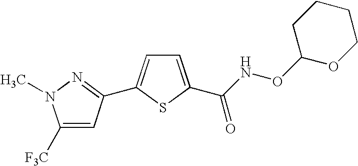

By proceeding in a similar manner to Reference Example 1(a) but using 5-(1-methyl-5-trifluoromethyl-1H-pyrazol-3-yl)-thiophene-2-carboxylic acid there was prepared 5-(1-methyl-5-trifluoromethyl-1H-pyrazol-3-yl)-thiophene-2-carboxylic acid (tetrahydro-pyran-2-yloxy)-amide (244 mg, 88%) as a yellow gum. LCMS (Method A): R_{T}=8.49 minutes; 376 (N+H)⁺.

(e) 5-(5-Trifluoromethyl-isoxazol-3-yl)-thiophene-2-carboxylic acid (tetrahydro-pyran-2-yloxy)-amide

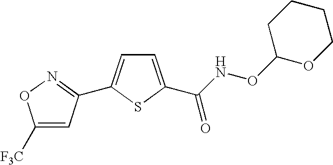

By proceeding in a similar manner to Reference Example 1(a) but using 5-(5-hydroxy-5-trifluoromethyl-4,5-dihydro-isoxazol-3-yl)-thiophene-2-carboxylic acid [Reference Example 2(b)] there was prepared a mixture of 5-(5-trifluoromethyl-isoxazol-3-yl)-thiophene-2-carboxylic acid (tetrahydro-pyran-2-yloxy)-amide and 5-(5-hydroxy-5-trifluoromethyl-4,5-dihydro-isoxazol-3-yl)-thiophene-2-carboxylic acid (tetrahydro-pyran-2-yloxy)-amide. The mixture was separated by flash chromatography on silica eluting with 28%-40% (v/v) ethyl acetate in petroleum ether fraction (b.p. 40-60° C.) to yield 5-(5-trifluoromethyl-isoxazol-3-yl)-thiophene-2-carboxylic acid (tetrahydro-pyran-2-yloxy)-amide (22 mg, 23%) as a white solid. LCMS (Method A): R_{T}=8.95 minutes; 363 (+H)⁺.

### (f) 5-Phenyl-thiophene-2-carboxylic acid (tetrahydro-pyran-2-yloxy)-amide

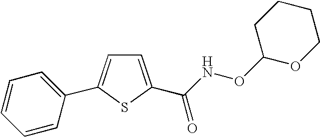

To a solution of 5-phenyl-thiophene-2-carboxylic acid (72 mg, 0.35 mmol) in N,N-dimethylformamide (3 ml) at 0° C. was added O-(tetrahydro-2H-pyran-2-yl)hydroxylamine (45 mg, 0.39 mmol), diisopropylethylamine (153 μl, 0.88 mmol), and O -(7-azabenzotriazol-1-yl)-N,N,N′,N′-tetramethyluronium hexafluorophosphate (148 mg, 0.39 mmol). The mixture was allowed to equilibrate to room temperature over 7 hours. The volatiles were evaporated and the residue was partitioned between ethyl acetate and water. The two phases were separated and the aqueous phase was extracted twice with ethyl acetate. The combined extracts were washed with water, then with 10% citric acid solution, then with saturated sodium bicarbonate solution, then with brine, then dried over magnesium sulfate and then evaporated. The residual yellow gum was subjected to column chromatography on silica eluting with ethyl acetate/pentane (1:3 v/v) to yield 5-phenol-thiophene-2-carboxylic acid (tetrahydro-pyran-2-yloxy)-amide (87 mg, 81%) as a white gum, which crystallised on standing. LCMS (Method A): R_{T}=8.48 minutes; 304 M+H)⁺.

### (g) 5-Pyridin-2-yl-thiophene-2-carboxylic acid (tetrahydro-pyran-2-yloxy)-amide

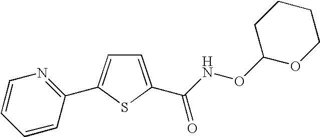

By proceeding in a similar manner to Reference Example 1(f) but using 5-pyridin-2-yl-thiophene-2-carboxylic acid there was prepared 5-pyridin-2-yl-thiophene-2-carboxylic acid (tetrahydro-pyran-2-yloxy)-amide (233 mg, 78%) as a pale yellow gum. LCMS (Method A): R_{T}=6.32 minutes; 305 (M+H)⁺.

### (h) [2,2′]Bithiophenyl-5-carboxylic acid (tetrahydro-pyran-2-yloxy)-amide

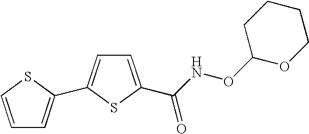

By proceeding in a similar manner to Reference Example 1(a) but using [2,2′]bithiophenyl-5-carboxylic acid there was prepared [2,2′]bithiophenyl-5-carboxylic acid (tetrahydro-pyran-2-yloxy)-amide (212 mg, 79%) as a colourless oil, which was used in the next step without further purification.

### (i) 5-(4-Methoxy-phenyl)-thiophene-2-carboxylic acid (tetrahydro-pyran-2-yloxy) -amide

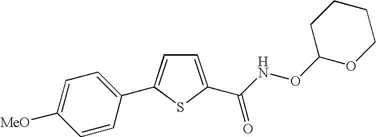

By proceeding in a similar manner to Reference Example 1(a) but using 5-(4-methoxy-phenyl)-thiophene-2-carboxylic acid there was prepared 5-(4-methoxy-phenyl)-thiophene-2-carboxylic acid (tetrahydro-pyran-2-yloxy-amide (195 mg, 84%) as a yellow foam. LCMS (Method A): R_{T}=8.47 minutes; 334 (M+H)⁺.

### (j) 5-(1H-Pyrazol-3-yl)-thiophene-2-carboxylic acid (tetrahydro-pyran-2-yloxy)-amide

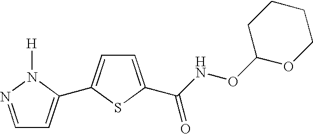

By proceeding in a similar manner to Reference Example 1(a) but using 5-(1H-pyrazol-3-yl)-thiophene-2-carboxylic acid [Reference Example 2(c)] there was prepared 5-(1H-pyrazol-3-yl)-thiophene-2-carboxylic acid (tetrahydro-pyran-2-yloxy-amide (66 mg, 55%) as a foam. LCMS (Method A): R_{T}=5.52 minutes; 294 (M+H)⁺.

### (k) 5-(1-Benzyl-1H-pyrazol-3-yl)-thiophene-2-carboxylic acid (tetrahydro-pyran-2-yloxy)-amide

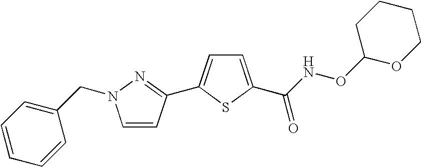

By proceeding in a similar manner to Reference Example 1(a) but using 5-(1-benzyl-1H-pyrazol-3-yl)-thiophene-2-carboxylic acid [Reference Example 2(d)] there was prepared 5-(1-benzyl-1H-pyrazol-3-yl)-thiophene-2-carboxylic acid (tetrahydro-p ran-2-yloxy-amide (65 mg, 91%) as a colourless oil. LCMS (Method A): R_{T}=8.39 minutes; 384 (M+H)⁺.

### (l) 5-(1-Phenethyl-1H-pyrazol-3-yl)-thiophene-2-carboxylic acid (tetrahydro-pyran-2-yloxy)-amide

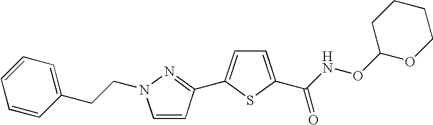

By proceeding in a similar manner to Reference Example 1(a) but using 5-(1-phenethyl-1H-pyrazol-3-yl)-thiophene-2-carboxylic acid [Reference Example 2(e)] there was prepared 5-(1-phenethyl-1H-pyrazol-3-yl)-thiophene-2-carboxylic acid (tetrahydro-pyran-2-yloxy)-amide (138 mg, 92%) as a colourless oil. LCMS (Method A): R_{T}=8.79 minutes; 398 W+H)⁺.

### (m) 5-(4-Trifluoromethyl-1H-imidazol-2-yl)-thiophene-2-carboxylic acid (tetrahydro-pyran-2-yloxy)-amide

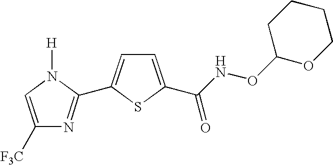

By proceeding in a similar manner to Reference Example 1(a) but using 5-(4-trifluoromethyl-1H-imidazol-2-yl)-thiophene-2-carboxylic acid [Reference Example 6(a)] there was prepared 5-(4-trifluoromethyl-1H-imidazol-2-yl)-thiophene-2-carboxylic acid (tetrahydro-pyran-2-yloxy)-amide (42 mg, 80%) as a colourless gum. LCMS (Method A): R_{T}=6.77 minutes; 362 (M+H)⁺.

### (n) 5-(3-Methyl-[1,2,4]oxadiazol-5-yl)-thiophene-2-carboxylic acid (tetrahydro-pyran-2-yloxy)-amide

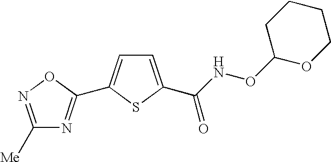

By proceeding in a similar manner to Reference Example 1(a) but using 5-(3-methyl-[1,2,4]oxadiazol-5-yl)-thiophene-2-carboxylic acid there was prepared 5-(3-methyl-[1,2,4]oxadiazol-5-yl)-thiophene-2-carboxylic acid (tetrahydro-pyran-2-yloxy)-amide (155 mg, 98%) as colourless gum, which was used directly without further purification.

### (o) 5-[1-(3-Phenyl-propyl-1H-pyrazol-3-yl]-thiophene-2-carboxylic acid (tetrahydro-pyran-2-yloxy)-amide

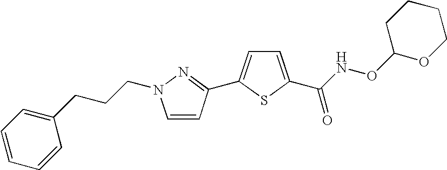

A solution of 5-[1-(3-phenyl-propyl)-1H-pyrazol-3-yl]-thiophene-2-carboxylic acid [272 mg, 0.87 mmol, Reference Example 2(f)] in dimethylformamide (10 ml) was treated with diisopropylethylamine (600 μl, 3.36 mmol), O-(tetrahydro-2H-pyran-2-yl)hydroxylamine (200 mg, 1.7 mmol) and O-(7-azabenzotriazol-1-yl)-N,N,N′,N′-tetramethyluronium hexafluorophosphate (700 mg, 1.8 mmol). The mixture was stirred at room temperature over the weekend, then was evaporated under reduced pressure, and the residue produced was partitioned between ethyl acetate and saturated sodium bicarbonate solution. The two phases were separated and the organic phase was evaporated under reduced pressure. The crude product was subjected to flash column chromatography on silica using a mixture of petroleum ether fraction (b.p. 30-50° C.) and ethyl acetate (gradient 9:1 to 7:3, v/v) as eluent, to provide 5-[1-(3-phenyl-propyl)-1H-pyrazol-3-yl]-thiophene-2-carboxylic acid (tetrahydro-pyran-2-yloxy)-amide (381 mg) as a yellow gum. LCMS (Method C): R_{T}=3.63 minutes; 412 (M+H)⁺.

### (p) 5-[1-(2,3-Dihydro-benzo[1,4]dioxin-2-ylmethyl)-1H-pyrazol-3-yl]-thiophene-2-carboxylic acid (tetrahydro-pyran-2-yloxy)-amide

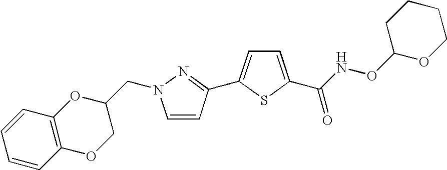

By proceeding in a similar manner to Reference Example 1(o) but using 5-[1-(2,3-dihydro-benzo[1,4]dioxin-2-ylmethyl)-1H-pyrazol-3-yl]-thiophene-2-carboxylic acid [56 mg, 0.16 mmol, Reference Example 2(g)], stirring overnight, and using a gradient (9:1 to 7:3, v/v) as eluent, there was prepared 5-[1-(2,3-dihydro-benzo[1,4]dioxin-2-ylmethyl)-1H-pyrazol-3-yl]-thiophene-2-carboxylic acid (tetrahydro-pyran-2-yloxy)-amide (67.5 mg) as a colourless oil. LCMS (Method C): R_{T}=3.41 minutes; 442 (M+H)⁺.

### q) 5-{1-[2-(4-Trifluoromethyl-phenyl-ethyl]-1H-pyrazol-3-yl}-thiophene-2-carboxylic acid (tetrahydro-pyran-2-yloxy)-amide

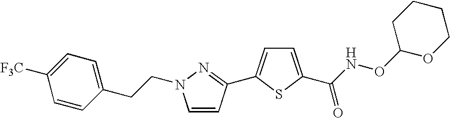

By proceeding in a similar manner to Reference Example 1(o) but using 5-{1-[2-(4-trifluoromethyl-phenyl)-ethyl]-1H-pyrazol-3-yl}-thiophene-2-carboxylic acid [181 mg, 0.54 mmol, Reference Example 2(h)], stirring overnight, and without chromatography, there was prepared 5-{1-[2-(4-trifluoromethyl-phenyl)-ethyl]-1H-pyrazol-3-yl}-thiophene-2-carboxylic acid (tetrahydro-pyran-2-yloxy)-amide (226 mg) as a colourless gum. LCMS (Method C): R_{T}=3.71 minutes; 466 (M+H)⁺.

### (r) 5-(1-Benzo[1,3]dioxol-5-ylmethyl-1H-pyrazol-3-yl)-thiophene-2-carboxylic acid (tetrahydro-pyran-2-yloxy)-amide

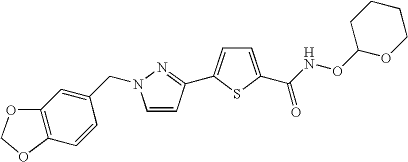

By proceeding in a similar manner to Reference Example 1(o) but using 5-(1-benzo[1,3]dioxol-5-ylmethyl-1H-pyrazol-3-yl)-thiophene-2-carboxylic acid [181 mg, 0.55 mmol, Reference Example 2(i)], stirring for 4 days, and using a gradient (9:1 to 2:8, v/v) as eluent, there was prepared 5-(1-benzo[1,3]dioxol-5-ylmethyl-1H-pyrazol-3-yl)-thiophene-2-carboxylic acid (tetrahydro-pyran-2-yloxy-amide (173 mg). LCMS (Method C): R_{T}=3.22 minutes; 428 (M+H)⁺.

### (s) 5-{1-[2-(4-Trifluoromethoxy-phenyl)-ethyl]-1H-pyrazol-3-yl}-thiophene-2-carboxylic acid (tetrahydro-pyran-2-yloxy)-amide

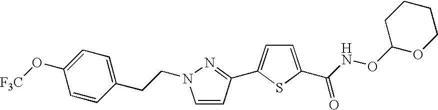

By proceeding in a similar manner to Reference Example 1(o) but using 5-{1-[2-(4-trifluoromethoxy-phenyl)-ethyl]-1H-pyrazol-3-yl}-thiophene-2-carboxylic acid [228 mg, 0.59 mmol, Reference Example 2(j)], stirring overnight, and using a gradient (65:35 to 60:40, v/v) as eluent, there was prepared 5-{1-[2-(4-trifluoromethoxy-phenyl)-ethyl]-1H-pyrazol-3-yl}-thiophene-2-carboxylic acid (tetrahydro-pyran-2-yloxy)-amide (243 mg) as a white foam. LCMS (Method C): R_{T}=3.79 minutes; 482 (M+H)⁺.

### (t) 5-{1-[2-(4-Fluoro-phenyl)-ethyl]-1H-pyrazol-3-yl}-thiophene-2-carboxylic acid (tetrahydro-pyran-2-yloxy)-amide

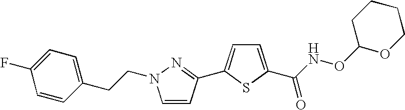

By proceeding in a similar manner to Reference Example 1(o) but using 5-{1-[2-(4-fluoro-phenyl)-ethyl]-1H-pyrazol-3-yl}-thiophene-2-carboxylic acid [113 mg, 0.36 mmol, Reference Example 2(k)], stirring for 4 hours, and without chromatography there was prepared 5-{1-[2-(4-fluoro-phenyl)-ethyl]-1H-pyrazol-3-yl}-thiophene-2-carboxylic acid (tetrahydro-pyran-2-yloxy)-amide (120 mg) as a gum. LCMS (Method C): R_{T}=3.41 minutes.

### (u) 5-[1-(1-Phenyl-ethyl-1H-pyrazol-3-yl]-thiophene-2-carboxylic acid (tetrahydro-pyran-2-yloxy)-amide

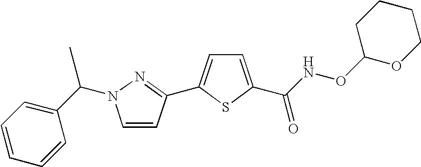

By proceeding in a similar manner to Reference Example 1(o) but using 5-[1-(1-phenyl-ethyl)-1H-pyrazol-3-yl]-thiophene-2-carboxylic acid [205 mg, 0.7 mmol, Reference Example 2(l)), stirring for 6 hours, partitioning between diethyl ether and water rather than ethyl acetate and water, and using ethyl acetate and cyclohexane (50:50, v/v) as eluent, here was prepared 5-[1-(1-phenyl-ethyl)-1H-pyrazol-3-yl]-thiophene-2-carboxylic acid (tetrahydro-pyran-2-yloxy)-amide (214 mg). LCMS (Method C): R_{T}=3.45 minutes; 398 (1+H)⁺.

### (v) 5-[1-(2-Morpholin-4-yl-ethyl)-1H-pyrazol-3-yl]-thiophene-2-carboxylic acid (tetrahydro-pyran-2-yloxy)-amide

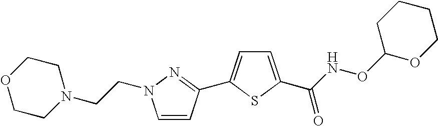

By proceeding in a similar manner to Reference Example 1(o) but using 5-[1-(2-morpholin-4-yl-ethyl)-1H-pyrazol-3-yl]-thiophene-2-carboxylic acid [118 mg, 0.38 mmol, Reference Example 2(m)], stirring for 6 hours, and without chromatography there was prepared 5-[1-(2-morpholin-4-yl-ethyl-1H-pyrazol-3-yl]-thiophene-2-carboxylic acid (tetrahydro-pyran-2-yloxy)-amide (58 mg) as a pale gum. LCMS (Method C): R_{T}=1.84 minutes; 407 (M+H)⁺.

### (w) 5-[1-(Tetrahydro-pyran-2-ylmethyl)-1H-pyrazol-3-yl]-thiophene-2-carboxylic acid (tetrahydro-pyran-2-yloxy)-amide

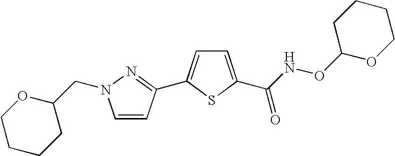

By proceeding in a similar manner to Reference Example 1(o) but using 5-[1-(tetrahydro-pyran-2-ylmethyl)-1H-pyrazol-3-yl]-thiophene-2-carboxylic acid [168 mg, 0.57 mmol, Reference Example 2(n)], stirring for 3 hours, and using 1:1 (v/v) as eluent, there was prepared 5-[1-(tetrahydro-pyran-2-ylmethyl)-1H-pyrazol-3-yl]-thiophene-2-carboxylic acid (tetrahydro-pyran-2-yloxy)-amide (216 mg) as a white foam. LCMS (Method C): R_{T}=3.15 minutes; 392 (M+H)⁺.

### (x) 5-(4-Benzyloxy-pyrimidin-2-yl)-thiophene-2-carboxylic acid (tetrahydro-pyran-2-yloxy)-amide

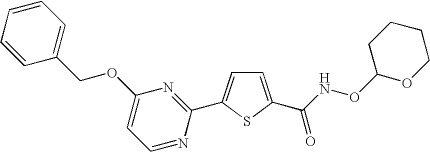

By proceeding in a similar manner to Reference Example 1(o) but using 5-(4-Benzyloxy-pyrimidin-2-yl)-thiophene-2-carboxylic acid [108 mg, 0.34 mmol, Reference Example 13(a)], stirring for 4 hours, partitioning between diethyl ether and water rather than ethyl acetate and water, and without chromatography, there was prepared 5-(4-Benzyloxy-pyrimidin-2-yl)-thiophene-2-carboxylic acid (tetrahydro-pyran-2-yloxy-amide (123 mg) as a gum. LCMS (Method C): R_{T}=3.67 minutes; 412 (M+H)⁺.

### (y) 5-(5-Phenethyl-1H-pyrazol-3-yl)-thiophene-2-carboxylic acid (tetrahydro-pyran-2-yloxy-amide

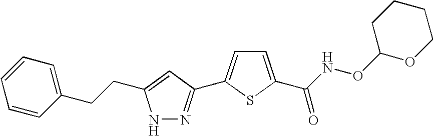

By proceeding in a similar manner to Reference Example 1(o) but using 5-(5-Phenethyl-1H-pyrazol-3-yl)-thiophene-2-carboxylic acid [1.19 g, 3.99 mmol, Reference Example 13(b)], stirring overnight, and using 3:7 (v/v) as eluent, there was prepared 5-(5-Phenethyl-1H-pyrazol-3-yl)-thiophene-2-carboxylic acid (tetrahydro-pyran-2-yloxy)-amide (1.34 g), which was used directly without further purification.

### (z) 5-(2-Phenethyl-3H-imidazol-4-yl)-thiophene-2-carboxylic acid (tetrahydro-pyran-2-yloxy)-amide

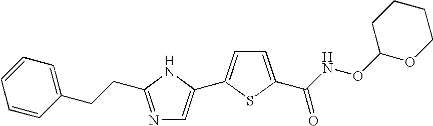

By proceeding in a similar manner to Reference Example 1(o) but using 5-(2-phenethyl-3H-imidazol-4-yl)-thiophene-2-carboxylic acid [74 mg, 0.25 mmol, Reference Example 2(o)], stirring overnight, and using cyclohexane and ethyl acetate (1:9, v/v) as eluent, there was prepared 5-(2-phenethyl-3H-imidazol-4-yl)-thiophene-2-carboxylic acid (tetrahydro-pyran-2-yloxy)-amide (16 mg) as a light brown oil. LCMS (Method C): R_{T}=2.19 minutes; 398 (M+H)⁺.

### (aa) 5-Pyrimidin-2-yl-thiophene-2-carboxylic acid (tetrahydro-pyran-2-yloxy-amide

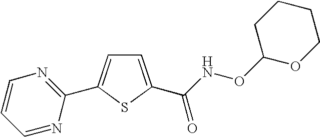

To a cooled (10° C.) suspension of 5-pyrimidin-2-yl-thiophene-2-carboxylic acid [300 mg, 1.45 mmol, Reference Example 14(a)] in dichloromethane (20 ml) was added oxalyl chloride (380 μl, 4.4 mmol) and N,N-dimethylformamide (1 drop). After no more gas was liberated from the mixture a fine precipitate was observed, and the solvent was removed in vacuo to give an off-white solid. To the solid was added dichloromethane (20 ml), diisopropylethylamine (1.26 ml, 7.25 mmol), and O-(tetrahydro-2H-pyran-2-yl)hydroxylamine (170 mg, 1.45 mmol). The mixture was stirred at room temperature overnight, then the solvent was evaporated under reduced pressure, and the residue was partitioned between ethyl acetate and saturated sodium bicarbonate solution. The two phases were separated; the organic phase was dried (MgSO₄), and evaporated under reduced pressure, to provide 5-pyrimidin-2-yl-thiophene-2-carboxylic acid (tetrahydro-pyran-2-yloxy)-amide (170 mg) as a gum. LCMS (Method C): R_{T}=2.51 minutes; 306 (M+H)⁺.

### (ab) 5-(1-Phenethyl-5-trifluoromethyl-1H-pyrazol-3-yl)-thiophene-2-carboxylic acid (tetrahydro-pyran-2-yloxy)-amide

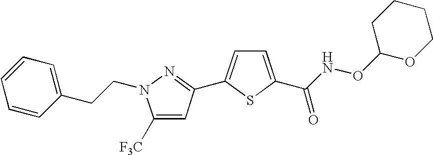

By proceeding in a similar manner to Reference Example 1(o) but using 5-(1-phenethyl-5-trifluoromethyl-1H-pyrazol-3-yl)-thiophene-2-carboxylic acid [125 mg, 0.34 mmol, Reference Example 2(p)], stirring overnight, without washing, and using pentane and ethyl acetate (9:1 to 7:3, v/v) as eluent, there was prepared 5-(1-phenethyl-5-trifluoromethyl-1H-pyrazol-3-yl)-thiophene-2-carboxylic acid (tetrahydro-pyran-2-yloxy-amide (122 mg). LCMS (Method C): R_{T}=4.02 minutes; 466 (M+H)⁺.

### (ac) 5-Pyridin-3-yl-thiophene-2-carboxylic acid (tetrahydro-pyran-2-yloxy)-amide

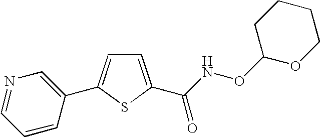

By proceeding in a similar manner to Reference Example 1(o) but using 5-pyridin-3-yl-thiophene-2-carboxylic acid [140 mg, 0.68 mmol, Reference Example 14(b)], stirring for 4 hours, without washing, and subjecting the crude reaction material to reverse phase purification using acetonitrile and water (gradient 0:100 to 100:0, v/v, in 10% intervals) as eluent, there was prepared 5-pyridin-3-yl-thiophene-2-carboxylic acid (tetrahydro-pyran-2-yloxy-amide (120 mg) as a colourless glass. LCMS (Method C): R_{T}=2.15 minutes; 305 (M+H)⁺.

### (ad) 5-Pyridin-4-yl-thiophene-2-carboxylic acid (tetrahydro-pyran-2-yloxy)-amide

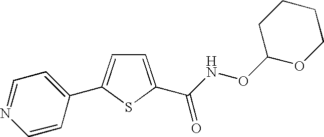

By proceeding in a similar manner to Reference Example 1(o) but using 5-pyridin-3-yl-thiophene-2-carboxylic acid [87 mg, 0.42 mmol, Reference Example 14(c)], stirring for 4 hours, without washing, and subjecting the crude reaction material to reverse phase purification using acetonitrile and water (gradient 0:100 to 100:0, v/v, in 10% intervals) as eluent, there was prepared 5-pyridin-4-yl-thiophene-2-carboxylic acid (tetrahydro-pyran-2-yloxy)-amide (147 mg) as a yellow glass. LCMS (Method C): R_{T}=1.79 minutes; 305 (M+H)⁺.

### (ae) 5-(5-Trifluoromethyl-1H-1,2,4]triazol-3-yl)-thiophene-2-carboxylic acid (tetrahydro-pyran-2-yloxy)-amide

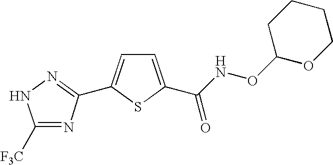

By proceeding in a similar manner to Reference Example 1(o) but using 5-(5-trifluoromethyl-1H-[1,2,4]triazol-3-yl)-thiophene-2-carboxylic acid [60 mg, 0.23 mmol, Reference Example 13(c)], stirring overnight, without washing, and using a gradient [pentane and ethyl acetate (7:3, v/v) to methanol] as eluent, there was prepared 5-(5trifluoromethyl-1H-[1,2,4]triazol-3-yl)-thiophene-2-carboxylic acid (tetrahydro-pyran-2-yloxy)-amide (67 mg). LCMS (Method C): R_{T}=2.88 minutes; 363 (M+H)⁺.

### (af) 4-Methyl-5-(5-trifluoromethyl-1H-pyrazol-3-yl)-thiophene-2-carboxylic acid hydroxyamide

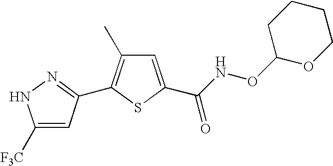

By proceeding in a similar manner to Reference Example 1(o) but using 4-methyl-5-(5-trifluoromethyl-1H-pyrazol-3-yl)-thiophene-2-carboxylic acid [300 mg, 1.09 mmol, Reference Example 13(d)], stirring overnight, and using pentane and ethyl acetate (1:1, v/v) as eluent, there was prepared 4-methyl-5-(5-trifluoromethyl-1H-pyrazol-3-yl)-thiophene-2-carboxylic acid hydroxyamide (232 mg), which was used directly without further purification.

### (ag) 5-(3-Benzyloxy-phenyl-thiophene-2-carboxylic acid (tetrahydro-pyran-2-yloxy)-amide

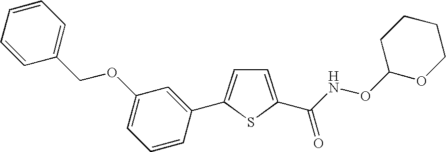

By proceeding in a similar manner to Reference Example 1(o) but using 5-(3-benzyloxy-phenyl)-thiophene-2-carboxylic acid [35 mg, 0.11 mmol, Reference Example 6(b)], and without chromatography, there was prepared 5-(3-benzyloxy-phenyl)-thiophene-2-carboxylic acid (tetrahydro-pyran-2-yloxy-amide (48 mg) as a milky gum. LCMS (Method C): R_{T}=3.98 minutes; 410 (M+H)⁺.

### (ai) 5-(6-{[(Pyridin-3-ylmethyl)-amino]-methyl}-pyridin-2-yl)-thiophene-2-carboxylic acid (tetrahydro-pyran-2-yloxy)-amide

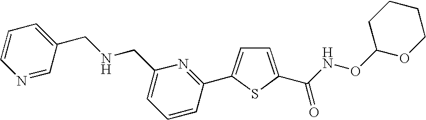

A solution of 5-(6-{[(pyridin-3-ylmethyl)-amino]-methyl}-pyridin-2-yl)-thiophene-2-carboxylic acid [123 mg, 0.38 mmol, Reference Example 32(j)] in dimethylformamide (2 ml) was treated with diisopropylethylamine (132 μl, 0.76 mmol), O-(tetrahydro-2H-pyran-2-yl)hydroxylamine (49 mg, 0.42 mmol) and O-(7-azabenzotriazol-1-yl)-N,N,N′,N′-tetramethyluronium hexafluorophosphate (152 mg, 0.4 mmol). The mixture was stirred at room temperature for 6 hours, then concentrated, to provide 5-(6-{[(pyridin-3-ylmethyl-amino]-methyl}-pyridin-2-yl)-thiophene-2-carboxylic acid (tetrahydro-pyran-2-yloxy)-amide which was used directly without further purification. LCMS (Method C): R_{T}=1.77 minutes; 425 (M+H)⁺.

### (aj) 5-{6-[(2-Pyridin-3-yl-ethylamino)-methyl]-pyridin-2-yl}-thiophene-2-carboxylic acid (tetrahydro-pyran-2-yloxy)-amide

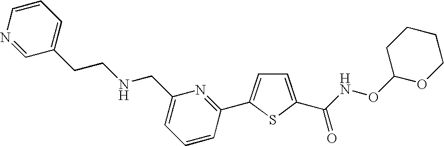

By proceeding in a similar manner to Reference Example 1(ai) but using 5-{6-[(2-pyridin-3-yl-ethylamino)-methyl]-pyridin-2-yl}-thiophene-2-carboxylic acid [129 mg, 0.38 mmol, Reference Example 32k)] there was prepared 5-{6-[(2-pyridin-3-yl-ethylamino)-methyl]-pyridin-2-yl}-thiophene-2-carboxylic acid (tetrahydro-pyran-2-yloxy)-amide which was used directly without further purification. LCMS (Method C): R_{T}=1.61 minutes; 439 (N+H)⁺.

### (ak) 5-{6-[(4-Fluoro-benzylamino-methyl]-pyridin-2-yl}-thiophene-2-carboxylic acid (tetrahydro-pyran-2-yloxy)-amide

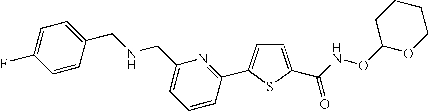

By proceeding in a similar manner to Reference Example 1(ai) but using 5-{6-[(4-fluoro-benzylamino)-methyl]-pyridin-2-yl}-thiophene-2-carboxylic acid [130 mg, 0.38 mmol, Reference Example 32(l)] there was prepared 5-{6-[(4-fluoro-benzylamino)-methyl]-pyridin-2-yl}-thiophene-2-carboxylic acid (tetrahydro-pyran-2-yloxy)-amide, which was used directly without further purification. LCMS (Method C): R_{T}=2.21 minutes; 442 (M+H)⁺.

### (al) 5-(6-{[(Benzo[1,3]dioxol-5-ylmethyl)-amino]-methyl}-pyridin-2-yl)-thiophene-2-carboxylic acid (tetrahydro-pyran-2-yloxy)-amide

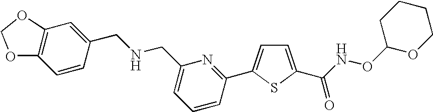

By proceeding in a similar manner to Reference Example 1(ai) but using 5-(6-{[(benzo[1,3]dioxol-5-ylmethyl)-amino]-methyl}-pyridin-2-yl)-thiophene-2-carboxylic acid [140 mg, 0.38 mmol, Reference Example 32(m)] there was prepared 5-(6-{[(benzo[1,3]dioxol-5-ylmethyl)-amino]-methyl}-pyridin-2-yl)-thiophene-2-carboxylic acid (tetrahydro-pyran-2-yloxy)-amide which was used directly without further purification. LCMS (Method C): R_{T}=2.18 minutes; 468 (M+H)⁺.

### (am) 5-(6-{[(1H-Benzoimidazol-2-ylmethyl)-amino]-methyl}-pyridin-2-yl)-thiophene-2-carboxylic acid (tetrahydro-pyran-2-yloxy)-amide

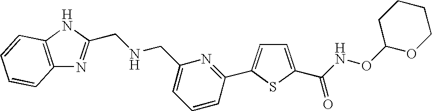

By proceeding in a similar manner to Reference Example 1(ai) but using 5-(6-{[(1H-benzoimidazol-2-ylmethyl)-amino]-methyl}-pyridin-2-yl)-thiophene-2-carboxylic acid [138 mg, 0.38 mmol, Reference Example 32(n)] there was prepared 5-(6-{[(1H-benzoimidazol-2-ylmethyl)-amino]-methyl}-pyridin-2-yl)-thiophene-2-carboxylic acid (tetrahydro-pyran-2-yloxy)-amide, which was used directly without further purification. LCMS (Method C): R_{T}=2.09 minutes; 464 (+H)⁺.

### (an) 5-{6-[(3-Imidazol-1-yl-propylamino)-methyl]-pyridin-2-yl}-thiophene-2-carboxylic acid (tetrahydro-pyran-2-yloxy)-amide

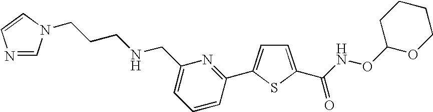

By proceeding in a similar manner to Reference Example 1(ai) but using 5-{6-[(3-imidazol-1-yl-propylamino)-methyl]-pyridin-2-yl}-thiophene-2-carboxylic acid [130 mg, 0.38 mmol, Reference Example 32(o)] there was prepared 5-{6-[(3-imidazol-1-yl-propylamino-methyl]-pyridin-2-yl}-thiophene-2-carboxylic acid (tetrahydro-pyran-2-yloxy)-amide, which was used directly without further purification. LCMS (Method C): R_{T}=1.51 minutes; 442 (M+H)⁺.

### (ap) 5-[6-(3-Phenyl-propionylamino)-pyridin-2-yl]-thiophene-2-carboxylic acid (tetrahydro-pyran-2-yloxy)-amide

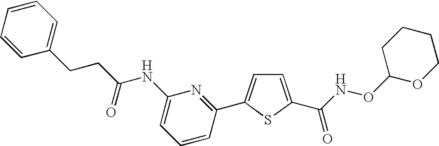

A solution of 5-[6-(3-phenyl-propionylamino)-pyridin-2-yl]-thiophene-2-carboxylic acid [84 mg, 0.23 mmol, Reference Example 6(h)] in dimethylformamide (6 ml) was treated with diisopropylethylamine (80 μl, 0.46 mmol), O-(tetrahydro-2H-pyran-2-yl)hydroxylamine (39 mg, 0.33 mmol) and O-(7-azabenzotriazol-1-yl)-N,N,N′,N′-tetramethyluronium hexafluorophosphate (87 mg, 0.23 mmol). The mixture was stirred at room temperature over the weekend, then diluted with water, and extracted with ethyl acetate (3×). The organic layers were combined, washed with water followed by brine, dried (Na₂SO₄), and concentrated under reduced pressure, to provide 5-[6-(3-phenyl-propionylamino-pyridin-2-yl]-thiophene-2-carboxylic acid (tetrahydro-pyran-2-yloxy)-amide (101 mg). LCMS (Method C): R_{T}=3.61 minutes; 452 (M+H)⁺.

### (aq) 5-{1-[(3-Methoxy-phenylcarbamoyl)-methyl]-1H-pyrazol-3-yl}-thiophene-2-carboxylic acid (tetrahydro-pyran-2-yloxy)-amide

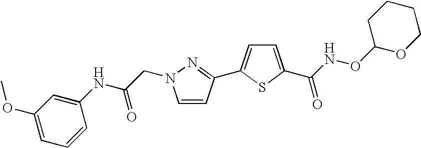

A solution of 5-{1-[(3-methoxy-phenylcarbamoyl)-methyl]-1H-pyrazol-3-yl}-thiophene-2-carboxylic acid [250 mg, 0.7 mmol, Reference Example 6(j)] in dimethylformamide (15 ml) was treated with diisopropylethylamine (366 μl, 2.1 mmol), O-(tetrahydro-2H-pyran-2-yl)hydroxylamine (94 mg, 0.8 mmol) and O-(7-azabenzotriazol-1-yl)-N,N,N′,N′-tetramethyluronium hexafluorophosphate (266 mg, 0.7 mmol). The mixture was stirred at room temperature for 30 minutes, and then concentrated to give a residue. The residue was partitioned between ethyl acetate and saturated sodium hydrogen carbonate solution. The aqueous layer was extracted with ethyl acetate (3×) followed by dichloromethane, and the organic layers were combined, dried (Na₂SO₄), and concentrated under reduced pressure, to provide 5-{1-[(3-methoxy-phenylcarbamoyl)-methyl]-1H-pyrazol-3-yl}-thiophene-2-carboxylic acid (tetrahydro-pyran-2-yloxy)-amide (391 mg) as a pale brown oil. LCMS (Method C): R_{T}=3.06 minutes; 457 (M+H)⁺.

### (ar) 5-[6-(3-Phenyl-propylamino-pyridin-2-yl]-thiophene-2-carboxylic acid (tetrahydro-pyran-2-yloxy)-amide

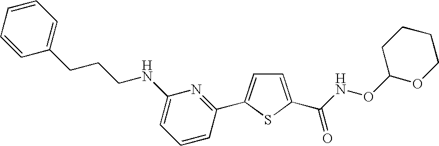

By proceeding in a similar manner to Reference Example 1(ap) but using 5-[6-(3-phenyl-propylamino)-pyridin-2-yl]-thiophene-2-carboxylic acid [200 mg, 0.59 mmol, Reference Example 6(i)] there was prepared 5-[6-(3-phenyl-propylamino)-pyridin-2-yl]-thiophene-2-carboxylic acid (tetrahydro-pyran-2-yloxy)-amide. LCMS (Method C): R_{T} 3.74 minutes; 438 (M+H)⁺.

### (at) 5-{6-[(Benzo[1,3]dioxol-5-ylmethl-methyl-amino)-methyl]-pyridin-2-yl}-thiophene-2-carboxylic acid (tetrahydro-pyran-2-yloxy)-amide

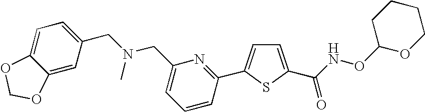

By proceeding in a similar manner to Reference Example 1(ap) but using 5-{6-[(benzo[1,3]dioxol-5-ylmethyl-methyl-amino)-methyl]-pyridin-2-yl}-thiophene-2-carboxylic acid [156 mg, 0.41 mmol, Reference Example 32(q)] there was prepared 5{6-[(benzo[1,3]dioxol-5-ylmethyl-methyl-amino)-methyl]-pyridin-2-yl}-thiophene-2-carboxylic acid (tetrahydro-pyran-2-yloxy)-amide. LCMS (Method C): R_{T} 2.22 minutes; 482 (M+H)⁺.

### (au) 5-[1-(1-Oxy-quinolin-2-ylmethyl)-1H-pyrazol-3-yl]-thiophene-2-carboxylic acid (tetrahydro-pyran-2-yloxy)-amide

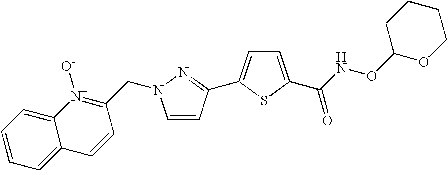

By proceeding in a similar manner to Reference Example 1(ap) but using 5-[1-(1-oxy-quinolin-2-ylmethyl)-1H-pyrazol-3-yl]-thiophene-2-carboxylic acid [418 mg, 1.2 mmol, Reference Example 6(k)] there was prepared 5-[1-(1-oxy-quinolin-2-ylmethyl)-1H-pyrazol-3-yl]-thiophene-2-carboxylic acid (tetrahydro-pyran-2-yloxy)-amide (596 mg) as a gum, which was used directly without further purification.

### Reference Example 2

### (a) 5-(2-Methyl-2H-pyrazol-3-yl)-thiophene-2-carboxylic acid and 5-(1-Methyl-1H-pyrazol-3-yl)-thiophene-2-carboxylic acid

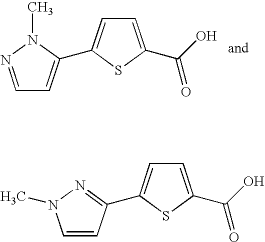

A mixture of 5-(2-methyl-2H-pyrazol-3-yl)-thiophene-2-carbonitrile and 5-(1-methyl-1H-pyrazol-3-yl)-thiophene-2-carbonitrile [0,7 g, 3.7 mmol, Reference Example 3(a)]) in sodium hydroxide solution (15 ml, 1M) was heated at reflux for 2 hours. The reaction mixture was cooled to room temperature, diluted with water, acidified with hydrochloric acid (1M) and extracted three times with ethyl acetate. The combined extracts were dried over magnesium sulfate and then evaporated under reduced pressure. The residue was subjected to flash column chromatography to give a mixture of 5-(2-methyl-2H-pyrazol-3-yl)-thiophene-2-carboxylic acid and 5-(1-methyl-1H-pyrazol-3-yl)-thiophene-2-carboxylic acid (94 mg, 12%) as a yellow solid. LCMS (Method B): R_{T}=1.48 minutes; 209 (M+H)⁺.

### (b) 5-(5-Hydroxy-5-trifluoromethyl-4,5-dihydro-isoxazol-3-yl)-thiophene-2-carboxylic acid

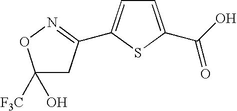

By proceeding in a similar manner to Reference Example 2(a) but using 5-(5-trifluoromethyl-isoxazol-3-yl)-thiophene-2-carbonitrile [Reference Example 7] there was prepared 5-(5-hydroxy-5-trifluoromethyl-4,5-dihydro-isoxazol-3-yl)-thiophene-2-carboxylic acid (85 mg, 74%) as a white solid. LCMS (Method A): R_{T}=6.34 minutes; 282 (M+H)⁺.

### (c) 5-(1H-Pyrazol-3-yl)-thiophene-2-carboxylic acid

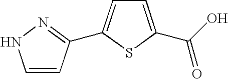

By proceeding in a similar manner to Reference Example 2(a) but using 5-(1H-pyrazol-3-yl)-thiophene-2-carbonitrile [Reference Example 3(b)] there was prepared 5-(1H-pyrazol-3-yl)-thiophene-2-carboxylic acid (97 mg, 97%) as a yellow solid. LCMS (Method A): R_{T} 10=4.79 minutes; 195 (M+H)⁺.

### (d) 5-(1-Benzyl-1H-Pyrazol-3-yl)-thiophene-2-carboxylic acid

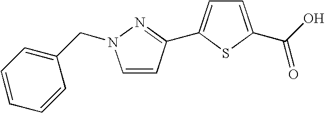

By proceeding in a similar manner to Reference Example 2(a) but using 5-(1-benzyl-1H-pyrazol-3-yl)-thiophene-2-carbonitrile [Reference Example 8(a)] there was prepared 5-(1-benzyl-1H-pyrazol-3-yl)-thiophene-2-carboxylic acid (59 mg, 96%) as a white powder. LCMS (Method A): R_{T}=7.98 minutes; 285 (M+H)⁺.

### (e) 5-(1-Phenethyl-1H-Pyrazol-3-yl)-thiophene-2-carboxylic acid

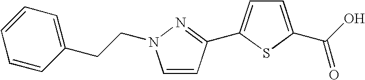

By proceeding in a similar manner to Reference Example 2(a) but using 5-(1-phenethyl-1H-pyrazol-3-yl)-thiophene-2-carbonitrile [Reference Example 8(b)] there was prepared 5-(1 phenethyl-1H-pyrazol-3-yl)-tiophene-2-carboxylic acid (116 mg, 97%) as white solid. LCMS (method A): R_{T}=8.44 minutes; 299 (M+H)⁺.

### (f) 5-[1-(3-Phenyl-propyl)-1H-pyrazol-3-yl]-thiophene-2-carboxylic acid

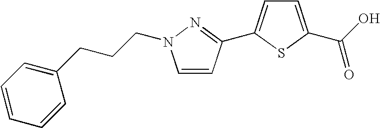

A solution of 5-[1-(3-phenyl-propyl)-1H-pyrazol-3-yl]-thiophene-2-carbonitrile [310 mg, 1.1 mmol, Reference Example 8(c)] in sodium hydroxide solution (10 ml, 1M) and dioxane (5 ml) was heated at 75° C. overnight, then 80° C. for a subsequent night. The reaction mixture was cooled to room temperature, diluted with water, acidified with hydrochloric acid (1M) and extracted three times with ethyl acetate. The combined extracts were then evaporated under reduced pressure, to provide 5-[1-(3-phenyl-propyl)-1H-pyrazol-3-yl]-thiophene-2-carboxylic acid (308 mg) as a yellow gum, which was used directly without further purification.

### (g) 5-[1-(2,3-Dihydro-benzo[1,4]dioxin-2-ylmethyl)-1H-pyrazol-3-yl]-thiophene-2-carboxylic acid

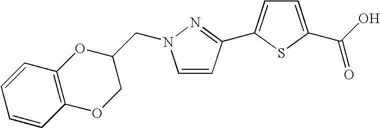

By proceeding in a similar manner to Reference Example 2(f) but using 5-[1-(2,3-dihydro-benzo[1,4]dioxin-2-ylmethyl)-1H-pyrazol-3-yl]-thiophene-2-carbonitrile [68 mg, 0.21 mmol, Reference Example 8(d)], and refluxing for 10 hours, there was prepared 5-[1-(2,3 dihydro-benzo[1,4]dioxin-2-ylmethyl)-1H-pyrazol-3-yl]-thiophene-2-carboxylic acid (67 mg) as a yellow solid. LCMS (method C): R_{T}=3.30 minutes; 343 (M+H)⁺.

### (h) 5-{1-[2-(4-Trifluoromethyl-phenyl)-ethyl]-1H-pyrazol-3-yl}-thiophene-2-carboxylic acid

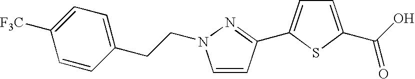

By proceeding in a similar manner to Reference Example 2(f) but using 5-{1-[2-(4-trifluoromethyl-phenyl)-ethyl]-1H-pyrazol-3-yl}-thiophene-2-carbonitrile [142 mg, 0.42 mmol, Reference Example 8(e)], and heating at 100° C. overnight, there was prepared 5-{1-[2-(4-trifluoromethyl-phenyl)-ethyl]-1H-pyrazol-3-yl}-thiophene-2-carboxylic acid (190 mg) as a white powder, which was used directly without further purification.

### (i) 5-(1-Benzo[1,3]dioxol-5-ylmethyl-1H-pyrazol-3-yl)-thiophene-2-carboxylic acid

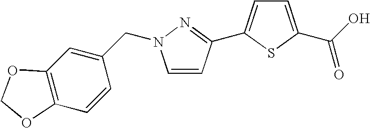

By proceeding in a similar manner to Reference Example 2(f) but using 5-(1-benzo[1,3]dioxol-5-ylmethyl-1H-pyrazol-3-yl)-thiophene-2-carbonitrile [407 mg, 1.31 mmol, Reference Example 8(f)], and heating at 90° C. for 4.5 hours, there was prepared 5-(1-benzo[1.3]dioxol-5-ylmethyl-1H-pyrazol-3-yl)-thiophene-2-carboxylic acid (432 mg). LCMS (Method C): R_{T}=3.10 minutes; 329 (M+H)⁺.

### (j) 5-{1-[2-(4-Trifluoromethoxy-phenyl)-ethyl]-1H-pyrazol-3-yl}-thiophene-2-carboxylic acid

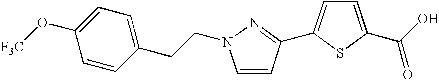

By proceeding in a similar manner to Reference Example 2(f) but using 5-{1-[2-(4-trifluoromethoxy-phenyl)-ethyl]-1H-pyrazol-3-yl}-thiophene-2-carbonitrile [238 mg, 0.82 mmol, Reference Example 8(g)], and heating at 90° C. for 24 hours, there was prepared 5-{1-[2-(4-trifluoromethoxy-phenyl)-ethyl]-1H-pyrazol-3-yl}-thiophene-2-carboxylic acid (250 mg) as a light brown solid. LCMS (Method C): R_{T}=3.66 minutes; 383 (M+H)⁺.

### (k) 5-{1-[2-(4-Fluoro-phenyl)-ethyl]-1H-pyrazol-3-yl}-thiophene-2-carboxylic acid

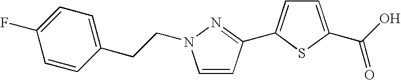

By proceeding in a similar manner to Reference Example 2(f) but using 5-{1-[2-(4-fluoro-phenyl)-ethyl]-1H-pyrazol-3-yl}-thiophene-2-carbonitrile [214 mg, 0.72 mmol, Reference Example 8(h)], and refluxing for 30 minutes, there was prepared 5-{1-[2-(4-fluoro-phenyl)-ethyl]-1H-pyrazol-3-yl}-thiophene-2-carboxylic acid (118 mg) as an off-white solid. LCMS (Method C): R_{T}=3.27 minutes; 317 (M+1H)⁺.

### (l) 5-[1-(1-Phenyl-ethyl)-1H-pyrazol-3-yl]-thiophene-2-carboxylic acid

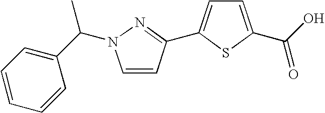

By proceeding in a similar manner to Reference Example 2(f) but using 5-[1-(1-phenyl-ethyl)-1H-pyrazol-3-yl]-thiophene-2-carbonitrile [371 mg, 1.33 mmol, Reference Example 8(i)], and refluxing for 3 hours, there was prepared 5-[1-(1-phenyl-ethyl)-1H-pyrazol-3-yl]-thiophene-2-carboxylic acid (255 mg) as a yellow powder. LCMS (Method C): R_{T} 3.34 minutes; 299 (M+H)⁺.

### (m) 5-[1-(2-Morpholin-4-yl-ethyl)-1H-Pyrazol-3-yl]-thiophene-2-carboxylic acid

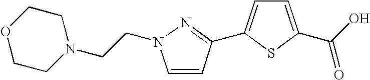

By proceeding in a similar manner to Reference Example 2(f) but using 5-[1-(2-morpholin-4-yl-ethyl)-1H-pyrazol-3-yl]-thiophene-2-carbonitrile [330 mg, 1.11 mmol, Reference Example 8(j)], and heating to 90° C. for 3 hours, there was prepared 5-[1-(2-morpholin-4-yl-ethyl)-1H-pyrazol-3-yl]-thiophene-2-carboxylic acid (374 mg), which was used directly without further purification.

### (n) 5-[1-(Tetrahydro-pyran-2-ylmethyl)-1H-pyrazol-3-yl]-thiophene-2-carboxylic acid

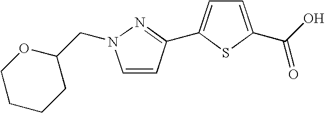

By proceeding in a similar manner to Reference Example 2(f) but using 5-[1-(tetrahydro-pyran-2-ylmethyl)-1H-pyrazol-3-yl]-thiophene-2-carbonitrile [185 mg, 0.67 mmol, Reference Example 8(k)], and heating to 90° C. overnight, there was prepared 5-[1-(tetrahydro-pyran-2-ylmethyl)-1H-pyrazol-3-yl]-thiophene-2-carboxylic acid (188 mg) as a white solid. LCMS (Method C): R_{T}=2.99 minutes; 293 (M+H)⁺.

### (o) 5-(2-Phenethyl-3H-imidazol-4-yl)-thiophene-2-carboxylic acid

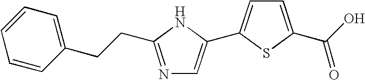

A mixture of 5-(2-phenethyl-3H-imidazol-4-yl)-thiophene-2-carboxylic acid methyl ester [100 mg, 0.32 mmol, Reference Example 18(a)] in sodium hydroxide solution (3 ml, 1M) and methanol (10 ml) was heated to 50° C. for 1 hour. The reaction mixture was allowed to cool to room temperature, concentrated to remove the methanol, and washed with dichloromethane. The aqueous phase was then acidified to low pH using concentrated hydrochloric acid, then extracted with ethyl acetate (3×). The organic phases were combined, dried Na₂SO₄), and concentrated to provide 5-(2-phenethyl-3H-imidazol-4-ly)-thiophene-2-carboxylic acid (79 mg) as a brown oily solid, which was used directly without farther purification.

### (p) 5-(1-Phenethyl-5-trifluoromethyl-1H-pyrazol-3-yl)-thiophene-2-carboxylic acid

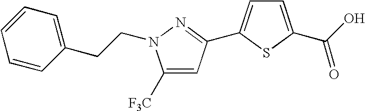

By proceeding in a similar manner to Reference Example 2(f) but using 5-(1-phenethyl-5-trifluoromethyl-1H-pyrazol-3-yl)-thiophene-2-carbonitrile [125 mg, 0.36 mmol, Reference Example 8(1)], and heating to 75° C. for 8 hours, there was prepared 5-(1-phenethyl-5-trifluoromethyl-1H-pyrazol-3-yl)-thiophene-2-carboxylic acid (150 mg). LCMS (Method C): R_{T}=3.91 minutes; 365 (M⁻).

### Reference Example 3

### (a) 5-(2-Methyl-2H-pyrazol-3-yl)-thiophene-2-carbonitrile and 5-(1-methyl-1H-pyrazol-3-yl)-thiophene-2-carbonitrile

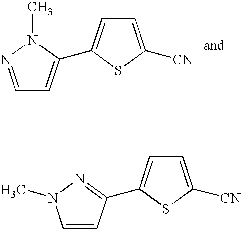

A solution of 5-(3-dimethylamino-acryloyl)-thiophene-2-carbonitrile [0.70 g, 3.34 mmol, Reference Example 4(a)]) in ethanol (30 ml) was treated with methylhydrazine (0.19 ml, 3.58 mmol). The mixture was heated to reflux for 7 hours then cooled to room temperature and then concentrated under reduced pressure to give a mixture of 5-(2-methyl-2H-pyrazol-3-yl)-thiophene-2-carbonitrile and 5-(1-methyl-1H-pyrazol-3-yl)-thiophene-2-carbonitrile (0.50 g) which was used directly in the next step.

### (b) 5-(1H-Pyrazol-3-yl)-thiophene-2-carbonitrile

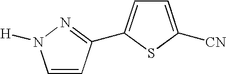

By proceeding in a similar manner to Reference Example 3(a) but using 1.19 g of 5-(3-dimethylamino-acryloyl)-thiophene-2-carbonitrile, 20 ml of ethanol and hydrazine hydrate (0.20 ml, 6.4 mmol), heating the reaction mixture at reflux for 16 hours and partitioning the reaction product between ethyl acetate and water there was prepared 5-(1H-Pyrazol-3-yl)-thiophene-2-carbonitrile (0.80 g, 89%) as a brown solid. LCMS (Method A): R_{T}=5.90 minutes; 176 (M+H)⁺.

### (c) 5-(5-Trifluoromethyl-1H-pyrazol-3-yl)-thiophene-2-carbonitrile

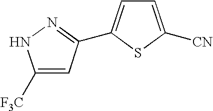

A solution of 5-(4,4,4-trifluoro-3-oxo-butyryl)-thiophene-2-carbonitrile [3,6 g, 14.6 mmol, Reference Example 5(a)] in ethanol (50 ml) was treated with hydrazine hydrate (2 ml). The resulting solution was heated to reflux for 4.5 hours, allowed to cool to room temperature overnight, then concentrated to give a residue. The residue was dissolved in ethyl acetate, washed with 1M hydrochloric acid, brine, then dried (MgSO₄), and concentrated, to provide 5-(5-trifluoromethyl-1H-pyrazol-3-yl)-thiophene-2-carbonitrile (2.7 g). LCMS (Method C): R_{T}=3.31 minutes; 242 (M⁻).

### Reference Example 4

### (a) 5-(3-Dimethylamino-acryloyl-thiophene-2-carbonitrile

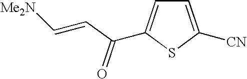

A solution of 5-acetylthiophene-2-carbonitrile (1.0 g, 6.6 mmol) in dimethylformamide (50 ml) was treated with tert-butoxybis(dimethylamino)methane (1.7 ml, 8.27 mmol). The resulting yellow solution was heated at 70° C. for 8 hours, then allowed to cool to room temperature and then concentrated under reduced pressure. The residue was triturated with diisopropyl ether, concentrated to about 5 ml and triturated again with pentane to give 5-(3-dimethylamino-acryloyl)-thiophene-2-carbonitrile (1.3 g, 95%) as a yellow solid. LCMS (Method A): R_{T}=5.63 minutes; 207 (M+H)⁺.

### (b) 5-(5-Hydroxy-5-trifluoromethyl-4,5-dihydro-isoxazol-3-yl)-thiophene-2-carbonitrile

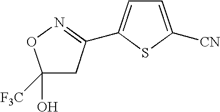

A solution of 5-(4,4,4-Trifluoro-3-oxo-butyryl)-thiophene-2-carbonitrile (200 mg, 0.81 mmol [Reference example 5]) in ethanol (4 ml) was treated with hydroxylamine hydrochloride (56 mg, 0.81 mmol) and acetic acid (4 ml). The resulting solution was heated to reflux for 2 hours at which time t.l.c. analysis [ethyl acetate/petroleum ether fraction (b.p. 40-60° C.) 7:3, v/v] indicated complete disappearance of the starting material. The mixture was allowed to cool to room temperature and then concentrated under reduced pressure. The residue was dissolved in ethyl acetate, and the solution was washed with saturated sodium bicarbonate solution and then concentrated in vacuo. The residue was subjected to column chromatography on silica eluting with 10%-19% (v/v) ethyl acetate in petroleum ether (bp 40-60° C.) to give 5-(5-hydroxy-5-trifluoromethyl-4,5-dihydro-isoxazol-3-yl)-thiophene-2-carbonitrile (162 mg, 82%) as an off-white solid. LCMS (Method A): R_{T}=7.63 minutes; 263 (M+H)⁺.

### Reference Example 5

### (a) 5-(4,4,4-Trifluoro-3-oxo-butyryl)-thiophene-2-carbonitrile

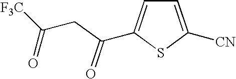

A suspension of sodium methoxide (384 mg, 17.3 mmol) in anhydrous diethyl ether (50 ml) under nitrogen was treated with ethyl trifluoroacetate (1.97 ml, 16.5 mmol) followed by 5-acetylthiophene-2-carbonitrile (2.5 g, 16.5 mmol). The solution was stirred vigorously for 4 days and then quenched by the addition of hydrochloric acid (1M). The reaction mixture was extracted with ethyl acetate and the organic phase was washed with brine, then dried over sodium sulfate and then evaporated to give 5-(4,4,4-trifluoro-3-oxo-butyryl)-thiophene-2-carbonitrile (4.07 g, 75%) as a brown solid, which was used without further purification. LCMS (Method C): R_{T}=2.68 minutes; (-ve ion) 246 (M⁻)

### Reference Example 6

### (a) 5-(4-Trifluoromethyl-1H-imidazol-2-yl)-thiophene-2-carboxylic acid

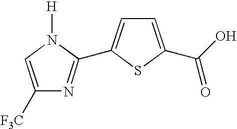

A suspension of 5-(4-trifluoromethyl-1H-imidazol-2-yl)-thiophene-2-carboxylic acid methyl ester [142 mg, 0.51 mmol, Reference Example 9(a)] in a mixture of sodium hydroxide solution (15 ml, 2M) and ethanol (15 ml) was heated to 50° C. for 15 minutes. The reaction mixture was allowed to cool to room temperature and then extracted five times with ethyl acetate. The combined extracts were dried over magnesium sulfate and then concentrated in vacuo to yield 5-(4-trifluoromethyl-1H-imidazol-2-yl)-thiophene-2carboxylic acid (115 mg, 85%) as a pale yellow powder. LCMS (Method A): R_{T}=6.04 minutes; 263 (M+H)⁺.

### (b) 5-(3-Benzyloxy-phenyl-thiophene-2-carboxylic acid

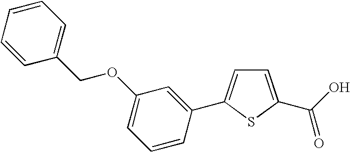

A mixture of 5-(3-benzyloxy-phenyl)-thiophene-2-carboxylic acid ethyl ester [137 mg, 0.41 mmol, Reference Example 21(a)], lithium hydroxide monohydrate (34 mg, 0.81 mmol), water (0.75 ml), methanol (5 ml) and tetrahydrofuran (2 ml) was stirred at room temperature overnight. The reaction mixture was partitioned between diethyl ether and water, and the aqueous phase was separated and washed again with diethyl ether. The aqueous phase was acidified using 1M hydrochloric acid, and then extracted with ethyl acetate (3×). The organic phases were combined, washed with water, followed by brine, dried (MgSO₄), and concentrated, to provide 5-(3-benzyloxy-phenyl)-thiophene-2-carboxylic acid (39 mg) as a white solid. LCMS (Method C): R_{T}=3.85 minutes.

### (c) 5-[1-(2-Benzylamino-ethyl)-1H-pyrazol-3-yl]-thiophene-2-carboxylic acid

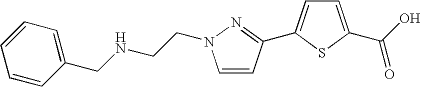

A mixture of 5-[1-(2-benzylamino-ethyl)-1H-pyrazol-3-yl]-thiophene-2-carboxylic acid methyl ester [60 mg, 0.18 mmol, Reference Example 28(f)], lithium hydroxide monohydrate (20 mg, 0.48 mmol), water (1.25 ml) and acetonitrile (3.75 ml) was stirred at room temperature overnight. The reaction mixture was concentrated, and the residual material was acidified with 1M hydrochloric acid to give a yellow solid which was filtered then dried under vacuum, to provide 5-[1-(2-benzylamino-ethyl)-1H-pyrazol-3-yl]-thiophene-2-carboxylic acid (80 mg) as a yellow solid. LCMS (Method C): R_{T}=1.89 minutes; 328 (M+H)⁺.

### (d) 5-(1-{3-[(Quinolin-2-ylmethyl)-amino]-propyl})-1H-pyrazol-3-yl)-thiophene-2-carboxylic acid

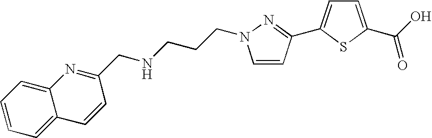

By proceeding in a similar manner to Reference Example 6(c) but using 5-(1-{3-[(quinolin-2-ylmethyl)-amino]-propyl}-1H-pyrazol-3-yl)-thiophene-2-carboxylic acid methyl ester [154 mg, 0.38 mmol, Reference Example 28(g)] there was prepared 5-(1-{3-[(quinolin-2-ylmethyl)-amino]-propyl}-1H-pyrazol-3-yl)-thiophene-2-carboxylic acid. LCMS (Method C): R_{T}=2.08 minutes; 393 (M+H)⁺.

### (e) 5-(1-{3-[(Benzo[1,3]dioxol-5-ylmethyl)-amino]-propyl}-1H-pyrazol-3-yl)-thiophene-2-carboxylic acid

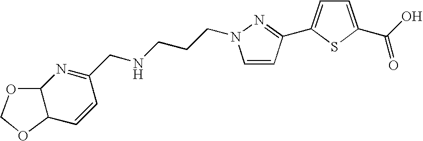

By proceeding in a similar manner to Reference Example 6(c) but using 5-(1-{3-[(benzo[1,3]dioxol-5-ylmethyl)-amino]-propyl}-1H-pyrazol-3-yl)-thiophene-2-carboxylic acid methyl ester [152 mg, 0.38 mmol, Reference Example 28(h)] there was prepared 5-(1-{3-[benzo[1,3]dioxol-5-ylmethyl)-amino]-propyl}-1H-pyrazol-3-yl)-thiophene-2-carboxylic acid. LCMS (Method C): R_{T}=1.98 minutes; 386 (M+H)⁺.

### (f) 5-(1-{2-[(Benzo[1,3]dioxol-5-ylmethyl)-amino]-ethyl}-1H-pyrazol-3-yl)-thiophene-2-carboxylic acid

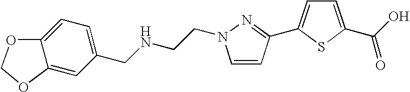

By proceeding in a similar manner to Reference Example 6(c) but using 5-(1-{2-[(benzo[1,3]dioxol-5-ylmethyl)-amino]-ethyl}-1H-pyrazol-3-yl)-thiophene-2-carboxylic acid methyl ester [154 mg, 0.4 mmol, Reference Example 28(i)] there was prepared 5-(1-{2-[(benzo[1,3]dioxol-5-ylmethl)-amino]-ethyl}-1H-pyrazol-3-yl)-thiophene-2-carboxylic acid. LCMS (Method C): R_{T}=1.97 minutes; 372 (M+H)⁺.

### (g) 5-(1-{2-[(Pyridin-4-ylmethyl)-amino]-ethyl}-1H-pyrazol-3-yl)-thiophene-2-carboxylic acid

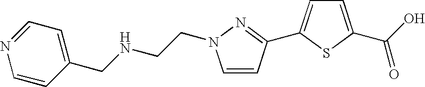

By proceeding in a similar manner to Reference Example 6(c) but using 5-(1-{2-[(pyridin-4-ylmethyl)-amino]-ethyl}-1H-pyrazol-3-yl)-thiophene-2-carboxylic acid methyl ester [137 mg, 0.4 mmol, Reference Example 28(j)] there was prepared 5-(1-{2-[(pyridin-4-ylmethyl)-amino]-ethyl}-1H-pyrazol-3-yl)-thiophene-2-carboxylic acid. LCMS (Method C): R_{T}=1.48 minutes; 329 (+H)⁺.

### (h) 5-[6-(3-Phenyl-propionylamino-pyridin-2-yl]-thiophene-2-carboxylic acid

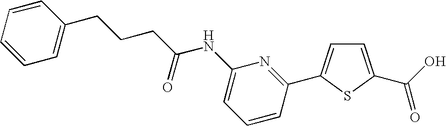

To a solution of 5-[6-(3-phenyl-propionylamino)-pyridin-2-yl]-thiophene-2-carboxylic acid methyl ester [338 mg, 0.92 mmol, Reference Example 11(f)] and tetrahydrofuran (10 ml) was added potassium trimethylsilanolate (709 mg, 5.5 mmol). The mixture was stirred at room temperature for 1.5 hours, concentrated, and the residue was subjected to reverse-phase preparative HPLC using acetonitrile and water (gradient 5:95 to 5:95, v/v, over 90 minutes) as eluent, to provide 5-[6-(3-phenyl-propionylamino-pyridin-2-yl]-thiophene-2-carboxylic acid (84 mg). LCMS (Method C): R_{T}=3.49 minutes; 353 (M+H)⁺.

### (i) 5-[6-(3-Phenyl-propylamino)-pyridin-2-yl]-thiophene-2-carboxylic acid

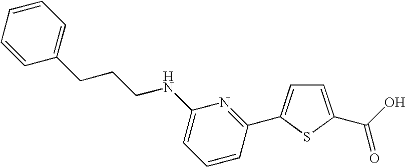

To a solution of 5-[6-(3-phenyl-propylamino)-pyridin-2-yl]-thiophene-2-carboxylic acid methyl ester [660 mg, 1.87 mmol, Reference Example 28(k)] and tetrahydrofuran (10 ml) was added potassium trimethylsilanolate (1.44 g, 11.3 mmol). The mixture was stirred at room temperature for 2.5 hours, concentrated, and the residue was subjected to reverse-phase preparative HPLC using acetonitrile and water (gradient 30:70 to 70:30, v/v, over 90 minutes) as eluent, to provide 5-[6-(3-phenyl-propylamino)-pyridin-2-yl]-thiophene-2-carboxylic acid (347 mg). LCMS (Method C): R_{T}=3.64 minutes; 339 (M+H)⁺.

### (j) 5-{1-[(3-Methoxy-phenylcarbamoyl)-methyl]-1H-pyrazol-3-yl}-thiophene-2-carboxylic acid

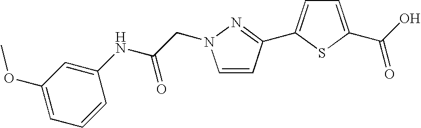

To a solution of 5-{1-[(3-methoxy-phenylcarbamoyl)-methyl]-1H-pyrazol-3-yl}-thiophene-2-carboxylic acid methyl ester [300 mg, 0.8 mmol, Reference Example 30(f)], tetrahydrofuran (10 ml) and water (10 ml), was added lithium hydroxide (90 mg, 2.2 mmol). The mixture was stirred at room temperature for 2 hours, then quenched with 1M hydrochloric acid (10 ml) and concentrated to give a residue. The residue was triturated with 1M hydrochloric acid and filtered, to provide 5-{1-[(3-methoxy-phenylcarbamoyl-methyl]-1H-pyrazol-3-yl}-thiophene-2-carboxylic acid (253 mg) as a white solid. LCMS (Method C): R_{T}=2.89 minutes; 358 (M+H)⁺.

### (k) 5-[1-(1-Oxy-quinolin-2-ylmethyl)-1H-pyrazol-3-yl]-thiophene-2-carboxylic acid

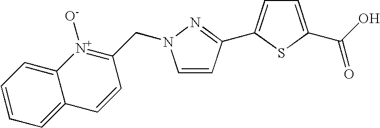

By proceeding in a similar manner to Reference Example 6(c) but using 5-[1-(1-oxy-quinolin-2-ylmethyl)-1H-pyrazol-3-yl]-thiophene-2-carboxylic acid methyl ester [700 mg, 0.1.92 mmol, Reference Example 10(ao)] there was prepared 5-[1-(1-oxy-quinolin-2-ylmethyl)-1H-pyrazol-3-yl]-thiophene-2-carboxylic acid (627 mg) as a white solid. LCMS (Method C): R_{T}=2.59 minutes; 352 (M+H)⁺.

### Reference Example 7

### (a) 5-(5-Trifluoromethyl-isoxazol-3-yl)-thiophene-2-carbonitrile

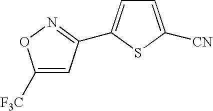

A solution of 5-(5-hydroxy-5-trifluoromethyl-4,5-dihydro-isoxazol-3-yl)-thiophene-2-carbonitrile [168 mg, 0.64 mmol, Reference example 4(b)]) in anhydrous dichloromethane (10 ml), under nitrogen, was treated with molecular sieves and 1,8-diazabicyclo[5.4.0]undec-7-ene (0.1 ml, 0.67 mmol). The mixture was refluxed for 2 hours and then the dichloromethane was evaporated and the residue was resuspended in dichloroethane. The mixture was refluxed for 2.5 days and then filtered. The filtrate was concentrated in vacuo and the residue was subjected to flash column chromatography on silica eluting with 10%-30% (v/v) ethyl acetate in petroleum ether fraction (b.p. 40-60° C.) to yield 5-(5-trifluoromethyl-isoxazol-3-yl)-thiophene-2-carbonitrile (136 mg, 87%) as a white solid. LCMS (Method A): R_{T}=9.83 minutes; 337.

### Reference Example 8

### (a) 5-(1-Benzyl-1H-pyrazol-3-yl)-thiophene-2-carbonitrile

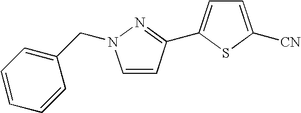

A solution of 5-(1H-pyrazol-3-yl)-thiophene-2-carbonitrile [98 mg, 0.55 mmol, Reference Example 3(b)]) in toluene (6 ml) was treated with potassium hydroxide (25 mg, 0.44 mmol), potassium carbonate (61 mg, 0.44 mmol), tetrabutylammonium hydrogen sulfate (23 mg, 0.066 mmol) and benzyl chloride (76 μl, 0.66 mmol). The mixture was refluxed overnight after which t.l.c. (ethyl acetate 3:2 petroleum ether, bp 40-60° C.) indicated the presence of remaining starting material. A further aliquot of benzyl chloride (76 μl, 0.66 mmol) was added and the mixture was refluxed for a further 40 hours. The reaction mixture was filtered and the residue was washed with toluene. The combined filtrate and washings were concentrated in vacuo and the residue was partitioned between ethyl acetate and brine. The two phases were separated and the organic phase was dried over sodium sulfate and then concentrated in vacuo. The crude product was subjected to column chromatography on silica eluting with 8% (v/v) ethyl acetate in petroleum ether fraction (b.p. 40-60° C.) to yield 5-(1-benzyl-1H-pyrazol-3-yl)-thiophene-2-carbonitrile (63 mg, 43%) as a yellow powder. LCMS (Method A): R_{T}=9.72 minutes; 266 (M+H)⁺.

### (b) 5-(1-Phenethyl-1H-pyrazol-3-yl)-thiophene-2-carbonitrile

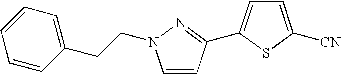

By proceeding in a similar manner to Reference Example 8(a) but using 2-bromoethyl benzene and subjecting the reaction product to column chromatography on silica eluting with 7.5%-12% (v/v) ethyl acetate in petroleum ether fraction (b.p. 40-60° C.) there was prepared 5-(1-phenethyl-1H-pyrazol-3-yl)-thiophene-2-carbonitrile (118 mg, 89%) as a white solid. LCMS (Method A): R_{T}=10.14 minutes; 280 (M+H)⁺.

### (c) 5-[1-(3-Phenyl-propyl)-1H-pyrazol-3-yl]-thiophene-2-carbonitrile

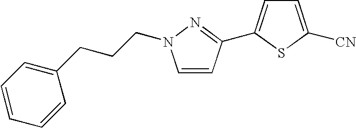

To a mixture of 5-(1H-pyrazol-3-yl)-thiophene-2-carbonitrile [244 mg, 1.39 mmol, Reference Example 3(b)], potassium carbonate (25 mg, 0.44 mmol) and N,N-dimethylformamide (7 ml) was added 1-bromo-3-phenylpropane (320 μl, 2.1 mmol). The resulting mixture was heated to 75° C. and stirred overnight. The reaction mixture was allowed to cool to room temperature and concentrated in vacuo to give a residue, which was then partitioned between ethyl acetate and water. The organic layer was isolated, and the aqueous phase was washed with ethyl acetate (2×). The organic phases were combined and concentrated, then subjected to flash column chromatography on silica using a mixture of pentane and ethyl acetate (gradient 9:1 to 1:1, v/v) as eluent, to provide 5-[1-(3-phenyl-propyl)-1H-pyrazol-3-yl]-thiophene-2-carbonitrile (342 mg), which was used directly without further purification.

### (d) 5-[1-(2,3-Dihydro-benzo[1,4]dioxin-2-ylmethyl)-1H-pyrazol-3-yl]-thiophene-2-carbonitrile

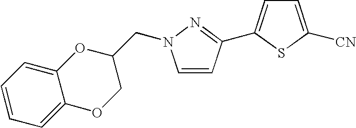

A solution of 5-(1H-pyrazol-3-yl)-thiophene-2-carbonitrile [82 mg, 0.46 mmol, Reference Example 3 (b)]) in toluene (7 ml) was treated with potassium hydroxide (29 mg, 0.51 mmol), potassium carbonate (71 mg, 0.51 mmol), tetrabutylammonium hydrogen sulfate (25 mg, 0.073 mmol) and 2-bromomethyl-1,4-benzodioxane (316 mg, 1.38 mmol). The mixture was heated to reflux for 24 hours, allowed to cool to room temperature, then filtered, and the residue was washed with toluene. The combined filtrate and washings were concentrated in vacuo and the residue was partitioned between ethyl acetate and brine. The two phases were separated; the organic phase was dried (Na₂SO₄), and then concentrated in vacuo. The crude product was subjected to flash column chromatography on silica using ethyl acetate and petroleum ether fraction (b.p. 40-60° C.) (6:4, v/v) as eluent, to provide 5-[1-(2,3-dihydro-benzo[1,4]dioxin-2-ylmethyl)-1H-pyrazol-3-yl]-thiophene-2-carbonitrile as an oil. LCMS (Method C): R_{T}=3.79 minutes; 324 (M+H)⁺.

### (e) 5-{1-[2-(4-Trifluoromethyl-phenyl)-ethyl]-1H-pyrazol-3-yl}-thiophene-2-carbonitrile

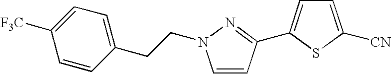

To a mixture of 5-(1H-pyrazol-3-yl)-thiophene-2-carbonitrile [400 mg, 2.28 mmol, Reference Example 3(b)], potassium carbonate (666 mg, 4.8 mmol) and N)N-dimethylformamide (8 ml) was added methanesulfonic acid 2-(4-trifluoromethyl-phenyl)-ethyl ester (612 mg, 2.3 mmol). The resulting mixture was heated to 70° C. and stirred overnight. The reaction mixture was then quenched in water, and extracted with ethyl acetate. The organic layer was dried (MgSO₄), concentrated, and then subjected to flash column chromatography using a mixture of cyclohexane and ethyl acetate (3:1, v/v) as eluent, to provide 5-[1-(3-phenyl-propyl-1H-pyrazol-3-yl]-thiophene-2-carbonitrile (145 mg), which was used directly without further purification.

### (f) 5-(1-Benzo[1,3]dioxol-5-ylmethyl-1H-pyrazol-3-yl)-thiophene-2-carbonitrile

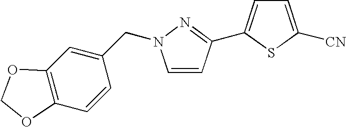

To a mixture of 5-(1H-pyrazol-3-yl)-thiophene-2-carbonitrile [500 mg, 2.85 mmol, Reference Example 3(b)], potassium carbonate (800 mg, 5.8 mmol) and N)N-dimethylformamide (20 ml) was added 5-bromomethyl-benzo[1,3]dioxole (900 mg, 4.2 mmol). The resulting mixture was heated to 75° C. and stirred overnight. The reaction mixture was allowed to cool to room temperature and concentrated in vacuo to give a residue, which was then partitioned between ethyl acetate and water. The organic layer was isolated, and the aqueous phase was washed with ethyl acetate. The combined organic phases were concentrated, and subjected to flash column chromatography using a mixture of cyclohexane and ethyl acetate (gradient 100:0 to 95:5 to 80:20 to 50:50, v/v) as eluent, to provide 5-(1-benzo[1,3]dioxol-5-ylmethyl-1H-pyrazol-3-yl)-thiophene-2-carbonitrile (704 mg), which was used directly without further purification.

### (g) 5-{1-[2-(4-Trifluoromethoxy-phenyl-ethyl]-1H-pyrazol-3-yl}-thiophene-2-carbonitrile

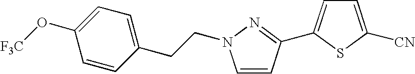

To a mixture of 5-(1H-pyrazol-3-yl)-thiophene-2-carbonitrile [250 mg, 1.42 mmol, Reference Example 3(b)], potassium carbonate (315 mg, 2.28 mmol) and N,N-dimethylformamide (13 ml) was added methanesulfonic acid 2-(4-trifluoromethoxy-phenyl)-ethyl ester (650 mg, 2.28 mmol). The resulting mixture was heated to 75° C. and stirred for 24 hours. The reaction mixture was allowed to cool to room temperature and concentrated in vacuo to give a residue. The residue was dissolved in ethyl acetate, washed with water (3×), followed by brine, then dried (Na₂SO₄), concentrated and subjected to flash column chromatography on silica, using ethyl acetate and petroleum ether fraction (b.p. 40-60° C.) (gradient 70:30 to 15:85, v/v) as eluent, to provide 5-{1-[2-(4-trifluoromethoxy-phenyl)-ethyl]-1H-pyrazol-3-yl}-thiophene-2-carbonitrile (305 mg). LCMS (Method C): R_{T}=4.18 minutes; 364 (M+H)⁺.

### (h) 5-{1-[2-(4-Fluoro-phenyl)-ethyl]-1H-pyrazol-3-yl}-thiophene-2-carbonitrile

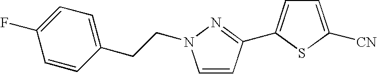

To a mixture of 5-(1H-pyrazol-3-yl)-thiophene-2-carbonitrile [300 mg, 1.72 mmol, Reference Example 3(b)], potassium carbonate (500 mg, 3.6 mmol) and N,N-dimethylformamide (4 ml) was added 1-(2-bromoethyl)-4-fluoro-benzene (566 mg, 3.4 mmol). The resulting mixture was heated to 80° C. and stirred overnight. The reaction mixture was then quenched in water and extracted with diethyl ether. The organic layer was dried (MgSO₄), concentrated, and then subjected to flash column chromatography using a mixture of cyclohexane and ethyl acetate (8:2, v/v) as eluent, to provide 5-{1-[2-(4-fluoro-phenyl)-ethyl]-1H-Dyrazol-3-yl}-thiophene-2-carbonitrile.

### (i) 5-[1-(1-Phenyl-ethyl)-1H-pyrazol-3-yl]-thiophene-2-carbonitrile

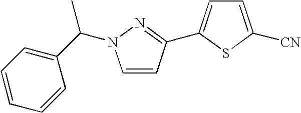

To a mixture of 5-(1H-pyrazol-3-yl)-thiophene-2-carbonitrile [500 mg, 2.86 mmol, Reference Example 3(b)], potassium carbonate (820 mg, 5.72 mmol) and N,N-dimethylformamide (5 ml) was added (1-bromoethyl)benzene (582 mg, 3.15 mmol). The resulting mixture was heated to 80° C. and stirred for 4 hours. The reaction mixture was concentrated in vacuo and then partitioned between diethyl ether and water. The organic layer was separated, dried (MgSO₄), concentrated, and then subjected to flash column chromatography using a mixture of cyclohexane and ethyl acetate (gradient 100:0 to 80:20, v/v) as eluent, to provide 5-[1-(1-phenyl-ethyl)-1H-pyrazol-3-yl]-thiophene-2-carbonitrile (371 mg).

### (j) 5-[1-(2-Morpholin-4-yl-ethyl)-1H-pyrazol-3-yl]-thiophene-2-carbonitrile

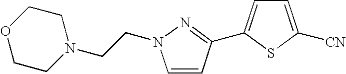

To a mixture of 5-(1H-pyrazol-3-yl)-thiophene-2-carbonitrile [400 mg, 2.28 mmol, Reference Example 3(b)], potassium carbonate (1.26 g, 9.12 mmol) and N,N-dimethylformamide (9.5 ml) was added 4-(2-chloroethyl)morpholine hydrochloride (640 mg, 3.44 mmol). The resulting mixture was heated to 75° C. and stirred overnight. The reaction mixture was allowed to cool to room temperature and then concentrated to give a residue. The residue was partitioned between ethyl acetate and water. The organic layer was separated, concentrated, and then subjected to flash column chromatography using a mixture of pentane and ethyl acetate (gradient 80:20 to 0:100, v/v) as eluent, to provide 5-[1-(2-morpholin-4-yl-ethyl)-1H-pyrazol-3-yl]-thiophene-2-carbonitrile (340 mg).

### (k) 5-[1-(Tetrahydro-pyran-2-ylmethyl)-1H-pyrazol-3-yl]-thiophene-2-carbonitrile

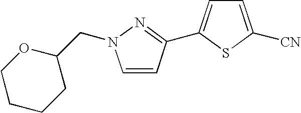

To a mixture of 5-(1H-pyrazol-3-yl)-thiophene-2-carbonitrile [200 mg, 1.13 mmol, Reference Example 3(b)], potassium carbonate (203 mg, 1.47 mmol) and N,N-dimethylformamide (10 ml) was added 2-(bromomethyl)tetrahydro-2H-pyran (264 mg, 1.47 mmol). The resulting mixture was heated to 80° C. and stirred for 28 hours. The reaction mixture was allowed to cool to room temperature and then concentrated to give a residue. The residue was partitioned between ethyl acetate and water. The organic layer was separated, washed with brine, dried (Na₂SO₄), concentrated, and then subjected to flash column chromatography using a mixture of petroleum ether fraction (b.p. 40-60° C.) and ethyl acetate (85:15, v/v) as eluent, to provide 5-[1-(tetrahydro-pyran-2-ylmethyl)-1H-pyrazol-3-yl]-thiophene-2-carbonitrile (201 mg). LCMS (Method C): R_{T}=2.52 minutes; 274 (M+H)⁺.

(l) 5-(1-Phenethyl-5-trifluoromethyl-1H-pyrazol-3-yl)-thiophene-2-carbonitrile

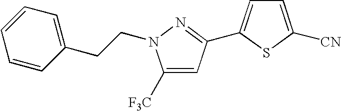

To a mixture of 5-(5-trifluoromethyl-1H-pyrazol-3-yl)-thiophene-2-carbonitrile [532 mg, 2.2 mmol, Reference Example 3(c)], potassium carbonate (820 mg, 5.72 mmol) and N,N-dimethylformamide (7 ml) was added (1-bromoethyl)benzene (511 mg, 2.76 mmol). The resulting mixture was heated to 75° C. and stirred overnight. The reaction mixture was concentrated in vacuo, and the residue generated was suspended in ethanol and filtered. The filtrate was concentrated and subjected to flash column using a mixture of cyclohexane and ethyl acetate (gradient 100:0 to 95:5, v/v, over 30 minutes) as eluent, to provide 5-(1-phenethyl-5-trifluoromethyl-1H-pyrazol-3-yl)-thiophene-2-carbonitrile (142 mg). LCMS (Method C): R_{T}=4.44 minutes.

### Reference Example 9

### (a) 5-(4-Trifluoromethyl-1H-imidazol-2-yl)-thiophene-2-carboxylic acid methyl ester

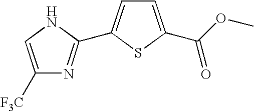

To a solution of sodium acetate (1.2 g, 13.9 mmol) in water (15 ml) was added 1,1-dibromo-3,3,3-trifluoroacetone (0.80 ml, 5.37 mmol) and the resulting mixture was heated to 80° C. for 45 minutes. The solution was cooled to 0° C. and 5-formyl-thiophene-2-carboxylic acid methyl ester (0.84 g, 4.92 mmol) in methanol (20 ml) was added followed by conc. ammonium hydroxide solution (25 ml) and the solution was allowed to warm to room temperature overnight. The reaction mixture was concentrated and the aqueous residue was extracted three times with ethyl acetate. The combined organic phase was evaporated and the crude product was purified by column chromatography on silica eluting with 10% v/v ethyl acetate in dichloromethane to yield 5-(4-trifluoromethyl-1H-imidazol-2-yl)-thiophene-2-carboxylic acid methyl ester (0.22 g, 16%) as a pale yellow powder. LCMS (Method A): R_{T}=7.55 minutes; 277 (M+H)⁺.

### Reference Example 10

### (a) 5-[1-(2-Benzyloxy-ethyl)-1H-pyrazol-3-yl]-thiophene-2-carboxylic acid methyl ester

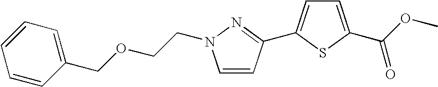

To a mixture of 5-(1H-pyrazol-3-yl)-thiophene-2-carboxylic acid methyl ester [250 mg, 1.20 mmol, Reference Example 12(a)], potassium carbonate (330 mg, 2.39 mmol) and N,N-dimethylformamide (5 ml) was added benzyl 2-bromoethyl ether (210 μl, 1.33 mmol). The resulting mixture was heated to 70° C. and stirred overnight. The reaction mixture was allowed to cool to room temperature and concentrated in vacuo to give a residue, which was then partitioned between ethyl acetate and water. The organic layer was isolated, and washed with 1M hydrochloric acid, dried (MgSO₄), and concentrated to give a yellow oil. The oil was treated with pentane and allowed to stand overnight. The supernatent was decanted and the remaining residue was dried, to provide 5-[1-(2-benzyloxy-ethyl-1H-pyrazol-3-yl]-thiophene-2-carboxylic acid methyl ester (330 mg) as a viscous yellow oil. LCMS (Method C): R_{T}=3.81 minutes; 343 (M+H)⁺.

### (b) 5-(1-Pent-4-ynyl-1H-pyrazol-3-yl)-thiophene-2-carboxylic acid methyl ester

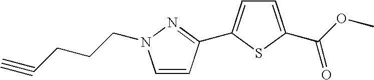

To a mixture of 5-(1H-pyrazol-3-yl)-thiophene-2-carboxylic acid methyl ester [65 mg, 0.31 mmol, Reference Example 12(a)], potassium carbonate (150 mg, 1.09 mmol) and N,N-dimethylformamide (2 ml) was added 5-chloro-1-pentyne (46 mg, 0.45 mmol). The resulting mixture was heated to 80° C. and stirred overnight. Water (2 ml) was added to the reaction mixture, which was allowed to stir for a further 30 minutes before being poured onto Isolute HM-N cartridges. The cartridges were washed with diethyl ether, then dichloromethane and methanol (90:10, v/v), and the resulting eluent was concentrated, to provide 5-(1-pent-4-ynyl-1H-pyrazol-3-yl)-thiophene-2-carboxylic acid methyl ester. LCMS (Method C): R_{T}=3.25 minutes; 275 (M+H)⁺.

### (c) 5-[1-(3-Phenyl-allyl)-1H-pyrazol-3-yl]-thiophene-2-carboxylic acid methyl ester

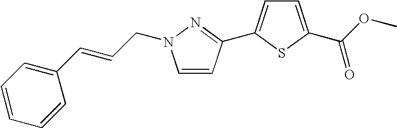

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and cinnamyl bromide (89 mg, 0.45 mmol), there was prepared 5-[1-(3-phenyl-allyl)-1H-pyrazol-3-yl]-thiophene-2-carboxylic acid methyl ester. LCMS (Method C): R_{T}=3.67 minutes; 325 (+H)⁺.

### (d) 5-[1-(3-Phenoxy-propyl)-1H-pyrazol-3-yl]-thiophene-2-carboxylic acid methyl ester

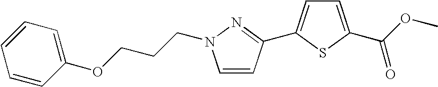

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and 3-phenoxypropyl bromide (97 mg, 0.45 mmol), there was prepared 5-[1-(3-phenoxy-propyl)-1H-pyrazol-3-yl]-thiophene-2-carboxylic acid methyl ester. LCMS (Method C): R_{T}=3.94 minutes; 375 (M+Na)⁺.

### (e) 5-[1-(2-Benzoylamino-ethyl)-1H-pyrazol-3-yl]-thiophene-2-carboxylic acid methyl ester

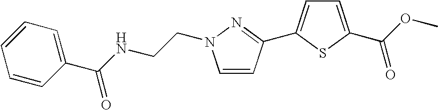

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and N-(2-chloroethyl)benzamide (82 mg, 0.45 mmol), there was prepared 5-[1-(2-benzoylamino-ethyl)-1H-pyrazol-3-yl]-thiophene-2-carboxylic acid methyl ester. LCMS (Method C): R_{T}=2.93 minutes; 356 (M+H)⁺.

### (f) 5-(1-Pyridin-4-ylmethyl-1H-pyrazol-3-yl)-thiophene-2-carboxylic acid methyl ester

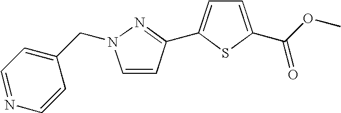

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and 4-picolyl chloride hydrochloride (74 mg, 0.45 mmol), there was prepared 5-(1-pyridin-4-ylmethyl-1H-pyrazol-3-yl)-thiophene-2-carboxylic acid methyl ester. LCMS (Method C): R_{T}=2.16 minutes; 300 (M+H)⁺.

### (g) 5-[1-(5-tert-Butyl-[1,2,4]oxadiazol-3-ylmethyl)-1H-pyrazol-3-yl-thiophene-2-carboxylic acid methyl ester

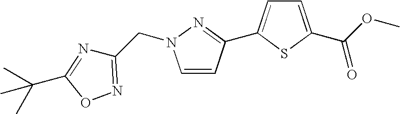

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and (5-tert-butyl)-3-(chloromethyl)-1,2,4-oxadiazole (78 mg, 0.45 mmol), there was prepared 5-[1-(5-tert-butyl-[1,2,4]oxadiazol-3-ylmethyl)-1H-pyrazol-3-yl]-thiophene-2-carboxylic acid methyl ester. LCMS (Method C): R_{T}=3.40 minutes; 347 (M+H)⁺.

### (h) 5-[1-(3-Pyrrol-1-yl-propyl)-1H-pyrazol-3-yl]-thiophene-2-carboxylic acid methyl ester

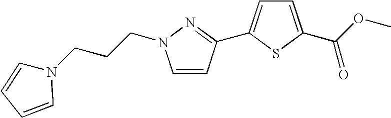

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and 1-(3-bromopropyl)pyrrole (85 mg, 0.45 mmol), there was prepared 5-[1-(3-pyrrol-1-yl-propyl-1H-pyrazol-3-yl]-thiophene-2-carboxylic acid methyl ester. LCMS (Method C): R_{T}=3.42 minutes; 316 (+H)⁺.

### (i) 5-(1-But-2-enyl-1H-pyrazol-3-yl)-thiophene-2-carboxylic acid methyl ester

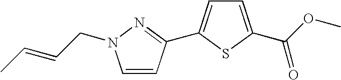

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and crotyl bromide (61 mg, 0.45 mmol), there was prepared 5-(1-but-2-enyl-1H-pyrazol-3-yl)-thiophene-2-carboxylic acid methyl ester. LCMS (Method C): R_{T}=3.30 minutes; 263 (M+H)⁺.

### (j) 5-(1-Quinolin-2-ylmethyl-1H-pyrazol-3-yl)-thiophene-2-carboxylic acid methyl ester

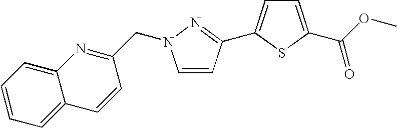

To a mixture of 5-(1H-pyrazol-3-yl)-thiophene-2-carboxylic acid methyl ester [400 mg, 1.9 mmol, Reference Example 12(a)], potassium carbonate (1.2 g, 8.7 mmol) and N,N-dimethylformamide (10 ml) was added 2-(chloromethyl)quinoline hydrochloride (400 mg, 2.0 mmol). The resulting mixture was heated to 80° C., stirred for 24 hours and left to stand over the weekend. The reaction mixture was concentrated, then partitioned between ethyl acetate and water, and the organic layer was isolated and evaporated to dryness to give a solid. The solid was redissolved in ethyl acetate, to which pentane and methanol were added, and the solution was allowed to stand overnight. The crystals that formed were filtered and dried under suction, to provide 5-(1-quinolin-2-yl]methyl-1H-pyrazol-3-yl)-thiophene-2-carboxylic acid methyl ester (345 mg) as a pale yellow solid. LCMS (Method C): R_{T}=3.49 minutes; 350 (M+H)⁺.

### (k) 5-(1-Phenylcarbamoylmethyl-1H-pyrazol-3-yl)-thiophene-2-carboxylic acid methyl ester

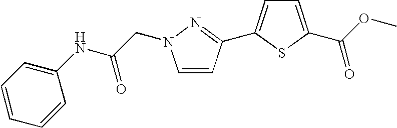

To a mixture of 5-(1H-pyrazol-3-yl)-thiophene-2-carboxylic acid methyl ester [200 mg, 0.96 mmol, Reference Example 12(a)], potassium carbonate (662 mg, 4.8 mmol) and N)N-dimethylformamide (3 ml) was added 2-chloro-N-phenylacetamide (148 mg, 0.88 mmol). The resulting mixture was heated to 80° C., and stirred overnight. The reaction mixture was concentrated, diluted with water, and extracted with ethyl acetate (3×). The organic layers were combined, dried (MgSO₄) and concentrated, to provide 5-(1-phenylcarbamoylmethyl-1H-pyrazol-3-yl)-thiophene-2-carboxylic acid methyl ester (325 mg).

### (l) 5-{1-[(5-Trifluoromethyl-[1,3,4]thiadiazol-2-ylcarbamoyl)-methyl]-1H-pyrazol-3-yl}-thiophene-2-carboxylic acid methyl ester

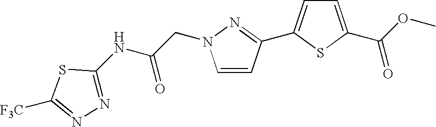

To a mixture of 5-(1H-pyrazol-3-yl)-thiophene-2-carboxylic acid methyl ester [100 mg, 0.48 mmol, Reference Example 12(a)], potassium carbonate (300 mg, 2.18 mmol) and N,N-dimethylformamide (2 ml) was added 2-chloro-N-[5-(trifluoromethyl)-1,3,4-thiadiazol-2-yl]acetamide (118 mg, 0.48 mmol). The resulting mixture was heated to 80° C. and stirred overnight. Water (4 ml) was added to the reaction mixture, which was allowed to stir for a further 30 minutes, before being extracted with ethyl acetate (2×). The combined organic layers were evaporated to dryness, to provide 5-{1-[(5-trifluoromethyl-[1,3,4]thiadiazol-2-ylcarbamoyl)-methyl]-1H-pyrazol-3-yl}-thiophene-2-carboxylic acid methyl ester. LCMS (Method C): R_{T}=2.99 minutes; 418 (M+H)⁺.

### (m) 5-{1-[(2-Methoxy-phenylcarbamoyl)-methyl]-1H-pyrazol-3-yl}-thiophene-2-carboxylic acid methyl ester

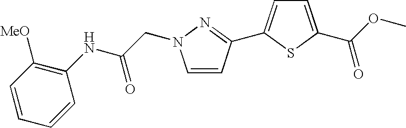

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-(2-methoxyphenyl)acetamide (96 mg, 0.48 mmol), there was prepared 5-{1-[(2-methoxy-phenylcarbamoyl)-methyl]-1H-pyrazol-3-yl}-thiophene-2-carboxylic acid methyl ester. LCMS (Method C): R_{T}=3.13 minutes; 372 (M+H)⁺.

### (n) 5-{1-[(4-Fluoro-phenylcarbamoyl)-methyl]-1H-pyrazol-3-yl}-thiophene-2-carboxylic acid methyl ester

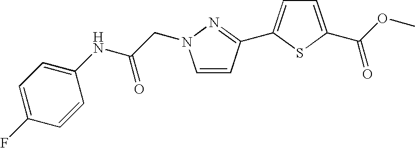

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-(4-fluorophenyl)acetamide (90 mg, 0.48 mmol), there was prepared 5-{1-[(4-fluoro-phenylcarbamoyl)-methyl]-1H-pyrazol-3-yl}-thiophene-2-carboxylic acid methyl ester. LCMS (Method C): R_{T}=2.99 minutes; 360 (M+H)⁺.

### (o) 5-{1-[(3-Fluoro-phenylcarbamoyl)-methyl]-1H-pyrazol-3-yl}-thiophene-2-carboxylic acid methyl ester

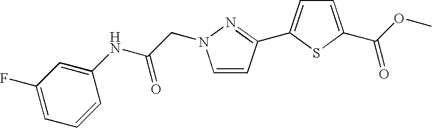

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-(3-fluorophenyl)acetamide (90 mg, 0.48 mmol), there was prepared 5-{1-[(3-fluoro-phenylcarbamoyl)-methyl]-1H-pyrazol-3-yl}-thiophene-2-carboxylic acid methyl ester. LCMS (Method C): R_{T}=3.06 minutes; 360 (M+H)⁺.

### (p) 5-(1-{2-[(Quinoline-2-carbonyl)-amino]-ethyl}-1H-pyrazol-3-yl)-thiophene-2-carboxylic acid methyl ester

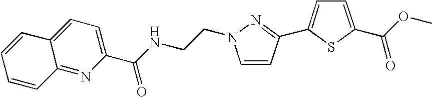

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and quinoline-2-carboxylic acid (2-chloroethyl)amide (112 mg, 0.48 mmol), there was prepared 5-(1-{2-[(quinoline-2-carbonyl)-amino]-ethyl})-1H-pyrazol-3-yl-thiophene-2-carboxylic acid methyl ester.

### (q) 5-[1-(Benzylcarbamoyl-methyl)-1H-pyrazol-3-yl]-thiophene-2-carboxylic acid methyl ester

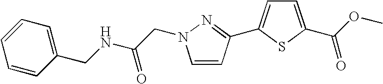

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and N-benzyl-2-chloroacetamide (88 mg, 0.48 mmol), there was prepared 5-[1-(benzylcarbamoyl-methyl)-1H-pyrazol-3-yl]-thiophene-2-carboxylic acid methyl ester.

### (r) 5-{1-[(N-Ethyl-N-phenyl-carbamoyl)-methyl]-1H-pyrazol-3-yl}-thiophene-2-carboxylic acid methyl ester

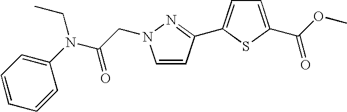

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-ethyl-N-phenylacetamide (95 mg, 0.48 mmol), there was prepared 5-{1-[(N-ethyl-N-phenyl-carbamoyl)-methyl]-1H-pyrazol-3-yl}-thiophene-2-carboxylic acid methyl ester.

### (s) 5-{1-[2-(1H-Indol-3-yl)ethyl]-1H-pyrazol-3-yl}-thiophene-2-carboxylic acid methyl ester

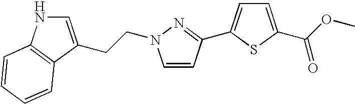

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 3-(2-bromoethyl)indole (108 mg, 0.48 mmol), there was prepared 5-{1-[2-(1H-indol-3-yl)-ethyl]-1H-pyrazol-3-yl}-thiophene-2-carboxylic acid methyl ester. LCMS (Method C): R_{T}=3.33 minutes; 352 (M+H)⁺.

### (t) 5-{1-[(2-Trifluoromethoxy-phenylcarbamoyl)-methyl]-1H-pyrazol-3-yl}-thiophene-2-carboxylic acid methyl ester

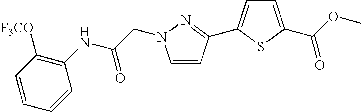

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-(2-trifluoromethoxyphenyl)acetamide (121 mg, 0.48 mmol), there was prepared 5-{1-[(2-trifluoromethoxy-phenylcarbamoyl)-methyl]-1H-pyrazol-3-yl}-thiophene-2-carboxylic acid methyl ester. LCMS (Method C): R_{T}=3.30 minutes; 426 (M+H)⁺.

### (u) 5-{1-[3-(4-Chloro-phenyl)-pronyl]-1H-pyrazol-3-yl}-thiophene-2-carboxylic acid methyl ester

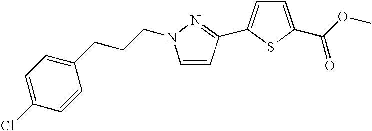

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 1-chloro-3-(4-chlorophenyl)propane (91 mg, 0.48 mmol), there was prepared 5-{1-[3-(4-chloro-phenyl)-propyl]-1H-pyrazol-3-yl}-thiophene-2-carboxylic acid methyl ester. LCMS (Method C): R_{T}=3.81 minutes; 361 & 363 (M+H)⁺.

### (v) 5-(1-{[2-(1H-Indol-3-yl)-ethylcarbamoyl]-methyl}-1H-pyrazol-3-yl)-thiophene-2-carboxylic acid methyl ester

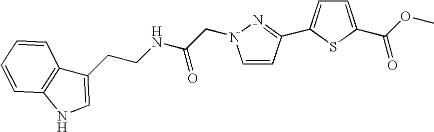

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 3-(chloroacetamidoethyl)indole (113 mg, 0.48 mmol), there was prepared 5-(1-{[2-(1H-indol-3-yl)-ethylcarbamoyl]-methyl}-1H-pyrazol-3-yl)-thiophene-2-carboxylic acid methyl ester. LCMS (Method C): R_{T}=2.95 minutes; 409 (M+H)⁺.

### (w) 5-[1-(Phenethylcarbamoyl-methyl)-1H-pyrazol-3-yl]-thiophene-2-carboxylic acid methyl ester

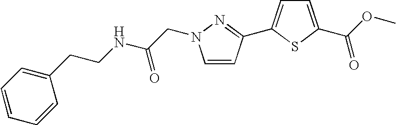

By proceeding in a similar manner to Reference Example 10(1) but using 5-(1H-pyrazol-3-yl)-thiophene-2-carboxylic acid methyl ester [100 mg, 0.48 mmol, Reference Example 12(a)] and 2-chloro-N-phenethylacetamide (95 mg, 0.48 mmol), there was prepared 5-[1-(phenethylcarbamoyl-methyl)-1H-pyrazol-3-yl]-thiophene-2-carboxylic acid methyl ester. LCMS (Method C): R_{T}=2.96 minutes; 370 (+H)⁺.

### (x) 5-(1-Isoquinolin-1-ylmethyl-1H-pyrazol-3-yl)-thiophene-2-carboxylic acid methyl ester

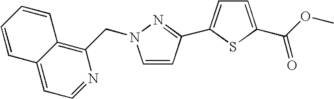

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 1-(bromomethyl)isoquinoline hydrobromide (145 mg, 0.48 mmol), there was prepared 5-(1-isoquinolin-1-ylmethyl-1H-pyrazol-3-yl)-thiophene-2-carboxylic acid methyl ester. LCMS (Method C): R_{T}=3.06 minutes; 350 (M+H)⁺.

### (y) 5-{1-[(2-Fluoro-phenylcarbamoyl)-methyl]-1H-pyrazol-3-yl}-thiophene-2-carboxylic acid methyl ester

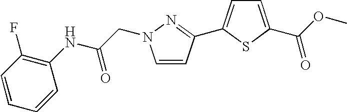

By proceeding in a similar manner to Reference Example 10(1) but using 5-(1H-pyrazol-3-yl)-thiophene-2-carboxylic acid methyl ester [100 mg, 0.48 mmol, Reference Example 12(a)] and 2-chloro-N-(2-fluorophenyl)acetamide (90 mg, 0.48 mmol), there was prepared 5-{1-[(2-fluoro-phenylcarbamoyl)-methyl]-1H-pyrazol-3-yl}-thiophene-2-carboxylic acid methyl ester. LCMS (Method C): R_{T}=3.02 minutes; 360 (M+H)⁺.

### (z) 5-[1-(2-Quinolin-2-yl-ethyl)-1H-pyrazol-3-yl]-thiophene-2-carboxylic acid methyl ester

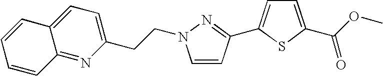

To a mixture of 5-(1H-pyrazol-3-yl)-thiophene-2-carboxylic acid methyl ester [235 mg, 1.30 mmol, Reference Example 12(a)], potassium carbonate (630 mg, 4.50 mmol) and N,N-dimethylformamide (10 ml) was added methanesulfonic acid 2-quinolin-2-yl-ethyl ester (287 mg, 1.14 mmol). The resulting mixture was heated to 80° C. and stirred overnight. The reaction mixture was then concentrated, and the residue was subjected to flash column chromatography using a mixture of ethyl acetate and cyclohexane (gradient 35:65 to 45:55, v/v, over 30 mintues) as eluent, to provide 5-[1-(2-quinolin-2-yl-ethyl)-1H-pyrazol-3-yl]-thiophene-2-carboxylic acid methyl ester. LCMS (Method C): R_{T}=2.64 minutes; 364 (M+H)⁺.

### (aa) 5-[1-(2-Hydroxy-ethyl)-H-pyrazol-3-yl]-thiophene-2-carboxylic acid methyl ester

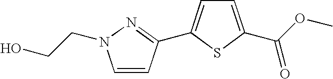

To a mixture of 5-(1H-pyrazol-3-yl)-thiophene-2-carboxylic acid methyl ester [1,0 g, 5.5 mmol, Reference Example 12(a)], potassium carbonate (2 g, 14.4 mmol) and N,N-dimethylformamide (30 ml) was added 2-bromoethanol (0.5 ml, 0.6 mmol). The resulting mixture was heated to 70° C. and stirred for 16 hours. The reaction mixture was then concentrated, and the residue was partitioned between ethyl acetate and water. The organic layer was separated, dried (MgSO₄) and concentrated, to provide 5-[1-(2-hydroxy-ethyl) -1H-pyrazol-3-yl]-thiophene-2-carboxylic acid methyl ester (1.94 g) as a yellow solid. LCMS (Method C): R_{T}=2.65 minutes; 253 (M+H)⁺.

### (ab) 5-(1-tert-Butoxycarbonylmethyl-1H-pyrazol-3-yl)-thiophene-2-carboxylic acid methyl ester

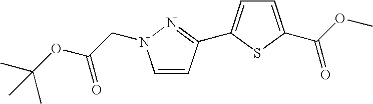

To a mixture of 5-(1H-pyrazol-3-yl)-thiophene-2-carboxylic acid methyl ester [3.96 g, 19 mmol, Reference Example 12(a)], potassium carbonate (7.88 g, 57 mmol) and N,N-dimethylformamide (100 ml) was added tert-butyl bromoacetate (3.06 μl, 21 mmol). The resulting mixture was heated to 80° C. and stirred overnight. The reaction mixture was then concentrated, and the residue was partitioned between ethyl acetate and water. The organic layer was separated, dried (MgSO₄) and concentrated, to provide 5-(1-tert-butoxycarbonylmethyl-1H-pyrazol-3-yl)-thiophene-2-carboxylic acid methyl ester (5.94 g) as a solid.

### (ac) 5-(1-Biphenyl-4-ylmethyl-1H-pyrazol-3-yl)-thiophene-2-carboxylic acid methyl ester

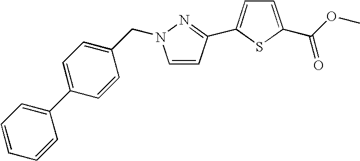

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and 4-phenylbenzyl chloride (91 mg, 0.45 mmol), there was prepared 5-(1-biphenyl-4-ylmethyl-1H-pyrazol-3-yl)-thiophene-2-carboxylic acid methyl ester. LCMS (Method C): R_{T}=3.94 minutes; 375 (M+H)⁺.

### (ad) 5-{1-[6-(2,2-Dimethyl-propionylamino)-pyridin-2-ylmethyl]-1H-pyrazol-3-yl}-thiophene-2-carboxylic acid methyl ester

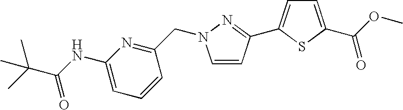

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and N-(6-bromomethyl-pyridin-2-yl)-2,2-dimethyl-propionamide (121 mg, 0.45 mmol), there was prepared 5-{1-[6-(2,2-dimethyl-propionylamino)-pyridin-2-ylmethyl]-1H-pyrazol-3-yl}-thiophene-2-carboxylic acid methyl ester. LCMS (Method C): R_{T}=3.33 minutes; 399 (M+H)⁺.

### (ae) 5-{1-[2-(Biphenyl-4-yloxy)-ethyl]-H-pyrazol-3-yl}-thiophene-2-carboxylic acid methyl ester

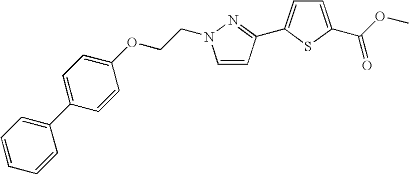

By proceeding in a similar manner to Reference Example 10(b) but using 5-(1H-pyrazol-3-yl)-thiophene-2-carboxylic acid methyl ester [65 mg, 0.31 mmol, Reference Example 12(a)] and 4-(2-bromoethoxy)-1,1′-biphenyl (124 mg, 0.45 mmol), there was prepared 5-{1-[2-(biphenyl-4-yloxy)-ethyl]-1H-pyrazol-3-yl}-thiophene-2-carboxylic acid methyl ester. LCMS (Method C): R_{T}=3.96 minutes; 405 (M+H)⁺.

### (ag) 5-[1-(3-Phenoxy-benzyl)-1H-pyrazol-3-yl)-thiophene-2-carboxylic acid methyl ester

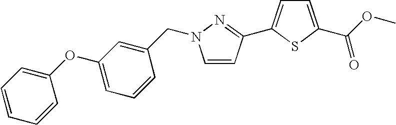

By proceeding in a similar manner to Reference Example 10(b) but using 5-(1H-pyrazol-3-yl)-thiophene-2-carboxylic acid methyl ester [65 mg, 0.3 mmol, Reference Example 12(a)] and 3-phenoxybenzyl chloride (98 mg, 0.45 mmol), there was prepared 5-[1-(3-phenoxy-benzyl)-1H-pyrazol-3-yl]-thiophene-2-carboxylic acid methyl ester. LCMS (Method C): R_{T}=3.87 minutes; 391 (M+H)⁺.

### (ah) 5-(1-{3-[4-(3-Chloro-phenyl)-piperazin-1-yl]-propyl}-1H-pyrazol-3-yl)-thiophene-2-carboxylic acid methyl ester

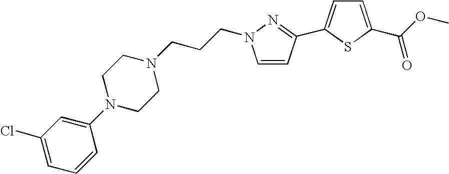

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 1-(3-chlorophenyl)-4-(3-chloropropyl) piperazine (131 mg, 0.48 mmol), there was prepared 5-(1-{3-[4-(3-chloro-phenyl)-piperazin-1-yl]-propyl}-1H-pyrazol-3-all-thiophene-2-carboxylic acid methyl ester. LCMS (Method C): R_{T}=2.40 minutes; 445 (M+H)⁺.

### (ai) 5-{1-[(4-Morpholin-4-yl-phenylcarbamoyl)-methyl]-1H-pyrazol-3-yl}-thiophene-2-carboxylic acid methyl ester

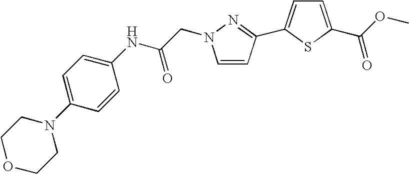

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-(4-morpholinophenyl)acetamide (122 mg, 0.48 mmol), there was prepared 5-{1-[(4-morpholin-4-yl-phenylcarbamoyl)-methyl]-1H-pyrazol-3-yl}-thiophene-2-carboxylic acid methyl ester. LCMS (Method C): R_{T}=2.70 minutes; 427 (N+H)⁺.

### (aj) 5-{1-[(2-Morpholin-4-yl-phenylcarbamoyl)-methyl]-1H-pyrazol-3-yl}-thiophene-2-carboxylic acid methyl ester

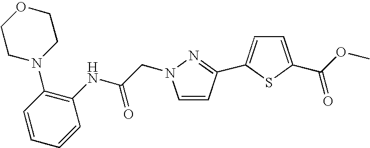

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-N-(2-morpholinophenyl)acetamide (122 mg, 0.48 mmol), there was prepared 5-{1-[(2-morpholin-4-yl-phenylcarbamoyl)-methyl]-1H-pyrazol-3-yl}-thiophene-2-carboxylic acid methyl ester. LCMS Method C): R_{T}=3.07 minutes; 427 (M+H)⁺.

### (ak) 5-{1-[(4-Oxazol-5-yl-phenylcarbamoyl)-methyl]-1H-pyrazol-3-yl}-thiophene-2-carboxylic acid methyl ester

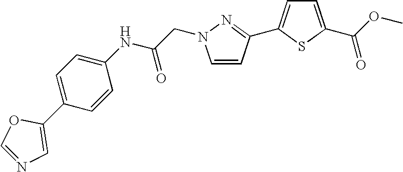

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and N-1-[4-(1,3-oxazol-5-yl)phenyl]-2-chloroacetamide (113 mg, 0.48 mmol), there was prepared 5-{1-[(4-oxazol-5-yl-phenylcarbamoyl)-methyl]-1H-pyrazol-3-yl}-thiophene-2-carboxylic acid methyl ester. LCMS (Method C): R_{T}=2.88 minutes; 409 (M+H)⁺.

### (al) 5-{1-[(L-Acetylamino-phenylcarbamoyl)-methyl]-1H-pyrazol-3-yl}-thiophene-2-carboxylic acid methyl ester

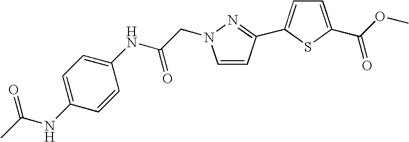

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and N-(4-acetylaminophenyl)-2-chloroacetamide (108 mg, 0.48 mmol), there was prepared 5-{1-[(4-acetylamino-phenylcarbamoyl)-methyl]-1H-pyrazol-3-yl}-thiophene-2-carboxylic acid methyl ester. LCMS (Method C): R_{T}=2.56 minutes; 399 (M+H)⁺.

### (ao) 5-[1-(1-Oxy-quinolin-2-ylmethyl)-1H-pyrazol-3-yl]-thiophene-2-carboxylic acid methyl ester

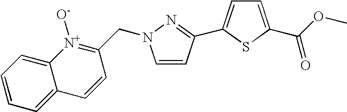

By proceeding in a similar manner to Reference Example 10(l) but using 5-(1H-pyrazol-3-yl)-thiophene-2-carboxylic acid methyl ester [511 mg, 2.46 mmol, Reference Example 12(a)] and 2-chloromethyl-quinoline 1-oxide (479 mg, 2.46 mmol), there was prepared 5-[1-(1-oxy-quinolin-2-ylmethyl)-H-pyrazol-3-yl]-thiophene-2-carboxylic acid methyl ester. LCMS (Method C): R_{T}=3.08 minutes; 366 (M+H)⁺.

### (ap) 5-(1-{2-Oxo-2-[4-(4-trifluoromethyl-pyrimidin-2-yl)-piperazin-1-yl)-ethyl}-1H-pyrazol-3-yl)-thiophene-2-carboxylic acid methyl ester

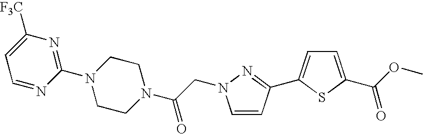

By proceeding in a similar manner to Reference Example 10(l) but using 5-(1H-pyrazol-3-yl)-thiophene-2-carboxylic acid methyl ester [100 mg, 0.48 mmol, Reference Example 12(a)] and 2-chloro-1-[4-[4-(trifluoromethyl)pyrimidin-2-yl]piperazino]ethan-1-one (148 mg, 0.48 mmol), there was prepared 5-(1-{2-oxo-2-[4-(4-trifluoromethyl-pyrimidin-2-yl)-piperazin-1-yl]-ethyl}-1H-pyrazol-3-yl)-thiophene-2-carboxylic acid methyl ester. LCMS (Method C): R_{T}=3.26 minutes; 481 (M+H)⁺.

### (aq) 5-[1-(2-tert-Butoxycarbonylamino-ethyl)-1H-pyrazol-3-yl]-thiophene-2-carboxylic acid methyl ester

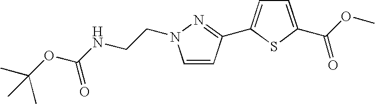

By proceeding in a similar manner to Reference Example 10(l) but using 5-(1H-pyrazol-3-yl)-thiophene-2-carboxylic acid methyl ester [1.17 g, 5.0 mmol, Reference Example 12(a)] and 2-(tert-butoxycarbonylamino)ethyl bromide (1.26 g, 5.5 mmol), there was prepared 5-[1-(2-tert-butoxycarbonylamino-ethyl)-1H-pyrazol-3-yl]-thiophene-2-carboxylic acid methyl ester (1.44 g) as a pale yellow solid. LCMS (Method C): R_{T}=3.33 minutes; 352 (M+H)⁺.

### (ar) 5-[1-(3-tert-Butoxycarbonylamino-propyl)-1H-pyrazol-3-yl]-thiophene-2-carboxylic acid methyl ester

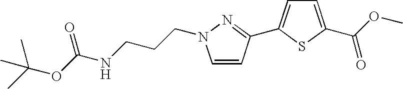

By proceeding in a similar manner to Reference Example 10(1) but using 5-(1H-pyrazol-3-yl)-thiophene-2-carboxylic acid methyl ester [1.87 g, 9.0 mmol, Reference Example 12(a)] and 3-(tert-butoxycarbonylamino)propyl bromide (2.5 g, 9.9 mmol), there was prepared 5-[1-(3-tert-butoxycarbonylamino-propyl)-1H-pyrazol-3-yl]-thiophene-2-carboxylic acid methyl ester (2.98 g) as a yellow oil. LCMS (Method C): R_{T}=3.48 minutes; 366 (M+H)⁺.

### Reference Example 11

### (a) 5-[5-(3-Phenyl-propionylamino)-pyridin-2-yl]-thiophene-2-carboxylic acid methyl ester

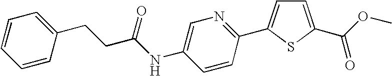

To a solution of 5-(5-amino-pyridin-2-yl)-thiophene-2-carboxylic acid methyl ester [50 mg, 0.2 mmol, Reference Example 15(a)] in acetonitrile (3 ml), was added hydrocinnamoyl chloride (34 μl, 0.24 mmol) followed by diisopropylethylamine (50 μl, 0.3 mmol). The mixture was stirred at room temperature for 1 hour, then saturated citric acid solution was added and the resulting mixture was extracted with chloroform. The organic phase was dried (MgSO₄), and evaporated under reduced pressure, to provide 5-[5-(3-phenyl-propionylamino)-pyridin-2-yl]-thiophene-2-carboxylic acid methyl ester (69 mg) as an off-white solid. LCMS (Method C): R_{T}=3.67 minutes; 367 (M+H)⁺.

### (b) 5-[5-(2-Phenoxy-acetylamino)-pyridin-2-yl]-thiophene-2-carboxylic acid methyl ester

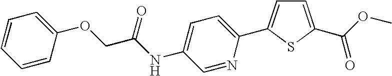

By proceeding in a similar manner to Reference Example 11(a) but using 5-(5-amino-pyridin-2-yl)-thiophene-2-carboxylic acid methyl ester [55 mg, 0.23 mmol, Reference Example 15(a)] and phenoxyacetyl chloride (35 μl, 0.25 mmol), there was prepared 5-[5-(2-phenoxy-acetylamino)-pyridin-2-yl]-thiophene-2-carboxylic acid methyl ester as an off-white powder. LCMS (Method C): R_{T}=3.67 minutes; 369 (M+H)⁺.

### (c) 5-(5-Phenylacetylamino-pyridin-2-yl)-thiophene-2-carboxylic acid methyl ester

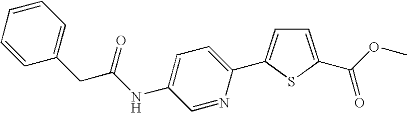

By proceeding in a similar manner to Reference Example 11(a) but using 5-(5-amino-pyridin-2-yl)-thiophene-2-carboxylic acid methyl ester [55 mg, 0.23 mmol, Reference Example 15(a)] and phenylacetyl chloride (34 μl, 0.25 mmol), there was prepared 5-(5phenylacetylamino-pyridin-2-yl)-thiophene-2-carboxylic acid methyl ester as an off-white powder. LCMS (Method C): R_{T}=3.52 minutes; 353 (+H)⁺.

### (d) 5-(5-Benzoylamino-pyridin-2-yl)-thiophene-2-carboxylic acid methyl ester

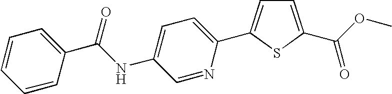

By proceeding in a similar manner to Reference Example 11(a) but using 5-(5-amino-pyridin-2-yl)-thiophene-2-carboxylic acid methyl ester [55 mg, 0.23 mmol, Reference Example 15(a)] and benzoyl chloride (29 μl, 0.25 mmol), there was prepared 5-(5-benzoylamino-pyridin-2-yl)-thiophene-2-carboxylic acid methyl ester as an off-white powder. LCMS (Method C): R_{T}=3.56 minutes; 339 (M+1)⁺.

### (e) 5-{5-[(Pyridine-4-carbonyl)-amino]-pyridin-2-yl}-thiophene-2-carboxylic acid methyl ester

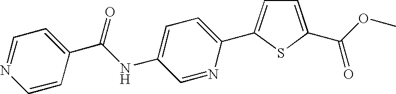

By proceeding in a similar manner to Reference Example 11(a) but using 5-(5-amino-pyridin-2-yl)-thiophene-2-carboxylic acid methyl ester [55 mg, 0.23 mmol, Reference Example 15(a)] and iso-nicotinoyl chloride hydrochloride (47 mg, 0.25 mmol), there was prepared 5-{5-[(pyridine-4-carbonyl)-amino]-pyridin-2-yl}-thiophene-2-carboxylic acid methyl ester as an off-white powder. LCMS (Method C): R_{T}=2.78 minutes; 340 (M+H)⁺.

### (f) 5-[6-(3-Phenyl-propionylamino)-pyridin-2-yl]-thiophene-2-carboxylic acid methyl ester

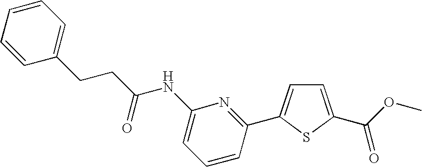

5-(6-amino-pyridin-2-yl)-thiophene-2-carboxylic acid methyl ester [258 mg 1.1 mmol, Reference Example 12(b)] and hydrocinnamoyl chloride (222 mg, 1.3 mmol) were heated to 160° C. for 2.5 hours. Saturated sodium carbonate solution was added to the reaction mixture, which was then extracted with dichloromethane (3×). The organic layers were combined, dried (Na₂SO₄) and concentrated, to provide 5-[6-(3-phenyl-propionylamino)-pyridin-2-yl]-thiophene-2-carboxylic acid methyl ester (330 mg). LCMS (Method C): R_{T} 3.99 minutes; 367 (M+H)⁺.

### Reference Example 12

### (a) 5-(1H-Pyrazol-3-yl)-thiophene-2-carboxylic acid methyl ester

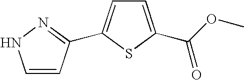

A suspension of 5-(1H-pyrazol-3-yl)-thiophene-2-carboxylic acid [1.09 g, 9.0 mmol, Reference Example 2(c)] in methanol (30 ml) and concentrated hydrochloric acid (1.32 ml), was heated to reflux overnight. The reaction mixture was concentrated to give a residue, which was partitioned between saturated aqueous sodium hydrogen carbonate solution and dichloromethane. The organic phase was separated and concentrated, to provide 5-(1H-pyrazol-3-yl)-thiophene-2-carboxylic acid methyl ester (1.04 g) as a beige solid, which was used directly without further purification.

### (b) 5-(6-Amino-pyridin-2-yl)-thiophene-2-carboxylic acid methyl ester

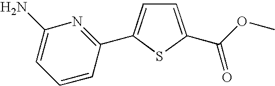

To a stirred suspension of 5-(6-amino-pyridin-2-yl)-thiophene-2-carboxylic acid [1.5 g, 6.8 mmol, Reference Example 14(f)] in toluene (57 ml) and methanol (12 ml), was added trimethysilyldiazomethane (6.8 ml, 13.6 mmol). The reaction was stirred over the weekend, and then concentrated, to provide 5-(6-amino-pyridin-2-yl)-thiophene-2-carboxylic acid methyl ester (1.35 g).

### Reference Example 13

### (a) 5-(4-Benzyloxy-pyrimidin-2-yl)-thiophene-2-carboxylic acid

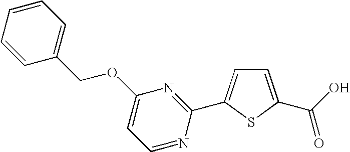

To a cold (−78° C.) solution of 4-benzyloxy-2-(5-bromo-thiophen-2-yl)-pyrimidine [226 mg, 0.65 mmol, Reference Example 22(a)] in tetrahydrofuran (20 ml) under a nitrogen atmosphere was added n-butyl lithium (390 μl, 0.98 mmol, 2.5M in hexanes). The reaction mixture was stirred for 50 minutes, then poured onto solid carbon dioxide pellets and vigorously stirred until the slurry had reached room temperature. The slurry was carefully acidified with concentrated hydrochloric acid, and then extracted with dichloromethane. The organic phase was separated and dried (MgSO₄), then concentrated in vacuo to provide 5-(4-benzyloxy-pyrimidin-2-yl)-thiophene-2-carboxylic acid (118 mg) as an off-white soild. LCMS (Method C): R_{T}=3.54 minutes; 313 (M+H)⁺.

### (b) 5-(5-Phenethyl-1H-pyrazol-3-yl)-thiophene-2-carboxylic acid

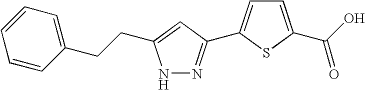

To a cold (−78° C.) solution of 5-phenethyl-3-thiophen-2-yl-1H-pyrazole [2,0 g, 7.87 mmol, Reference Example 17(a)] in tetrahydrofuran (100 ml) under a nitrogen atmosphere was added n-butyl lithium (6.9 ml, 17.32 mmol, 2.5M in hexanes). The reaction mixture was stirred for 2 hours, and then carbon dioxide (100 ml of carbon dioxide pellets were placed in a separate flask, and a purge line was attached to the reaction mixture) was bubbled through the solution. The reaction mixture was then allowed to warm to room temperature, concentrated and treated with 1M sodium hydroxide solution. The resulting solution was extracted with ethyl acetate, then acidified and extracted again with ethyl acetate. The organic phases were combined and washed with brine, dried (MgSO₄) and concentrated in vacuo to provide 5-(5-phenethyl-1H-pyrazol-3-yl]-thiophene-2-carboxylic acid (1.6 g), which was used directly without further purification.

### (c) 5-(5-Trifluoromethyl-1H-[1,2,4]triazol-3-yl)-thiophene-2-carboxylic acid

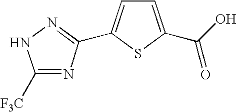

To a cold (−78° C.) solution of 3-thiophen-2-yl-5-trifluoromethyl-1H-[1,2,4]triazole [262 g, 1.2 mmol, Reference Example 19(a)]) in tetrahydrofuran (5 ml) under a nitrogen atmosphere was added n-butyl lithium (2.5 ml, 6.25 mmol, 2.5M in hexanes). The reaction mixture was stirred for 1 hour, and then carbon dioxide gas was bubbled through the solution for 1 hour. The reaction mixture was then allowed to warm to room temperature, water was added, and then the mixture was acidified and extracted with ethyl acetate (2×). The organic phases were combined, dried (MgSO₄), and concentrated to give a residue which was subjected to flash column chromatography on silica using a mixture of pentane and ethyl acetate (gradient 10:1 to 1:1, v/v) as eluent, to provide 5-(5-trifluoromethyl-1H-[1,2,4]triazol-3-yl)-thiophene-2-carboxylic acid (68 mg). LCMS (Method C): R_{T}=2.88 minutes; 363 (+H)⁺.

### (d) 4-Methyl-5-(5-trifluoromethyl-1H-pyrazol-3-yl)-thiophene-2-carboxylic acid

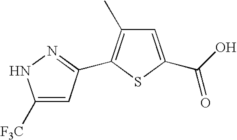

To a cold (−78° C.) solution of 3-(3-methyl-thiophen-2-yl)-5-trifluoromethyl-1H-pyrazole [500 mg, 2.16 mmol, Reference Example 17(b)] in tetrahydrofuran (25 ml) under a nitrogen atmosphere was added n-butyl lithium (1.9 ml, 4.74 mmol, 2.5M in hexanes). The reaction mixture was stirred for 2 hours, and then carbon dioxide gas was bubbled through the solution for a further 2 hours. After allowing to warm to room temperature the reaction mixture was stirred overnight, and then concentrated and treated with 1M sodium hydroxide solution. The resulting solution was extracted with ethyl acetate, then acidified and extracted again with ethyl acetate, the organic phases were combined and washed with brine, dried (Na₂SO₄) and concentrated in vacuo, to provide 4-methyl-5-(5-trifluoromethyl-1H-pyrazol-3-yl)-thiophene-2-carboxylic acid (518 g), which was used directly without further purification.

### Reference Example 14

### (a) 5-Pyrimidin-2-yl-thiophene-2-carboxylic acid

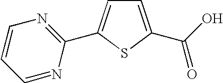

Acetonitrile (29 mL) and a solution of 0.4 M aqueous sodium carbonate (29 mL) were degassed (via nitrogen purge), then combined under a nitrogen atmosphere. 2-Bromopyrimidine (924 mg, 5.8 mmol) and 5-(dihydroxyboryl)-2-thiophenecarboxylic acid (1.0 g, 5.8 mmol) were added to the solution, which was heated to 80° C., followed by the addition of tetrakis(triphenylphosphine)palladium(0) (336 mg, 0.29 mmol). After stirring for 3 hours the reaction mixture was partitioned between ethyl acetate and saturated sodium hydrogen carbonate solution. The aqueous layer was isolated and acidified with concentrated hydrochloric acid to give a white paste which was filtered, washed with water and dried under vacuum, to provide 5-pyrimidin-2-yl-thiophene-2-carboxylic acid (1.28 g) as a white solid. LCMS (Method C): R_{T}=2.33 minutes; 207 (M+H)⁺.

### (b) 5-Pyridin-3-yl-thiophene-2-carboxylic acid

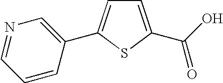

A mixture of N,N-dimethylformamide (7 ml), ethanol (2 ml) and water (3 ml) was added to 3-bromopyridine (398 mg, 2.52 mmol), 5-(dihydroxyboryl)-2-thiophenecarboxylic acid (518 mg, 2.7 mmol), cesium carbonate (1.64 g, 5.04 mmol) and tetrakis(triphenylphosphine)palladium(0) (216 mg, 0.188 mmol). The suspension was subjected to microwave irradiation, heating to 150° C. for 10 minutes, and was then partitioned between saturated sodium hydrogen carbonate solution and ethyl acetate. The aqueous layer was isolated and acidified with 1M hydrochloric acid then filtered, to provide 5-pyridin-3-yl-thiophene-2-carboxylic acid (140 mg) as an off-white powder. LCMS (Method C): R_{T}=1.76 minutes; 206 (M+H)⁺.

### (c) 5-Pyridin-4-yl-thiophene-2-carboxylic acid

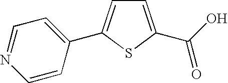

A mixture of N,N-dimethylformamide (7 ml), ethanol (2 ml) and water (3 ml) was added to 4-bromopyridine hydrogen chloride (490 mg, 2.52 mmol), 5-(dihydroxyboryl)-2-thiophenecarboxylic acid (518 mg, 2.7 mmol) cesium carbonate (2.46 g, 7.56 mmol) and tetrakis(triphenylphosphine)palladium(0) (216 mg, 0.188 mmol). The suspension was subjected to microwave irradiation, heating to 150° C. for 10 minutes, and then partitioned between saturated sodium hydrogen carbonate solution and ethyl acetate. The aqueous layer was isolated and acidified with 1M hydrochloric acid then filtered, to provide 5-pyridin-4-yl-thiophene-2-carboxylic acid (87 mg) as a brown powder. LCMS (Method C): R_{T}=0.32 minutes; 206 (M+H)⁺.

### (d) 5-(5-Nitro-pyridin-2-yl)-thiophene-2-carboxylic acid

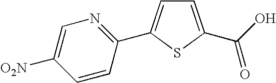

Acetonitrile (50 mL) and a solution of 0.4 M aqueous sodium carbonate solution (50 ml) were degassed (via nitrogen purge), then combined under a nitrogen atmosphere. 2-Bromo-5-nitropyridine (3.48 g, 17.0 mmol) and 5-(dihydroxyboryl)-2-thiophenecarboxylic acid (2.96 g, 17.0 mmol) were added to the solution, which was heated to 90° C., followed by the addition of tetrakis(triphenylphosphine)palladium(0) (0.98 g, 0.85 mmol). After stirring overnight the reaction mixture was partitioned between ethyl acetate and saturated sodium hydrogen carbonate solution. The aqueous layer was separated and acidified with concentrated hydrochloric acid to give a green precipitate, which was washed with water, dichloromethane, and chloroform to provide 5-(5-nitro-pyridin-2-yl)-thiophene-2-carboxylic acid (2.45 g). LCMS (Method C): R_{T}=2.97 minutes.

### (e) 5-(6-Formyl-pyridin-2-yl)-thiophene-2-carboxylic acid

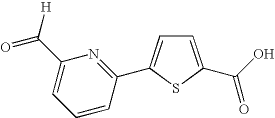

Acetonitrile (125 mL) and a solution of 0.4 M aqueous sodium carbonate (125 mL) were degassed (via nitrogen purge), then combined under a nitrogen atmosphere. 6-Bromopyridine-2-carboxaldehyde (5.6 g, 30 mmol) and 5-(dihydroxyboryl)-2-thiophenecarboxylic acid (4.3 g, 25 mmol) were added to the solution, which was heated to 80° C., followed by the addition of tetrakis(triphenylphosphine)palladium(0) (585 mg, 0.51 mmol). After stirring for 1 hour the reaction mixture was partitioned between ethyl acetate and saturated ammonium chloride solution. The aqueous layer was isolated and acidified with 1M hydrochloric acid to give a white solid which was collected by filtration, and dried under vacuum, to provide 5-(6-formyl-pyridin-2-yl)-thiophene-2-carboxylic acid (4.29 g) as a white solid. LCMS (Method C): R_{T}=2.75 minutes; 233 (M)⁺.

### (f) 5-(6-Amino-pyridin-2-yl)-thiophene-2-carboxylic acid

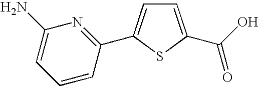

Acetonitrile (5 mL) and a solution of 0.4 M aqueous sodium carbonate (5 mL) were degassed (via nitrogen purge), then combined under a nitrogen atmosphere. 2-Amino-6-bromopyridine (173 mg, 1.0 mmol) and 5-(dihydroxyboryl)-2-thiophenecarboxylic acid (173 mg, 1.0 mmol) were added to the solution, which was heated to 80° C., followed by the addition of tetrakis(triphenylphosphine)palladium(0) (23 mg, 0.02 mmol). After stirring for 1 hour the reaction mixture was partitioned between ethyl acetate and saturated ammonium chloride solution. The aqueous layer was isolated and acidified with 1M hydrochloric acid to give a fine white solid which was collected by filtration, and dried under vacuum, to provide 5-(6-amino-pyridin-2-yl)-thiophene-2-carboxylic acid (90 mg) as a white solid. LCMS (Method C): R_{T}=1.53 minutes; 221 (M+H)⁺.

### (g) 5-(6-Bromo-pyridin-2-yl)-thiophene-2-carboxylic acid

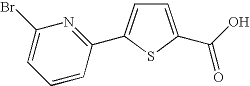

Acetonitrile (150 mL) and a solution of 0.4 M aqueous sodium carbonate (150 mL) were degassed (via nitrogen purge), then combined under a nitrogen atmosphere. 2,6-Dibromopyridine (14.2 g, 60 mmol) and 5-(dihydroxyboryl)-2-thiophenecarboxylic acid (5.16 g, 30 mmol) were added to the solution, which was heated to 80° C., followed by the addition of tetrakis(triphenylphosphine)palladium(0) (1.06 g, 0.92 mmol). After stirring for 3 hours the reaction mixture was partitioned between ethyl acetate and saturated sodium hydrogen carbonate solution. The aqueous layer was isolated and acidified with 1M hydrochloric acid to give a white solid which was collected by filtration, and dried under vacuum, to provide 5-(6-bromo-pyridin-2-yl)-thiophene-2-carboxylic acid (3.02 g) as a white solid.

### Reference Example 15

### (a) 5-(5-Amino-pyridin-2-yl)-thiophene-2-carboxylic acid methyl ester

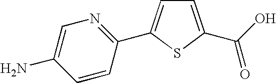

A suspension of 5-(5-nitro-pyridin-2-yl)-thiophene-2-carboxylic acid methyl ester [1.78 g, 6.7 mmol, Reference Example 20(a)], palladium (5 wt. % on activated carbon) (500 mg) and acetonitrile (300 ml) was stirred under a hydrogen atmosphere for 90 minutes. The mixture was then filtered through Hyflo, and the solvent was removed in vacuo, to provide 5-(5-amino-pyridin-2-yl)-thiophene-2-carboxylic acid methyl ester (1.40 g) as a yellow solid. LCMS (Method C): R_{T}=2.54 minutes; 235 (M+H)⁺.

### Reference Example 16

### (a) 4-Benzyloxy-2-(5-bromo-thiophen-2-yl)-pyrimidine

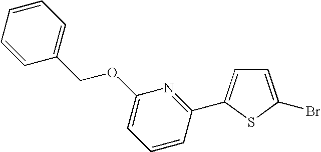

A mixture of 1,4-dioxane (4 ml), N-bromosuccinimide (551 mg, 3.1 mmol) and 4-benzyloxy-2-thiophen-2-yl-pyrimidine [265 mg, 0.99 mmol, Reference Example 22(a)] was subjected to microwave irradiation, heating to 100° C. for 20 minutes. The reaction mixture was then poured onto saturated sodium hydrogen carbonate solution and extracted with diethyl ether. The organic phase was washed with saturated aqueous sodium hydrogen carbonate solution, dried (MgSO₄), concentrated in vacuo, and then subjected to flash column chromatography using a mixture of cyclohexane and dichloromethane (gradient 100:0 to 0:100, v/v, over 20 minutes) as eluent, to provide 4-benzyloxy-2-(5-bromo-thiophen-2-yl)-pyrimidine (226 mg) as a gum. LCMS (Method C): R_{T}=4.70 minutes; 347 & 349 (M+H)⁺.

### Reference Example 17

### (a) 5-Phenethyl-3-thiophen-2-yl-1H-pyrazole

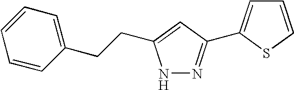

A solution of 5-phenyl-1-thiophen-2-yl-pentane-1,3-dione [4.37 g, 15.17 mmol, Reference Example 23(a)] in ethanol (50 ml) was treated with hydrazine hydrate (5 ml). The resulting solution was heated to reflux for 6 hours and then allowed to stand at room temperature for 2 days. The mixture was concentrated to give a residue, which was dissolved in ethyl acetate, and washed with 1M hydrochloric acid. The organic phase was separated, dried (Na₂SO₄), and concentrated to provide 5-phenethyl-3-thiophen-2-yl-1H-pyrazole (3.86 g) as a brown solid, which was used directly without further purification.

### (b) 3-(3-Methyl-thio hen-2-yl)-5-trifluoromethyl-1H-pyrazole

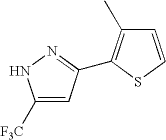

A solution of 1-(3-methyl-thiophen-2-yl)-butane-1,3-dione [3.72 g, 15.76 mmol, Reference Example 23(b)] in ethanol (45 ml) was treated with hydrazine hydrate (3.2 ml). The resulting solution was heated to reflux overnight, and then concentrated to give a residue. The residue was dissolved in ethyl acetate, washed with 1 M hydrochloric acid, followed by brine, dried (Na₂SO₄), and concentrated to provide 3-(3-methyl-thiophen-2-yl)-5-trifluoromethyl-1H-pyrazole as a yellow solid, which was used directly without further purification.

### Reference Example 18

### (a) 5-(2-Phenethyl-3H-imidazol-4-yl)-thiophene-2-carboxylic acid methyl ester

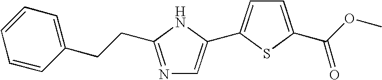

5-(2,2-Dibromo-acetyl)-thiophene-2-carboxylic acid methyl ester [0.68 g, 2.0 mmol, Reference Example 24(a)] was added to a solution of sodium acetate (0.28 g, 3.4 mmol) in water (10 ml), and the resulting mixture was stirred at 90° C. for 45 min, then allowed to cool to room temperature. Methanol (15 ml) was then added, followed by hydrocinnamaldehyde (0.24 g, 1.8 mmol) and concentrated ammonium hydroxide (15 ml). The mixture was then stirred at room temperature for 4 hours, and then partitioned between ethyl acetate and brine. The organic layer was separated and the aqueous phase was extracted with ethyl acetate (2×). The organic phases were combined, washed with brine, dried (Na₂SO₄), and concentrated to give a dark brown oil, which was subjected to flash column chromatography using a mixture of cyclohexane and ethyl acetate (1:1, v/v) as eluent, to provide 5-(2-phenethyl-3H-imidazol-4-yl)-thiophene-2-carboxylic acid methyl ester, (109 mg) as a brown oil.

### Reference Example 19

### (a) 3-Thiophen-2-yl-5-trifluoromethyl-1H-[1,2,4]triazole

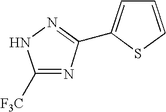

A solution of thiophene-2-carboxylic acid N′-(2,2,2-trifluoro-1-imino-ethyl)-hydrazide [261 mg, 1.1 mmol, Reference Example 25(a)] and N,N-dimethylformamide (3 ml) was subjected to microwave irradiation, heating to 220° C. for 15 minutes. The reaction mixture was concentrated to give a yellow gum, which was subjected to flash column chromatography using a mixture of pentane and ethyl acetate (gradient 100:0 to 50:50, v/v) as eluent, to provide 3-thiophen-2-yl-5-trifluoromethyl-1H-[1,2,4]triazole (516 mg) as a white powder.

### Reference Example 20

### (a) 5-(5-Nitro-pyridin-2-yl)-thiophene-2-carboxylic acid methyl ester

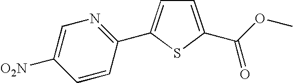

To a suspension of 5-(5-nitro-pyridin-2-yl)-thiophene-2-carboxylic acid [2.25 g, 9.0 mmol, Reference Example 14(d)] in methanol (50 ml) at 60° C., was added concentrated hydrochloric acid (2 ml). The reaction mixture was stirred under reflux for 48 hours, and then concentrated to give a yellow powder. The yellow powder was basified using sodium carbonate solution and aqueous sodium hydroxide solution and filtered, to provide 5-(5-nitro-pyridin-2-yl)-thiophene-2-carboxylic acid methyl ester (1.78 g) as a solid. LCMS (Method C): R_{T}=3.56 minutes.

### Reference Example 21

### (a) 5-(3-Benzyloxy-phenyl)-thiophene-2-carboxylic acid ethyl ester

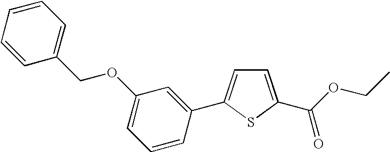

To a mixture of 5-(3-hydroxy-phenyl)-thiophene-2-carboxylic acid ethyl ester [124 mg, 0.50 mmol, Reference Example 27(a)], potassium carbonate (83 mg, 0.60 mmol) and N,N-dimethylformamide (1.5 ml) was added benzyl chloride (63 μl, 0.55 mmol). The resulting mixture was heated to 70° C. and stirred overnight. After allowing the reaction mixture to cool, it was partitioned between ethyl acetate and water. The organic phase was separated, and the aqueous phase was extracted with ethyl acetate (3×). The organic phases were combined, washed with 10% aqueous citric acid, followed by saturated aqueous sodium hydrogen carbonate solution, brine, then dried (MgSO₄), and concentrated to provide 5-(3-benzyloxy-phenyl)-thiophene-2-carboxylic acid ethyl ester (154 mg) as a brown oil. LCMS (Method C): R_{T}=4.64 minutes.

### Reference Example 22

### (a) 4-Benzyloxy-2-thiophen-2-yl-pyrimidine

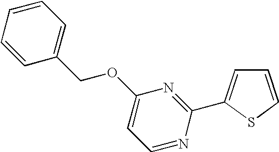

To a mixture of 2-thiophen-2-yl-pyrimidin-4-ol [240 mg, 1.35 mmol, Reference Example 26(a)], potassium carbonate (371 mg, 2.69 mmol) and N,N-dimethylformamide (3 ml) was added benzyl bromide (170 μl, 2.44 mmol). The resulting mixture was heated to 70° C. and stirred for 1 hour. The reaction mixture was allowed to cool, then poured into water and extracted with diethyl ether. The organic phase was separated, dried (MgSO₄) and concentrated to give a yellow oil which was subjected to flash column chromatography using dichloromethane as eluent, to provide 4-benzyloxy-2-thiophen-2-yl-pyrimidine (265 mg), which was used directly without further purification.

### Reference Example 23

### (a) 5-Phenyl-1-thiophen-2-yl-pentane-1,3-dione

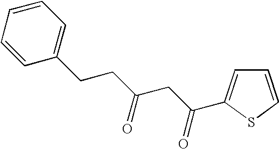

To a solution of 2-acetylthiophene (2.0 g, 15.87 mmol) and tetrahydrofuran, was added sodium hydride (0.70 g, 17.46 mmol), followed by methyl 3-phenylproprionate (2.86 g, 17.46 mmol). The reaction mixture was then heated to reflux and stirred for 5 hours, and was subsequently stirred at room temperature over the weekend. The mixture was concentrated to give a residue, which was treated with hydrochloric acid (1M) and extracted into ethyl acetate. The organic phase was separated, washed with brine, dried (MgSO₄), and evaporated to provide 5-phenyl-1-thiophen-2-yl-pentane-1,3-dione (4.37 g), which was used directly without further purification.

### (b) 4,4,4-Trifluoro-1-(3-methyl-thiophen-2-yl)-butane-1,3-dione

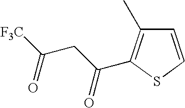

To a solution of 2-acetyl-3-methylthiophene (2.0 g, 14.29 mmol) and tetrahydrofuran (40 ml), was added sodium hydride (0.86 g, 21.43 mmol), followed by ethyl trifluoroacetate (3.04 g, 21.43 mmol). Once the reaction mixture had gone clear, the solvent was removed under reduced pressure to give a residue, which was treated with hydrochloric acid (1M), and extracted into ethyl acetate. The organic phase was separated, washed with brine, dried (MgSO₄) and evaporated, to provide 4,4,4-trifluoro-1-(3-methyl-thio hen-2-yl)-butane-1,3-dione (3.72 g), which was used directly without further purification.

### Reference Example 24

### (a) 5-(2,2-Dibromo-acetyl)-thiophene-2-carboxylic acid methyl ester

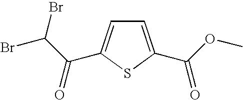

To a solution of 5-acetylthiophene-2-carboxylic acid (2.5 g, 14.7 mmol) in methanol (40 ml) at 50° C. was added bromine (4 ml, 58.8 mmol). The reaction mixture was stirred overnight at this temperature. The reaction mixture was concentrated to give a residue, which was dissolved in ethyl acetate, washed with saturated aqueous sodium hydrogen carbonate solution, brine, then dried (MgSO₄) and concentrated, to provide 5-(2,2-dibromo-acetyl)-thiophene-2-carboxylic acid methyl ester (4.82 g), which was used directly without further purification.

### Reference Example 25

### (a) Thiophene-2-carboxylic acid N′-(2,2,2-trifluoro-1-imino-ethyl)-hydrazide

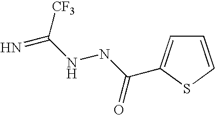

To a solution of trifluoroacetamidine (1.12 g, 10.0 mmol) in ethanol (20 ml) was added 2-thiophenecarboxylichydrazide (1.11 g, 7.85 mmol). The resulting solution was stirred for 2 hours, and then concentrated to give a residue. The residue was dissolved in ethyl acetate, passed through a pad of silica and concentrated, to provide thiophene-2-carboxylic acid N′-(2,2,2-trifluoro-1-imino-ethyl)-hydrazide (1.93 g) as an off-white powder. LCMS (Method C): R_{T}=2.19 minutes; 238 (M+H)⁺.

### Reference Example 26

### (a) 2-Thiophen-2-yl-pyrimidin-4-ol

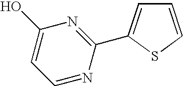

A mixture of 2-thiophenecarboxamidine (0.5 g, 3.09 mmol) and ethyl 3,3-diethoxypropionate (1.18 ml, 6.07 mmol) was subjected to microwave irradiation, heating to 180° C. for 5 minutes. The reaction mixture was then triturated with methanol and filtered, to provide 2-thiophen-2-yl-pyrimidin-4-ol (238 mg) as a yellow powder, which was used directly without further purification.

### Reference Example 27

### (a) 5-(3-Hydroxy-phenyl)-thiophene-2-carboxylic acid ethyl ester

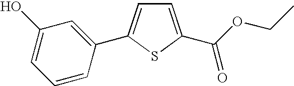

To a solution of ethyl 5-bromothiophene-2-carboxylate (155 mg, 0.66 mmol) in dimethoxyethane/ethanol/water (7:2:3, v/v/v) was added 3-(4,4,5,5-tetramethyl-1,3,2-dioxaborolan-2-yl) phenol (174 mg, 0.79 mmol), tetrakis(triphenylphosphine)palladium(0) (15 mg, 0.013 mmol) and cesium carbonate (169 mg, 0.52 mmol). The mixture was subjected to microwave irradiation, heating to 150° C. for 5 minutes. The reaction mixture was then partitioned between ethyl acetate and 10% aqueous citric acid, and the two phases were separated. The aqueous phase was extracted with ethyl acetate (2×) and the combined organic phases were washed with water, followed by brine, dried (MgSO₄) and concentrated to give an off-white oily solid. The solid was triturated with diethyl ether and pentane (1:1, v/v) and filtered, to provide 5-(3-hydroxy-phenyl)-thiophene-2-carboxylic acid ethyl ester (140 mg) as a white solid. LCMS (Method C): R_{T}=3.53 minutes; 247 (M⁻).

### Reference Example 28

### (a) 5-(5-Phenethylamino-pyridin-2-yl)-thiophene-2-carboxylic acid methyl ester

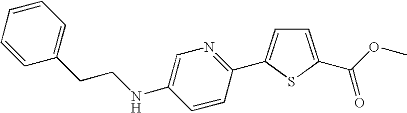

A solution of 5-(5-amino-pyridin-2-yl)-thiophene-2-carboxylic acid methyl ester [50 mg, 0.21 mmol, Reference Example 15(a)] and phenylacetaldehyde (28 mg, 0.21 mmol) in anhydrous tetrahydrofuran (2.5 ml), was stirred for 16 hours. Glacial acetic acid (13 ml, 0.23 mmol) and sodium triacetoxyborohydride (89 mg, 0.42 mmol) was then added to the reaction mixture. After stirring for a further 20 hours the reaction mixture was concentrated. The residue was dissolved in ethyl acetate and the resultant solution washed with 10% citric acid solution, followed by saturated sodium hydrogen carbonate solution, then brine. The organic layer was separated, dried (MgSO₄) and evaporated to give a residue, which was subjected to flash column chromatography using a mixture of cyclohexane and ethyl acetate (75:25, v/v) as eluent, to provide 5-(6-phenethylamino-pyridin-2-yl)-thiophene-2-carboxylic acid hydroxyamide (39 mg) as a yellow oil. LCMS (Method C): R_{T}=3.90 minutes; 339 (M+H)⁺.

### (b) 5-{5-[(Quinolin-2-ylmethyl)-amino]-pyridin-2-yl}-thiophene-2-carboxylic acid methyl ester

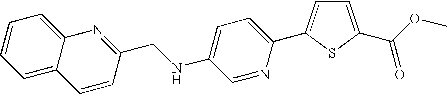

A mixture of 5-(5-amino-pyridin-2-yl)-thiophene-2-carboxylic acid methyl ester [80 mg, 0.34 mmol, Reference Example 15(a)], 2-quinolinecarboxaldehyde (69 mg, 0.44 mmol), and 4A molecular sieves in anhydrous tetrahydrofuran (3.3 ml), was stirred for 20 hours. Glacial acetic acid (22 ml, 0.37 mmol) and sodium triacetoxyborohydride (108 mg, 0.51 mmol) were then added to the reaction mixture. After stirring for a further 16 hours the reaction mixture was concentrated, the residue was dissolved in ethyl acetate and the solution washed with 10% citric acid solution, followed by saturated sodium hydrogen carbonate solution, then brine. The organic layer was separated, dried (MgSO₄) and evaporated to dryness to provide, 5-{5-[(quinolin-2-ylmethyl)-amino]-pyridin-2-yl}-thiophene-2-carboxylic acid methyl ester which was used directly without further purification. LCMS (Method C): R_{T}=3.15 minutes; 375 (M+H)⁺.

### (c) 5-{5-[(2,3-Dihydro-benzo[1,4]dioxin-6-ylmethyl)-amino]-pyridin-2-yl}-thiophene-2-carboxylic acid methyl ester

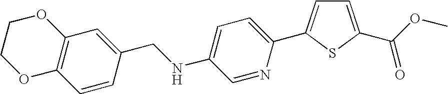

By proceeding in a similar manner to Reference Example 28(b) but using 5-(5-amino-pyridin-2-yl)-thiophene-2-carboxylic acid methyl ester [80 mg, 0.34 mmol, Reference Example 15(a)] and 1,4-benzodioxin-6-carboxaldehyde (72 mg, 0.44 mmol), there was prepared 5-{5-[(2,3-dihydro-benzo[1,4]dioxin-6-ylmethyl)-amino]-pyridin-2-yl}-thiophene-2-carboxylic acid methyl ester which was used directly without further purification. LCMS (Method C): R_{T}=3.60 minutes; 383 (M+H)⁺.

### (d) 5-{5-[(Benzofuran-2-ylmethyl)-amino]-pyridin-2-yl}-thiophene-2-carboxylic acid methyl ester

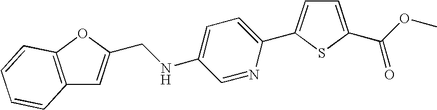

By proceeding in a similar manner to Reference Example 28(b) but using 5-(5-amino-pyridin-2-yl)-thiophene-2-carboxylic acid methyl ester [55 mg, 0.23 mmol, Reference Example 15(a)] and benzo[b]furan-2-carboxaldehyde (38 mg, 0.25 mmol), there was prepared 5-{5-[(benzofuran-2-ylmethyl)-amino]-pyridin-2-yl}-thiophene-2-carboxylic acid methyl ester which was used directly without further purification. LCMS (Method C): R_{T}=3.87 minutes; 365 (M+H)⁺.

### (f) 5-[1-(2-Benzylamino-ethyl)-1H-pyrazol-3-yl]-thiophene-2-carboxylic acid methyl ester

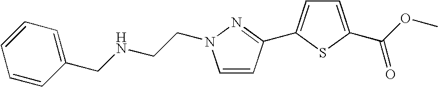

A solution of 5-[1-(3-amino-ethyl)-1H-pyrazol-3-yl]-thiophene-2-carboxylic acid methyl ester [126 mg, 0.5 mmol, Reference Example 35(b)] and benzaldehyde (42 μl, 0.4 mmol) in anhydrous methanol (4 ml) was stirred overnight. Sodium borohydride (24 μg, 0.63 mmol) was then added to the reaction mixture, which was stirred for a further 2 hours before being concentrated. The residue was treated with water (2 ml) and saturated sodium hydrogen carbonate solution (1 ml) then loaded onto an Isolute® HM-N cartridge (5 ml). After 30 minutes the cartridge was washed with chloroform and the solvent concentrated, to provide 5-[1-(2-benzylamino-ethyl)-1H-pyrazol-3-yl]-thiophene-2-carboxylic acid methyl ester (60 mg) as a yellow gum. LCMS (Method C): R_{T}=2.19 minutes; 342 (M+H)⁺.

### (g) 5-(1-{3-[(Quinolin-2-ylmethyl)-amino]-propyl}-1H-pyrazol-3-yl)-thiophene-2-carboxylic acid methyl ester

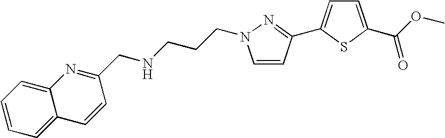

A solution of 5-[1-(3-amino-propyl)-1H-pyrazol-3-yl]-thiophene-2-carboxylic acid methyl ester [100 mg, 0.38 mmol, Reference Example 35(a)] and 2-quinolinecarboxaldehyde (49 mg, 0.31 mmol) in anhydrous methanol (3 ml) was stirred overnight. Sodium borohydride (19 mg, 0.5 mmol) was then added to the reaction mixture, which was stirred for a further 2 hours before being concentrated. The residue was treated with water (2 ml) and saturated sodium hydrogen carbonate solution (1 ml) then loaded onto an Isolute® HM-N cartridge (5 ml). After 30 minutes the cartridge was washed with chloroform and the solvent concentrated, to provide 5-(1-{3-[(quinolin-2-ylmethyl)-amino]-propyl}-1H-pyrazol-3-yl)-thiophene-2-carboxylic acid methyl ester. LCMS (Method C): R_{T}=2.29 minutes; 407 (M+H)⁺.

### (h) 5-(1-{3-[(Benzo[1,3]dioxol-5-ylmethyl)-amino]-propyl}-1H-pyrazol-3-yl)-thiophene-2-carboxylic acid methyl ester

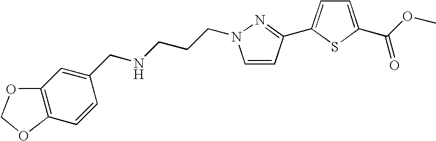

By proceeding in a similar manner to Reference Example 28(g) but using 5-[1-(3-amino-propyl)-1H-pyrazol-3-yl]-thiophene-2-carboxylic acid methyl ester [100 mg, 0.38 mmol, Reference Example 35(a)] and piperonal (47 mg, 0.31 mmol), there was prepared 5-(1-{3-[(benzo[1,3]dioxol-5-ylmethyl)-amino]-propyl}-1H-pyrazol-3-yl)-thiophene-2-carboxylic acid methyl ester. LCMS (Method C): R_{T}=2.20 minutes; 400 (M+H)⁺.

### (i) 5-(1-{2-[(Benzo[1,3]dioxol-5-ylmethyl)-amino]-ethyl}-1H-pyrazol-3-yl)-thiophene-2-carboxylic acid methyl ester

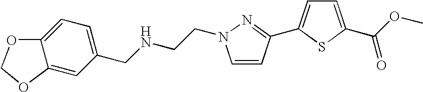

By proceeding in a similar manner to Reference Example 28(g) but using 5-[1-(3-amino-ethyl)-1H-pyrazol-3-yl]-thiophene-2-carboxylic acid methyl ester [100 mg, 0.4 mmol, Reference Example 35(b)] and piperonal (50 mg, 0.33 mmol), there was prepared 5-(1-{2-[(benzo[1,3]dioxol-5-ylmethyl)-amino]-ethyl}-1H-pyrazol-3-yl)-thiophene-2-carboxylic acid methyl ester. LCMS (Method C): R_{T}=2.20 minutes; 386 (M+H)⁺.

### (o) 5-(1-{2-[(Pyridin-4-ylmethyl)-amino]-ethyl}-H-pyrazol-3-yl)-thiophene-2-carboxylic acid methyl ester

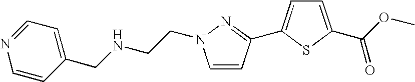

By proceeding in a similar manner to Reference Example 28(g) but using 5-[1-(3-amino-ethyl)-1H-pyrazol-3-yl]-thiophene-2-carboxylic acid methyl ester [100 mg, 0.4 mmol, Reference Example 35(b)] and iso-nicotinaldehyde (35 mg, 0.33 mmol), there was prepared 5-(1-{2-[(pyridin-4-ylmethyl)-amino]-ethyl}-1H-pyrazol-3-yl)-thiophene-2-carboxylic acid methyl ester. LCMS (Method C): R_{T}=1.71 minutes; 343 (M+H)⁺.

### (k) 5-[6-(3-Phenyl-propylamino)-pyridin-2-yl]-thiophene-2-carboxylic acid methyl ester

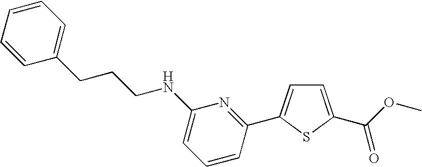

A mixture of 5-(6-amino-pyridin-2-yl)-thiophene-2-carboxylic acid methyl ester [400 mg, 1.7 mmol, Reference Example 12(b)] and 3-phenylpropionaldehyde (320 mg, 2.4 mmol), in dichloroethane (6 ml), was stirred for 5 minutes. Sodium triacetoxyborohydride (720 mg, 3.4 mmol) was then added to the reaction mixture, which was stirred over the weekend. The reaction mixture was diluted with dichloromethane and washed with saturated sodium carbonate solution. The organic layer was isolated, dried (MgSO₄) and concentrated, to provide 5-[6-(3-phenyl-propylamino)-pyridin-2-yl]-thiophene-2-carboxylic acid methyl ester (660 mg), which was used directly without further purification. LCMS (Method C): R_{T}=4.32 minutes; 353 (M+H)⁺.

### Reference Example 29

### (a) 5-{1-[2-(4-Fluoro-benzyloxy)-ethyl]-1H-pyrazol-3-yl}-thiophene-2-carboxylic acid methyl ester

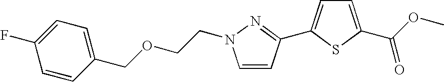

To a solution of 5-[1-(2-hydroxy-ethyl)-1H-pyrazol-3-yl]-thiophene-2-carboxylic acid methyl ester [100 mg, 0.4 mmol, Reference Example 10(aa)] in anhydrous tetrahydrofuran (6 ml) was added sodium hydride (30 mg, 0.6 mmol) and the suspension was stirred for 30 minutes. 4-Fluorobenzylbromide (200 μl, 1.6 mmol) was then added dropwise, and the reaction mixture was left to stir overnight. 1 M Hydrochloric acid solution was added to the reaction mixture which was then extracted with ethyl acetate (3×). The combined organic layers were washed with brine, then dried (gSO₄), and concentrated to give a pale yellow oil. The oil was subjected to flash column chromatography using a mixture of ethyl acetate and dichloromethane (gradient 5:95 to 25:75, v/v) as eluent, to provide 5-{1-[2-(4-fluoro-benzyloxy-ethyl]-1H-pyrazol-3-yl}-thiophene-2-carboxylic acid methyl ester (72 mg) as a yellow oil. LCMS (Method C): R_{T}=3.76 minutes; 361 (N+H)⁺.

### (b) 5-(6-Benzyloxymethyl-pyridin-2-yl)-thiophene-2-carboxylic acid methyl ester

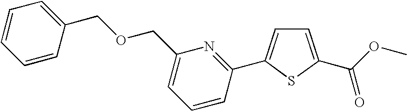

To a cooled (0° C.) suspension of sodium hydride (55 mg, 1.38 mmol) in anhydrous N,N-dimethylformamide (1 ml) was added a solution of 5-(6-hydroxymethyl-pyridin-2-yl)-thiophene-2-carboxylic acid methyl ester [220 mg, 0.88 mmol, Reference Example 34(a)] in anhydrous N,N-dimethylformamide (2 ml), followed by a solution of benzyl bromide (120 μl, 1.0 mmol) in anhydrous N,N dimethylformamide (1 ml). The reaction mixture was allowed to warm to room temperature and stirred overnight, before being diluted with diethyl ether and washed with water (x2), followed by brine. The organic layer was isolated, dried (MgSO₄) and concentrated, to provide 5-(6-benzyloxymethyl-pyridin-2-yl)-thiophene-2-carboxylic acid methyl ester (155 mg) as a light brown oil. LCMS (Method C): R_{T}=4.23 minutes; 340 (N+H)⁺.

### Reference Example 30

### (a) 5-(1-{[(Pyridin-2-ylmethyl)-carbamoyl]-methyl}-1H-pyrazol-3-yl)-thiophene-2-carboxylic acid methyl ester

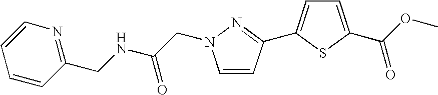

A solution of 5-(1-carboxymethyl-1H-pyrazol-3-yl)-thiophene-2-carboxylic acid methyl ester [50 mg, 0.18 mmol, Reference Example 31(a)] in dimethylformamide (1.8 ml) was treated with diisopropylethylamine (94 μl, 0.54 mmol), 2-(aminomethyl)pyridine (25 μl, 0.24 mmol) and O-(7-azabenzotriazol-1-yl)-N,N,N′,N′-tetramethyluronium hexafluorophosphate (71 mg, 0.18 mmol). The mixture was stirred at room temperature for 6 hours, before being evaporated under reduced pressure. The residue was dissolved in ethyl acetate and washed with saturated sodium hydrogen carbonate solution, followed by brine. The organic layer was separated, dried (Na₂SO₄) and concentrated to provide a yellow residue, which was subjected to flash column chromatography on silica using a mixture of ethyl acetate and cyclohexane (gradient 95:5 to 100:0, v/v) as eluent, to provide 5-(1-{[(pyridin-2-ylmethyl)-carbamoyl]-methyl}-1H-pyrazol-3-yl-thiophene-2-carboxylic acid methyl ester (53 mg) as a white solid. LCMS (Method C): R_{T}=2.15 minutes; 356 (N+H)⁺.

### (b) 5-[1-(Quinolin-8-ylcarbamoylmethyl-1H-pyrazol-3-yl]-thiophene-2-carboxylic acid methyl ester

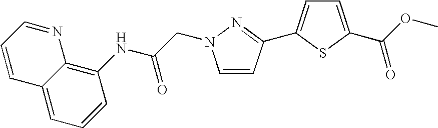

By proceeding in a similar manner to Reference Example 30(a) but using 5-(1-carboxymethyl-1H-pyrazol-3-yl)-thiophene-2-carboxylic acid methyl ester [300 mg, 1.12 mmol, Reference Example 31(a)] and 8-aminoquinoline (211 mg, 1.46 mmol), (gradient 20:80 to 30:70, v/v), there was prepared 5-[1-(quinolin-8-ylcarbamoylmethyl)-1H-pyrazol-3-yl]-thiophene-2-carboxylic acid methyl ester (320 mg) as a light brown solid. LCMS (Method C): R_{T}=3.64 minutes; 393 (M+H)⁺.

### (c) 5-[1-(Quinolin-3-ylcarbamoylmethyl)-1H-pyrazol-3-yl]-thiophene-2-carboxylic acid methyl ester

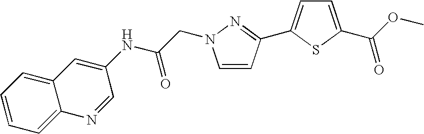

A solution of 5-(1-carboxymethyl-1H-pyrazol-3-yl)-thiophene-2-carboxylic acid methyl ester [80 mg, 0.30 mmol, Reference Example 31(a)] in dimethylformamide (3 ml) was treated with diisopropylethylamine (157 μl, 0.9 mmol), 3-aminoquinoline (45 mg, 0.31 mmol) and O-(7-azabenzotriazol-1-yl)-N,N,N′,N′-tetramethyluronium hexafluorophosphate (114 mg, 0.30 mmol). The mixture was stirred at room temperature for 2 hours, before being evaporated under reduced pressure. The residue was dissolved in ethyl acetate and the resultant solution washed with water (2×). The organic layer was separated, dried (Na₂SO₄) and concentrated to provide a residue, 5-[1-(quinolin-3-ylcarbamoylmethyl)-1H-pyrazol-3-yl]-thiophene-2-carboxylic acid methyl ester which was used directly without further purification. LCMS (Method C): R_{T}=3.02 minutes; 393 (M+H)⁺.

### (d) 5-[1-(Pyridin-3-ylcarbamoylmethyl)-1H-pyrazol-3-yl]-thiophene-2-carboxylic acid methyl ester

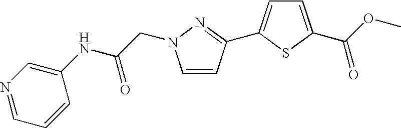

By proceeding in a similar manner to Reference Example 30(c) but using 5-(1-carboxymethyl-1H-pyrazol-3-yl)-thiophene-2-carboxylic acid methyl ester [70 mg, 0.26 mmol, Reference Example 31(a)] and 3-aminopyridine (26 mg, 0.27 mmol), there was prepared 5-[1-(pyridin-3-ylcarbamoylmethyl)-1H-pyrazol-3-yl]-thiophene-2-carboxylic acid methyl ester which was used directly without further purification. LCMS (Method C): R_{T}=2.17 minutes; 343 (M+H)⁺.

### (e) 5-(1-{[(Pyridin-3-ylmethyl)-carbamoyl]-methyl}-1H-pyrazol-3-yl)-thiophene-2-carboxylic acid methyl ester

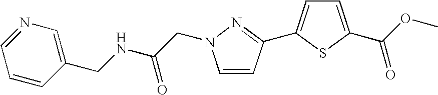

By proceeding in a similar manner to Reference Example 30(c) but using 5-(1-carboxymethyl-1H-pyrazol-3-yl)-thiophene-2-carboxylic acid methyl ester [50 mg, 0.18 mmol, Reference Example 31(a)] and 3-aminomethylpyridine (20 μl, 0.19 mmol), there was prepared 5-(1-{[(Pyridin-3-ylmethyl)-carbamoyl]-methyl}-1H-pyrazol-3-yl-thiophene-2-carboxylic acid methyl ester which was used directly without further purification. LCMS (Method C): R_{T}=1.96 minutes; 357 (M+I)⁺.

### (f) 5-{1-[(3-Methoxy-phenylcarbamoyl)-methyl]-1H-pyrazol-3-yl}-thiophene-2-carboxylic acid methyl ester

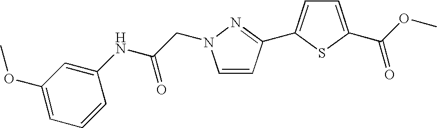

A solution of 5-(1-carboxymethyl-1H-pyrazol-3-yl)-thiophene-2-carboxylic acid methyl ester [400 mg, 1.5 mmol, Reference Example 31(a)] in dimethylformamide (4 ml) was treated with diisopropylethylamine (780 μl, 4.5 mmol), 3-aminoanisole (210 μl, 1.8 mmol) and O-(7-azabenzotriazol-1-yl)-N,N,N′N′-tetramethyluronium hexafluorophosphate (570 mg, 1.5 mmol). The mixture was stirred at room temperature overnight, then concentrated under reduced pressure to give a residue. The residue was subjected to flash column chromatography on silica using a mixture of ethyl acetate and cyclohexane (gradient 0:100 to 50:50, v/v) as eluent, to provide 5-{1-[(3-methoxy-phenylcarbamoyl)-methyl]-1H-pyrazol-3-yl}-thiophene-2-carboxylic acid methyl ester (416 mg) as a solid. LCMS (Method C): R_{T}=3.32 minutes; 372 (M+1H)⁺.

### (g) 5-{1-[(3-Chloro-phenylcarbamoyl)-methyl]-1H-pyrazol-3-yl}-thiophene-2-carboxylic acid methyl ester

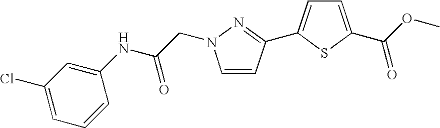

A solution of 5-(1-carboxymethyl-1H-pyrazol-3-yl)-thiophene-2-carboxylic acid methyl ester [100 mg, 0.38 mmol, Reference Example 31(a)] in dimethylformamide (3 ml) was treated with diisopropylethylamine (196 μl, 1.1 mmol), 3-chloroaniline (53 mg, 0.41 mmol) and O-(7-azabenzotriazol-1-yl)-N,N,N′,N′-tetramethyluronium hexafluorophosphate (143 mg, 0.38 mmol). The mixture was stirred at room temperature overnight, then concentrated under reduced pressure to give a residue. The residue was dissolved in ethyl acetate, washed with saturated sodium hydrogen carbonate solution, then 1M hydrochloric acid. The organic layer was separated, concentrated and triturated with ethyl acetate, to provide 5-{1-[(3-chloro-phenylcarbamoyl)-methyl]-1H-pyrazol-3-yl}-thiophene-2-carboxylic acid methyl ester as a white solid, which was used directly without further purification. LCMS (Method C): R_{T}=3.58 minutes; 376 (M+H)⁺.

### (h) 5-{1-[(3,5-Difluoro-phenylcarbamoyl)-methyl]-1H-pyrazol-3-yl}-thiophene-2-carboxylic acid methyl ester

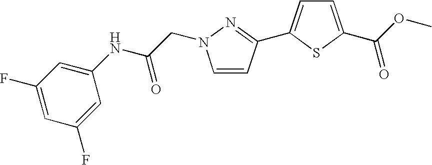

By proceeding in a similar manner to Reference Example 30(g) but using 5-(1-carboxymethyl-1H-pyrazol-3-yl)-thiophene-2-carboxylic acid methyl ester [100 mg, 0.38 mmol, Reference Example 31(a)] and 3,5-difluoroaniline (53 mg, 0.41 mmol), there as prepared 5-{1-[(3,5-difluoro-phenylcarbamoyl)-methyl]-1H-pyrazol-3-yl}-thiophene-2-carboxylic acid methyl ester as a white solid, which was used directly without further purification. LCMS (Method C): R_{T}=3.55 minutes; 378 (M+H)⁺.

### (i) 5-{1-[(3-Sulfamoyl-phenylcarbamoyl)-methyl]-1H-pyrazol-3-yl}-thiophene-2-carboxylic acid methyl ester

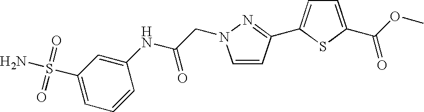

By proceeding in a similar manner to Reference Example 30(g) but using 5-(1-carboxymethyl-1H-pyrazol-3-yl)-thiophene-2-carboxylic acid methyl ester [100 mg, 0.38 mmol, Reference Example 31(a)] and 3-aminobenzenesulfonamide (71 mg, 0.41 mmol), there was prepared 5-{1-[(3-sulfamoyl-phenylcarbamoyl)-methyl]-1H-pyrazol-3-yl}-thiophene-2-carboxylic acid methyl ester as a white solid, which was used directly without further purification. LCMS (Method C): R_{T}=2.87 minutes; 421 (M+H)⁺.

### (j) 5-{1-[(H-Indazol-7-ylcarbamoyl)-methyl]-1H-pyrazol-3-yl}-thiophene-2-carboxylic acid methyl ester

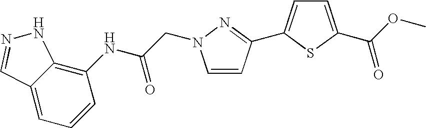

By proceeding in a similar manner to Reference Example 30(g) but using 5-(1-carboxymethyl-1H-pyrazol-3-yl)-thiophene-2-carboxylic acid methyl ester [100 mg, 0.38 mmol, Reference Example 31(a)] and 1H-indazol-7-amine (55 mg, 0.41 mmol), there was prepared 5-{1-[(1H-indazol-7-ylcarbamoyl)-methyl]-1H-pyrazol-3-yl}-thiophene-2-carboxylic acid methyl ester as a white solid, which was used directly without further purification. LCMS (Method C): R_{T}=2.99 minutes; 382 (M+H)⁺.

### (k) 5-{1-[(1H-Indol-7-ylcarbamoyl)-methyl]-1H-pyrazol-3-yl}-thiophene-2-carboxylic acid methyl ester

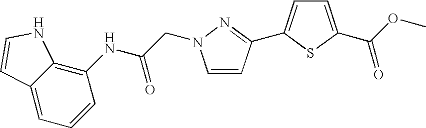

By proceeding in a similar manner to Reference Example 30(g) but using 5-(1-carboxymethyl-1H-pyrazol-3-yl)-thiophene-2-carboxylic acid methyl ester [100 mg, 0.38 mmol, Reference Example 31(a)] and 7-aminoindole (55 mg, 0.41 mmol), there was prepared 5-{1-[(1H-indol-7-ylcarbamoyl)-methyl]-1H-pyrazol-3-yl}-thiophene-2-carboxylic acid methyl ester as a white solid, which was used directly without further purification. LCMS (Method C): R_{T}=3.18 minutes; 381 (M+H)⁺.

### (1) 5-(5-Methyl-4-phenethylcarbamoyl-1H-imidazol-2-yl)-thiophene-2-carboxylic acid methyl ester

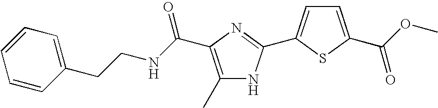

A solution of 2-(5-methoxycarbonyl-thiophen-2-yl)-5-methyl-1H-imidazole-4-carboxylic acid [467 mg, 1.23 mmol, Reference Example 31(b)] in dimethylformamide (10 ml) was treated with diisopropylethylamine (857 μl, 4.9 mmol), phenethylamine (154 μl, 1.23 mmol), a catalytic quantity of 4-dimethylaminopyridine and O-(7-azabenzotriazol-1-yl)-N,N,N′,N′-tetramethyluronium hexafluorophosphate (467 mg, 1.23 mmol). The mixture was stirred at room temperature overnight, then concentrated under reduced pressure to give a residue. The residue was passed through an SCX-2 column and eluted with 2M ammonia, and the fractions were collected and concentrated and diluted with water. The aqueous layer was extracted with dichloromethane and the organic layers were combined, dried (MgSO₄) and concentrated, to provide 5-(5-methyl-4-phenethylcarbamoyl-1H-imidazol-2-yl)-thiophene-2-carboxylic acid methyl ester (39 mg) as a solid, which was used directly without further purification. LCMS (Method C): R_{T}=3.33 minutes; 370 (M+W)⁺.

### (m) 5-(4-Benzylcarbamoyl-5-methyl-1H-imidazol-2-yl)-thiophene-2-carboxylic acid methyl ester

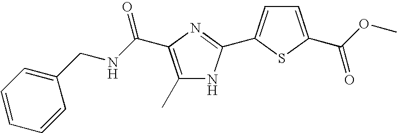

A solution of 2-(5-methoxycarbonyl-thiophen-2-yl)-5-methyl-1H-imidazole-4-carboxylic acid [467 mg, 1.23 mmol, Reference Example 31(b)] in dimethylformamide (10 ml) was treated with diisopropylethylamine (857 μl, 4.9 mmol), benzylamine (134 μl, 1.2 mmol), one crystal of 4-dimethylaminopyridine and O-(7-azabenzotriazol-1-yl)-N,N,N′,N′-tetramethyluronium hexafluorophosphate (467 mg, 1.23 mmol). The mixture was stirred at room temperature overnight, then concentrated under reduced pressure to give a residue. The residue was partitioned between water and ethyl acetate, and the organic layer was isolated and concentrated to give a yellow gum. The gum was triturated with acetonitrile, to provide 5-(5-methyl-4-benzylcarbamoyl-1H-imidazol-2-yl)-thiophene-2-carboxylic acid methyl ester (39 mg) as a yellow powder, which was used directly without further purification. LCMS (Method C): R_{T}=3.24 minutes; 356 (M+H)⁺.

### Reference Example 31

### (a) 5-(1-Carboxymethyl-1H-pyrazol-3-yl)-thiophene-2-carboxylic acid methyl ester

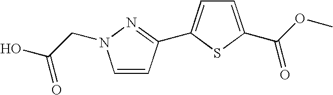

To a solution of 5-(1-tert-butoxycarbonylmethyl-1H-pyrazol-3-yl)-thiophene-2-carboxylic acid methyl ester [70 mg, 0.21 mmol, Reference Example 10(ab)], triethylsilane (840 μl, 0.52 mmol) and dichloromethane (430 μl) was added trifluoroacetic acid (216 μl, 2.82 mmol). The mixture was stirred at room temperature for 16 hours, before being evaporated under reduced pressure to provide, 5-(1-carboxymethyl-1H-pyrazol-3-yl-thiophene-2-carboxylic acid methyl ester which was used directly without further purification. LCMS (Method C): R_{T}=2.61 minutes; 267 (M+H)⁺.

### (b) 2-(5-Methoxycarbonyl-thiophen-2-yl)-5-methyl-1H-imidazole-4-carboxylic acid

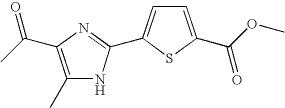

A solution of 2-(5-methoxycarbonyl-thiophen-2-yl)-5-methyl-1H-imidazole-4-carboxylic acid tert-butyl ester [1.0 g, 3.10 mmol, Reference Example 36(a)] and 96% trifluoroacetic acid in water (60 ml), was stirred at room temperature for 3 days, before being concentrated to a gum. The gum was subsequently triturated with diethyl ether followed by methanol and diethyl ether, to provide 2-(5-methoxycarbonyl-thiophen-2-yl)-5-methyl-1H-imidazole-4-carboxylic acid (1.17 g) as a brown gum, which was used directly without further purification. LCMS (Method C): R_{T}=2.08 minutes; 267 (N+H)⁺.

### Reference Example 32

### (b) 5-{6-[(4-Methoxy-phenylamino)-methyl]-pyridin-2-yl}-thiophene-2-carboxylic acid

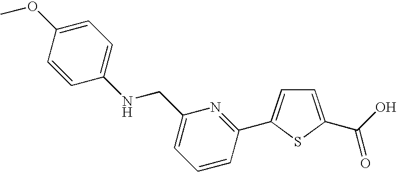

To a mixture of 5-(6-formyl-pyridin-2-yl)-thiophene-2-carboxylic acid [88 mg, 0.38 mmol, Reference Example 14(e)], 4-methoxyaniline (46 mg, 0.38 mmol), and dichloroethane (4 ml), was added sodium triacetoxyborohydride (97 mg, 0.46 mmol). After stirring overnight the reaction mixture was concentrated, to provide 5-{6-[(4-methoxy-phenylamino)-methyl]-pyridin-2-yl}-thiophene-2-carboxylic acid as a purple solid, which was used directly without further purification. LCMS (Method C): R_{T}=2.65 minutes; 341 (M+H)⁺.

### (c) 5-{6-(Methyl-pyridin-3-ylmethyl-amino)-methyl]-pyridin-2-yl}-thiophene-2-carboxylic acid

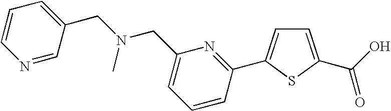

To a mixture of 5-(6-formyl-pyridin-2-yl)-thiophene-2-carboxylic acid [88 mg, 0.38 mmol, Reference Example 14(e)], methyl-pyridin-3-ylmethyl-amine (46 mg, 0.38 mmol), and dichloroethane (4 ml), was added sodium triacetoxyborohydride (97 mg, 0.46 mmol). After stirring overnight the reaction mixture was concentrated, to provide 5-{6-[(methyl-pyridin-3-ylmethyl-amino)-methyl]-pyridin-2-yl}-thiophene-2-carboxylic acid, which was used directly without further purification. LCMS (Method C): R_{T}=1.59 minutes; 340 (M+H)⁺.

### (d) 5-[6-(3,4-Tetrahydro-1H-isoquinolin-2-ylmethyl)-pyridin-2-yl]-thiophene-2-carboxylic acid

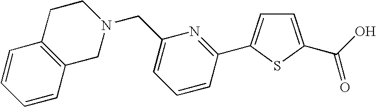

To a mixture of 5-(6-formyl-pyridin-2-yl)-thiophene-2-carboxylic acid [66 mg, 0.28 mmol, Reference Example 14(e)], 1,2,3,4-tetrahydroisoquinoline (37 mg, 0.28 mmol), and dichloroethane (4 ml), was added sodium triacetoxyborohydride (60 mg, 0.28 mmol). After stirring overnight the reaction mixture was concentrated, to provide 5-[6-(3,4-tetrahydro-1H-isoquinolin-2-ylmethyl)-pyridin-2-yl]-thiophene-2-carboxylic acid as an off-white solid, which was used directly without further purification. LCMS (Method C): R_{T}=1.99 minutes; 351 (M+H)⁺.

### (e) 5-{6-[(Methyl-naphthalen-1-ylmethl-amino-methyl]-pyridin-2-yl}-thiophene-2-carboxylic acid

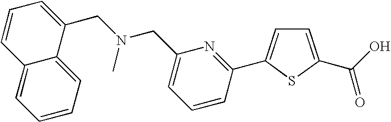

By proceeding in a similar manner to Reference Example 32(d) but using 5-(6-formyl-pyridin-2-yl)-thiophene-2-carboxylic acid [66 mg, 0.28 mmol, Reference Example 14(e)] and N-methyl-1-naphthylmethylamine (48 mg, 0.28 mmol), there was prepared 5-{6-[(methyl-naphthalen-1-ylmethyl-amino)-methyl]-pyridin-2-yl}-thiophene-2-carboxylic acid as an off-white solid, which was used directly without further purification. LCMS (Method C): R_{T}=2.24 minutes; 389 (M+H)⁺.

### (g) 5-[6-(4-Phenethyl-piperazin-1-ylmethyl)-pyridin-2-yl]-thiophene-2-carboxylic acid

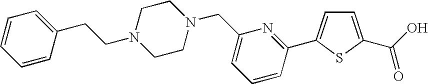

By proceeding in a similar manner to Reference Example 32(d) but using 5-(6-formyl-pyridin-2-yl)-thiophene-2-carboxylic acid [66 mg, 0.28 mmol, Reference Example 14(e)] and 1-(2-phenylethyl)-piperazine (53 mg, 0.28 mmol), there was prepared 5-[6-(4-phenethyl-piperazin-1-ylmethyl)-pyridin-2-yl]-thiophene-2-carboxylic acid as an off-white solid, which was used directly without further purification. LCMS (Method C): R_{T}=2.05 minutes; 408 (M+H)⁺.

### (h) 5-[6-(4-Pyridin-2-yl-piperazin-1-ylmethyl-pyridin-2-yl]-thiophene-2-carboxylic acid

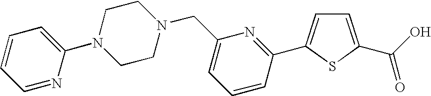

By proceeding in a similar manner to Reference Example 32(d) but using 5-(6-formyl-pyridin-2-yl)-thiophene-2-carboxylic acid [66 mg, 0.28 mmol, Reference Example 14(e)] and 1-(2-pyridyl)piperazine (46 mg, 0.28 mmol), there was prepared 5-[6-(4-pyridin-2-yl-piperazin-1-ylmethyl-pyridin-2-yl]-thiophene-2-carboxylic acid as an off-white solid, which was used directly without further purification. LCMS (Method C): R_{T}=1.55 minutes; 381 (M+H)⁺.

### (j) 5-(6-{[(Pyridin-3-ylmethyl)-amino]-methyl}-pyridin-2-yl)-thiophene-2-carboxylic acid

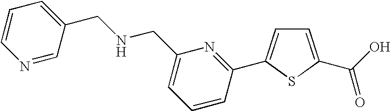

A mixture of 5-(6-formyl-pyridin-2-yl)-thiophene-2-carboxylic acid [88 mg, 0.38 mmol, Reference Example 14(e)], 3-picolylamine (41 mg, 0.38 mmol) and anhydrous ethanol (4 ml) was stirred at room temperature for 2 hours, before sodium borohydride (30 mg, 0.76 mmol) was added. After stirring overnight the reaction mixture was concentrated, to provide 5-(6-{[(pyridin-3-ylmethyl)-amino]-methyl}-pyridin-2-yl)-thiophene-2-carboxylic acid as an off-white solid, which was used directly without further purification. LCMS (Method C): R_{T}=1.56 minutes; 326 (M+H)⁺.

### (k) 5-{6-[(2-Pyridin-3-yl-ethylamino)-methyl]-pyridin-2-yl}-thiophene-2-carboxylic acid

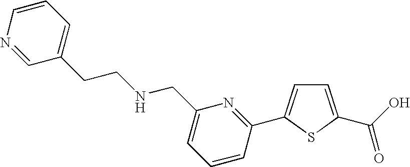

A mixture of 5-(6-formyl-pyridin-2-yl)-thiophene-2-carboxylic acid [88 mg, 0.38 mmol, Reference Example 14(e)], 3-(2-aminoethyl)pyridine (46 mg, 0.38 mmol) and anhydrous ethanol (4 ml) was stirred at room temperature for 2 hours, before sodium borohydride (30 mg, 0.76 mmol) was added. After stirring overnight the reaction mixture was concentrated, to provide 5-{6-[(2-pyridin-3-yl-ethylamino)-methyl]-pyridin-2-yl}-thiophene-2-carboxylic acid as an off-white solid, which was used directly without further purification. LCMS (Method C): R_{T}=0.34 minutes; 340 (M+H)⁺.

### (l) 5-{6-[(4-Fluoro-benzylamino)-methyl]-pyridin-2-yl}-thiophene-2-carboxylic acid

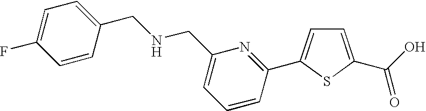

A mixture of 5-(6-formyl-pyridin-2-yl)-thiophene-2-carboxylic acid [88 mg, 0.38 mmol, Reference Example 14(e)], 4-fluorobenzylamine (48 mg, 0.38 mmol) and anhydrous ethanol (4 ml) was stirred at room temperature for 2 hours, before sodium borohydride (30 mg, 0.76 mmol) was added. After stirring overnight the reaction mixture was concentrated, to provide 5-{6-[(4-fluoro-benzylamino)-methyl]-pyridin-2-yl}-thiophene-2-carboxylic acid as an off-white solid, which was used directly without further purification. LCMS (Method C): R_{T}=2.05 minutes; 343 (M+H)⁺.

### (m) 5-(6-{[(Benzo[1,3]dioxol-5-ylmethyl)-amino]-methyl}-pyridin-2-yl)-thiophene-2-carboxylic acid

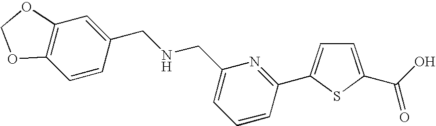

A mixture of 5-(6-formyl-pyridin-2-yl)-thiophene-2-carboxylic acid [88 mg, 0.38 mmol, Reference Example 14(e)], benzo[1,3]dioxol-5-yl-methylamine (57 mg, 0.38 mmol) and anhydrous ethanol (4 ml) was stirred at room temperature for 2 hours, before sodium borohydride (30 mg, 0.76 mmol) was added. After stirring overnight the reaction mixture was concentrated, to provide 5-(6-{[(benzo[1,3]dioxol-5-ylmethyl)-amino]-methyl}-pyridin-2-yl)-thiophene-2-carboxylic acid as an off-white solid, which was used directly without further purification. LCMS (Method C): R_{T}=2.05 minutes; 369 (M+H)⁺.

### (n) 5-(6-{[(1H-Benzoimidazol-2-ylmethyl-amino]-methyl}-pyridin-2-yl)-thiophene-2-carboxylic acid

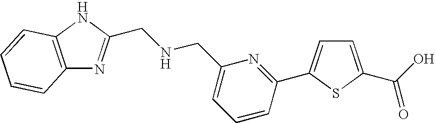

A mixture of 5-(6-formyl-pyridin-2-yl)-thiophene-2-carboxylic acid [88 mg, 0.38 mmol, Reference Example 14(e)], (1H-benzoimidazol-2-yl)-methylamine (60 mg, 0.38 mmol) and anhydrous ethanol (4 ml) was stirred at room temperature for 2 hours, before sodium borohydride (30 mg, 0.76 mmol) was added. After stirring overnight the reaction mixture was concentrated, to provide 5-(6-{[(1H-benzoimidazol-2-ylmethyl)-amino]-methyl}-pyridin-2-yl)-thiophene-2-carboxylic acid as an off-white solid, which was used directly without further purification. LCMS (Method C): R_{T}=1.89 minutes; 365 (M+H)⁺.

### (o) 5-{6-[(3-Imidazol-1-yl-propylamino)-methyl]-pyridin-2-yl}-thiophene-2-carboxylic acid

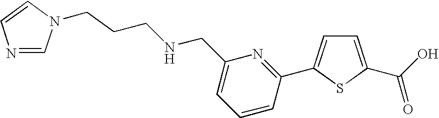

A mixture of 5-(6-formyl-pyridin-2-yl)-thiophene-2-carboxylic acid [88 mg, 0.38 mmol, Reference Example 14(e)], N-(3-aminopropyl)imidazole (48 mg, 0.38 mmol) and anhydrous ethanol (4 ml) was stirred at room temperature for 2 hours, before sodium borohydride (30 mg, 0.76 mmol) was added. After stirring overnight the reaction mixture was concentrated, to provide 5-{6-[(3-imidazol-1-yl-propylamino-methyl]-pyridin-2-yl}-thiophene-2-carboxylic acid as an off-white solid, which was used directly without further purification. LCMS (Method C): R_{T}=0.35 minutes; 343 (M+H)⁺.

### (q) 5-{6-[(Benzo[1,3]dioxol-5-ylmethyl-methyl-amino)-methyl]-pyridin-2-yl}-thiophene-2-carboxylic acid

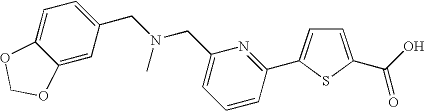

A mixture of 5-(6-formyl-pyridin-2-yl)-thiophene-2-carboxylic acid [132 mg, 0.56 mmol, Reference Example 14(e)], piperonylamine (86 mg, 0.56 mmol) and anhydrous ethanol (8 ml) was stirred at room temperature for 50 minutes, before formaldehyde (37% w/w, in water) (45 mL, 0.60 mmol) then sodium triacetoxyborohydride (356 mg, 1.68 mmol) were added. After stirring overnight the reaction mixture was concentrated, dissolved in methanol and water and loaded onto an SCX-2 cartridge. The cartridge was washed with ethyl acetate, methanol and 2M ammonia in methanol solution, to provide 5-{6-[(benzo[1,3]dioxol-5-ylmethyl-methyl-amino)-methyl]-pyridin-2-yl}-thiophene-2-carboxylic acid (139 mg) as a viscous brown oil. LCMS (Method C): R_{T}=2.10 minutes; 383 (M+H)⁺.

### Reference Example 33

### (a) 5-[6-(Methyl-phenethyl-amino)-pyridin-2-yl]-thiophene-2-carboxylic acid

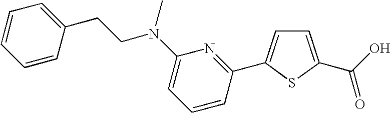

A mixture of 5-(6-bromo-pyridin-2-yl)-thiophene-2-carboxylic acid [85 mg, 0.30 mmol, Reference Example 14(g)], N-methylphenethylamine (243 mg, 1.8 mmol) and anhydrous N,N-dimethylformamide (2 ml) was subjected to microwave irradiation, heating to 210° C. for 10 minutes. Water was added to the reaction mixture, which was then extracted with ethyl acetate (2×). The combined organic layers were dried (MgSO₄) and concentrated, to provide 5-[6-(methyl-phenethyl-amino)-pyridin-2-yl]-thiophene-2-carboxylic acid (108 mg) as a brown oil, which was used directly without further purification. LCMS (Method C): R_{T}=3.99 minutes; 339 (M+H)⁺.

### Reference Example 34

### (a) 5-(6-Hydroxymethyl-pyridin-2-yl)-thiophene-2-carboxylic acid methyl ester

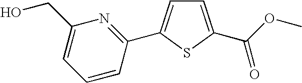

To a cool (0° C.) stirred suspension of 5-(6-formyl-pyridin-2-yl)-thiophene-2-carboxylic acid [247 mg, 1.0 mmol, Reference Example 14(e)] in methanol (10 ml) was added sodium borohydride (41 mg, 1.08 mmol). After stirring for 2 hours, 1M hydrochloric acid was added until pH ˜2 was obtained. Saturated sodium hydrogen carbonate solution was then added to basify the reaction mixture, which was extracted with ethyl acetate. The organic layer was isolated, washed with brine, dried Na₂SO₂) and concentrated, to provide 5-(6hydroxymethyl-pyridin-2-yl)-thiophene-2-carboxylic acid methyl ester (222 mg) as a light brown solid, which was used directly without further purification. LCMS (Method C): R_{T} 2.85 minutes; 250 (+H)⁺.

### Reference Example 35

### a) 5-[1-(3-Amino-propyl)-1H-pyrazol-3-yl]-thiophene-2-carboxylic acid methyl ester

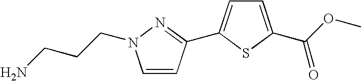

To a cooled (0° C.) solution of 5-[1-(3-tert-butoxycarbonylamino-propyl)-1H-pyrazol-3-yl]-thiophene-2-carboxylic acid methyl ester [128 mg, 0.35 mmol, Reference Example 10(ar)] in dichloromethane (2.5 ml), was added trifluoroacetic acid (2.5 ml). The reaction was stirred for 2 hours then concentrated to give a residue, which was partitioned between dichloromethane and saturated sodium hydrogen carbonate solution. The aqueous layer was further extracted with dichloromethane (x2) and the organic layers were combined, dried (MgSO₄) and concentrated, to provide 5-[1-(3-amino-propyl]-1H-pyrazol-3-yl-thiophene-2-carboxylic acid methyl ester (47 mg) as a yellow oil. LCMS (Method C): R_{T}=1.85 minutes; 266 (M+H)⁺.

### (b) 5-[1-(2-Amino-ethyl-1H-pyrazol-3-yl]-thiophene-2-carboxylic acid methyl ester

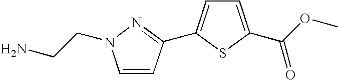

By proceeding in a similar manner to Reference Example 35(a) but using 5-[1-(3-tert-butoxycarbonylamino-ethyl)-1H-pyrazol-3-yl]-thiophene-2-carboxylic acid methyl ester [137 mg, 0.4 mmol, Reference Example 10(aq)], there was prepared 5-[1-(2-amino-ethyl)-1H-pyrazol-3-yl]-thiophene-2-carboxylic acid methyl ester (73 mg) as a yellow oil. LCMS (Method C): R_{T}=1.78 minutes; 252 (M+H)⁺.

### Reference Example 36

### (a) 2-(5-Methoxycarbonyl-thiophen-2-yl)-5-methyl-1H-imidazole-4-carboxylic acid tert-butyl ester

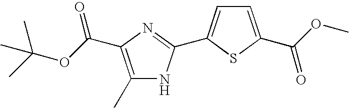

A mixture of 5-formyl-thiophene-2-carboxylic acid methyl ester [3.37 g, 19.8 mmol, Reference Example 37(a)], 2,3-dioxo-butyric acid tert-butyl ester (3.4 g, 19.8 mmol), ammonium acetate (15.25 g, 198 mmol) and acetonitrile (38 ml) was subjected to microwave irradiation, heating to 150° C. for 5 minutes. The reaction mixture was concentrated and the residue was partitioned between 2M sodium carbonate solution and ethyl acetate. The organic layer was separated, dried (MgSO4) and concentrated to give an orange residue, which was subjected to flash column chromatography on silica using a mixture of pentane and ethyl acetate (gradient, 4:1 to 1:1, v/v) as eluent, to provide 2-(5-methoxycarbonyl-thiophen-2-yl)-5-methyl-1H-imidazole-4-carboxylic acid tert-butyl ester (1.0 g) as a yellow powder.

### Reference Example 37

### (a) 5-Formyl-thiophene-2-carboxylic acid methyl ester

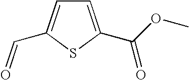

To a cooled (−78° C.) solution of 2-(5-bromo-thiophen-2-yl)-[1,3]dioxolane (5.0 g, 21.3 mmol) in tetrahydrofuran (150 ml) was added n-butyl lithium (8.52 ml, 21.3 mmol, 2.5M in hexanes) whilst keeping the temperature below 70° C. After 45 minutes methylchloroformate (1.65 ml, 21.3 mmol) in tetrahydrofuran (5 ml) was added, and the reaction mixture was stirred for a further 4 hours. 1M hydrochloric acid (500 ml) was added and the resultant mixture was extracted with diethyl ether (2×250 ml). The organic layers were combined, dried (MgSO₄) and concentrated to give an oil which was subjected to flash column chromatography on silica using a mixture of cyclohexane and ethyl acetate (4:1, v/v) as eluent. The resulting fractions were concentrated and dissolved in 1,2-dimethoxyethane and water, to which concentrated sulphuric acid was added. After 1 hour the mixture was concentrated, to provide 5-formyl-thiophene-2-carboxylic acid methyl ester as a black solid, which was used in the next reaction without further purification.

Biological Activity

Compounds are tested for their capacity to inhibit histone deacetylase activity (primary assay) and for their biological effects on growing cells (secondary assay).

Deacetylase Assay

Total lysates from K562 chronic human myelogenous leukemia cells (obtained from American Type Culture Collection, Rockville, Md.) are used as source of HDAC activity. Cells are grown in RPMI media supplied with 10% FCS, harvested by centrifugation, washed once in PBS and resuspended at a density of 24×10⁶/ml in HDA buffer (15 mM Potassium phosphate pH 7.5, 5% glycerol, 0.2 mM EDTA). After sonication, lysates are centrifuged at 1000 g for 20 minutes and the resulting supernatant is aliquoted and stored at −80° C. Alternatively, commercially available HeLa nuclear extracts (BIOMOL) are used as source of histone deacetylase activity.

The assay was carried out for 30 minutes using 116 μM of a fluorescent substrate containing an acetylated lysine (BIOMOL). When deacetylation of the lysine occurs, the substrate can react with the added developer producing a fluorophore. The amount of fluorophore produced is proportional to the HDAC activity in the sample and is quantified using a multiwell fluorimeter capable of excitation at 360 nm and detection at 450 nm.

Compounds are diluted in DMSO prior to addition to assay buffer, the final DMSO concentration in the assay being 1%.

The percent activity of the compounds in reducing histone deacetylase enzymatic activity is calculated as follow:
% activity={(F^{S}−B)/(F^{C}−B)}×100
where:

F^{S} is the fluorescence at 450 nm in the presence of the tested compound (Sample).

F^{C} is the fluorescence at 450 nm in the presence of vehicle 1% DMSO (Control). B is the fluorescence at 450 nm in the absence of enzyme (Background fluorescence)

The IC₅₀ is defined as the concentration at which a given compound achieves 50% activity. IC₅₀ values are calculated using the XLfit software package (version 2.0.5).

Table 1 shows the results obtained for the compounds of the present invention.

TABLE 1

| Sample | IC₅₀/μM |
|---|---|
| Example 1 (a) | 0.750 |
| Example 1 (g) | 0.900 |
| Example 1 (l) | 0.153 |
| Example 1 (z) | 0.076 |
| Example 1 (ba) | 0.062 |
| Example 1 (dc) | 0.242 |

Secondary Assay

Compounds are tested in a cell proliferation assay using the following cell lines:

| | |
|---|---|
| MCF-7 | human mammary gland adenocarcinoma (ATCC) |
| MDA-MB-231 | human mammary gland adenocarcinoma (ATCC) |

Both cell lines are free of Mycoplasma contamination (PCR Mycoplasma Detection Set, Takara). MCF-7 are kept in MEM medium (Gibco) supplemented with 10% FCS and 1% Non Essential Amino Acids at 37° C. in a 5% CO₂ humidified incubator.

MDA-MB-231 are kept in L-15 (Leibovitz) medium (Gibco) supplemented with 15% FCS at 37° C. in a non-modified atmosphere, humidified incubator.

Cells are seeded in 96-well plates at a density of 20,000 cells/ml (3,000 cells/well) and after 24 h they are exposed to different concentrations of compounds in 0.1% DMSO. Cells are grown for a further 72h, the media is removed and the cells are frozen at −80° C. for at least 30 minutes and lysed in a solution containing the CyQUANT dye. This is a fluorescent molecule that specifically binds nucleic acids and whose fluorescence is greatly enhanced upon binding nucleic acids. Therefore the fluorescence intensity is proportional to the number of cells present in each well and can be quantified using a multiwell fluorimeter by measuring the fluorescence of the solution at 520 nm.

The percent activity of the compounds in reducing cell number is calculated as follow:
% activity={(A^{S}−B)/(A^{C}−B)}×100
where:

A^{S} is the fluorescence at 520 nm in the presence of the tested compound (Sample).

A^{C} is the fluorescence at 520 nm in the presence of vehicle 0.1% DMSO (Control).

B is the fluorescence at 520 nm in the absence of cells (Background fluorescence).

The IC₅₀ is defined as the concentration at which a given compound achieves 50% activity. IC₅₀ values are calculated using the XLfit software package (version 2.0.5).

Table 2 shows the results obtained for the compounds of the present invention.

TABLE 2

| | MCF-7 | MDA-MB-231 |
|---|---|---|
| Sample | IC₅₀/μM | IC₅₀/μM |
| Example 1 (a) | 11 | 32 |
| Example 1 (g) | 31 | 44 |
| Example 1 (l) | 2.1 | 4.5 |
| Example 1 (z) | 2.3 | 7.3 |
| Example 1 (ba) | 0.6 | 2.0 |

1. A compound of formula (I): 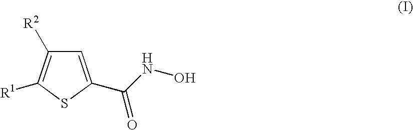
in which
R¹ represents aryl or heteroaryl, each optionally substituted by one or more groups selected from R³, alkylenedioxy, carboxy, cyano, halo, hydroxy, nitro, haloalkyl, haloalkoxy, —C(═O)—R³, —C(O)OR³, —C(═Z)—NR⁴R⁵, —NR⁴R⁵, —NR⁶—C(═O)—OR³, —NR⁶—C(═O)—NR⁴R⁵, —NR⁶—C(═Z)—R³, —O—C(═O)—NR⁴R⁵, —NR⁶—SO₂—R³, —OR³, —O—C(═O)R³, —SH, —SR³, —SOR³, —SO₂R³ and —SO₂—NR⁴R⁵;
R represents hydrogen, chloro, cyano, fluoro, alkoxy, alkyl, or haloalkyl;
R³ represents aryl, heteroaryl, cycloalkyl, cycloalkenyl, heterocycloalkyl or R⁷;
R⁴ and R5 independently represent a group selected from hydrogen, alkyl, alkenyl, aryl, heteroaryl, cycloallcyl, cycloalkenyl or heterocycloalkyl, wherein said alkyl or alkenyl are optionally substituted by aryl, heteroaryl, cycloalkyl, cycloalkenyl or heterocycloalkyl; or the group —NR⁴R⁵ may form a cyclic amine;
R⁶ represents hydrogen or lower alkyl;
R⁷ represents alkyl, alkenyl and alkynyl, wherein said alkyl, alkenyl or alkynyl are optionally substituted by one or more groups selected from aryl, heteroaryl, cycloalkyl, cycloalkenyl, heterocycloalkyl, hydroxy, —C(═Z)—NR⁴R⁵, —NR⁴R⁵, —NR⁶—C(═Z)—R⁸, —O—C(═O)—NR⁴R⁵, —NR⁶—C(═O)—OR⁸, —NR⁶—C(═O)—NR⁴R⁵, —NR⁶—SO₂—R⁸, —OR⁸, —SOR⁸, SO₂R⁸ and —SO₂—NR⁴R⁵; R⁸ represents alkyl, alkenyl or alkynyl, optionally substituted by one or more groups selected from aryl, heteroaryl, cycloalkyl, cycloalkenyl, heterocycloalkyl, hydroxy and halogen; or R⁸ represents aryl, heteroaryl, cycloalkyl, cycloalkenyl or heterocycloalkyl; and
Z is O or S,
and corresponding N-oxides, pharmaceutically acceptable salts, solvates and prodrugs of such compounds.

2. A compound according to claim 1 wherein R¹ is optionally substituted phenyl.

3. A compound according to claim 1 wherein R¹ is 4-methoxyphenyl.

4. A compound according to claim 1 wherein R¹ is selected from optionally substituted monocyclic heteroaryl.

5. A compound according to claim 1 wherein R¹ is selected from optionally substituted imidazolyl, isoxazolyl, oxadiazolyl, pyrazolyl, pyridinyl, thienyl and pyrimidinyl.

6. A compound according to claim 1 wherein R¹ is selected from optionally substituted imidazolyl, pyrazolyl, pyridinyl and pyrimidinyl.

7. A compound according to claim 1 wherein R¹ is substituted by a haloalkyl group.

8. A compound according to claim 1 wherein R¹ is substituted by an optionally substituted alkyl, alkenyl or alkynyl group.

9. A compound according to claim 1 wherein R¹ is substituted by an optionally substituted alkyl group.

10. A compound according to claim 8 wherein said alkyl, alkenyl or alkynyl group is substituted by one or more groups selected from optionally substituted aryl, heteroaryl, cycloalkyl, cycloalkenyl and heterocycloalkyl, and from hydroxy, —C(═Z)—NR⁴R⁵, —NR⁴R⁵, —NR⁶—C(═Z)—R⁸, —O—C(═O)—NR⁴R⁵, —NR⁶—C(═O)—OR⁸, —NR⁶—C(═O)—NR⁴R⁵, —NR⁶—SO₂—R⁸, —OR⁸, —SOR⁸, SO₂R⁸ and —SO₂—NR⁴R⁵.

11. A compound according to claim 8 wherein said alkyl, alkenyl or alkynyl group is substituted by a group selected from optionally substituted aryl, heteroaryl and heterocycloalkyl, and from —C(O)—NR⁴R⁵, —NR⁴R⁵, —NR⁶—C(O)—R⁸, —NR⁶—SO₂—R⁸, OR⁸ and —SO₂—NR⁴R⁵.

12. A compound according to claim 8 wherein said alkyl, alkenyl or alkynyl group is substituted by optionally substituted aryl and heteroaryl.

13. A compound according to claim 1 wherein Z is O.

14. A compound according to claim 5 wherein R¹ is substituted by a group X wherein X is selected from the group consisting of optionally substituted aryl, optionally substituted heteroaryl, optionally substituted heterocycloalkyl, —C(O)—NR⁴R⁵, —NR⁴R⁵, —NR⁶—C(O)—R⁸, —NR⁶—SO₂—R⁸, —OR⁸, —SO₂—NR⁴R⁵ and alkyl substituted by a group selected from optionally substituted aryl, optionally substituted heteroaryl, optionally substituted heterocycloalkyl, —C(O)—NR⁴R⁵, —NR⁴R⁵, —NR⁶—C(O)—R, —NR⁶—SO₂—R⁸, —R⁸ and —SO₂—NR⁴R⁵.

15. A compound according to claim 14 wherein X is selected from:
—(CH₂)ₙCONR⁴(CH₂)ₘAr,
—(CH₂)ₙSO₂NR⁴(CH₂)ₘAr
—(CH₂)ₙNR⁶CO(CH₂)ₘAr,
—(CH₂)ₙNR⁶SO₂(CH₂)ₘAr,
—(CH₂)ₙNR⁴(CH₂)ₘAr,
—(CH₂)ₙO(CH₂)ₘAr, and
—(CH₂)ₙAr;
wherein Ar is optionally substituted aryl, heteroaryl or heterocycloalkyl;
n is 0, 1, 2 or 3; and
m is 0, 1, 2, 3 or 4.

16. A compound according to claim 1 wherein said R⁴ and R⁶ groups are independently selected from hydrogen; and/or wherein said R⁵ and R⁸ groups are independently selected from optionally substituted aryl, heteroaryl and heterocycloalkyl, and from alkyl substituted by optionally substituted aryl, heteroaryl or heterocycloalkyl.

17. A compound according to claim 10 wherein the substituent(s) on said optionally substituted aryl, heteroaryl and heterocycloalkyl groups are selected from halogen, CF₃, OCF₃, alkyl, acylamino, arylalkyl, aryloxy, aryl, cyclic amino, heteroaryl, alkylenedioxy and aminosulphonyl.

18. A compound according to claim 10 wherein said optionally substituted aryl is selected from phenyl; said optionally substituted heteroaryl is selected from quinolinyl, isoquinolinyl, pyridyl, oxadiazolyl, thiadiazolyl, imidazolyl, indolyl, indazolyl, pyrolyl and benzofuranyl; and said optionally substituted heterocycloalkyl is selected from either (i) an optionally substituted saturated multicyclic heterocarbocyclic moiety in which an aryl or heteroaryl ring and a heterocycloalkyl group are fused together to form a cyclic structure, or (ii) piperazinyl substituted on nitrogen by aryl, arylalkyl, heteroarylalkyl or heteroaryl.

19. A compound according to claim 5 wherein R¹ is selected from 1-(2-phenylethyl)-1H-pyrazol-3-yl, 1-benzyl-1H-pyrazol-3-yl, 4-trifluoromethyl-1H-imidazol-2-yl, pyridin-2-yl, 5-trifluoromethyl-1H-pyrazol-3-yl, 1-methyl-1H-pyrazol-3-yl, 2-methyl-2H-pyrazol-3-yl, 1-methyl-5-trifluoromethyl-1H-pyrazol-3-yl, 2-methyl-5-trifluoromethyl-2H-pyrazol-3-yl, 1H-pyrazol-3-yl, pyridin-4-yl, 5-trifluoromethylisoxazol-3-yl, 3-methyl[1,2,4]oxadiazol-5-yl, or thiophene-2-yl.

20. A compound according to claim 1 wherein R² is hydrogen.

21. A compound according to claim 1 wherein R³ and R⁸ are independently selected from alkyl.

22. A compound according to claim 1 wherein R³ and R⁸ are independently selected from methyl and ethyl.

23. A compound according to claim 1 wherein R⁴ and R⁵ are independently selected from hydrogen, alkyl, arylalkyl and heteroarylalkyl.

24. A compound according to claim 1 selected from:
5-(4-trifluoromethyl-1H-imidazol-2-yl)-thiophene-2-carboxylic acid hydroxyamide;
5-(1-benzyl-1H-pyrazol-3-yl)-thiophene-2-carboxylic acid hydroxyamide;
5-(1-phenethyl-1H-pyrazol-3-yl)-thiophene-2-carboxylic acid hydroxyamide;
5-pyridin-2-yl-thiophene-2-carboxylic acid hydroxyamide;
and corresponding N-oxides, pharmaceutically acceptable salts, solvates and prodrugs of such compounds.

25. A compound according to claim 1 selected from:
5-[1-(2,3-dihydro-benzo[1,4]dioxin-2-ylmethyl)-1H-pyrazol-3-yl]-thiophene-2-carboxylic acid hydroxyamide;
5-(5-phenethyl-1H-pyrazol-3-yl)-thiophene-2-carboxylic acid hydroxyamide;
5-pyrimidin-2-yl-thiophene-2-carboxylic acid hydroxyamide;
5-(1-benzo [1,3]dioxol-5-ylmethyl-1H-pyrazol-3-yl)-thiophene-2-carboxylic acid hydroxyamide;
5-(1-phenethyl-5-trifluoromethyl-1H-pyrazol-3-yl)-thiophene-2-carboxylic acid hydroxyamide;
5-(4-benzyloxy-pyrimidin-2-yl)-thiophene-2-carboxylic acid hydroxyamide;
5-(2-phenethyl-3H-imidazol-4-yl)-thiophene-2-carboxylic acid hydroxyamide;
5-[1-(5-tert-butyl-[1,2,4] oxadiazol-3-ylmethyl)-1H-pyrazol-3-yl]-thiophene-2-carboxylic acid hydroxyamide;
5-{1-[6-(2,2-dimethyl-propionylamino)-pyridin-2-ylmethyl]-1H-pyrazol-3-yl}-thiophene-2-carboxylic acid hydroxyamide;
5-(5-phenylacetylamino-pyridin-2-yl)-thiophene-2-carboxylic acid hydroxyamide;
5-(1-quinolin-2-ylmethyl-1H-pyrazol-3-yl)-thiophene-2-carboxylic acid hydroxyamide;
5-[5-(2-benzyloxy-ethylamino)-pyridin-2-yl]-thiophene-2-carboxylic acid hydroxyamide;
5-{5-[(2,3-dihydro-benzo [1,4] dioxin-6-ylmethyl)-amino]-pyridin-2-yl}-thiophene-2-carboxylic acid hydroxyamide;
5-{5-[(benzofuran-2-ylmethyl)-amino]-pyridin-2-yl}-thiophene-2-carboxylic acid hydroxyamide;
5-{1-[2-(4-fluoro-benzyloxy)-ethyl]-1H-pyrazol-3-yl}-thiophene-2-carboxylic acid hydroxyamide;
5-(1-phenylcarbamoylmethyl-1H-pyrazol-3-yl)-thiophene-2-carboxylic acid hydroxyamide;
5-[1-(quinolin-8-ylcarbamoylmethyl)-1H-pyrazol-3-yl]-thiophene-2-carboxylic acid hydroxyamide;
5-{1-[(4-fluoro-phenylcarbamoyl)-methyl]-1H-pyrazol-3-yl}-thiophene-2-carboxylic acid hydroxyamide;
5-{1-[(4-oxazol-5-yl-phenylcarbamoyl)-methyl]-1H-pyrazol-3-yl}-thiophene-2-carboxylic acid hydroxyamide;
quinoline-2-carboxylic acid {2-[3-(5-hydroxycarbamoyl-thiophen-2-yl)-pyrazol-1 yl]-ethyl}-amide;
5-{1-[(2-morpholin-4-yl-phenylcarbamoyl)-methyl]-1H-pyrazol-3-yl}-thiophene-2-carboxylic acid hydroxyamide;
5-(1-{[2-(1H-indol-3-yl)-ethylcarbamoyl]-methyl}-1H-pyrazol-3-yl)-thiophene-2-carboxylic acid hydroxyamide;
5-{1-[(2-fluoro-phenylcarbamoyl)-methyl]-1H-pyrazol-3-yl}-thiophene-2-carboxylic acid hydroxyamide;
5-[1-quinolin-3-ylcarbamoylmethyl)-1H-pyrazol-3-yl]-thiophene-2-carboxylic acid hydroxyamide;
2-(5-hydroxycarbamoyl-thiophen-2-yl)-5-methyl-1H-imidazole-4-carboxylic acid phenethyl-amide;
2-(5-hydroxycarbamoyl-thiophen-2-yl)-5-methyl-1H-imidazole-4-carboxylic acid benzylamide;
5-(6-benzyloxymethyl-pyridin-2-yl)-thiophene-2-carboxylic acid hydroxyamide;
5-{1-[1H-indol-7-ylcarbamoyl)-methyl]-1H-pyrazol-3-yl}-thiophene-2-carboxylic acid hydroxyamide;
5-{1-[(3-chloro-phenylcarbamoyl)-methyl]-1H-pyrazol-3-yl}-thiophene-2-carboxylic acid hydroxyamide;
5-{1-[(3-methoxy-phenylcarbamoyl)-methyl]-1H-pyrazol-3-yl}-thiophene-2-carboxylic acid hydroxyamide;
5-[1-(1-oxy-quinolin-2-ylmethyl)-1H-pyrazol-3-yl]-thiophene-2-carboxylic acid hydroxyamide;
5-(1-{2-[(benzo[1,3]dioxol-5-ylmethyl)-amino]-ethyl}-1H-pyrazol-3-yl)-thiophene-2-carboxylic acid hydroxyamide;
5-[1-(2-benzylamino-ethyl)-1H-pyrazol-3-yl]-thiophene-2-carboxylic acid hydroxyamide; and corresponding N-oxides, pharmaceutically acceptable salts, solvates and prodrugs of such compounds.

26. A pharmaceutical composition comprising a compound according to claim 1 and a pharmaceutically acceptable carrier.

27. (canceled)

28. A method for treating a disease in a patient in which inhibition of histone deacetylase can prevent, inhibit or ameliorate the pathology and/or symptomatology of the disease, which method comprises administering to the patient a therapeutically effective amount of a compound according to claim 1.

29. A method according to claim 28 wherein said disease is a disease caused by increased cell proliferation.

30. A method according to claim 28 wherein said disease is cancer, psoriasis, fibroproliferative disorders, smooth muscle cell proliferation disorders, inflammatory diseases and conditions treatable by immune modulation, neurodegenerative disorders, diseases involving angiogenesis, fungal and parasitic infections and haematopoietic disorders.

31. A method according to claim 28 wherein said disease is liver fibrosis, arteriosclerosis, restenosis, rheumatoid arthritis, autoimmune diabetes, lupus, allergies, Huntington's disease, retinal diseases, protozoal infections, anaemia, sickle cell anaemia and thalassemia.

32. A method according to claim 31 wherein said protozoal infection is malaria, toxoplasmosis or coccidiosis.

33. A method according to claim 31 wherein said retinal disease is diabetic retinopathy, age-related macular degeneration, interstitial keratitis or rubeotic glaucoma.

34. A method according to claim 28 wherein said disease is congestive heart failure due to cardiomyocyte hypertrophy.
